SEQUENCE LISTING (338 sequences)

SEQ ID NO: 1 - protein, 213 aa, unidentified
  source 1..213: note = Siraitia grosvenorii
MALQVWETLKEAIVAYTGLSPATFFTVLALGLAVYYVISGFFGTSDYGSHSRDFGEQMQPLPPPVQVGEISEEELKQYDGSDSKKPLLMAIKGQIYDVSQSRMFYGPGGPYALFAGKDASRALAKMSFEEKDLTGDISGLGPFELEALQDWEYKFMSKYVKVGTVEKPVSETDAAPAGESAESTNAEVSKPAEDGPSESQADKAEETVAAKVE

SEQ ID NO: 2 - protein, 280 aa, unidentified
  source 1..280: note = Siraitia grosvenorii
MALQVWETLKEAIVAYTGLSPATFFTVLALGLAVYYVISGFFGTSDYGSHSRDFGEQMQPLPPPVQVGEISEEELKQYDGSDSKKPLLMAIKGQIYDVSQSRMFYGPGGPYALFAGKDASRALAKMSFEEKDLTGDISGLGPFELEALQDWEYKFMSKYVKVGTVEKPVSETDAAPAGESAESTNAEVSKPAEDGPSESQADKAEETVAAKQKKEKKKGRRRVEGVLMEIRGEAVGGRKGEGMSREIGRGGGGADDGNLKGGGIDHGERERERERESGMS

SEQ ID NO: 3 - protein, 209 aa, unidentified
  source 1..209: note = Siraitia grosvenorii
MELYWKAMEQIAWYTGLSPTAFFTILASMIFVFQMVSSMFVSPEEFNKPPTVPVSSSNPANSNLFVNDSVADASQAVQIGQLTEQQLRAYDGSDPNKPLLMAIKGQIYDVSSGRMFYGPGSPYAMFAGKDASRALALLSFKPEDITGNIEGLSEEELVILQDWEYKFMEKYVKVGELVLEGGGEGMNEHSENGHRNFEIDQEEERIEAR

SEQ ID NO: 4 - protein, 332 aa, unidentified
  source 1..332: note = Siraitia grosvenorii
MTATITAQHRNGCGPSPPKKLDILRVSFRKYRKAIEQPTEAISRLIAKRDRCRAFRPSIRFKKSHSTVPTTIVYDLTSYLDEHPGGDDVILAATGRDATDDFEDAGHSKDARELMQKFYIGLLDTSSSASPELETKQPRGYAARVQGLTKQYWAAPVAILGISLAVQKNTLYVGGLAEEVNESILHAAFIPFGDIKDVKTPLDQATQKHRSFGFVTFLEKEDASAAMDNMDGAELYGRVLTVNYALPERIKGGEQGWAAQPIWADADTWFERQQQEEEMQRIQAENRAAMQAAEELHRKKLAQEREGEKEDEIDTKDDPMAKAEAEVLRQNS

SEQ ID NO: 5 - protein, 158 aa, unidentified
  source 1..158: note = Siraitia grosvenorii
MVGKAMRRNRAHKDRVFNFLLPDPKVFVFDEVAKHNHLRDCWIIISGKVYDVTPFLEEHPGGDEVLLLAAEKDATDDFENVGHSLAAIEQMEKYYVGKVDMSTVPKPIDYRPPASHPATTPSAQSSGSLIRLLQFLIPLLILGVAFYLQFYGKKQQSN

SEQ ID NO: 6 - protein, 700 aa, unidentified
  source 1..700: note = Siraitia grosvenorii
MKDFQDEIAASGAVPAADHKLLRTEPAECMAAWRQRRALVPHHANAAAELSASSVVFLFVIVVFVGVVVEIFFVAHSSISDILTGCDVGDDSDPVVDSSLSFSSSLFSSCSFSSSSAASAAAAALLLHRLLLVLLRLPSRTLFSPPIRRCLSVGLGCRVLVLRLERGSIRTSHRAGTSLNRRSCSVWNSGEMFGDGKRISTILGTVETQAEPARFSRSLSLKDFQVLLSLPKRPLPALGSSSRIMSSRVRLRISAARPVAGGPSLFRRQPPSVLPIEAGHRKKRKCQMELDAGMASARTWELGRRKKKKKIFRSKFENLESKMQDEEKRGSGWCENAIASASPTHTQHSIDCRTSLPLFHAATMGGRSVYTLAEVASHDNRNDCWLIIENKVYDVTKFLEDHPGGDEVLLSAIGKDATNDFDDVGHSSAARAMMDEFYVGDIDSSTIPAKRKYTPPKQPLYNQDKTPEFIIKVLQFLVPLVCSHRPRKNLTTHCGALVAQNTGLEPCTGGEGAHFEQHEHQPLARASGNQLVCRASSCLSLIDPDEPVKVIKSSNIQLGSYPSLEVKSASNSAIKHHSTSQKPEKEIDWNSQFFNWQKADYCLNNNLPELLYSKREKLEAICSEQAFDAIASMQRLTTSAHRQEGVNSHIAATLQHTFQGSWRRYSQDILQLANQDYPLRTLQSFQDNQIHNIAAQHFLE

SEQ ID NO: 7 - protein, 126 aa, unidentified
  source 1..126: note = Siraitia grosvenorii
MASDPKVQVFEEVAKHNKPKIVYDVTPFMEDHPGGDEVLLSATGKDATNDFEDVGHSDSAREMMDKYYIGEIDPSTVPLKKIYIPTQQTQYTPDKTPEFVIKILQILVPILILGLAFAVRHYTKNE

SEQ ID NO: 8 - protein, 308 aa, unidentified
  source 1..308: note = Siraitia grosvenorii
MPSISTLYSIQEVSQHSTNDDCWIVIDGKVYDLTSYLDEHPGGDDVILAATGRDATDDFEDAGHSKDARELMQKFYIGLLDTSSSASPELETKQPRGYAARVQGLTKQYWAAPVAILEKTTTKKNLKELQKWRINASNGAGSSEKHFVRRWFGGGGERVYPTCSLHTLRRHQRRQDSIGSSHPEAPFLRLRHFLGKEDASAAMDNMDGAELYGRVLTVNYALPERIKGGEQGWAAQPIWADADTWFERQQQEEEMQRIQAENRAAMQAAEELHRKKLAQEREGEKEDEIDTKDDPMAKAEAEVLRQNS

SEQ ID NO: 9 - protein, 861 aa, unidentified
  source 1..861: note = Siraitia grosvenorii
MTVREIVDISNLAESCTSLLCRRQNNRFVHIAARFVVDEDVEGVWLEELLEALTLVCEEGLRLSLSDASCQGRSVLIYLREDAHERERLSFSSVKRWRERSCEPRPPLSGFWRLWWQLLRFSSASPSNPDIRSPPPPPSSPLNRSVFDVTKGKSHYGVGGGYNHFAGRDASRAFVSGNFTGEGLTDSLRGLSNAEIKSVVEWRNFYNKTYTFVGKLVGLHYDDQGNPTKHLKGAEAKAARGAQLLKKQKEEEDKLPSCNSRWSQGEGGEVWCDDGFPRLVQRPLEIALSGKMSKRCACFREDQLAEPGLEVYEAALFPKRTNDDGAAKILPPSDPCCFSCFPSGWCAGSIRNWISKSISSDFNTTTLPQQFLYVRSFPEGDKNCYIIKLAQGKGYKYLIRASFMYGNYDGQGKAPAFDLHMGVNKWDSVILNNESSIIIKEVIHALPTSSICICLVNTGFGSPFISALELRLLKNATYVTDFELLALHRRLDIGSTTNKTVRYNDDDCDRIWLPFNFPNYKIVSASSTVDSGVTALMNIKSLYGVRKNWQGDPCMPKSYVWHGLNCSYDSHSPNRITSLNLSSSRLVGETAAYVSELTSLQYLDLSNNSLSGPVPGFLSELHSLKVLDLRDNTLLGSIPSELMERSTNVKEIDGSDYYSNLSLDIVNCFMEGLEETPIFVLHLLAKTRRRVTLFQSNCSNSFIIAGPLSCNSSLDHEMMKTRYYASGRLTEKSDVYSFGALILEIITSRPVLKINRASSEKCHVGQWAMHLMKTGDIRSIVDERLRGNFDLSSAWKAVEIAMTCLSQTSIERPSMKEVVMELSECLALEKARKRKNIDSNTRSSNAVSRNFSESEVTPLAR

SEQ ID NO: 10 - protein, 134 aa, unidentified
  source 1..134: note = Siraitia grosvenorii
MGSGEKVFTLKEVAEHNNQKDCWLVISGKVYDVTKFLDDHPGGDDVLLSATGKDATDDFEDVGHSDNAREMMDQYYVGEIDASTIPKKVAYAPPKQPHYNQDKTSEFIIKLLQFLVPLAILGLAVAIRFYTKQA

SEQ ID NO: 11 - DNA, 642 bp, Synthetic construct
  source 1..642: mol_type = other DNA
atggccttgcaggtgtgggaaaccttaaaagaggctattgtagcttatacggggctttcccctgcaacatttttcactgtcctagctttgggtctggcggtttactacgttatctcaggtttctttggaacctctgattatggttctcacagtcgtgacttcggtgaacaaatgcaacccttaccaccaccagtccaagttggcgaaatatccgaagaggaattgaagcaatacgacggtagcgattcaaagaagccattgctaatggctatcaaaggccaaatttacgatgtctcgcaatctaggatgttctatggtcccggtggtccatacgccctctttgctggtaaggacgcttctcgagctttggcaaaaatgtccttcgaagaaaaggacttgactggtgatatttctggtttgggccctttcgaattagaagccttacaggattgggaatacaagtttatgtccaaatacgttaaggtcggtactgttgaaaagccagtttcagagacagatgctgcccctgctggtgaatctgcggagtctactaacgctgaagtctcgaaaccagcagaagacggtccttctgaatcccaagctgataaggctgaagagactgtagccgctaaggttgaatag

SEQ ID NO: 12 - DNA, 843 bp, Synthetic construct
  source 1..843: mol_type = other DNA
atggccttgcaggtatgggagactctaaaagaagcaattgtcgcttataccggtcttagtccagctacgttctttacagtgttggctctgggattagccgtttactacgtcatatctggtttctttggtacctcggactatggctcacattctcgtgatttcggggaacaaatgcaacctttgccaccgccagttcaagttggtgaaatctccgaagaggaattaaagcaatacgatggttctgacagcaagaagccattgttgatggctattaaaggtcaaatctacgacgtttcccaatctaggatgttttacggtccaggtggtccctatgcgcttttcgctggcaaggatgcctcgcgcgctttggctaagatgagttttgaagaaaaagacctgactggggatatttcaggattaggccctttcgaattggaagctctccaggattgggaatataagttcatgtctaagtacgtcaaagtgggtactgttgaaaagccagtttccgagacagacgcagccccagctggtgaatctgccgaaagtaccaacgctgaagtctccaaacctgctgaagatggtccaagcgaatcccaagctgacaaggcggaggaaactgtagccgctaagcaaaaaaaggaaaaaaagaagggtagaagaagagttgaaggtgtcctaatggaaatcagaggtgaagcggttggtgggagaaagggtgagggaatgtcaagagaaatcggtcggggtggtggtggtgctgatgatggtaatttgaaaggtggcggtattgaccacggtgaacgtgagcgagagagagaaagagagtctggtatgtcttag

SEQ ID NO: 13 - DNA, 630 bp, Synthetic construct
  source 1..630: mol_type = other DNA
atggaattgtactggaaagcaatggagcagattgcctggtataccggcttatctcctacagctttctttactatactggcttcgatgatcttcgtgtttcaaatggtcagttccatgttcgtttctccggaagaatttaataagcccccaactgtcccagtttcctcatctaacccagctaactctaatctattcgtaaacgacagcgttgcggatgccagccaagctgttcaaattggtcaattgacggaacaacagttgagggcttacgatggatccgacccaaacaagccattacttatggctatcaaaggtcaaatttatgatgtctctagtggtagaatgttctacggtcctggtagtccatacgcaatgttcgccggcaaggacgcttcaagagctttggcattgctctcttttaagccagaagacatcaccggtaacattgaaggtttgtcagaagaggaattagttatcttacaagattgggagtacaagttcatggaaaagtatgttaaagtgggggaattggttctcgaaggtggtggtgaaggtatgaatgaacactctgagaacggccatcgtaattttgaaatcgatcaagaagaagaaagaattgaggctagatag

SEQ ID NO: 14 - DNA, 630 bp, Synthetic construct
  source 1..630: mol_type = other DNA
atggaactttattggaaggctatggagcaaatagcctggtacacagggttatcgcctacggcattctttactattctagctagtatgattttcgtctttcagatggtgtcatctatgttcgttagcccagaagaatttaataaaccaccaaccgtacccgtttcctcttccaaccctgctaactctaatttgttcgtcaacgatagcgttgctgacgcctctcaagcggtccaaatcggtcaattgaccgaacaacaattgcgtgcttacgatggttccgacccaaacaagccattactgatggccatcaagggtcagatttatgatgtttcctctggaaggatgttctacggcccgggttcaccatacgctatgttcgccggtaaagacgcgtcgagagctctcgctttgttgtcctttaagccagaagatatcactggtaacattgagggtttgagtgaagaagaattggttatcttacaagactgggaatacaagttcatggagaagtatgtgaaagtcggtgaacttgtcttggaaggtggaggtgaaggtatgaatgaacattcagagaatggccacagaaactttgaaattgatcaggaagaagaaagaatcgaagctagatag

SEQ ID NO: 15 - DNA, 999 bp, Synthetic construct
  source 1..999: mol_type = other DNA
atgaccgccactatcacagcacagcaccgtaacggctgcggaccttccccacccaaaaagttggacattctgagagtaagtttccgaaagtatagaaaggctatagaacaaccaacggaggctatttcgagactaattgctaaaagagatcgctgtagagcgtttaggccatctatcagattcaagaaatcacattccaccgtgccaactactatcgtttacgatttgacctcttacttagacgaacaccctggtggtgatgacgttatcttggccgccactggtcgtgatgctacagacgattttgaagatgctggtcattctaaggatgctagagaacttatgcaaaagttctacattggtttattggacaccagctcttccgcttccccagagttggaaacgaaacaaccgagaggttatgctgcaagagtccaagggttgactaagcaatactgggcagctccagttgccatactcggtatttcattagccgtccaaaagaatactttgtacgtcggcggtctggctgaagaagttaacgaaagtatcttgcacgctgctttcattccttttggtgacattaaagacgttaagaccccgttagaccaagctacccagaagcatagatcattcgggtttgttactttcctagaaaaggaagatgcctctgcggctatggataatatggacggtgcggaattgtatggtcgtgtcttgactgtaaactacgccttgccagagagaatcaagggtggagaacaaggttgggctgcgcaaccaatctgggctgatgctgatacctggttcgagagacaacaacaagaagaagaaatgcagagaatacaagccgaaaaccgcgccgctatgcaagctgctgaagagctacacagaaaaaagcttgctcaggaaagagaaggtgaaaaagaagatgaaattgataccaaagacgacccgatggccaaggcagaagctgaggtcctacgacaaaatagttag

SEQ ID NO: 16 - DNA, 477 bp, Synthetic construct
  source 1..477: mol_type = other DNA
atggttggaaaggccatgaggagaaatagagctcataaagatcgtgtcttcaactttttgctgccagaccctaaggtattcgtttttgatgaggttgcaaagcacaaccacttaagagactgctggatcataatttcaggcaaagtctatgatgttacaccattcttggaagaacatccgggtggtgacgaagtgcttctattagctgctgaaaaggatgcgactgacgattttgaaaatgtcggtcacagcttggctgccatcgaacaaatggagaagtactacgttggtaaagtcgacatgtccacggtcccaaagccaattgactatcggccccctgcttctcatccagctaccaccccatctgcccagagttctggttcgttgattcgattgcttcaattcttgatcccattattgattttaggtgttgctttctacttacaattttacgggaagaaacaacaatccaactag

SEQ ID NO: 17 - DNA, 2103 bp, Synthetic construct
  source 1..2103: mol_type = other DNA
atgaaggattttcaggacgagatcgcagccagtggtgctgtccctgctgctgaccacaaactactgcgtacagaacccgctgaatgcatggccgcatggagacaacgcagagctttggtgccacatcacgccaacgctgctgccgaattgtctgctagctcagttgttttcttgttcgttatagtagtctttgttggcgtcgttgtcgaaattttcttcgttgcgcattcttccatttctgatatcttaaccggttgtgacgttggtgatgattccgacccagtagtggattcgtctttgtccttttctagcagtcttttctcttcctgttctttttcctcatcatcggccgctagtgctgcggctgctgcattattgctccacaggttgttacttgttttgttgagactaccatctcggactttgttctcgccacctattagaagatgtttgtccgtcggacttggttgtagagtcttagttttaagattggaaagaggttctattcgaacttcacatagagctggtacgtctttaaatagaagatcatgttccgtatggaactcaggggaaatgttcggtgacggtaagcgtatcagcaccattctagggactgttgagacacaagcagaaccagcaagattttctcgctctttgtccttgaaggatttccaagtcctactgagcttgcctaaaagaccattgccagctttgggtagctcttcccgtatcatgtcttctcgcgtcagactgagaataagtgcggccagaccagttgctggtggtccgagtctattcaggcgacaaccaccgtctgtactgccaatcgaagctggacacagaaagaaaagaaagtgccaaatggaattggacgctggtatggcctccgctagaacttgggagttaggtagacgtaagaagaaaaagaaaatttttagaagcaagttcgaaaacttggaatctaagatgcaggatgaagaaaaaagaggtagtggttggtgtgagaatgctatcgcctccgcttctccaacccacacccaacactcaattgactgccggacgtccctaccactctttcatgccgcgactatgggcggtagatccgtttacactttggctgaagttgccagtcatgataatagaaacgattgttggttaattatagaaaacaaggtttatgatgttactaagttcttagaagaccatccaggtggcgacgaagtcttgttgtccgctatcggtaaggatgctaccaatgactttgacgatgtgggtcacagttccgctgctagagctatgatggatgaattttacgttggtgacatcgattcttctactattcccgccaagcgtaaatacacccctcctaagcaaccattgtataaccaagacaaaaccccagaatttattataaaggtacttcaattcttggtccctctagtttgttctcatagaccaagaaaaaatcttacaactcactgcggagcactcgtcgcgcaaaacaccggtttggaaccatgtactggcggggaaggtgctcatttcgaacagcacgaacaccaacccttggccagagcttctggtaatcaattggtttgtcgggcttcctcctgtctctctttgatcgatccagatgagccagttaaggttatcaagtcttctaacattcaactaggttcatacccctccttggaggtgaaaagtgcttcgaacagtgctatcaagcaccatagtacatctcagaagcctgaaaaagaaatagattggaatagccaattcttcaactggcaaaaggctgattattgtttaaacaacaatctgccagaattgttatacagcaaaagagaaaagttggaagctatttgctctgaacaagcttttgacgcaattgcctccatgcaaagactaactacaagcgcccaccgtcaagaaggtgttaactctcacatagcggccactctacaacacacgtttcaaggttcgtggagaagatactcacaagatatcttgcaattagccaaccaagactatccattgagaaccttgcaaagtttccaagacaaccaaattcataatattgctgctcaacatttcctcgaatag

SEQ ID NO: 18 - DNA, 381 bp, Synthetic construct
  source 1..381: mol_type = other DNA
atggcctctgatccaaaggtgcaagtcttcgaagaggttgctaaacacaacaagcctaaaattgtctacgacgtaaccccatttatggaagatcatccaggaggtgacgaagttttactttccgcaactggtaaggatgctacaaatgacttcgaagatgttggccactcagattcggcgagagaaatgatggacaagtattacatcggtgaaatagaccccagcactgtcccgctgaagaaaatttacatcccaacccagcaaactcaatatacgcctgacaagactccagagtttgttataaagattttgcaaatcttggtcccaattttgatcttaggtttggctttcgctgttcgtcattacaccaagaacgaatag

SEQ ID NO: 19 - DNA, 927 bp, Synthetic construct
  source 1..927: mol_type = other DNA
atgccttcaatctctacgctctactcgatacaggaggtatctcaacatagtacaaatgatgactgctggattgtgattgatggcaaggtttatgacttgacttcctacctagatgaacacccgggtggagatgacgttatcttagctgccaccggtcgtgatgcaaccgacgactttgaagatgctggtcactccaaagacgctagagaattgatgcaaaagttctatatcggtcttttagacactagctcttcagcgtcccccgaattggagactaagcaaccaagagggtacgccgctagggtccaaggtctgaccaaacaatactgggccgcaccagtcgctattttggaaaagactaccactaagaaaaacttgaaggaattgcaaaagtggagaattaacgcctctaatggtgctggatcttccgaaaagcatttcgttcggcgctggtttggtggtggtggtgaaagagtctatccaacatgttcactacataccttgagaagacaccagcgtagacaagacagtataggtagctctcacccagaggctcctttcctgagattacgtcacttcctaggcaaagaagatgcctccgcagcgatggataacatggatggtgctgaactttacggtagagttttaactgttaactacgctttgccagaaagaatcaagggtggtgaacaaggatgggctgcccaaccaatttgggctgacgctgatacatggtttgaaagacaacaacaagaagaggaaatgcaaaggatccaagccgaaaacagagctgctatgcaggctgccgaagaattgcatagaaaaaagttggctcaggaaagagagggtgagaaggaagatgaaattgacactaaagatgatccaatggctaaagcagaagctgaagttttacgacaaaattcttag

SEQ ID NO: 20 - DNA, 2586 bp, Synthetic construct
  source 1..2586: mol_type = other DNA
atgaccgtcagagagatagttgacatttcaaacctagctgaatcctgcacttctcttttatgtcgtaggcaaaataacagattcgtgcatatcgccgcaagatttgttgttgatgaagatgtagaaggagtctggctggaagaattgttggaagctttgacgttggtttgtgaagaaggtctaagactcagcttatcggacgcttcttgtcagggtagatccgtcttgatctacttgcgagaggatgcccacgaacgcgaaagactctctttctctagtgttaaaagatggcgtgagagaagctgtgaacccaggccacctttatctgggttttggagattatggtggcaattgttgagattctccagtgcttctccatccaatccagacattagatcaccgcctccaccaccctcctctccactgaacagaagcgttttcgatgtcacaaagggtaagtctcactatggcgtaggtggtggttacaaccattttgcgggcagagatgctagtagagccttcgtttccggtaattttactggtgaaggtctaaccgactctttgcgtggtttgagtaacgctgaaattaaaagtgttgtggagtggagaaacttctacaataagacttataccttcgtcggaaagttagttggtttgcactacgacgaccaaggtaacccaacaaagcatttgaaaggtgctgaagctaaggctgccaggggtgcacaactattgaagaaacaaaaggaagaagaagataaattgccctcttgcaactccagatggtcacaaggagaaggtggtgaggtctggtgtgatgacggttttcctcggcttgttcagcgcccattagaaatcgccttgtccggcaagatgtctaagagatgtgcttgtttccgtgaagaccaacttgctgaaccagggttggaagtctacgaggcagctctttttcctaagagaactaatgatgatggcgctgcaaagattttgcctccatcagatccatgctgtttcagctgcttcccatccggttggtgtgccggttccatacgtaactggatcagcaaatctatttcttctgactttaataccactacattgccacaacaatttctctatgtcagaagtttcccagaaggtgataaaaactgttacataatcaagttggcgcagggtaagggatataaatacttaatccgggcttcattcatgtacggtaactatgacggtcaaggtaaggctccagccttcgacttacacatgggggttaataagtgggattcggttattttgaacaatgaatcatctatcattattaaggaagtcatccacgctttaccaacttcttctatatgtatttgtttggtgaataccggtttcggtagcccattcatttccgccctcgaactaagattgttaaagaacgccacttacgtcaccgattttgaattacttgctttgcatcgtcgtttagacatcggttccacgactaacaaaactgttagatacaatgatgacgattgcgacagaatctggttgcctttcaacttccctaattataagatagtctctgcttcttcaaccgtggatagcggcgttacggctctgatgaacattaagtctttgtacggtgttagaaagaactggcaaggtgacccatgtatgccaaaatcctatgtctggcacggtttgaactgttcgtatgattcgcactcaccgaacagaattacttctcttaatttgagctcgtctaggctggttggtgaaactgctgcttacgtcagtgaattaacctccttgcaatacttggatctaagtaacaactctttgtcgggtcccgtgccgggttttctttccgaattacattccttgaaggttctggatttgagagacaataccttgcttggatctatcccatccgaattgatggaaagatcgacaaacgtaaaagagatcgacggttctgattactactctaatttaagccttgatattgttaattgttttatggaaggattggaagagacaccaatctttgtcttgcatcttttggcaaagactagaagaagagttactttattccaatccaactgttctaattcgttcattatcgcgggcccactctcttgtaactcaagcttggaccatgaaatgatgaagaccagatattatgcatctggcagattaacagaaaagagtgacgtatactctttcggggctttgattttggagattatcacttcaaggccagtcttgaaaatcaaccgtgcttccagcgaaaaatgccacgttggtcaatgggcaatgcacctcatgaaaactggtgatatcagatcaattgttgatgagagactcagaggtaactttgacctgtcctctgcttggaaggctgttgaaattgcaatgacctgtctatctcagacatcaatagaaagaccctctatgaaggaagtggtcatggaattatctgaatgtcttgctttggaaaaggcccgtaagagaaaaaatattgattctaacactagaagctccaacgctgttagtagaaacttcagtgaaagtgaagtgacgccattggccagatag

SEQ ID NO: 21 - DNA, 405 bp, Synthetic construct
  source 1..405: mol_type = other DNA
atgggctctggtgagaaagtgtttacactgaaggaagtcgctgaacataacaatcaaaaggactgctggctagtaatcagcggtaaggtttatgatgttaccaaattcttagacgatcacccaggtggagatgacgttttgctttcggcaactggtaaggatgcgactgacgatttcgaagacgtcggtcactccgataacgctcgtgaaatgatggaccagtactacgtcggtgagattgatgcttcaacgatacctaagaaagttgcctatgcccccccaaagcaaccacattacaatcaagacaaaacctccgaatttattatcaagttgttgcaattcttggttccgctcgctatcttagggttggctgttgccattagattctacactaagcaagcttag

SEQ ID NO: 22 - DNA, 642 bp, Synthetic construct
  source 1..642: mol_type = other DNA
atggctctgcaagtctgggagactctcaaggaggccattgtcgcttacaccggtctgtctcccgctaccttcttcactgtgctcgctcttggtttggccgtctactacgtcatttcgggcttcttcggcacctcggactacggctctcactctcgagatttcggtgagcagatgcagcccctgcctcctcccgtccaggttggtgagatctctgaggaggagctcaagcagtacgacggcagtgactccaagaagcctcttctcatggccatcaagggccagatttacgatgtcagccagagccgaatgttctacggacccggtggcccttacgctctgtttgccggcaaggatgcttctcgagctctggccaaaatgtcatttgaagagaaggacctgactggtgacatttccggactcggcccattcgagcttgaggccctccaggattgggaatacaagttcatgtccaagtacgttaaggtcggcaccgttgagaagcccgtcagcgagacagacgctgctcccgccggtgagtctgctgagtccaccaacgccgaggtgtccaagcccgctgaggacggtccttccgagtcccaggccgacaaggctgaggagacagtcgctgccaaggttgagtaa

SEQ ID NO: 23 - DNA, 843 bp, Synthetic construct
  source 1..843: mol_type = other DNA
atggctctgcaagtctgggagactctcaaggaggccattgtcgcttacaccggtctgtctcccgctaccttcttcactgtgctcgctcttggtttggccgtctactacgtcatttcgggcttcttcggcacctcggactacggctctcactctcgagatttcggtgagcagatgcagcccctgcctcctcccgtccaggttggtgagatctctgaggaggagctcaagcagtacgacggcagtgactccaagaagcctcttctcatggccatcaagggccagatttacgatgtcagccagagccgaatgttctacggacccggtggcccttacgctctgtttgccggcaaggatgcttctcgagctctggccaaaatgtcatttgaagagaaggacctgactggtgacatttccggactcggcccattcgagcttgaggccctccaggattgggaatacaagttcatgtccaagtacgttaaggtcggcaccgttgagaagcccgtcagcgagacagacgctgctcccgccggtgagtctgctgagtccaccaacgccgaggtgtccaagcccgctgaggacggtccttccgagtcccaggccgacaaggctgaggagacagtcgctgccaagcagaagaaggagaagaagaagggccgacgacgagtcgagggtgttctcatggagatccgaggcgaggccgttggtggccgaaagggcgagggcatgtcgagggagattggccgaggtggtggtggtgccgacgacggcaacctcaagggtggtggtatcgaccacggtgagcgagaacgagagcgagaacgagagtccggcatgtcttaa

SEQ ID NO: 24 - DNA, 630 bp, Synthetic construct
  source 1..630: mol_type = other DNA
atggagctgtactggaaggccatggagcagatcgcctggtacactggtctgtcccccaccgccttcttcaccattctggcctccatgatctttgtgttccagatggtcagctccatgttcgtttctcccgaggagttcaacaagcctcccaccgtccccgtcagctcttccaaccccgccaactccaacctcttcgtcaacgactctgtggctgatgcttcccaggccgtccagattggtcagctcaccgagcagcagctccgtgcttatgacggcagtgaccccaacaagcctcttctcatggccatcaagggccagatctacgacgtttcttctggacgaatgttctacggaccgggctctccttacgctatgtttgccggcaaggatgcttctcgagctctggctctgctgtccttcaagcccgaggacatcaccggtaacatcgagggtttgtccgaggaggagctcgttatccttcaggactgggagtacaagttcatggagaagtacgtcaaggttggtgagcttgtccttgagggaggaggagaagggatgaacgagcactccgagaacggtcaccgaaactttgagattgaccaggaggaggagcgaatcgaggctcgataa

SEQ ID NO: 25 - DNA, 1545 bp, Synthetic construct
  source 1..1545: mol_type = other DNA
atgcagattcttggtaagaccctgaccgctaccgcgatggtgggagcggttgcgcttttcgtggccgccaacatcttggtgcgtgtcatccagagaatgttcttccacgccttctccccctaccccggcccttggctcaactctgtttccccgctgccctccgcttggaaccttctgcgaggccgccagcacatctacaactaccagctgcataagaagtacggccccgtcgtccgcgttgcacccgacgagatttcttacactatccctgaggcttgggacgagatttacggcttccgaggaggtgccactatggagaagtctcccatcttcatcggcgtggtttctcctattgacggctatgtcggtgtcggtatggctaagggtaaggagcacgctagacagcgacgagccctggccccctctctgtccaaaaacgccctttacggtcagcgagacattatcctcacccatatcaagaagctcatcggctccatgcgagagcagacccggaacggtaaggccatcgacatgtctgactggtactcttatatcacctttgacattattggagatctttgttttgccgagccctttggctgcctcgataactcctctgcttccgagtggtctaccagcatcatcaacgtgctggtgtctgctacctgggagcaggccatccgacgagtcgccggcatcaacaccctcggctcgaagcttctggccaagctcatcatccccgccaccgcctctggatggcgaaagctgcatttctccaagacccgagagaagaccctgtcccgtctggaaaagacagcccttgagcgagaggatttcatgagacacatcctccgcaacgaggaccccaagaaggcactcacccctaccgagatcatcctcaacatggtcttgtttgtgtccgctggctccgaaaccacctccattttgatgacctcgctgacttacctcttgtgtactaacccctctgcctacaccaagttcgctgctgagatccgaggtgcctttgctaaggaggaggatatcaccgttgacgccgtgatggacctgccctacctgggtggagccatcaacgaggccatgcgtctgtttccccccggcgccgttaaccagcagcgtatcgttccgccccagggcgcccttattaacggaaaacaggttcccggaggactcaccctctcagttgccccttgggcatcaggtcgaagcccgcttaacttccacgagcctaacgagtttcgacccgagcggtggctcaaggactgcgatgctaagtacgccaacgacgctctccatgcgtcgaagcccttcggccatggtcctaaggcctgcctcggccaaaacctgtcctacatggagactagactcgttatgtcccatatcttctttaacttcgacatggagttggcccaggatggtcactctaagacgcttcagtggtccatggagccggacatcggttccgtgaagtcttttcaggctctgatgaagcctagcttgatgattaacattaccgagcgtaaataa

SEQ ID NO: 26 - DNA, 1269 bp, Synthetic construct
  source 1..1269: mol_type = other DNA
atggctttggctcgatttctttccatcttcttccccacatcccagagcgagttcgtcgccaacaccaactcgggtattcccactcgagagctgcccgcagcccctcctcaccctaaggtcgaggctccccctcgagatgtttggtactacccccctgatattgccaacgaccttcagggcgtcgacctgcccgaacgagtcaaaggagaggttttcgcctgcgcctgggagtacactcgatgtgtgattcctcagtacactaactggcctcgatacgttgccttcatgcgaattattgtcatgggcatcattgcagagttcaagggccacatggtcgacgtgacagccggtgatgacatcttgggatacgacctccaagctactctggacgagctgtttaagggtaccgttggccacgacgctatgagcaaggagtaccgatccttcctgctgattacagctgagaaatcctcggcccgacgagatggtgagacttttcgacgatacgttaacgccctgtctaagtctccccgacagtggttccgaatgcgagatgccgacgcccttgcacgattcactattgcatctgctctggcttgcaacgacctggacgacatttggttcactgaggagcagttcgaaatcatgactgagatcggagacactctttacgatgctgttgccttttttaagcaccgagccgagggtgaaactaacaacacctttgcctacatgcccgctgagatgcgagtgaaggccttccactacagccgagagattctgtgggccctggacgtggcatgggcaaaccgacccgagcttcagagcgtttccaactttattcgattcttcggtggccctatccacatgatgatgcgacgataccgattcgtcgaggagaaccttaccatcggtcagaccgaatccgacatggtcattaaccagacacgacaaaacttcaagctttggaaccgagttgatgttaaccgaggaaacggaaagcacgcaggccgatacaagaccgtgatggcttccgctaagaaaaacatgtacgacggactggaggacttcttgaagacctctgacgacgaccgatgcactcagtgccgatttcgagcttcgtacggagccgaaacccgacaccagttcggcggtgtccaggtctgtgacaactgcaaggttaaatggcgagagtacctggtttctttccctgaacgagctattaaggctttccccgaactgtccggagttgtctctctgagcccctaa

SEQ ID NO: 27 - DNA, 777 bp, Synthetic construct
  source 1..777: mol_type = other DNA
atgtcatcccagccctttaccaacaaggtgattgccctcactggatccgcctccggtatcggccttgagactgctaagctcctcgcctcccgaggcgctagactgtctcttgctgacattcaggaggacaaactcaaggagctccaggcccagcttgagtctgaatactacgtcgacgtcattaccactaaggttgacgtccgaaagtttggcgaggttgaagcttggattaacaagaccatcgacaacttcggaaagctggacggctccgcaaacctggctggagtcgcacctgagtccattggcctcaagggaatcgttgagcaggacctcgacgagtgggagttcgtgttgggcgtcaacctcaccggcactatgaactctctcaaggcccagcttaaagtcatggctaacaacggctctattgttaatgcttcgtccatccgaggtcttactggtgccgctaagaacgcttcctactcttcggcaaagcacggcattatcggactgacccgaactgctgccaaggaggtgggcggaaagggcattagagtcaacgctatctgtcccggaagaatctccacccccatgttgaagaccgcagagaactctattggtctgcaccttcagcctggctccgccaactacccccctattgccctgggcagagatggcgaagccaaggaggtcgctcaactcgtcgccttccttctctcggacgagagcacttatatcagcggcgctgacatctctattgacggcggatggcggtgctaa

SEQ ID NO: 28 - DNA, 2079 bp, Synthetic construct
  source 1..2079: mol_type = other DNA
atggccgagctcgatacactggatattctcgttttgggtgccatcctgttgggaaccgttgcatactttactaagggcaagtactggggcgtcacaaaggacccttacgcctcggccttcgccgcatccggaggtgctaagcctgccggcacccgaaacatcgtggaaaagatggaagaatctggcaagaactgtattatcttttacggatcgcaaaccggaaccgccgaagactacgcctctcgattggctaaagagggcaaatcccgatttggtctggacaccatggtcgctgacctggaggagtttgacttcgacaacctcgacactgtccccaacgacaagattgtgatgtttatcctcgccacttacggagaaggtgagcccactgacaacgctgtggatttttacgaatttatcacaggagatgatgtgtctttctctgagtccaacgacactccccttgccaaccttaactacgtgacttttggcctgggaaacaacacctacgaacactacaactctatggtgcgaaacgttgacaaggccctgcaaaagtttggtgcccaccgaattggcgacgccggtgaaggcgacgatggtgctggtactatggaggaagactttctggcctggaaggaccccatgtggtcgcaacttgctactaagatgggactcgaggaacgagatgctgtgtacgaacctgtcttctcgattgtggaccgagagggacttacacccgattcccctgaagtgtacctgggtgagcccaacaagtcccaccttgacggcaaaggccagggacctttcaactcccacaacccttacattgctcccattgctgagacacgagagctgttttctgtcaaagaccgaaactgcattcacctggacatcgacatctcgggatcgaacctgacttaccagaccggcgaccacattgctatctggcccaccaaccctggagctgaggtcgaccgattccttgacattattggactgactgaaaagcgaaaccaggtcatttcggtgaaacccctggaacctactgccaaggtgcccttccctacccccactacctacgacgctattattcgataccaccttgaggtctgtgcccccatgagccgacagtttgtttccaccctggcagccttcgctcccaacgacgagatcaagaccgaaatgactcgacttggcagcgacaaggactacttccacgagaagacctcgccccacttctacaacatcgcccgatttttggaagctgtgggaaagggacagaagtgggacaacattcccttctcggccttcattgaaggactcaccaagctgcaacctcgatactacagcatttcttcttcctcccgagttcagcccaagaaggtgtctgtgacagcagtcgttgagtctcaggcaatccctggtcgagatgagcccttccgaggcgtcgccaccaactacctcctggccctgaagcagaagcagaacggtgaccccaaccctgagccttttggacagtcctacgaactcaacggcccccgaaacaagtacgatggagttcacgtgcccgttcacatccgacactcgaacttcaagctcccctctgaccccgctaagcctgtgatcatgatcggacctggtaccggtgttgctcccttccgaggattcgtccaagagcgagccaagctcgcagaggagggcgtggaagtgggccccaccctgctctttttcggttgtcgaaagtccactgaagatttcatgtacaagtccgagtgggaacagtacaagaaagctctgggagataagttcgagttcatcactgccttttctcgagatggacccaacaaggtttacgttcagcaccgactgaaagagaaggccaaagaggtcaacgagctcctttctaagaaggcctacttctacgtttgcggcgacgctgccaacatggctcgagaggtgaacgctgtcctcgcacagatcattgctgagggtcgaggagtctctgaggccaaaggagaagaaatcgtcaagaacatgcgagcagccaaccaataccaggaggacgtgtggtcctaa

SEQ ID NO: 29 - protein, 134 aa, Catharanthus roseus
MASDQKLHKFDEVSKHNKTKDCWLIINGKVYDVTPFMDDHPGGDEVLLSATGKDATNDFEDVGHSDSAREMMDKYYIGEMDMATVPLKRTYIPPQQAQYNPDKTPEFVIKILQFLVPLLILGLAFAVRHYTKEK

SEQ ID NO: 30 - protein, 141 aa, unidentified
  source 1..141: note = Fusarium fujikuroi
MSAKKEFTMQDVAEHNTSSDIYMVVHDKVYDCTKFLDEHPGGEEVMLDVAGQDATEAFEDVGHSDEAREVLDGLLVGELKRLPGDEGPKRQIANSNQGSGKADPAGSSLNTYAIVVAVGFIAYVAYNYLQKQQEAQGQASA

SEQ ID NO: 31 - protein, 13 aa, Synthetic construct
  VARIANT 4: note = E, D, or N
  VARIANT 5: note = A or D
  VARIANT 7: note = E or D
  VARIANT 9: note = V or A
DATXXFXBXVGHS

SEQ ID NO: 32 - protein, 387 aa, unidentified
  source 1..387: note = Siraitia grosvenorii
MAVSNSSKKLLLSLFFFTSIVRLRPKVSVDANSVDDRLVVTCNSDLKDILPPPYSDLSSITCSPIWHQTQNVTTIVLSGKHDNRWIGIGFSREGKMVGSSAVVGWVERDGVSGFRQYYLEGKDISQVIPDKGDLRFTNAKPVVAVRGNLIYIAFQLQFPTSLAFQPILLAVGAGNPLRNGSLPKHINSTSTFIEFSSAHLAGQPTDPNELRRYHGAAAIIGWGILTPAGGLIPRYFRHLEPSWYYLHSSIQFVGFFVGLISISIGRNLYQKIDAVFVAHKFIGFCVFILAGLEVCQFVGRPSSDSKFRRYWNFAHYWVGRTAMVMGVANVFIGFHGIKADRSLKLGFAGAFAALLMTVIVLEGRLRRENAIPKTIDEPPVFQLTEKR

SEQ ID NO: 33 - protein, 220 aa, unidentified
  source 1..220: note = Siraitia grosvenorii
MNCLELCCWTREVANLRLNILRWTVTMTSHFVRWVYLPTMTVMRKMIKRQRILHQIVLLPGMNCRMALLVVRMLFGMIICQMISLPASRQRWVLCISMLLICHLQNNQVHCHQKRCRKMLERLKIQENRGLLRASLQLEQRFLLKRGIAKWIGLSLLEPILILQDKDSQIKGLFLVKLNNIRPRAPCGLFRVVCTISLHINFTLEVLIYSRQWEKIVHLY

SEQ ID NO: 34 - protein, 307 aa, unidentified
  source 1..307: note = Siraitia grosvenorii
MHSRNPSSVASASPETQALKRGGGPEFLTVAVALVAIGVGTAYYFYISKKPKGCLDPHNFKEFKLIERTQLSHNVAKFRFSLPTPTSVLGLPIGQHMSCSYIIISSSTEEKTAKVKKLSNRTLQPPWIQMLDVSTRKDVPPLREMRVGDYLAVKGPKGRFKYKANEVEAFGMLAGGTGITPMFQVTRAILENPVDRTNVHLIYANVTYQDILLKEELDSLATRFPTRFTIYYVLNQPPEGWVGGVGFVTEQMLRLHCPPPAPNVRILRCGPPPMNKAMAAYLNSIGYTPQMQFQFNLTSPPSPATFI

SEQ ID NO: 35 - DNA, 405 bp, Synthetic construct
  source 1..405: mol_type = other DNA
atggcctcagatcagaaactgcacaaattcgatgaagtgtcaaagcataacaaaaccaaggattgttggctaattattaatggtaaggtctatgatgtaactccatttatggatgatcatcctggtggtgatgaagttttgttgagcgccactggcaaggacgctacaaacgactttgaagatgtaggtcattctgactccgctagagaaatgatggacaagtactacattggtgaaatggatatggccactgtcccactaaaaagaacttacattccacctcaacaagcccaatacaatcctgataagacaccagaatttgttatcaagattttacaattcttggttcctttgttgattttgggtttggctttcgccgtgcgtcattacactaaggaaaagtaa

SEQ ID NO: 36 - DNA, 426 bp, Synthetic construct
  source 1..426: mol_type = other DNA
atgtccgccaagaaagaattcaccatgcaagatgttgctgaacacaatacctcttccgatatctacatggttgttcacgataaggtttacgattgcaccaagttcttggatgaacatccaggtggtgaagaagttatgttggacgttgctggtcaagatgctactgaagcttttgaagatgttggtcattctgatgaagccagagaagttttggatggtttgttggttggtgaattgaaaagattgccaggtgatgaaggtccaaagagacaaattgctaactccaatcaaggttctggtaaagctgatccagctggttcttctttgaatacttatgctatcgttgttgccgttggtttcattgcttatgttgcttacaactacttgcaaaagcaacaagaagctcaaggtcaagcttctgcttag

SEQ ID NO: 37 - DNA, 426 bp, Synthetic construct
  source 1..426: mol_type = other DNA
atgagcgcgaagaaagaatttaccatgcaagacgttgcagagcacaatacgtcgagtgatatatatatggtagtccatgataaagtgtacgattgcactaaattcctagacgaacatcctggaggggaagaggttatgctggatgtagccggtcaggatgctacagaagcttttgaagacgtcggccactctgatgaagcacgcgaggttttagatggtttgcttgtgggtgaactcaagcgattacccggagatgaaggtccgaagagacaaattgcaaactccaatcaagggtcaggaaaagccgacccagctggttcatctcttaacacatatgcaatcgttgtcgctgtgggcttcattgcatacgttgcttataattacttgcagaagcaacaggaggcgcaaggccaagcgagtgcctag

SEQ ID NO: 38 - DNA, 1164 bp, Synthetic construct
  source 1..1164: mol_type = other DNA
atggccgtatcaaattccagcaagaaactattactgtctctttttttcttcacctctattgttagattgaggcccaaggtcagtgttgacgcgaactcggtggatgacagattggttgtcacatgtaactccgatttgaaggatatattaccgcctccatactctgacctgtcctctatcacgtgctcaccaatttggcatcagactcaaaatgtcaccactatcgttttatcgggtaaacacgacaaccgttggatcggcattggttttagtagagaaggaaagatggttggttcctctgctgttgtcggttgggtagagagagatggtgtgtccggtttcagacaatattacttggaaggtaaggatatcagccaagtcattccagacaaaggtgatttacgatttactaacgcaaagcctgttgttgctgttcgtggcaatttgatctatattgctttccaattgcaatttccaacgtctttggctttccaaccaatcttgctcgccgtcggtgctggaaacccacttagaaacgggtctttgcctaagcacattaatagcacctctactttcattgaattttcatctgcacatttggctggtcagccaaccgacccaaacgaactcagaagataccacggcgctgccgcgataatcggttgggggattttaactccagctggtggtcttattccaagatacttccggcatttggaaccaagttggtactatttgcactcctctatccaatttgtaggtttcttcgttggtctaatatctatttctatcggcagaaacttgtaccaaaagattgacgctgtcttcgtcgcccataaatttataggtttctgtgtgttcattttggctggtctggaagtttgtcaattcgttggtagaccatccagtgattccaaattccgtcgttattggaattttgcacactactgggtgggaagaacagctatggttatgggtgtcgctaacgtcttcataggttttcatggtatcaaggccgaccggtctttaaagcttggatttgctggtgcgttcgctgctttgttaatgactgttattgttttggaaggtagattgcgcagagagaacgccatcccaaagaccatcgatgaaccccccgtttttcagctgactgaaaagagatag

SEQ ID NO: 39 - DNA, 690 bp, Synthetic construct
  source 1..690: mol_type = other DNA
atgaactgcttggagttatgttgttggacgcgtgaagtggcatgaaatctgagactaaacatcttgagatggacagttaccatgacttctcacttcgtacgatgggtctatttgcctaccatgactgttatgagaaagatgattaaacgccagagaatactccatcaaatttaggtcttgcttccctaaggcatgaattgtagaatggccctattggttgtcaggatgttatttggtatgatcatttgccaaatgatatcattgccagcttcccggcaacgttgggttttatgtatcagcatgttgttgatttgtcaccttcaaaacaaccaagttcattgtcaccaaaagagatgtcgcaagatgctggaaagattgtgaaaaattcaggaaaacagagggttgttaagagctagtttgcaattggaacaaagattcctcctaaagagaggtatcgctaagtggataggtttatctttattagaaccaattttgatcttgcaagactgaaaagattcgcaaatcaaaggtctatttttgtgagtgaagttgaataacattagaccacgggccccttgcggacttttctgaagagtcgtttgtactatctctttgcacatttgaaacttcactctggaagtattgatctactcctgaagacagtgggagaagattgttcatctatactag

SEQ ID NO: 40 - DNA, 924 bp, Synthetic construct
  source 1..924: mol_type = other DNA
atgcactctcgtaaccctagctccgtcgcctcagcatctccagagactcaggctttaaagcgaggaggcggtcccgaatttctgaccgtggctgttgcgcttgttgctattggtgtaggtacagcttactatttctacatctccaaaaagccaaagggttgcttggatccacataatttcaaagaatttaagctaatagaaagaactcaattgtctcacaacgttgccaagttcagattctcgttaccaaccccgacgagtgtcttgggtttgcctattggtcaacatatgtcttgttcatatatcattatctcaagttccactgaagagaaaaccgctaaggttaaaaagctatccaatagaaccttgcaaccaccatggattcaaatgcttgacgtcagtactagaaaggatgttcctcccttaagggaaatgagagtcggtgactacttggccgtcaaggggccaaagggacgctttaaatacaaggctaacgaagttgaagcttttggcatgttggcgggtggtactgggattacaccaatgttccaagttactagagctatattggaaaacccagtagatcgtacaaatgtgcacctgatctacgcaaacgtcacttatcaagacatcttattgaaggaggaattggattccttagctactagattcccgaccagattcaccatttattacgttctaaaccaaccaccagaaggttgggttggtggtgtgggttttgttacggaacaaatgttaagattgcactgtccaccacctgctccaaacgtacgtatcctaagatgtggtccaccaccaatgaataaagccatggctgcctacttgaatagcatcggttacactccccagatgcaattccagttcaatttgacctctccaccttctcccgctacatttatttag

SEQ ID NO: 41 - protein, 512 aa, Catharanthus roseus
MELYYFSTFAFLLFCFILAKTLKKSGQSNLKLPLGPPPIPILGNAHQLIGGHTHHILRDLAKKYGPLMHLKTGEVSTIVASSPEIAEEMFKTHDVLFADRPSNIVAFKILSYDYSDVVISPYGNYWRQLRKISMMELFSQRSVQSFRSIREEEVLNFIKSIGSREGTKINLSKEISLLIYGITTRAAFGEKNKNTEEFIRLLDQLTVAVAEPNIADMFPSINFLKLISRSKYKIEKIHKNFDAIVQTILNHHKDRLANHKSSSHEENGEQNKDLVDVLLNIQQRGDFDTPLGDRSVKAVIFNIFSAGTETSSTTVDWAMCEMIKNPTIMKKAQEEVRKVYNEEGNVNETKLHQLKYLKAVIKETLRLHPPVPLLLPRECREQCEIKGYTIPSKSRVIVNAWAIGRDPNYWIEPENFNPERFLESEVDFKGNSFEYLPFGGGRRICPGITFALANIELPLAQLLFHFDWKLASDETNIDKLDMTESRGVTVRREDDLCLIPFPYSASSLKGKY

SEQ ID NO: 42 - protein, 355 aa, Catharanthus roseus
MDVQSEEFRGAQAQIWSQSCSFITSASLKCAVKLGIPDTIDNHGKPITLSELTNALVPPVHPSKAPFIYRLMRVLAKNGFCSEEQLDGETEPLYSLTPSSRILLKKEPLNLRGIVLTMADPVQLKAWESLSDWYQNEDDSSTAFETAHGKNFWGYSSEHMEHAEFFNEAMASDSQLISKLLIGEYKFLFEGLASLVDIGGGTGTIAKAIAKNFPQLKCTVFDLPHVVANLESKENVEFVAGDMFEKIPSANAIFLKWILHDWNDEDCVKILKSCKKAIPAKGGKVIIIDMVMYSDKKDDHLVKTQTSMDMAMLVNFAAKERCEKEWAFLFKEAGFSDYKIYPKLDFTRSLIEVYP

SEQ ID NO: 43 - protein, 506 aa, Catharanthus roseus
MEFHESSPFVFITRGFIFIAISIAVLRRIISKKTKTLPPGPWKLPLIGNLHQFLGSVPYQILRDLAQKNGPLMHLQLGEVSAIVAASPQMAKEITKTLDLQFADRPVIQALRIVTYDYLDISFNAYGKYWRQLRKIFVQELLTSKRVRSFCSIREDEFSNLVKTINSANGKSINLSKLMTSCTNSIINKVAFGKVRYEREVFIDLINQILALAGGFKLVDLFPSYKILHVLEGTERKLWEIRGKIDKILDKVIDEHRENSSRTGKGNGCNGQEDIVDILLRIEEGGDLDLDIPFGNNNIKALLFDIIAGGTETSSTAVDWAMSEMMRNPHVMSKAQKEIREAFNGKEKIEENDIQNLKYLKLVIQETLRLHPPAPLLMRQCREKCEIGGYHIPVGTKAFINVWAIGRDPAYWPNPESFIPERFDDNTYEFTKSEHHAFEYLPFGAGRRMCPGISFGLANVELPLALLLYHFNWQLPDGSTTLDMTEATGLAARRKYDLQLIATSYA

SEQ ID NO: 44 - protein, 352 aa, Catharanthus roseus
MAAKSVKALGLALKDSSGLFSPFNFSRRATGEHDVQLKVLYCGVCNFDNLMRRNKYGRTKFPYVFGHEIVGVVTEVGSNVKKFKIGNKVGVSFIVDTCRECERCKIGQQIACKKAVSSDGFFETPGYGGCSNIFVADENYVILWPENLPMDSGAPLLCIGITCYNPLRRFGLDKPGVRVGIVGLGAVGHLAIKFAKAFGARVTLISSSPGKKDEAFQKFGVDSFLVSSNAEEMQAAAETLDGILDTVPVVHPLEPLFALLKPLGKLIIIGEPHKPFEVSAMSLMEGGKIISASTGGSIKDTQEIVDFAAEHNVVADVEVIPVDYVNTAMERLDKADVKYRFVIDIGNTFKSP

SEQ ID NO: 45 - protein, 289 aa, Catharanthus roseus
MEEKQEKVAEFYDKVTGAWDLFYGVHLHDGYYEPGTTATMAISQDAVIRMIDELLRFAGVSEDPAKKPRSMLDVGSGLGGTCVYVAKKYDIQCTGITISPNQVKYAQDYAATEGVENKVSFDCGDALDMPYSDGKFDVVFTINCIKHVHDKEKFIREMVRVAAPGAAIIIASQAHPNLSPGESLKPRDKKILQKICDGAGAVSLCSSDDYVRWLTPLPVKEIKAADWTQNITPLYPLLMKEAFTWKGFTSIVLKGGWRAINLINAVRLVAKAANDGILKFAVVTGRKSI

SEQ ID NO: 46 - DNA, 24 bp, Synthetic construct
  source 1..24: mol_type = other DNA
gatgcacgagcgcaacgctcacaa

SEQ ID NO: 47 - protein, 6 aa, Synthetic construct
YTGLSP

SEQ ID NO: 48 - protein, 15 aa, Synthetic construct
KPLLMAIKGQIYDVS

SEQ ID NO: 49 - protein, 9 aa, Synthetic construct
LQDWEYKFM

SEQ ID NO: 50 - protein, 47 aa, synthetic construct
  VARIANT 2: note = L or V
  VARIANT 3: note = Y or W
  VARIANT 4: note = W or E
  VARIANT 5: note = K or T
  VARIANT 6: note = A or L
  VARIANT 7: note = M or K
  VARIANT 9: note = Q or A
  VARIANT 11: note = A or V
  VARIANT 12: note = W or A
  VARIANT 19: note = T or A
  VARIANT 20: note = A or T
  VARIANT 24: note = I or V
  VARIANT 27: note = S or L
  VARIANT 28: note = M or G
  VARIANT 30: note = F or A
  VARIANT 32: note = F or Y
  VARIANT 33: note = Q or Y
  VARIANT 34: note = M or V
  VARIANT 35: note = V or I
  VARIANT 37: note = S or G
  VARIANT 38: note = M or F
  VARIANT 40: note = V or G
  VARIANT 41: note = S or T
  VARIANT 42: note = P or S
  VARIANT 43: note = E or D
  VARIANT 44: note = E or Y
  VARIANT 45: note = F or G
  VARIANT 46: note = N or S
  VARIANT 47: note = K or H
ZXXXXXXEXIXXYTGLSPXXFFTXLAXXJXVXXXXSXXFXXXXXXXX

SEQ ID NO: 51 - protein, 41 aa, Synthetic construct
  VARIANT 1: note = P or A
  VARIANT 4: note = V or I
  VARIANT 8: note = S or T
  VARIANT 13: note = K or R
  VARIANT 14: note = Q or A
  VARIANT 20: note = S or P
  VARIANT 21: note = K or N
  VARIANT 37: note = Q or S
  VARIANT 38: note = S or G
XVQXGZJXEZZLXXYDGSDXXKPLLMAIKGQIYDVSXXRMF

SEQ ID NO: 52 - protein, 43 aa, Synthetic construct
  VARIANT 3: note = K or L
  VARIANT 4: note = M or L
  VARIANT 7: note = E or K
  VARIANT 8: note = E or P
  VARIANT 9: note = K or E
  VARIANT 16: note = S or E
  VARIANT 19: note = G or S
  VARIANT 20: note = P or E
  VARIANT 21: note = F or E
  VARIANT 24: note = E or V
  VARIANT 25: note = A or I
  VARIANT 35: note = S or E
  VARIANT 42: note = T or E
  VARIANT 43: note = V or L
LAXXSFXXXDJTGBIXGLXXXELXXLQDWEYKFMXKYVKVGXX

SEQ ID NO: 53 - protein, 13 aa, synthetic construct
  VARIANT 1: note = K or E
  VARIANT 2: note = P or H
  VARIANT 3: note = A or S
  VARIANT 7: note = P or H
  VARIANT 8: note = S or R
  VARIANT 9: note = E or N
  VARIANT 10: note = S or F
  VARIANT 12: note = A or I
XXXEBGXXXXZXD

SEQ ID NO: 54 - protein, 15 aa, Synthetic construct
ILRVSFRKYRKAIEQ

SEQ ID NO: 55 - protein, 18 aa, Synthetic construct
RAFRPSIRFKKSHSTVPT

SEQ ID NO: 56 - protein, 8 aa, Synthetic construct
KNTLYVGG

SEQ ID NO: 57 - protein, 20 aa, Synthetic construct
DQATQKHRSFGFVTFLEKED

SEQ ID NO: 58 - protein, 47 aa, Synthetic construct
QVWETLKEAIVAYTGLSPATFFTVLALGLAVYYVISGFFGTSDYGSH

SEQ ID NO: 59 - protein, 47 aa, Synthetic construct
ELYWKAMEQIAWYTGLSPTAFFTILASMIFVFQMVSSMFVSPEEFNK

SEQ ID NO: 60 - protein, 41 aa, Synthetic construct
PVQVGEISEEELKQYDGSDSKKPLLMAIKGQIYDVSQSRMF

SEQ ID NO: 61 - protein, 41 aa, Synthetic construct
AVQIGQLTEQQLRAYDGSDPNKPLLMAIKGQIYDVSSGRMF

SEQ ID NO: 62 - protein, 43 aa, Synthetic construct
LAKMSFEEKDLTGDISGLGPFELEALQDWEYKFMSKYVKVGTV

SEQ ID NO: 63 - protein, 43 aa, Synthetic construct
LALLSFKPEDITGNIEGLSEEELVILQDWEYKFMEKYVKVGEL

SEQ ID NO: 64 - protein, 13 aa, Synthetic construct
KPAEDGPSESQAD

SEQ ID NO: 65 - protein, 13 aa, Synthetic construct
EHSENGHRNFEID

SEQ ID NO: 66 - protein, 376 aa, Catharanthus roseus
MKDLNFHAATLSEEESLRELKAFDETKAGVKGIVDTGITKIPRIFIDQPKNLDRISVCRGKSDIKIPVINLNGLSSNSEIRREIVEKIGEASEKYGFFQIVNHGIPQDVMDKMVDGVRKFHEQDDQIKRQYYSRDRFNKNFLYSSNYVLIPGIACNWRDTMECIMNSNQPDPQEFPDVCRDILMKYSNYVRNLGLILFELLSEALGLKPNHLEEMDCAEGLILLGHYYPACPQPELTFGTSKHSDSGFLTILMQDQIGGLQILLENQWIDVPFIPGALVINIADLLQLITNDKFKSVEHRVLANKVGPRISVAVAFGIKTQTQEGVSPRLYGPIKELISEENPPIYKEVTVKDFITIRFAKRFDDSSSLSPFRLNN

SEQ ID NO: 67 - protein, 439 aa, Catharanthus roseus
MESGKISVETETLSKTLIKPSSPTPQSLSRYNLSYNDQNIYQTCVSVGFFYENPDGIEISTIREQLQNSLSKTLVSYYPFAGKVVKNDYIHCNDDGIEFVEVRIRCRMNDILKYELRSYARDLVLPKRVTVGSEDTTAIVQLSHFDCGGLAVAFGISHKVADGGTIASFMKDWAASACYLSSSHHVPTPLLVSDSIFPRQDNIICEQFPTSKNCVEKTFIFPPEAIEKLKSKAVEFGIEKPTRVEVLTAFLSRCATVAGKSAAKNNNCGQSLPFPVLQAINLRPILELPQNSVGNLVSIYFSRTIKENDYLNEKEYTKLVINELRKEKQKIKNLSREKLTYVAQMEEFVKSLKEFDISNFLDIDAYLSDSWCRFPFYDVDFGWGKPIWVCLFQPYIKNCVVMMDYPFGDDYGIEAIVSFEQEKMSAFEKNEQLLQFVSN

SEQ ID NO: 68 - protein, 688 aa, Arabidopsis thaliana
MTSALYASDLFKQLKSIMGTDSLSDDVVLVIATTSLALVAGFVVLLWKKTTADRSGELKPLMIPKSLMAKDEDDDLDLGSGKTRVSIFFGTQTGTAEGFAKALSEEIKARYEKAAVKDDYAADDDQYEEKLKKETLAFFCVATYGDGEPTDNAARFYKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSSPRLAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 69 - DNA, 1539 bp, Synthetic construct
  source 1..1539: mol_type = other DNA
atggaattgtattacttctctactttcgcctttttgttgttttgcttcatcttagccaagactttgaagaagtccggtcagtccaacctaaagttgccactgggtccaccaccaattccaatcttgggtaatgctcatcaattaattggcggtcacacccaccatattttaagagatttggccaaaaaatatggtccattgatgcatttgaaaaccggtgaagtttctacaatcgttgcatcctcgccagaaattgccgaagaaatgttcaagactcatgatgtattgttcgctgacagaccatcgaatatcgttgctttcaaaatactaagctacgattacagcgatgtagttatatctccatacggtaattactggagacaactaagaaagatttcgatgatggaattatttagtcaaagatcagttcaatctttcagatctatcagagaagaagaggtcttgaacttcattaagtctattggctccagagaaggtaccaagatcaacttgagcaaggaaatttctttgttgatttacggtatcactactagggctgctttcggtgaaaagaataagaatacggaagaatttatcagattgttagaccaattgaccgttgccgttgctgagcctaacattgctgatatgtttccttctataaatttcttgaagttaatctcaagaagcaagtacaaaatcgaaaagatccacaaaaattttgacgccattgtccaaaccatcttaaaccaccacaaagacagactagctaaccacaaatcctccagtcatgaagaaaatggtgaacaaaacaaggacttagtcgacgttttgttaaacatccaacaaagaggcgatttcgatacaccattaggtgatagaagtgttaaagccgttatatttaacattttttctgctggtaccgaaacctcttccacaactgttgattgggccatgtgtgaaatgatcaaaaacccaactataatgaagaaagctcaagaagaagtcagaaaggtttataacgaagaaggtaacgtcaatgaaactaagctgcatcaattaaagtacttaaaggccgtaataaaagaaactctaagattgcatccaccagttccccttcttcttccaagggaatgtagagaacaatgtgagatcaaaggttacaccataccttcaaaatccagagttatcgtgaacgcttgggccataggtagagatccaaactactggattgaaccagaaaattttaacccagaaagatttttagaaagtgaagtcgacttcaagggtaactcctttgaatacctaccattcggtggtggtcgtcgtatttgcccaggtattacctttgctttagctaatattgaattgcctttagctcaattattgttccactttgactggaaattggctagcgacgaaactaacatcgacaagttagatatgacagaatccagaggtgttactgtcagaagagaagatgatttgtgtctgattccattcccatattcagcatcttctttgaagggaaagtactaa

SEQ ID NO: 70 - DNA, 1068 bp, Synthetic construct
  source 1..1068: mol_type = other DNA
atggacgttcagtctgaagagttcagaggtgctcaagctcaaatctggagtcaatcctgctctttcattaccagtgcatccttgaagtgtgctgttaaattgggtatcccagatacaattgataatcacggcaagccaatcactttgtctgagttgacgaatgccttagttccaccagtccacccatctaaagcccctttcatatatcgtttgatgagagtattggcaaagaacggtttttgctctgaagaacaattggacggtgaaaccgaaccattgtactctttgactccatcatcaagaatcctgttaaaaaaggaaccacttaatttgagaggtattgtcttgacaatggctgacccagtacagctaaaagcctgggagtcacttagcgactggtatcagaacgaagatgattcttctaccgccttcgaaacggctcacggtaagaacttttggggttactcgagtgaacacatggaacatgctgaatttttcaacgaagctatggcctccgattctcaattaatctccaaattgttaataggtgaatataagttcctttttgaaggtctagccagtttggtcgatattggtggtggtaccggtactattgcaaaggctatcgctaaaaacttcccacaactaaagtgtaccgtctttgatttaccacacgttgttgcaaacttggaatcaaaagaaaacgtcgaatttgtcgctggtgatatgttcgaaaagatcccttcagctaacgccattttcctaaagtggatccttcatgactggaacgatgaagattgtgtcaaaattttgaagtcctgcaagaaggctatcccagccaagggtggtaaagtcattatcattgatatggttatgtacagcgacaagaaggatgaccacctagttaagacacaaacttcaatggatatggctatgttggtcaatttcgctgccaaggaaagatgtgaaaaggaatgggccttcttgttcaaagaggctggtttctctgattacaagatttacccaaagcttgatttcactcgttctttgatcgaggtttatccataa

SEQ ID NO: 71 - DNA, 1521 bp, Synthetic construct
  source 1..1521: mol_type = other DNA
atggagtttcacgaatcctccccatttgtgtttattactcgtggttttatcttcattgctatatccattgccgttttgcgtagaatcatctccaagaaaactaagacgttaccaccaggtccatggaagttgcctttgattggtaacctacatcaattcttgggttccgttccatatcaaatattacgtgatttggctcaaaagaacggtcctttgatgcacttgcaacttggtgaagtttctgcaatcgtcgctgcatccccacaaatggccaaagaaatcactaaaaccttagatctacaattcgctgatagacctgtcatacaagctctaagaatcgtaacctacgattacttagatatttcctttaacgcctacggtaagtactggagacaattgagaaagatcttcgtccaggaattactgactagcaagagagtcagatctttttgtagtattagagaagatgagttctccaacttagtcaagactataaattctgctaacggtaagtctattaatttgtccaagcttatgacctcttgtactaactctattatcaataaggtcgcttttggtaaggttagatacgagagagaggtttttattgacttgattaatcaaattttagctttggctggtggttttaaattggtggatctattcccatcatacaagatccttcatgttttggaaggtactgaaagaaagttatgggaaattaggggcaaaatcgataagatcttggataaagttattgacgaacaccgtgaaaattcctccagaacaggtaagggtaacggttgtaacggtcaagaagatatcgttgacattctattgcgtattgaagagggcggtgatttagacttggatattccatttggtaacaacaatatcaaggcattgttgttcgatatcattgctggtggtaccgaaacttcctcaacggctgtcgattgggccatgagtgaaatgatgagaaacccacacgttatgtcaaaggctcaaaaggagattagagaagcttttaacggtaaggaaaagatcgaagaaaacgatatacaaaacttgaagtaccttaaattggtgattcaagagactttaagactgcacccacccgccccacttttgatgagacaatgtagggaaaagtgtgaaattggtggttatcacataccagtgggtacaaaggctttcattaacgtatgggcaattggtagagatccagcttattggcctaacccagaatcttttattccagaacgtttcgacgataacacatacgagtttaccaagtccgaacatcacgcttttgaatatttgccattcggtgctggtagaaggatgtgtcccggtatttctttcggtttagctaacgttgaattgccattggctcttttgttgtaccatttcaactggcaattacctgatggttctactacgttggatatgaccgaagcaacgggtttggccgccagaagaaaatacgatttacaactgatcgctacttcctacgcctaa

SEQ ID NO: 72 - DNA, 1059 bp, Synthetic construct
  source 1..1059: mol_type = other DNA
atggccgctaagtctgttaaggctttaggattggccttgaaagactcttcaggcttgttttcccctttcaatttctccaggagagctactggtgagcacgatgttcaattgaaagtgctttattgcggtgtctgtaactttgacaacctgatgagaagaaacaagtatggtagaaccaagttcccttacgttttcggtcacgaaatagttggtgtcgtcaccgaagttggtagcaacgttaagaagttcaagattggtaataaggtaggtgttagtttcatcgttgatacctgtagagaatgtgaacgttgcaagattggtcaacaaattgcttgtaagaaggctgtctcttctgatggttttttcgaaaccccaggttatggtggctgttcgaacatctttgttgccgatgaaaactacgttattttatggcccgagaatttaccaatggattccggtgccccattactttgtatcggtattacttgttataatcctttaagaagatttggtttagataaaccaggcgttagagttggtatagtgggtttgggcgccgtgggtcatttggctattaagtttgccaaggcattcggtgctagagttactttgatttcttccagtcctggtaagaaggatgaggcttttcaaaagtttggtgttgattccttcttagtttcttcgaatgctgaagaaatgcaagctgccgccgagactttagatggtatcttagacacggttcctgtcgtccaccctttagagccattgtttgctttgttgaaaccattgggaaaattgattattattggtgaaccacataagccttttgaggtatcggccatgtctttgatggaaggtggtaaaatcatttctgcctctaccggtggttcaatcaaggacacccaggaaattgttgactttgctgctgaacataatgttgtcgctgatgttgaagtgatcccagtcgattatgttaatactgctatggaaaggttggacaaagccgacgtgaagtacagatttgttattgacatcggtaatacctttaagtccccataa

SEQ ID NO: 73 - DNA, 870 bp, Synthetic construct
  source 1..870: mol_type = other DNA
atggaagagaaacaagaaaaggtcgctgagttctacgacaaagttactggtgcttgggacttgttctatggtgtacacttgcatgacggttattatgaaccaggcaccactgctaccatggctattagccaagatgctgttattaggatgattgatgaactattaagatttgctggtgtttcagaagatcctgctaagaaaccaagaagcatgctagatgttggttcaggcttaggtggtacttgtgtttatgttgcaaagaagtacgatattcaatgtactggtatcacaatctctccaaaccaagtcaaatacgctcaagactatgctgctactgaaggtgtcgaaaacaaagtttcttttgactgtggtgacgctttagatatgccatactctgatggtaaatttgacgttgtgttcactatcaactgtatcaaacacgtccatgataaagaaaagtttatcagagaaatggtccgtgttgctgctccaggtgctgctatcatcattgcttctcaggcccatccaaacttatcccctggtgaatctctaaagccaagagacaagaagattttgcaaaagatctgtgatggtgctggtgcagtctctttgtgttcttcggacgactatgtccgttggttaacccctctaccagtcaaagaaattaaggccgccgactggacacaaaacatcactcccttatacccattgttgatgaaggaagctttcacctggaaaggtttcacttctattgtcttgaagggtggttggagagccattaacttgatcaacgctgttagattggttgctaaagccgctaacgatggtattttaaagtttgctgtcgtcactggtcgtaagtctatttaa

SEQ ID NO: 74 - DNA, 1131 bp, Synthetic construct
  source 1..1131: mol_type = other DNA
atgaaagatttgaatttccatgctgcaactctttctgaagaagaatcgttgagagaattgaaggcctttgacgaaaccaaggctggtgttaagggtatcgtcgacactggtatcacaaagattcctagaattttcatcgatcaacctaagaatttggatagaattagcgtttgtcgtggtaagtcagatattaagattccagttattaacctaaacggtttgtcatctaactccgaaattagaagagaaattgttgaaaaaattggcgaagcctctgaaaaatatggtttctttcagatcgttaaccatggtatcccacaggacgtcatggacaagatggttgacggtgttcgtaaattccatgaacaggatgatcaaattaagagacaatattactcgcgtgaccgttttaataagaacttcttgtactcttctaattatgtcttgatccccggcattgcttgcaactggagggatactatggaatgcattatgaactcaaatcaaccagacccacaagaatttcctgatgtctgtcgtgatatactaatgaagtactctaactacgttagaaatcttggtttgattcttttcgagttgttatctgaagctttaggtttgaagcctaatcatttggaagaaatggattgcgctgagggtttgatcttgttgggtcactactatccagcttgtccacaaccagagttgacctttggtactagcaaacactctgattccggtttccttactattttgatgcaagaccaaatcggtggtttgcaaatattgttagagaaccaatggattgatgttccttttatcccaggtgctctggttattaatatcgccgatcttttgcaattaattacaaatgacaaattcaagtctgttgaacacagagttttggccaataaggttggtccacgtatttctgttgcagtagctttcggtattaaaacccagactcaagaaggtgtaagccctaggttgtatggtccaattaaagaattgatttctgaggaaaacccaccaatatataaagaagttacggttaaagatttcatcaccatcagattcgctaagcgtttcgatgattctagttctttatcgccattcagattaaacaactaa

SEQ ID NO: 75 - DNA, 1320 bp, Synthetic construct
  source 1..1320: mol_type = other DNA
atggaaagcggcaaaatctcagttgagacagagactttatctaagacactaatcaagccgtcctctcctactccccagagcctatctaggtacaacctgagctataatgatcaaaatatttaccaaacttgtgtatcagtcgggtttttttatgagaatcccgacggtattgagatttcaacgatcagagaacaactgcaaaatagcctaagcaaaaccctagtctcttactatccctttgcaggtaaagtggtcaagaacgattacattcattgcaacgacgatgggatagagttcgtggaagttcgtatcagatgcagaatgaatgacattctgaaatacgagctgaggtcttacgccagagacttagtattgccaaaacgtgtcacagtagggtcagaggatacaactgcaatcgtccaattgagccacttcgattgcggtgggttggcagtcgcgtttggcataagccataaggttgccgacggtgggactattgcctctttcatgaaggattgggcagcaagcgcgtgttacctgtcatctagccatcatgtcccgacgccacttctagtgagtgacagcatcttcccacgtcaggacaacataatttgtgaacagttcccgacctccaagaactgcgtggaaaagacgttcatctttcccccagaggctatcgagaagctaaagagcaaggccgtggaattcggcatcgaaaaacctacacgtgtcgaagtgttaacggcgtttttatctaggtgcgcaactgtagccggtaaatccgcagcgaaaaacaacaactgcggccaaagtctgcccttccctgtgctgcaggcgattaacctacgtccaattcttgaactaccccaaaactctgtagggaacttggtatccatatattttagtagaacaatcaaagaaaacgactacttaaatgagaaggagtacaccaagttggtgataaatgagcttaggaaggaaaagcaaaaaataaaaaacctatctcgtgaaaaactaacgtatgtcgcgcaaatggaggaattcgttaagtcccttaaagagttcgacatctccaattttttggacatcgatgcttacttgtcagacagttggtgtaggtttcccttttatgacgtggactttggctggggcaagcccatttgggtgtgtttattccaaccttacatcaagaattgcgttgtaatgatggactacccatttggggacgattatgggatcgaagccatagtatcattcgagcaggagaaaatgtcagcctttgaaaagaacgagcaattgcttcagtttgtttcaaactaa

SEQ ID NO: 76 - DNA, 2067 bp, Synthetic construct
  source 1..2067: mol_type = other DNA
atgacatcagcactatatgcgagcgatctgtttaaacaactgaagagtataatgggcacagacagtttgtctgacgatgtagttttagttatcgcaaccacatcactggctctggtagcgggatttgtcgtattactatggaaaaaaacaactgcagacaggagcggcgaattaaagcctctaatgataccaaagtctcttatggccaaagacgaggacgacgatctagatctagggtctggaaaaacgcgtgtgtccatcttttttgggacccagactggaacagccgagggtttcgctaaggctctttctgaggagataaaggctaggtatgagaaagcggctgtgaaggacgattatgcagcagacgacgaccagtacgaagaaaagctaaaaaaggagacattggccttcttttgtgttgcgacttacggcgacggcgaacctacagacaatgcggctcgtttttataagtggttcacggaagaaaacgagagagatataaagctgcaacagttagcttatggagtcttcgcactgggaaatagacagtacgaacatttcaacaagatcgggattgtcttagatgaagaactgtgcaaaaaaggcgccaaacgtttaatcgaggtcggtcttggcgacgacgaccagtccatagaggacgattttaacgcttggaaggagagtttatggagcgagcttgacaaacttctaaaggatgaggacgacaagagcgttgctaccccgtacactgcggtgattccggagtaccgtgtcgtgactcatgatcccaggtttaccacgcaaaaaagtatggagtccaacgtggcgaacgggaatacaactatcgacattcaccacccgtgcagggtagacgttgcggttcaaaaggaattgcacacacatgagagtgataggtcatgtattcacttagagtttgatatatctaggactggaataacttacgaaaccggggaccatgtgggagtctatgccgagaatcacgttgaaattgtagaggaggccggcaagttactgggccactccttggacctagtattttccatacatgctgacaaagaagatggtagtcccctagagagcgcggtaccccctcctttcccgggcccatgtacactgggtaccggattggcccgttatgcagacttactgaatcctcctagaaaatcagcgttagtggctcttgcggcgtacgcaactgagcccagtgaagcggagaagttgaagcatttgactagtcccgatggtaaggacgaatatagccagtggatagtcgcgtcccagaggagcctactagaagtgatggcggcttttccgagtgcaaaacccccattaggggtattttttgccgcaattgcacctagattgcaacctcgttattattcaatcagtagttccccgcgtttggcaccatcccgtgtccacgtaacgtcagctttagtctatggcccgaccccaactggtcgtatacacaagggtgtgtgttcaacctggatgaagaatgccgttcccgcagagaaatctcacgagtgtagcggagccccgatcttcatcagagcatcaaactttaagcttccttccaatccgtcaacgccgatcgttatggtgggtccaggaaccggcttggcaccatttcgtggatttctgcaagagcgtatggctctaaaagaagatggggaggaactaggttcaagtttattgttttttgggtgcaggaacaggcagatggattttatatacgaagacgaactaaacaatttcgtagaccagggcgtgatttcagaattaatcatggcatttagtagagaaggtgcccaaaaagaatatgttcagcataagatgatggaaaaagccgcccaggtttgggatttaataaaggaagaaggttacctatatgtatgcggtgacgcgaaggggatggcgagggatgtacataggacattacacacgatcgtccaagagcaggaaggggtgagtagtagtgaggcagaggccatagtaaaaaagctgcagacagaggggaggtacttacgtgatgtgtggtaa

SEQ ID NO: 77 - protein, 850 aa, unidentified
  source 1..850: note = Myrothecium indicum
MKHQLVRQCSRALAGSIRSSYATRALKYRTYSTHPPNAKLNLPVDYGSTPLLAHGSQAVLGNRELPSEVRNGPTKKMNLFQAINDALSISLSEDENVLVFGEDVAFGGVFRCTMKLQETYGSERVFNTPLSEQGILGFAVGLAAEGQRPVAEIQFADYVFPAFDQLVNEAAKVRYREGATGRSAGSLTVRMPAGGVGHGGLYHSQSPEAVFTHIPGLRVIMPRSPIQAKGLLLSAIRSNDPCLFLEPKILYRAAVEQVPIDAYELPLSKAEVVKEGKDVTIISYGQPIYTCLAAIRKAEEDFGISAELVDLRTVYPWDKQTVFESVRKTGRAIVVHESCLNAGVGAEVAAAIQGDKDTFLRLEAPVSRVTGWDIHNPLIFEKFHIPDVARVYDRIKTVLERIASNMSHIDTVASFVEGAPPGELADVVADIKTLTLDSPNIVSELAPAFEKYNEEQFTTVKLPGSSQQVVITSHNSLGGGRYYDVESSSSFAFDHTTQKASAVQSYVVEGAQAELTKSTLKSLASYASDHFPNAAYGVYPVESDSKIAIIVVSNKYSPNNYWNGRWRSLYVFDSASGSLDGSVKVDVHYYEDGNVRLLTNKAISAQVSSGTGAGIVKEIAAAEKKYQEDLNKGFVSLSEGAFKGLRRQLPVTRQKIEWDKVTGYRLGQDIGGGSGSPAGTQGSPTTQSTIVAPNTPTTQLSFSPQQASLQAPSVKMSKEFTFQDVAEHNTKKDLFLVIHDKVYDSSKFVDEHPGGEEVMLDVGGQDATEAFEDVGHSDEARETLEKLLVGTLKRQPGDPEPSKPQYTANAVNGAKSESGGLGIAAYAVILIGGVLAYVGYQYTQNKEGSA

SEQ ID NO: 78 - protein, 1001 aa, unidentified
  source 1..1001: note = Myrothecium indicum
MPSRTKPTLTRAEVESHSTASSCYVTIGSNVYDMTEFMDDHPGGGSLILDYAGRDVKDILNDRDSHFHSEAAYEILDDYHVGFVVNQKSDQGLNGAAKVGELEKTEADGVHPRTGMSCEEDLSKETDYNQDYKRHKFLDLNRPLFPQIWFSGFSKDFYLEQVHRPRHYKGGQSAPLFGNFLEPLTKTPWYVVPSLWLPCVAYGTYLASDGLGSTSLVGYWIFGLFIWTLVEYCLHRFLFHLDDYLPDNRVGISLHFLLHGIHHYLPMDKYRLVMPPTLLVLLATPFWKLAHVLFAYSWYAGTAVYCGGIFGYVCYDMTHYFLHHENLPLWYKQLKKYHLEHHFLDYENGFGVTSRFWDDIFGTGLDKPPVIKTQHYDAFGKAPTRVPARQKFAGRGNTAKCGWSRETNYTEPNPTSSLPQLLDIWEKLYSLSTPTTRDVEALLDATAKSSADKNPNKSKSDGSPREDKHASRDSGRDSGRDRDGSAPRDDRDYDRRRRADRDVSPGRGRRDSPDDGHVGTPRSDAGSHKGRRRSRSRGGYSRRHRDGDYYRSGRGGRSRSRSPNRHYRPRDDRDRRDRRDRSDRRSQEYGRGRDDRNEQRSDGPPQLTEDERDRRTVFVQQLAARLRTRELKEFFEKVGPVNEAQIVKDRISQRSKGVGYVEFKEEESVSKALQLTGQKLLGIPVIVQVTEAEKNRQARNPESGGAHPNSIPFHRLYVGNIHFNVTEEDLQAVFEPFGELEFVQLQKDENGRSRGYGFVQFRDAGQAREALEKMNGFDLAGRPIRVGLGNDKFTPESTANLLQRFTGPNPNFQGSAFSGAGGRGSQKSTFDRAGGRDSEKTGGASALDDTDVAGVNFNNYSRDALMRKLARTDESATNGTEERQTLKPKTETKPLPVNVNMASRCVVLHNMFDPDEEEGDDWVKELEDDIRQEAEQKYGHVVHISVDPNSKGDVYLKFDKVQGGENAIKGLNGRYFGGRMIDASPVVDAVYSSLFSRTRAI

SEQ ID NO: 79 - protein, 472 aa, unidentified
  source 1..472: note = Myrothecium indicum
MSSSAAQAKAQAFPDGTTDYKPLRSSKYDPKKVHISEQPMTWSNWHQHINWLNTILIIGVPMMGFISSYWVPLRLKTLLFSIFYYFHCGMGITAGYHRLWSHTSYKATTPLKVYLAAVGAGAVQGSIRWWSMKHRAHHRYTDTEKDPYSVRKGLLYSHLGWMVFKQNPKMTGRTDITDLNEDPVVVWQHRHYLKSVIFMALVLPTLVCGLGWGDWVGGYVYAGILRICFIQQATFCVNSLAHWLGDQPFDDRNSPRDHVITALVTLGEGYHNFHHEFPSDYRNAIEWYQYDPTKWSICFWKFFGLAYDLKQFRANEIEKGRVQQMQKKLDRQRATLDWGTPLEQLPVVSWDDFVDQSKNGKALIAIAGVIHDVTDFIKDHPGGKALISSAIGKDATSIFNGGVYNHSNAAHNLLSTMRTGVLRGGCEVEIWKRAQFENKDVTYMNDSEGQRIVRAGSQVTRLSQPVASADAA

SEQ ID NO: 80 - protein, 313 aa, unidentified
  source 1..313: note = Myrothecium indicum
MATVQSESLLARHYIDYIYVPAALLIFGTAIVKRGWLPYSVLLAVTLGAYNFWKFQIKKVLKPDVFQEFELQEKIIISHNVAIYRVKLPTPRSILGLPIGTHVSIGADLPQPDGSTKEIVRSYTPISGDHQPGYVDLLIKSYPQGNISKHMASLNVGQKVRLRGPKGAFNYTPNMVRHFGMIAGGTGITPMLQIIRAIVRGRPDGDVTKVDLIFANVSPQDILLKEDLDALANEDADIHIHYVLDKPPEGWTGGVGYVTADMITKWLPKPADDVKILLCGPPPMISGLKKTTESLGFNKARPVSKLEDQVFAF

SEQ ID NO: 81 - protein, 625 aa, unidentified
  source 1..625: note = Myrothecium indicum
MAPRVVVVGGGLSGLSAAHTIYLAGGNVVVLDKQGFFGGNSTKATSGINGALTRTQVDLNIGDSVKQFYDDTMKSARDKARPDLIKVLTYKSAAAVEWLQDVFNLDLTLVSRLGGHSQPRTHRGHDAKFPGMAITYALMQRLEELAENEPERVQIIKKARVTGLNKDGNTITGVTYEYNGESASVEGPVVLATGGYAADFGEGSLLKQHRPDTYGLATTNGTHATGDGQKMVMAIGGNGIDMDKVQVHPTGLVDPKDPGSKWKFLAAEALRGEGGLLLNADGDRFCDELGHRDYVSGMMWKEKDKGKFPIRLILNSKASNVLDFHTRHYSGRGLMKKMTGQELAKEIGCKPDHLQGTFKTYNAIADGKEKDPWGKKFFHNLPFDINDDYHVALMEPVLHFTMGGIEINDKAQVLNKEQKPFDGLFACGELAGGVHGANRLGGSSLLGCVVYGRVAGDTASNYLFQQALQGTGSAAQRLGQISLHLDPSAPGKLTVEWANGGSGSGASAGPAPTTAATGPTPGPEGAKASKPNNPKEFKVPEQEFTMEEVAKHNKKDDLWVVVKGVVMDLSNWLDEHPGGPQAIMNFMGRDATEEFEMLHDDEVIPKYAPQQVIGRVKGQTPSLEL

SEQ ID NO: 82 - protein, 570 aa, unidentified
  source 1..570: note = Myrothecium indicum
MSNAQELATASPAVPARERTYKLISRDEIEALIADGRSIIIVDQHVLKIDPFLKYHPGGDKAMMHMVGRDATDEVTVLHSAEAKQLMTRYRIGKIEGHWVNFVPPIQGGVFRTAAQQAKDKAIVDGGRDQESSSSSSREASPVFDDATYTRGIRHRGGAPGSPSSASSVSVNEEEMDGMSYLDTITKEHIKLDLDMYPDIDQVTQEAIVDKYRELHQRIKDEGLYECRYWNYYVECFRYAFFFGGMLLCLHFEQYLLAGISLGAMWHQLVFSAHDAGHMGITHDFQIDTIIGIIIADFLGGLSMGWWKRNHNVHHIVTNAPEHDPDIEHMPLFAISHRFLGNLRSTFYDRLMEYDAAAKLLLRVQAWTYYPFLALGRFNLYFLSWDFLIAGRGPRKGPAAWHRWLEVAGQVFFWTWYGYGVMYCMIPDNRSRLVFFIVSNMTPSPLHLQIVLSHFAMSTSDLGPQESFPQKMLRTTMDIDCPEWLDFIHGGLQFQVIHHLFPRVPRHNLRRTQKLVQEFCNEVGIPYALYGFVNSNQRVLGRLSDVSKQAAILAKCQRTIVSKGDPFGHH

SEQ ID NO: 83 - DNA, 2553 bp, Synthetic construct
  source 1..2553: mol_type = other DNA
atgaagcaccagctggtccgacagtgctctcgagctcttgctggatcgatccgatcttcctacgccacccgagcccttaagtaccgaacctacagcactcaccctcccaacgccaagctgaaccttcctgtggactacggttctactcccctgcttgctcacggtagccaagctgtgctcggcaaccgagaactcccttctgaggtgcgaaacggacccacaaaaaagatgaacctgttccaagccatcaacgacgcactgagcatcagcctgtctgaggacgagaacgtccttgtgttcggagaagacgtcgcattcggaggcgtcttccgatgcactatgaagcttcaggagacttacggatcggaacgagttttcaacacccccctgtctgagcaaggcatcctcggctttgctgtcggccttgccgctgagggccagcgacccgtggctgagatccagttcgccgattacgtcttccccgcttttgaccagcttgtcaacgaggccgccaaggtgcgataccgagaaggagccaccggacgatccgcaggctccttgactgtgcgaatgcccgccggcggagtcggccacggcggcctgtaccactcccagtcccctgaggcagttttcacccacatccctggcttgcgagttattatgccccgatctcctatccaggccaagggtttgctgctctcggctatccgatctaacgatccctgcttgtttctcgagcccaagatcctgtaccgagccgcagtggagcaggtccctatcgatgcatacgagctccccctttctaaggccgaagtcgtgaaggagggcaaggacgttaccattatttcttacggtcagcctatttacacctgtcttgccgccattcgaaaggcagaggaggattttggaatttcggccgagcttgttgaccttcgaactgtttacccttgggataagcaaactgtgttcgagagcgttcgaaagaccggccgagccattgtggtccacgagtcctgcctgaacgccggcgtgggcgctgaggtggccgctgccattcagggtgacaaggacacctttctgcgacttgaggcccccgtttcgcgagttactggctgggatattcacaaccctttgattttcgagaagttccacatccccgatgtcgctcgagtctacgaccgaattaagaccgtgctcgagcgaattgcaagcaacatgtcgcacatcgatactgtggcctcttttgtggagggagcaccccctggagaactggctgatgtggtggccgacattaaaaccctgactctcgactcgcccaacatcgttagcgagctggctcctgcctttgagaagtacaacgaagagcagttcaccaccgttaagcttcccggctcctcccagcaggtcgtgattacttcccacaactctctcggaggtggccgatactacgatgtggagagctcttcgtcgttcgcatttgatcacactacccagaaggcttccgccgtgcaatcgtacgttgtcgagggcgcccaagctgaactgactaagtctaccttgaagtccctggcttcctacgcttccgaccactttcctaacgccgcctacggcgtgtaccccgttgagtccgattccaagatcgccattattgttgtgtctaacaaatactcgcctaacaactactggaacggacgatggcgatctctgtacgtcttcgatagcgcttccggctccctggacggctcggtcaaagtggatgttcactactacgaggacggcaacgtccgactcctcacaaacaaggccattagcgctcaggtttcgtccggaaccggagccggtattgtcaaggagattgccgccgctgaaaagaagtaccaggaagacctcaacaagggcttcgtcagcctgtccgagggtgcctttaagggattgcgacgacaactgcctgtgacccgacagaaaattgagtgggataaggtgaccggctaccgactgggacaggatattggaggtggcagcggttctcccgctggtactcaaggaagccccacaacccagtctacaattgtcgctcctaacactcctacaactcaattgtccttttcgcctcagcaggcctccctccaggctccctctgtgaagatgtcgaaagagtttacctttcaagatgtcgcagagcacaacactaagaaagatctgttcctcgtcatccacgataaggtgtacgactcttctaagttcgtggacgaacaccccggaggcgaagaggttatgctcgatgtgggaggccaagacgccactgaagcttttgaggacgtgggccacagcgacgaagctcgagagactctcgagaagttgctcgtgggcactctcaagcgacagcctggtgaccctgagccttctaaacctcagtacaccgctaacgccgtgaacggagctaagtccgagagcggcggactgggtattgctgcctacgctgtcatcctgatcggcggcgtcttggcctacgtgggataccaatacacccagaacaaggaaggatcggcctaa

SEQ ID NO: 84 - DNA, 3006 bp, Synthetic construct
  source 1..3006: mol_type = other DNA
atgccctctcgaaccaagcccaccctcactcgagctgaggtcgagtcccactctacagcttcgtcgtgttacgttactatcggttctaacgtttacgacatgacagagttcatggacgaccaccctggcggcggatccctcatcctggactacgctggccgagatgttaaggacattctgaacgatcgagactcccacttccactctgaagccgcctacgagattctggacgactaccacgtgggcttcgtcgtgaaccagaagagcgatcagggtctgaacggcgctgccaaggtgggagagctggagaagaccgaggcagatggcgttcaccctcgaaccggtatgtcctgcgaggaggacctgtcgaaggaaactgattacaaccaggattacaagcgacacaagttcctggacctgaaccgacctctgtttccccagatctggttctccggcttcagcaaggacttctacctggaacaggtccaccgacctcgacactacaagggcggccagtctgcccccctgtttggtaacttcctcgagcccctcaccaagaccccctggtacgtggttccctccttgtggctgccttgtgtggcttacggcacctacctcgcctccgatggcctgggttcgacctccctcgtcggatactggatcttcggattgtttatttggactctggtggaatactgcctgcaccgattcctcttccacttggatgactacctccccgacaaccgagtcggcatttcccttcactttctcctccacggtatccaccactaccttcccatggacaagtaccgactggttatgcctcctaccctgctggtcctgctcgctacccctttttggaaattggctcacgtgctgtttgcctactcttggtacgccggcaccgctgtctactgcggaggtatctttggatacgtttgctacgacatgacccactacttcctccaccacgaaaacttgcccctgtggtacaaacagctgaaaaagtaccacttggagcaccacttcctggattacgagaacggcttcggagttacaagccgattttgggacgacattttcggaaccggcctggataagcctcccgtgatcaagactcaacactacgatgcatttggaaaggcacctactcgagtgcctgctcgacaaaagtttgccggtcgaggtaacaccgccaagtgcggctggtcccgagaaaccaactacaccgagcccaaccccacctcctccctgccccagctcttggacatttgggagaaactctactcgctgtctactcccacaacccgagatgttgaggcccttcttgacgccactgccaaaagctccgccgataagaaccccaacaagtctaaatctgacggatctccccgagaggacaagcacgcctcgcgagattctggtcgagactccggccgagatcgagatggcagcgccccccgagatgaccgagactacgaccgacgacgacgagccgatcgagatgtctctcccggccgaggtcgacgagattctcccgacgacggtcacgtgggaactcctcgatcggacgcaggatcgcacaagggacgacgacgatcccgatcccgaggaggctacagccgacgacaccgagatggtgactactaccgatccggccgaggaggtcgatcgcgatctcgaagccccaaccgacactaccgacctcgagatgaccgagatcgacgagatcgacgagatcgaagcgaccgacgatcccaggagtacggccgaggacgagatgaccgaaacgagcagcgatcggacggccctcctcagctgacagaggacgagcgagatcgacgaaccgtttttgtccagcaactggccgctcgattgcgaactcgagagctgaaggagtttttcgagaaggttggacctgtcaacgaagctcaaattgtcaaggaccgaatctctcagcgatccaagggtgttggatacgttgagttcaaggaagaggagtccgtttcgaaggctctccagctgacaggacagaagctgctgggcatccccgttatcgttcaagttaccgaggcagagaagaaccgacaggcccgaaaccctgagtccggaggcgctcaccccaacagcattcccttccaccgactttacgtcggcaacatccacttcaacgttaccgaggaggacctccaggcagtttttgagcctttcggtgagctcgagtttgtccagctccagaaggacgagaacggacgatctcgaggctacggcttcgtgcaattccgagatgccggccaagcacgagaggccctggagaagatgaacggttttgatctcgcaggtcgacctattcgagtgggcctgggaaacgataaatttacacctgagagcactgcaaaccttttgcagcgattcaccggacccaaccctaacttccagggctccgccttctctggcgcaggcggccgaggctctcagaagagcaccttcgatcgagccggaggccgagactccgagaagaccggtggtgcctcggccctcgacgataccgatgtggctggcgtcaactttaacaactacagccgagatgccctcatgcgaaagcttgcccgaaccgacgagagcgctaccaacggtactgaggagcgacagaccctgaaacccaagactgaaaccaagcccttgcctgtcaacgtcaacatggcctcgcgatgtgtggtgctgcacaacatgttcgaccctgacgaggaggaaggcgacgactgggttaaagagctcgaagacgatatccgacaggaagctgagcaaaagtacggacacgtggtccacatttccgtggaccccaactccaagggcgacgtctacttgaagtttgacaaggtccagggcggcgagaacgctattaagggcctgaacggacgatacttcggaggccgaatgatcgatgcctcccccgtggtggacgctgtgtactcttccctcttctcccgaacccgagctatttaa

SEQ ID NO: 85 - DNA, 1419 bp, Synthetic construct
  source 1..1419: mol_type = other DNA
atgtcctctagcgctgcacaagccaaggcacaggctttccctgacggcactaccgactacaagcccttgcgatcctccaaatacgatcctaagaaggtccacatctccgagcaacctatgacttggtctaactggcaccagcacatcaactggctgaacacaattctcattattggcgtgcctatgatgggattcatttcttcctactgggtccccctccgactgaagactctcctgttttctatcttctactacttccactgcggaatgggtatcaccgccggttaccaccgactctggtcgcacacctcgtacaaggccactacccccctcaaggtgtacctcgccgccgttggcgctggagccgtccagggctctatccgatggtggtctatgaagcaccgagctcaccaccgatacacagacaccgagaaggatccttactctgtgcgaaagggtctgctgtactcgcacctgggttggatggtctttaagcagaaccccaagatgaccggtcgaaccgatattaccgacttgaacgaagaccctgtggtggtttggcagcaccgacactaccttaagtctgtgatctttatggccctcgttctgcccaccctcgtttgcggactgggctggggtgactgggtgggaggctacgtttacgccggaattttgcgaatctgttttattcaacaagccactttctgtgtgaactcgctcgcccactggctgggtgaccagccctttgatgaccgaaactcccctcgagatcacgtgatcaccgccctggttaccctgggcgagggataccacaactttcaccacgagtttccctctgattaccgaaacgccatcgaatggtaccagtacgatcctaccaagtggtccatctgtttctggaaattctttggtttggcctacgaccttaagcagttccgagctaacgagattgagaagggccgagtccaacaaatgcagaagaaactggaccgacagcgagccacattggactggggaactcctctcgaacagctgcccgttgtgtcttgggacgacttcgtcgaccaaagcaagaacggaaaggccctgattgctattgccggtgtgattcacgacgtcaccgacttcatcaaggaccaccccggaggtaaggcactgatctcttctgcaattggcaaggatgcaactagcatcttcaacggtggagtgtacaaccacagcaacgccgctcacaacctgctgtccaccatgcgaactggcgttctgcgaggtggttgtgaggtcgagatctggaagcgagctcagtttgaaaacaaagacgtgacatacatgaacgactccgagggacagcgaattgtgcgagccggcagccaggttactcgactgtcccaacccgtggcatcggccgacgcagcttaa

SEQ ID NO: 86 - DNA, 942 bp, Synthetic construct
  source 1..942: mol_type = other DNA
atggccaccgtccagtctgaatccctgctggctcgacactacatcgactacatctacgtgcccgccgcactcctcatttttggcaccgccattgtgaagcgaggctggctgccctactccgtgctgttggctgtgacccttggcgcttacaacttttggaagttccagatcaagaaggtcttgaagcctgatgttttccaggagttcgaactccaggagaagatcattattagccacaacgttgctatttaccgagttaagctgcctaccccccgatctatcctcggtctgcctatcggcactcacgtgtccattggtgccgacctccctcaacccgacggttctacaaaggagattgtgcgatcctacacccccatttctggcgatcaccagcccggttacgtggatcttctcattaagtcttacccccaaggcaacatttctaagcacatggcttcgctgaacgttggccagaaagtccgacttcgaggtcccaagggtgccttcaactacactcccaacatggtgcgacacttcggtatgattgccggcggtaccggtatcacccccatgcttcagatcattcgagctattgtgcgaggacgacccgacggcgacgtgacaaaagtcgaccttatcttcgccaacgttagcccccaggatatcctcctcaaggaggatctcgatgccctggctaacgaggacgctgatattcacattcactacgtccttgacaagccccccgagggatggaccggaggcgtcggatacgtcacagccgatatgattactaagtggctccctaagcctgccgacgacgtcaagattctgctttgtggtcctccccccatgatttccggcctgaaaaagaccaccgaatctctgggattcaacaaggctcgacctgtctctaagctggaagatcaggtgtttgccttctaa

SEQ ID NO: 87 - DNA, 1878 bp, Synthetic construct
  source 1..1878: mol_type = other DNA
atggctccccgagtcgtcgtcgtgggtggaggtctgtccggtctttctgccgcccacaccatctaccttgctggaggtaacgttgtcgtgcttgacaagcagggcttttttggcggtaactcgaccaaggctaccagcggcattaacggagctctgactcgaacccaggtcgacctgaacatcggcgattcggtcaagcagttctacgacgacacaatgaaatctgcacgagacaaagcacgacccgacctgatcaaggtcctgacatacaagagcgccgccgctgtggagtggctgcaagacgtcttcaacctcgatctcacactcgtgtcgcgattgggtggacactcccagcctcgaactcaccgaggtcacgacgccaaattccccggtatggccattacctacgccttgatgcagcgactcgaagaactggcagagaacgagcctgagcgagtccagatcatcaagaaggcccgagttaccggattgaacaaggacggtaacaccatcaccggcgtgacctacgagtacaacggagagtcggcttccgtcgagggacctgtcgtgctcgcaaccggaggctacgcagccgactttggcgaaggttcgctgttgaagcagcaccgacctgatacctacggactggctacaacaaacggaacccacgccactggagatggtcagaaaatggtcatggctattggaggaaacggaatcgacatggataaagtgcaggtccaccccacaggacttgtggatcccaaggaccctggttccaaatggaagttcctggccgcagaagctctgcgaggtgaaggcggcttgctgcttaacgctgatggagatcgattctgtgacgaacttggacaccgagattacgtgtccggaatgatgtggaaagagaaggacaaaggaaagttccccatccgactgattctcaactcgaaggcctctaacgtcctcgactttcacactcgacactactcgggacgaggtctgatgaagaagatgaccggccaggaactggcaaaagaaatcggttgcaagcctgatcaccttcagggcacctttaagacttacaacgctatcgccgatggaaaggaaaaggacccctggggtaagaagttcttccacaaccttcccttcgacatcaacgatgattaccacgttgcacttatggaacctgttcttcactttaccatgggcggaatcgaaatcaacgacaaggctcaggttcttaacaaggagcaaaagccttttgatggcttgttcgcttgtggcgagctcgccggaggcgtgcacggagctaaccgacttggaggatcctctctgctgggatgtgtggtttacggacgagtggccggcgacactgcatctaactacctcttccagcaggctctccagggcactggatctgctgcacagcgactgggccagatctctctgcacctggacccctccgctcccggtaagctcaccgtggaatgggctaacggcggctcgggcagcggagcatctgctggccccgcccctacaacagctgctaccggacctactcccggacccgagggtgccaaagcatccaagcccaacaaccccaaggagttcaaggtgcccgagcaagagttcactatggaggaggtggccaagcacaacaagaaggacgacctgtgggtcgtcgtcaagggtgtggtcatggatctgtctaactggcttgacgagcaccccggcggcccccaagcaatcatgaactttatgggacgagatgcaactgaggaatttgagatgttgcacgacgatgaggttatccctaagtacgcccctcagcaggtcattggacgagtgaaaggtcagacccccagcctggagctgtaa

SEQ ID NO: 88 - DNA, 1713 bp, Synthetic construct
  source 1..1713: mol_type = other DNA
atgtctaacgcccaagagctggcaacagcctcgcccgctgtgcccgctcgagagcgaacctacaaactcatctctcgagatgagattgaggctctgatcgcagacggacgatcgatcattattgtcgatcagcacgtcctcaagatcgaccccttcctcaagtaccaccctggcggagacaaggctatgatgcacatggtcggccgagatgccaccgacgaggtgacagtgcttcactccgctgaggccaagcagcttatgactcgataccgaatcggtaagatcgagggccactgggtgaacttcgtgcctcccattcagggcggagtgtttcgaaccgctgcccagcaagccaaagacaaggccatcgtcgacggtggacgagatcaggagtcttcttcctcctcttcccgagaagcctcgcctgtcttcgatgatgctacatacacccgaggtatccgacaccgaggcggtgctcctggaagcccttcttccgcaagctccgtgtcggtcaacgaggaggagatggacggtatgtcctacctcgacactatcactaaggagcacattaagctcgacctggatatgtaccccgatattgatcaggtcacccaggaggccatcgtcgacaagtaccgagaactgcaccagcgaattaaggacgaaggactctacgaatgtcgatactggaactactacgtcgagtgttttcgatacgccttcttctttggtggaatgcttctgtgcctgcactttgagcagtacctgctggcaggcatttccctgggtgctatgtggcaccagctcgtcttctctgcccacgacgccggtcacatgggcattacacacgattttcagatcgatacaattatcggaatcatcattgctgactttctgggcggactttccatgggctggtggaaacgaaaccacaacgtgcaccacatcgtcactaacgcacctgaacacgaccctgatattgagcacatgcctctctttgccattagccaccgatttctgggaaacctccgaagcacattctacgatcgacttatggaatacgacgccgccgccaaacttctgctgcgagtccaggcctggacttactaccccttcctcgcactgggtcgatttaacctttacttcctgtcgtgggatttcctcattgccggccgaggcccccgaaagggtcctgctgcttggcaccgatggcttgaagtggccggtcaagttttcttctggacttggtacggttacggagtgatgtactgtatgattcccgacaaccgatcgcgattggtgttcttcattgtcagcaacatgaccccttctcccctccacctccaaattgtgctctcccactttgccatgtcgacttcggacctgggacctcaggagtcgttccctcagaagatgttgcgaaccaccatggatattgattgccccgaatggctggatttcatccacggcggccttcagttccaggtcatccaccacttgttcccccgagtgcctcgacacaaccttcgacgaacacagaagctggtccaggagttttgcaacgaggtcggcatcccctacgctctgtacggtttcgttaactccaaccaacgagtgctcggtcgactgtctgacgtgtctaagcaggccgctatcttggctaagtgccaacgaaccattgtttccaagggagatccctttggacaccactaa

SEQ ID NO: 89 - protein, 509 aa, unidentified
  source 1..509: note = Botrytis cinerea
MSNSILNLGSFACLLSLGSIVLWYTISAVLAWYPLRKIPAPSFLATFSYLWLAKTTYSGKQYWIQRDLHKKYGPLVRIGPTDIITDDPEIIKKISSARSSHRRGDWYLTGRFNPYYDNMFTMLEPGPHAKAKARTAAAYSGRDMPDLEVGVNAQLQTLIGLMRSKYASNTVKPHQPLLDLGQVSCFFTMDVITRLAFGEEFGYLKEETDQYGFLGEVRELWPRMSTSADTPWIRKFLFSPPFLKVLGPKPTDKTGFGALMAVAEHHVGKRFAPDAKKKEDMLGSFIRHGLNQQECEVEGLFMIVAGTESTASAIRSTLVHVMTCPRVYQKLKTEINLAVEEGKVSSPIKLEEAKLLPFLQAVIYEGIRMRPPLLGLFPKIVPDGGEEFHGMFIPAGTAICMNTSSLLRSTALFGDDAEVYRPERFMELEKSKRGEMERNVELAFGYGQYMCVGKTVAFMELNKSIFEILRAFDLQLLSPAKPCDVLSYGIFLESNMLVKVTESEGTEYK

SEQ ID NO: 90 - protein, 514 aa, unidentified
  source 1..514: note = Myrothecium indicum
MQILGKTLTATAMVGAVALFVAANILVRVIQRMFFHAFSPYPGPWLNSVSPLPSAWNLLRGRQHIYNYQLHKKYGPVVRVAPDEISYTIPEAWDEIYGFRGGATMEKSPIFIGVVSPIDGYVGVGMAKGKEHARQRRALAPSLSKNALYGQRDIILTHIKKLIGSMREQTRNGKAIDMSDWYSYITFDIIGDLCFAEPFGCLDNSSASEWSTSIINVLVSATWEQAIRRVAGINTLGSKLLAKLIIPATASGWRKLHFSKTREKTLSRLEKTALEREDFMRHILRNEDPKKALTPTEIILNMVLFVSAGSETTSILMTSLTYLLCTNPSAYTKFAAEIRGAFAKEEDITVDAVMDLPYLGGAINEAMRLFPPGAVNQQRIVPPQGALINGKQVPGGLTLSVAPWASGRSPLNFHEPNEFRPERWLKDCDAKYANDALHASKPFGHGPKACLGQNLSYMETRLVMSHIFFNFDMELAQDGHSKTLQWSMEPDIGSVKSFQALMKPSLMINITERK

SEQ ID NO: 91 - protein, 422 aa, unidentified
  source 1..422: note = Myrothecium indicum
MALARFLSIFFPTSQSEFVANTNSGIPTRELPAAPPHPKVEAPPRDVWYYPPDIANDLQGVDLPERVKGEVFACAWEYTRCVIPQYTNWPRYVAFMRIIVMGIIAEFKGHMVDVTAGDDILGYDLQATLDELFKGTVGHDAMSKEYRSFLLITAEKSSARRDGETFRRYVNALSKSPRQWFRMRDADALARFTIASALACNDLDDIWFTEEQFEIMTEIGDTLYDAVAFFKHRAEGETNNTFAYMPAEMRVKAFHYSREILWALDVAWANRPELQSVSNFIRFFGGPIHMMMRRYRFVEENLTIGQTESDMVINQTRQNFKLWNRVDVNRGNGKHAGRYKTVMASAKKNMYDGLEDFLKTSDDDRCTQCRFRASYGAETRHQFGGVQVCDNCKVKWREYLVSFPERAIKAFPELSGVVSLSP

SEQ ID NO: 92 - protein, 258 aa, unidentified
  source 1..258: note = Botrytis cinerea
MSSQPFTNKVIALTGSASGIGLETAKLLASRGARLSLADIQEDKLKELQAQLESEYYVDVITTKVDVRKFGEVEAWINKTIDNFGKLDGSANLAGVAPESIGLKGIVEQDLDEWEFVLGVNLTGTMNSLKAQLKVMANNGSIVNASSIRGLTGAAKNASYSSAKHGIIGLTRTAAKEVGGKGIRVNAICPGRISTPMLKTAENSIGLHLQPGSANYPPIALGRDGEAKEVAQLVAFLLSDESTYISGADISIDGGWRC

SEQ ID NO: 93 - protein, 692 aa, unidentified
  source 1..692: note = Myrothecium indicum
MAELDTLDILVLGAILLGTVAYFTKGKYWGVTKDPYASAFAASGGAKPAGTRNIVEKMEESGKNCIIFYGSQTGTAEDYASRLAKEGKSRFGLDTMVADLEEFDFDNLDTVPNDKIVMFILATYGEGEPTDNAVDFYEFITGDDVSFSESNDTPLANLNYVTFGLGNNTYEHYNSMVRNVDKALQKFGAHRIGDAGEGDDGAGTMEEDFLAWKDPMWSQLATKMGLEERDAVYEPVFSIVDREGLTPDSPEVYLGEPNKSHLDGKGQGPFNSHNPYIAPIAETRELFSVKDRNCIHLDIDISGSNLTYQTGDHIAIWPTNPGAEVDRFLDIIGLTEKRNQVISVKPLEPTAKVPFPTPTTYDAIIRYHLEVCAPMSRQFVSTLAAFAPNDEIKTEMTRLGSDKDYFHEKTSPHFYNIARFLEAVGKGQKWDNIPFSAFIEGLTKLQPRYYSISSSSRVQPKKVSVTAVVESQAIPGRDEPFRGVATNYLLALKQKQNGDPNPEPFGQSYELNGPRNKYDGVHVPVHIRHSNFKLPSDPAKPVIMIGPGTGVAPFRGFVQERAKLAEEGVEVGPTLLFFGCRKSTEDFMYKSEWEQYKKALGDKFEFITAFSRDGPNKVYVQHRLKEKAKEVNELLSKKAYFYVCGDAANMAREVNAVLAQIIAEGRGVSEAKGEEIVKNMRAANQYQEDVWS

SEQ ID NO: 94 - DNA, 1530 bp, Synthetic construct
  source 1..1530: mol_type = other DNA
atgtctaactccatcctgaacctgggttcatttgcgtgtctgctctctctgggatccatcgtcctctggtacaccatctccgctgtccttgcctggtatcctcttcgaaagatccctgccccctccttccttgcgaccttctcctacctgtggctcgccaagaccacctactctggaaagcagtattggattcaacgagatctgcataagaagtacggtcctctggtcagaatcggccccacagacattatcaccgacgacccagagatcattaagaagatctcctctgcgcgttccagccaccgacgaggagactggtacctgactggacgatttaacccgtattacgacaacatgttcaccatgctggagcctggtccccatgccaaagctaaggcacgaaccgctgccgcctactctggtcgagacatgcccgatctggaggttggtgttaacgcccagctccagaccctcatcggtctgatgcgaagcaagtacgcctccaacacggtgaagcctcatcagcctctgctggatcttggtcaggtttcttgtttcttcacgatggacgttattacccggctcgctttcggtgaggagttcggttaccttaaggaggagactgaccagtacggtttcctcggtgaagtccgagaactgtggccccgaatgtccacctctgctgataccccctggatccgtaagttcctcttctcccctcccttcctcaaggtcctcggtccaaagcccacggacaagaccggcttcggtgctctcatggccgtcgcggagcaccacgtcggcaagagattcgcacccgatgccaagaagaaggaggatatgctgggatccttcattcgacatggactcaaccagcaggaatgcgaagtcgagggactgttcatgattgtcgctggaaccgaatccactgccagcgctattcgatctaccctggtccacgtcatgacctgtcctcgagtttaccaaaagctgaagaccgagattaacctcgccgttgaagagggcaaagtctcctcgcccatcaagctggaggaggccaagctcctccccttcctccaagctgtcatctatgagggcattagaatgcgaccccctctcctcggccttttccccaagatcgtccccgatggcggtgaggagttccacggcatgttcatccctgctggaaccgccatctgcatgaacacctcctccctcctccgaagcaccgctctctttggtgacgacgctgaggtctaccggcctgaacgattcatggagctggagaagtccaagcggggtgagatggagcgaaacgtcgagctcgccttcggatacggtcagtatatgtgtgtcggcaagacggtcgccttcatggaacttaacaagtctatctttgaaatcctgcgtgcgttcgatctccagctcctgtcccccgccaagccgtgtgacgtcctctcatacggcatcttcctggagagcaacatgcttgtgaaggtcactgagagcgagggcacagagtacaagtaa

SEQ ID NO: 95 - DNA, 630 bp, Synthetic construct
  source 1..630: mol_type = other DNA
atggaattgtactggaaagcaatggagcagattgcctggtataccggcttatctcctacagctttctttactatactggcttcgatgatcttcgtgtttcaaatggtcagttccatgttcgtttctccggaagaatttaataagcccccaactgtcccagtttcctcatctaacccagctaactctaatctattcgtaaacgacagcgttgcggatgccagccaagctgttcaaattggtcaattgacggaacaacagttgagggcttacgatggatccgacccaaacaagccattacttatggctatcaaaggtcaaatttatgatgtctctagtggtagaatgttctacggtcctggtagtccatacgcaatgttcgccggcaaggacgcttcaagagctttggcattgctctcttttaagccagaagacatcaccggtaacattgaaggtttgtcagaagaggaattagttatcttacaagattgggagtacaagttcatggaaaagtatgttaaagtgggggaattggttctcgaaggtggtggtgaaggtatgaatgaacactctgagaacggccatcgtaattttgaaatcgatcaagaagaagaaagaattgaggctagataa

SEQ ID NO: 96 - DNA, 999 bp, Synthetic construct
  source 1..999: mol_type = other DNA
atgaccgccactatcacagcacagcaccgtaacggctgcggaccttccccacccaaaaagttggacattctgagagtaagtttccgaaagtatagaaaggctatagaacaaccaacggaggctatttcgagactaattgctaaaagagatcgctgtagagcgtttaggccatctatcagattcaagaaatcacattccaccgtgccaactactatcgtttacgatttgacctcttacttagacgaacaccctggtggtgatgacgttatcttggccgccactggtcgtgatgctacagacgattttgaagatgctggtcattctaaggatgctagagaacttatgcaaaagttctacattggtttattggacaccagctcttccgcttccccagagttggaaacgaaacaaccgagaggttatgctgcaagagtccaagggttgactaagcaatactgggcagctccagttgccatactcggtatttcattagccgtccaaaagaatactttgtacgtcggcggtctggctgaagaagttaacgaaagtatcttgcacgctgctttcattccttttggtgacattaaagacgttaagaccccgttagaccaagctacccagaagcatagatcattcgggtttgttactttcctagaaaaggaagatgcctctgcggctatggataatatggacggtgcggaattgtatggtcgtgtcttgactgtaaactacgccttgccagagagaatcaagggtggagaacaaggttgggctgcgcaaccaatctgggctgatgctgatacctggttcgagagacaacaacaagaagaagaaatgcagagaatacaagccgaaaaccgcgccgctatgcaagctgctgaagagctacacagaaaaaagcttgctcaggaaagagaaggtgaaaaagaagatgaaattgataccaaagacgacccgatggccaaggcagaagctgaggtcctacgacaaaatagttaa

SEQ ID NO: 97 - DNA, 924 bp, Synthetic construct
  source 1..924: mol_type = other DNA
atgcactctcgtaaccctagctccgtcgcctcagcatctccagagactcaggctttaaagcgaggaggcggtcccgaatttctgaccgtggctgttgcgcttgttgctattggtgtaggtacagcttactatttctacatctccaaaaagccaaagggttgcttggatccacataatttcaaagaatttaagctaatagaaagaactcaattgtctcacaacgttgccaagttcagattctcgttaccaaccccgacgagtgtcttgggtttgcctattggtcaacatatgtcttgttcatatatcattatctcaagttccactgaagagaaaaccgctaaggttaaaaagctatccaatagaaccttgcaaccaccatggattcaaatgcttgacgtcagtactagaaaggatgttcctcccttaagggaaatgagagtcggtgactacttggccgtcaaggggccaaagggacgctttaaatacaaggctaacgaagttgaagcttttggcatgttggcgggtggtactgggattacaccaatgttccaagttactagagctatattggaaaacccagtagatcgtacaaatgtgcacctgatctacgcaaacgtcacttatcaagacatcttattgaaggaggaattggattccttagctactagattcccgaccagattcaccatttattacgttctaaaccaaccaccagaaggttgggttggtggtgtgggttttgttacggaacaaatgttaagattgcactgtccaccacctgctccaaacgtacgtatcctaagatgtggtccaccaccaatgaataaagccatggctgcctacttgaatagcatcggttacactccccagatgcaattccagttcaatttgacctctccaccttctcccgctacatttatttaa

SEQ ID NO: 98 - DNA, 927 bp, Synthetic construct
  source 1..927: mol_type = other DNA
atgccttcaatctctacgctctactcgatacaggaggtatctcaacatagtacaaatgatgactgctggattgtgattgatggcaaggtttatgacttgacttcctacctagatgaacacccgggtggagatgacgttatcttagctgccaccggtcgtgatgcaaccgacgactttgaagatgctggtcactccaaagacgctagagaattgatgcaaaagttctatatcggtcttttagacactagctcttcagcgtcccccgaattggagactaagcaaccaagagggtacgccgctagggtccaaggtctgaccaaacaatactgggccgcaccagtcgctattttggaaaagactaccactaagaaaaacttgaaggaattgcaaaagtggagaattaacgcctctaatggtgctggatcttccgaaaagcatttcgttcggcgctggtttggtggtggtggtgaaagagtctatccaacatgttcactacataccttgagaagacaccagcgtagacaagacagtataggtagctctcacccagaggctcctttcctgagattacgtcacttcctaggcaaagaagatgcctccgcagcgatggataacatggatggtgctgaactttacggtagagttttaactgttaactacgctttgccagaaagaatcaagggtggtgaacaaggatgggctgcccaaccaatttgggctgacgctgatacatggtttgaaagacaacaacaagaagaggaaatgcaaaggatccaagccgaaaacagagctgctatgcaggctgccgaagaattgcatagaaaaaagttggctcaggaaagagagggtgagaaggaagatgaaattgacactaaagatgatccaatggctaaagcagaagctgaagttttacgacaaaattcttaa

SEQ ID NO: 99 - DNA, 2103 bp, Synthetic construct
  source 1..2103: mol_type = other DNA
atgaaggattttcaggacgagatcgcagccagtggtgctgtccctgctgctgaccacaaactactgcgtacagaacccgctgaatgcatggccgcatggagacaacgcagagctttggtgccacatcacgccaacgctgctgccgaattgtctgctagctcagttgttttcttgttcgttatagtagtctttgttggcgtcgttgtcgaaattttcttcgttgcgcattcttccatttctgatatcttaaccggttgtgacgttggtgatgattccgacccagtagtggattcgtctttgtccttttctagcagtcttttctcttcctgttctttttcctcatcatcggccgctagtgctgcggctgctgcattattgctccacaggttgttacttgttttgttgagactaccatctcggactttgttctcgccacctattagaagatgtttgtccgtcggacttggttgtagagtcttagttttaagattggaaagaggttctattcgaacttcacatagagctggtacgtctttaaatagaagatcatgttccgtatggaactcaggggaaatgttcggtgacggtaagcgtatcagcaccattctagggactgttgagacacaagcagaaccagcaagattttctcgctctttgtccttgaaggatttccaagtcctactgagcttgcctaaaagaccattgccagctttgggtagctcttcccgtatcatgtcttctcgcgtcagactgagaataagtgcggccagaccagttgctggtggtccgagtctattcaggcgacaaccaccgtctgtactgccaatcgaagctggacacagaaagaaaagaaagtgccaaatggaattggacgctggtatggcctccgctagaacttgggagttaggtagacgtaagaagaaaaagaaaatttttagaagcaagttcgaaaacttggaatctaagatgcaggatgaagaaaaaagaggtagtggttggtgtgagaatgctatcgcctccgcttctccaacccacacccaacactcaattgactgccggacgtccctaccactctttcatgccgcgactatgggcggtagatccgtttacactttggctgaagttgccagtcatgataatagaaacgattgttggttaattatagaaaacaaggtttatgatgttactaagttcttagaagaccatccaggtggcgacgaagtcttgttgtccgctatcggtaaggatgctaccaatgactttgacgatgtgggtcacagttccgctgctagagctatgatggatgaattttacgttggtgacatcgattcttctactattcccgccaagcgtaaatacacccctcctaagcaaccattgtataaccaagacaaaaccccagaatttattataaaggtacttcaattcttggtccctctagtttgttctcatagaccaagaaaaaatcttacaactcactgcggagcactcgtcgcgcaaaacaccggtttggaaccatgtactggcggggaaggtgctcatttcgaacagcacgaacaccaacccttggccagagcttctggtaatcaattggtttgtcgggcttcctcctgtctctctttgatcgatccagatgagccagttaaggttatcaagtcttctaacattcaactaggttcatacccctccttggaggtgaaaagtgcttcgaacagtgctatcaagcaccatagtacatctcagaagcctgaaaaagaaatagattggaatagccaattcttcaactggcaaaaggctgattattgtttaaacaacaatctgccagaattgttatacagcaaaagagaaaagttggaagctatttgctctgaacaagcttttgacgcaattgcctccatgcaaagactaactacaagcgcccaccgtcaagaaggtgttaactctcacatagcggccactctacaacacacgtttcaaggttcgtggagaagatactcacaagatatcttgcaattagccaaccaagactatccattgagaaccttgcaaagtttccaagacaaccaaattcataatattgctgctcaacatttcctcgaataa

SEQ ID NO: 100 - DNA, 642 bp, Synthetic construct
  source 1..642: mol_type = other DNA
atggccttgcaggtgtgggaaaccttaaaagaggctattgtagcttatacggggctttcccctgcaacatttttcactgtcctagctttgggtctggcggtttactacgttatctcaggtttctttggaacctctgattatggttctcacagtcgtgacttcggtgaacaaatgcaacccttaccaccaccagtccaagttggcgaaatatccgaagaggaattgaagcaatacgacggtagcgattcaaagaagccattgctaatggctatcaaaggccaaatttacgatgtctcgcaatctaggatgttctatggtcccggtggtccatacgccctctttgctggtaaggacgcttctcgagctttggcaaaaatgtccttcgaagaaaaggacttgactggtgatatttctggtttgggccctttcgaattagaagccttacaggattgggaatacaagtttatgtccaaatacgttaaggtcggtactgttgaaaagccagtttcagagacagatgctgcccctgctggtgaatctgcggagtctactaacgctgaagtctcgaaaccagcagaagacggtccttctgaatcccaagctgataaggctgaagagactgtagccgctaaggttgaataa

SEQ ID NO: 101 - DNA, 690 bp, Synthetic construct
  source 1..690: mol_type = other DNA
atgaactgcttggagttatgttgttggacgcgtgaagtggcatgaaatctgagactaaacatcttgagatggacagttaccatgacttctcacttcgtacgatgggtctatttgcctaccatgactgttatgagaaagatgattaaacgccagagaatactccatcaaatttaggtcttgcttccctaaggcatgaattgtagaatggccctattggttgtcaggatgttatttggtatgatcatttgccaaatgatatcattgccagcttcccggcaacgttgggttttatgtatcagcatgttgttgatttgtcaccttcaaaacaaccaagttcattgtcaccaaaagagatgtcgcaagatgctggaaagattgtgaaaaattcaggaaaacagagggttgttaagagctagtttgcaattggaacaaagattcctcctaaagagaggtatcgctaagtggataggtttatctttattagaaccaattttgatcttgcaagactgaaaagattcgcaaatcaaaggtctatttttgtgagtgaagttgaataacattagaccacgggccccttgcggacttttctgaagagtcgtttgtactatctctttgcacatttgaaacttcactctggaagtattgatctactcctgaagacagtgggagaagattgttcatctatactaa

SEQ ID NO: 102 - DNA, 477 bp, Synthetic construct
  source 1..477: mol_type = other DNA
atggttggaaaggccatgaggagaaatagagctcataaagatcgtgtcttcaactttttgctgccagaccctaaggtattcgtttttgatgaggttgcaaagcacaaccacttaagagactgctggatcataatttcaggcaaagtctatgatgttacaccattcttggaagaacatccgggtggtgacgaagtgcttctattagctgctgaaaaggatgcgactgacgattttgaaaatgtcggtcacagcttggctgccatcgaacaaatggagaagtactacgttggtaaagtcgacatgtccacggtcccaaagccaattgactatcggccccctgcttctcatccagctaccaccccatctgcccagagttctggttcgttgattcgattgcttcaattcttgatcccattattgattttaggtgttgctttctacttacaattttacgggaagaaacaacaatccaactaa

SEQ ID NO: 103 - DNA, 405 bp, Synthetic construct
  source 1..405: mol_type = other DNA
atgggctctggtgagaaagtgtttacactgaaggaagtcgctgaacataacaatcaaaaggactgctggctagtaatcagcggtaaggtttatgatgttaccaaattcttagacgatcacccaggtggagatgacgttttgctttcggcaactggtaaggatgcgactgacgatttcgaagacgtcggtcactccgataacgctcgtgaaatgatggaccagtactacgtcggtgagattgatgcttcaacgatacctaagaaagttgcctatgcccccccaaagcaaccacattacaatcaagacaaaacctccgaatttattatcaagttgttgcaattcttggttccgctcgctatcttagggttggctgttgccattagattctacactaagcaagcttaa

SEQ ID NO: 104 - DNA, 381 bp, Synthetic construct
  source 1..381: mol_type = other DNA
atggcctctgatccaaaggtgcaagtcttcgaagaggttgctaaacacaacaagcctaaaattgtctacgacgtaaccccatttatggaagatcatccaggaggtgacgaagttttactttccgcaactggtaaggatgctacaaatgacttcgaagatgttggccactcagattcggcgagagaaatgatggacaagtattacatcggtgaaatagaccccagcactgtcccgctgaagaaaatttacatcccaacccagcaaactcaatatacgcctgacaagactccagagtttgttataaagattttgcaaatcttggtcccaattttgatcttaggtttggctttcgctgttcgtcattacaccaagaacgaataa

SEQ ID NO: 105 - DNA, 630 bp, Synthetic construct
  source 1..630: mol_type = other DNA
atggaactttattggaaggctatggagcaaatagcctggtacacagggttatcgcctacggcattctttactattctagctagtatgattttcgtctttcagatggtgtcatctatgttcgttagcccagaagaatttaataaaccaccaaccgtacccgtttcctcttccaaccctgctaactctaatttgttcgtcaacgatagcgttgctgacgcctctcaagcggtccaaatcggtcaattgaccgaacaacaattgcgtgcttacgatggttccgacccaaacaagccattactgatggccatcaagggtcagatttatgatgtttcctctggaaggatgttctacggcccgggttcaccatacgctatgttcgccggtaaagacgcgtcgagagctctcgctttgttgtcctttaagccagaagatatcactggtaacattgagggtttgagtgaagaagaattggttatcttacaagactgggaatacaagttcatggagaagtatgtgaaagtcggtgaacttgtcttggaaggtggaggtgaaggtatgaatgaacattcagagaatggccacagaaactttgaaattgatcaggaagaagaaagaatcgaagctagataa

SEQ ID NO: 106 - DNA, 843 bp, Synthetic construct
  source 1..843: mol_type = other DNA
atggccttgcaggtatgggagactctaaaagaagcaattgtcgcttataccggtcttagtccagctacgttctttacagtgttggctctgggattagccgtttactacgtcatatctggtttctttggtacctcggactatggctcacattctcgtgatttcggggaacaaatgcaacctttgccaccgccagttcaagttggtgaaatctccgaagaggaattaaagcaatacgatggttctgacagcaagaagccattgttgatggctattaaaggtcaaatctacgacgtttcccaatctaggatgttttacggtccaggtggtccctatgcgcttttcgctggcaaggatgcctcgcgcgctttggctaagatgagttttgaagaaaaagacctgactggggatatttcaggattaggccctttcgaattggaagctctccaggattgggaatataagttcatgtctaagtacgtcaaagtgggtactgttgaaaagccagtttccgagacagacgcagccccagctggtgaatctgccgaaagtaccaacgctgaagtctccaaacctgctgaagatggtccaagcgaatcccaagctgacaaggcggaggaaactgtagccgctaagcaaaaaaaggaaaaaaagaagggtagaagaagagttgaaggtgtcctaatggaaatcagaggtgaagcggttggtgggagaaagggtgagggaatgtcaagagaaatcggtcggggtggtggtggtgctgatgatggtaatttgaaaggtggcggtattgaccacggtgaacgtgagcgagagagagaaagagagtctggtatgtcttaa

SEQ ID NO: 107 - DNA, 1164 bp, Synthetic construct
  source 1..1164: mol_type = other DNA
atggccgtatcaaattccagcaagaaactattactgtctctttttttcttcacctctattgttagattgaggcccaaggtcagtgttgacgcgaactcggtggatgacagattggttgtcacatgtaactccgatttgaaggatatattaccgcctccatactctgacctgtcctctatcacgtgctcaccaatttggcatcagactcaaaatgtcaccactatcgttttatcgggtaaacacgacaaccgttggatcggcattggttttagtagagaaggaaagatggttggttcctctgctgttgtcggttgggtagagagagatggtgtgtccggtttcagacaatattacttggaaggtaaggatatcagccaagtcattccagacaaaggtgatttacgatttactaacgcaaagcctgttgttgctgttcgtggcaatttgatctatattgctttccaattgcaatttccaacgtctttggctttccaaccaatcttgctcgccgtcggtgctggaaacccacttagaaacgggtctttgcctaagcacattaatagcacctctactttcattgaattttcatctgcacatttggctggtcagccaaccgacccaaacgaactcagaagataccacggcgctgccgcgataatcggttgggggattttaactccagctggtggtcttattccaagatacttccggcatttggaaccaagttggtactatttgcactcctctatccaatttgtaggtttcttcgttggtctaatatctatttctatcggcagaaacttgtaccaaaagattgacgctgtcttcgtcgcccataaatttataggtttctgtgtgttcattttggctggtctggaagtttgtcaattcgttggtagaccatccagtgattccaaattccgtcgttattggaattttgcacactactgggtgggaagaacagctatggttatgggtgtcgctaacgtcttcataggttttcatggtatcaaggccgaccggtctttaaagcttggatttgctggtgcgttcgctgctttgttaatgactgttattgttttggaaggtagattgcgcagagagaacgccatcccaaagaccatcgatgaaccccccgtttttcagctgactgaaaagagataa

SEQ ID NO: 108 - protein, 12 aa, Synthetic construct
DATEAFEDVGHS

SEQ ID NO: 109 - protein, 12 aa, Synthetic construct
DATDDFENVGHS

SEQ ID NO: 110 - protein, 12 aa, Synthetic construct
DATDDFEDVGHS

SEQ ID NO: 111 - protein, 12 aa, Synthetic construct
DATDDFEDAGHS

SEQ ID NO: 112 - protein, 12 aa, Synthetic construct
DATNDFDDVGHS

SEQ ID NO: 113 - protein, 42 aa, Synthetic construct
MALELWETLKEAITAYTGLSPATFFTVAALGLAVYYVVSGLF

SEQ ID NO: 114 - protein, 42 aa, Synthetic construct
MALQLWETLKEAITAYTGLSPAAFFTVVALLWAIYYVLSGMF

SEQ ID NO: 115 - protein, 42 aa, Synthetic construct
MALQVWETLKEAIVAYTGLSPATFFTVLALGLAVYYVISGFF

SEQ ID NO: 116 - protein, 267 aa, Mirabilis jalapa
MKGTYYINHGDPLMYLKKHIKLRQFLEGWQENVVIEKPKSILIISAHWDTNVPTVNFVEHCDTIHDFDDYPDPLYQIQYRAPGAPNLAKKVEELLKESGMECEIDTKRGLDHAAWFPLMFMYPEANIPICELSVQPSKDGIHHYNVGKALSPLLQQGVLIIGSGGTVHPSDDTPHCPNGVAPWAIEFDNWLEDALLSGRYEDVNNFKKLAPNWEISHPGQEHLYPLHVALGAAGKNPKTQLIHRSWAANGVFGYSTYNFTPTTQKTD

SEQ ID NO: 117 - protein, 501 aa, Beta vulgaris
MDNTTLALILSSLFVCFQLIRSFINHAKKSNKLPPGPKRMPIFGNIFDLGEKPHRSFANLAKIHGPLVSLQLGSVTTVVVSSADVAKEMFLKNDQALANRTIPDSVRAGDHDKLSMSWLPVSAKWRNLRKISAVQLLSTQRLDASQAHRQSKVQQLLEYVHDCSKKGQPVDIGRAAFTTSLNLLSNTFFSVELASHESSASQEFKQLMWNIMEEIGRPNYADFFPILGYLDPFGIRRRLAGYFDQLIAVFQDIIGERQKIRSANLSGGKQTTNDILDTLLNLYDEKELSMGEINHLLVDIFDAGTDTTASTLEWAMAELVKNPDMMVKVQDEIEQAIGKGCSMVQESDISKLPYLQAIIKETLRLHPPTVFLLPRKADADVELYGYVVPKNAQVLVNLWAIGRDPKVWKNPEVFSPERFLESNIDYKGRDFELLPFGAGRRICPGLTLAYRMLNLMMANFLHSYDWKLEDGMHPKDLDMDEKFGITLQKVKPLQVIPVPRK

SEQ ID NO: 118 - protein, 120 aa, Saccharomyces cerevisiae
MPKVYSYQEVAEHNGPENFWIIIDDKVYDVSQFKDEHPGGDEIIMDLGGQDATESFVDIGHSDEALRLLKGLYIGDVDKTSERVSVEKVSTSENQSKGSGTLVVILAILMLGVAYYLLNE

SEQ ID NO: 119 - protein, 769 aa, Panax quinquefolius
MWKLKVAQGNDPYLYSTNNFVGRQYWEFQPDAGTPEEREEVEKARKDYVNNKKLHGIHPCSDMLMRRQLIKESGIDLLSIPPVRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGATETRFITTGNGEKALQIMSWWAEDPNGDEFKHHLARIPDFLWIAEDGMTVQSFGSQLWDCILATQAIIATNMVEEYGDSLKKAHFFIKESQIKENPRGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAILGLMFGGQAERDPTPLHRAAKLLINAQMDNGDFPQQEITGVYCKNSMLHYAEYRNIFPLWALGEYRKRVWLPKHQQLKI

SEQ ID NO: 120 - protein, 486 aa, Panax ginseng
MAAAMVLFFSLSLLLLPLLLLFAYFSYTKRIPQKENDSKAPLPPGQTGWPLIGETLNYLSCVKSGVSENFVKYRKEKYSPKVFRTSLLGEPMAILCGPEGNKFLYSTEKKLVQVWFPSSVEKMFPRSHGESNADNFSKVRGKMMFLLKVDGMKKYVGLMDRVMKQFLETDWNRQQQINVHNTVKKYTVTMSCRVFMSIDDEEQVTRLGSSIQNIEAGLLAVPINIPGTAMNRAIKTVKLLTREVEAVIKQRKVDLLENKQASQPQDLLSHLLLTANQDGQFLSESDIASHLIGLMQGGYTTLNGTITFVLNYLAEFPDVYNQVLKEQVEIANSKHPKELLNWEDLRKMKYSWNVAQEVLRIIPPGVGTFREAITDFTYAGYLIPKGWKMHLIPHDTHKNPTYFPSPEKFDPTRFEGNGPAPYTFTPFGGGPRMCPGIEYARLVILIFMHNVVTNFRWEKLIPNEKILTDPIPRFAHGLPIHLHPHN

SEQ ID NO: 121 - protein, 678 aa, Panax ginseng
MAAMPTSLAMLVENQELLMLLTTSVAILIGCVVFLLWRRSSSQRSAKSLEPPKLVIPKIEPEEEVDDGKKKVTIFFGTQTGTAEGFAKAFSEEARARYEKAKFKVIDLDDYAGDDDEYESKLKKESLAFFFLATYGDGEPTDNAARFYKWFSEGKENREWLSNLQYGVFGLGNRQYEHFNKIAKVVDDGLAEQGAKRLVQVGMGDDDQCIEDDFTAWRELVWPELDQLLLDDEAAVATPYTAAVLEYRVVVHDRSDSSLLDRSLSMQNGHAVFDAQHPCRSNVAVTRELHTPASDRSCIHLEFDISQTGLAYETGDHVGVYCENLIETVEEAEKLLGISPATYFSIHTDKEDGTPLSGGSLPPPFPPCTLRTALTRYADLLSSPKKSALLALAAHASDPSEADRLRFLASPGGKDEYAQWLVANQRSLLEVLAEFPSAKPPLGVFFASVVPRLQPRYYSISSSPRMAPSRIHVTCALVYEKTPTGRIHKGVCSTWMKNAVSLEESHDCSWAPIFVRQSNFKLPSDTKVPILMIGPGTGLAPFRGFLQERLALKETGAELGPAVLYFGCRNRKLDFIYEEELNNFVDSGAISELVVAFSREGPTKEYVQHKMSQKASEVWNMISEGAYIYVCGDAKGMARDVHRMLHTIAQEQGALDSSKAESLVKNLQMTGRYLRDVW

SEQ ID NO: 122 - protein, 457 aa, Panax ginseng
MEREMLSKTHIMFIPFPAQGHMSPMMQFVKRLAWKGVRITIVLPAEIRDSMQINNSLINTECISFDFDKDDEMPYSMRAYMGVVKLKVTNKLSDLLEKQKTNGYPVNLLVVDSLYPSRVEMCHQLGVKGAPFFTHSCAVGAIYYNARLGKLKLPPEEGLTSVSLPSIPLLGRNDLPIIRTGTFPDLFEHLGNQFSDLDKADWIFFNTFDKLENEEAKWLSSHWPITSIGPLIPSMYLDKQLPNDKDNDINFYKADVGSCIKWLDAKDPGSVVYASFGSVKHNLGDDYMDEVAWGLLHSKYHFIWVVIESERTKLSSDFLAEVEAEEKGLIVSWCPQLEVLSHKSIGSFMTHCGWNSTVEALSLGVPMVAVPQQFDQPVNAKYIVDVWRIGVQVPIGENGVLLRGEVANCIKDVMEGEIGDELRGNALKWKGLAVEAMEKGGSSDKNIDEFISKLVSS

SEQ ID NO: 123 - DNA, 2307 bp, Synthetic construct
  source 1..2307: mol_type = other DNA
atgtggaaattgaaggttgctcaaggtaacgatccttatttgtactctactaataattttgttggtagacaatactgggagttccaacctgatgctggtactccagaagaaagagaagaagttgaaaaggctagaaaggattatgttaacaacaagaagttgcacggtattcatccatgttctgatatgttgatgcgtagacaattgatcaaggaatctggtattgatttgttgtccattccaccagttagattggatgaaaatgaacaagttaattatgatgctgttaccactgctgtcaagaaggctttgagattgaacagagctattcaagctcacgatggtcattggccagctgaaaatgctggttctttgttgtacactccaccattgattattgctttgtatatttctggtaccattgacactattttgaccaagcaacacaagaaggaattgatcagatttgtttacaaccatcaaaatgaagatggtggttggggttcttacattgaaggtcactctactatgattggttctgttttatcatatgttatgttgagattgttgggtgaaggtttggctgaatctgatgatggtaacggtgctgttgaaagaggtagaaaatggattttggatcatggtggtgctgctggtattccatcttggggtaagacttacttggctgttttgggtgtttatgaatgggaaggttgtaacccattgccaccagaattttggttattcccatcttctttcccattccatccagctaagatgtggatttactgtcgttgtacttacatgccaatgtcttacttgtacggtaagagataccacggtccaatcactgatttggttttgtctttgagacaagaaatttacaacattccatatgaacaaatcaaatggaaccaacaaagacacaactgttgtaaagaagatttgtattatcctcacactttggttcaagatttggtttgggatggtttgcattatttttctgaaccatttttgaagagatggccattcaacaagttgagaaagagaggtttgaagagagttgttgaattgatgagatacggtgctactgaaacaagatttatcactactggtaacggtgaaaaggctttgcaaatcatgtcttggtgggctgaagatccaaacggtgatgaatttaagcatcatttggctagaatcccagattttttatggattgctgaagatggtatgactgttcaatcatttggttctcaattatgggactgtattttggctactcaagctattattgctaccaacatggttgaagaatatggtgactctttgaagaaggctcacttcttcatcaaggaatctcaaatcaaggaaaacccaagaggtgatttcttgaaaatgtgtagacaattcaccaagggtgcttggactttctctgaccaagatcatggttgtgttgtttctgactgtactgctgaagctttgaaatgtttgttgttgttgtctcaaatgcctcaagatattgttggtgaaaagccagaagttgaaagattatatgaagctgtcaacgttttgttgtatttgcaatccagagtttctggtggttttgctgtttgggaaccaccagttccaaagccatatttggaaatgttgaacccatctgaaatttttgctgatattgttgttgaaagagaacatattgaatgtactgcttctgttatcaagggtttgatggctttcaaatgtttgcatccaggtcacagacaaaaggaaattgaagattctgttgccaaggctatcagatacttggaaagaaaccaaatgcctgatggttcttggtacggtttctggggtatttgtttcttgtacggtactttcttcactttgtctggttttgcttctgctggtagaacttacgacaactctgaagctgtcagaaagggtgtcaagtttttcttgtctactcaaaatgaagaaggtggttggggtgaatctttggaatcttgtccatctgaaaagttcactccattgaagggtaacagaaccaacttggttcaaacttcttgggccattttgggtttgatgtttggtggtcaagctgaaagagatccaactccattgcacagagctgctaagttgttgatcaatgctcaaatggacaacggtgatttcccacaacaagaaatcactggtgtttactgtaaaaattcaatgttgcattatgctgaatacagaaatattttcccattatgggctttgggtgaatacagaaagagagtttggttgccaaagcatcaacaattgaaaatt

SEQ ID NO: 124 - DNA, 1458 bp, Synthetic construct
  source 1..1458: mol_type = other DNA
atggctgctgctatggttttgtttttctctttgtctttgttgttgttgccattgttgttgttgtttgcttatttttcttacaccaagagaatcccacaaaaggaaaatgattccaaggctccattgccaccaggtcaaactggttggccattgattggtgaaactttgaactatttgtcttgtgttaaatctggtgtttctgaaaattttgtcaagtacagaaaggaaaaatattctccaaaggttttcagaacttctttgttgggtgaaccaatggctattttgtgtggtccagaaggtaacaagttcttgtactctactgaaaagaagttggttcaagtttggttcccatcttctgttgaaaagatgttcccaagatctcatggtgaatccaatgctgataatttttccaaggtcagaggtaagatgatgtttttgttgaaggttgatggtatgaagaaatatgttggtttgatggacagagtcatgaaacaatttttggaaactgactggaacagacaacaacaaatcaacgttcacaacactgtcaagaagtacactgtcaccatgtcttgtagagttttcatgtccattgatgatgaagaacaagttaccagattgggttcttccattcaaaatattgaagctggtttgttggctgttccaatcaacattccaggtactgctatgaacagagctatcaagactgtcaagttgttgaccagagaagttgaagctgtcatcaagcaaagaaaggttgatttgttggaaaacaagcaagcttctcaacctcaagatttgttgtctcatttgttgttgactgctaaccaagatggtcaatttttgtctgaatctgatattgcttctcatttgattggtttgatgcaaggtggttacaccactttgaacggtaccatcacttttgttttgaactatttggctgaatttccagatgtttacaaccaagttttgaaggaacaagttgaaattgctaactccaagcatccaaaggaattgttgaactgggaagatttgagaaagatgaaatattcttggaacgttgctcaagaagttttgagaattattccaccaggtgttggtactttcagagaagctatcactgatttcacttacgctggttacttgattccaaagggttggaaaatgcatttgattccacatgacactcacaagaacccaacttacttcccatctccagaaaaatttgatccaaccagatttgaaggtaacggtccagctccatacactttcactccatttggtggtggtccaagaatgtgtccaggtattgaatatgcaagattggttattttgattttcatgcataatgttgttaccaacttcagatgggaaaagttgattccaaatgaaaaaattttgactgacccaattccaagatttgctcacggtttgccaattcatttacatcctcacaac

SEQ ID NO: 125 - DNA, 2034 bp, Synthetic construct
  source 1..2034: mol_type = other DNA
atggctgccatgccaacttcattggctatgttagttgaaaaccaagaattattgatgttgcttaccaccagtgttgccattttaatcggttgtgtcgtgttcttattatggcgtagaagctcatctcaaaggagtgctaagtctttggaaccaccaaaacttgtgattccaaagatcgaaccagaagaagaagtcgatgatggtaagaagaaggtcaccatcttcttcggtactcaaaccggtactgccgaaggtttcgctaaggccttttctgaagaagctagagcaagatacgaaaaagctaagtttaaagttattgacttggatgattacgctggtgacgatgacgaatatgaatctaagttaaagaaggaatctttggcattcttcttcttggcaacatacggtgatggtgaacctactgataatgccgctagattttacaagtggttttctgaaggtaaggaaaatagggaatggttgtctaatttacaatatggagtttttggtctaggtaataggcaatacgagcactttaacaaaattgccaaggtcgttgacgatggtctggccgaacaaggtgctaaacgtttagtgcaagtgggaatgggtgacgacgatcaatgtatcgaagatgattttaccgcttggagagaattggtttggccagagttggatcaattgttgttggacgatgaggctgctgtggccactccatacacggctgctgttttggaatatagagttgttgtacacgacagatcagactcctctttattggatagatctctgtccatgcaaaacggtcacgccgttttcgacgctcaacacccttgtagatctaacgttgccgttacaagagaattgcatactccagcttctgacagaagttgtattcatttggaatttgacatctcgcaaaccggtttggcctatgaaactggtgaccatgtcggcgtttattgtgaaaatttgattgaaactgttgaggaagctgaaaaactattgggtatctcgccagccacatatttcagcattcatactgacaaggaagatggtaccccattatctggtggttcattgccaccaccattcccaccatgtactctgcgtacagcccttactcgttacgccgatttgctttcttctccaaagaagtccgctttgttagccttggctgctcatgcttctgatcctagcgaggcagatagattgagatttttggcttctccaggtggtaaggatgaatatgcccaatggttggtagccaatcaacgttcattgttagaagttttggctgaatttccatccgccaagccacccttgggtgttttctttgcttctgttgtaccaagattacaaccaagatactactctatatcttcttctccaaggatggctccatcaagaatccacgtgacctgtgctttggtatacgaaaagaccccaacgggtagaatccataagggtgtttgttccacctggatgaaaaatgctgtttccttggaagagagtcacgattgcagttgggctcctatcttcgttagacaatctaactttaaactgccttccgatacaaaagtacctatcttgatgattggtcctggtactggcttggctccttttagaggttttttacaagaaagattagctttgaaggaaactggtgctgaattaggtccagctgtcttatacttcggttgcagaaacagaaagttggacttcatctatgaagaagaattgaacaacttcgtcgactctggtgccatctctgaattggttgtcgctttctccagagaaggtcccaccaaagagtatgtacaacacaagatgtcacaaaaggcctctgaagtttggaatatgatctctgaaggtgcatatatctacgtctgtggtgatgcaaaaggaatggcccgtgatgtacacagaatgttgcacaccattgcccaagaacaaggtgccctagactcttccaaggcagagtctttggtcaagaacttgcaaatgaccggtagatacttaagagatgtatgg

SEQ ID NO: 126 - DNA, 1371 bp, Synthetic construct
  source 1..1371: mol_type = other DNA
atggaacgtgaaatgttatctaagacccatattatgtttattccatttccagctcaaggtcacatgtcccctatgatgcaattcgtcaagagactggcctggaagggtgtaagaatcactattgtcctgccagctgagatccgtgattccatgcaaataaataactccttgattaacaccgaatgcatctctttcgatttcgacaaggatgatgaaatgccatactccatgagggcttacatgggtgttgtcaaattaaaggttactaataagttgtccgacttgttagaaaagcaaaaaactaatggttaccctgtcaatttgttggtcgttgacagcctttatccaagcagagttgaaatgtgtcatcagttaggagtgaagggtgccccattcttcacccattcttgtgctgttggtgccatttattacaacgctagattgggtaagttgaaattaccaccagaggaaggtttgacttccgttagcttgccatctattcctttgctaggtagaaacgacttacccatcataagaactggcacctttccagacctttttgaacacttgggtaaccaattctcagacttggataaggccgattggatctttttcaatacgttcgataagttggaaaatgaagaagctaagtggttgtcctcccattggccaatcacatctattggtcccctaatccctagtatgtatttggataagcaacttccaaatgataaagacaacgatataaacttctacaaggctgacgtcggatcttgtattaaatggttggatgctaaggacccaggtagtgtggtctatgcttctttcggcagcgttaagcacaacttaggtgatgattacatggatgaagtcgcttggggtttattgcactccaagtatcacttcatctgggttgttattgaatccgaaagaaccaagttatcttccgattttttggctgaagtcgaagccgaggaaaaaggtttgattgttagttggtgtccacaattggaagttcttagtcacaaatcgattggttctttcatgactcactgtggttggaactccaccgtcgaagctttaagtctgggtgttccaatggtcgctgttccacaacaattcgatcaaccagtcaacgctaaatacatcgtggatgtttggagaatcggagttcaagtccctataggtgaaaatggtgttctattaagaggtgaggttgccaactgcatcaaggatgttatggaaggagaaatcggtgacgaattgagaggtaacgccttgaaatggaagggcttggctgtagaggccatggaaaaaggtggaagcagtgacaaaaatatcgacgaatttatctctaagttagtatcttcc

SEQ ID NO: 127 - DNA, 1674 bp, Synthetic construct
  source 1..1674: mol_type = other DNA
atgttcatcttggtcttgctcacaggtgctttaggcctggctgctttctcatgggcatccatagcgttcttcagtctttacctcgctccgaggcgatcttcactgtataaccttcagggcccgaatcataccaactactttacaggcaattttttagacatcctctcagctcgtacaggtgaagagcatgcgaagtacagagaaaaatacggaagcaccctccggtttgctgggatcgctggagcacccgtcttgaactcgaccgatccgaaagtcttcaaccatgtgatgaaagaagcctacgactatccgaaacctggtatggccgctcgagtgctcagaattgctaccggagatggtgttgttacggcggaaggtgaagctcataagcgacatcgaaggatcatgatcccctctctgtccgctcaggccgttaagtcgatggtcccaattttcttagaaaaaggtatggaacttgtcgacaagatgatggaggatgcggctgagaaggatatggccgtgggagagtcggccggtgaaaagaaggcaaccagactcgagaccgaaggagtcgatgtaaaggattgggtcggtcgagctactctggacgtcatggctcttgcaggatttgactataagagcgactcgctccagaacaagaccaatgagctctatgtcgcttttgtcggacttaccgatgggtttgctcctaccttggactcgttcaaggctatcatgtgggattttgtaccttacttccgaactatgaaacggagacatgagatacctttgactcaaggattagcagtttcccgacgagttgggatcgagcttatggagcaaaagaagcaggccgtgcttggctcagcttccgatcaggctgttgataaaaaggatgttcaaggtcgggatatcctaagtctcctagtgagagcaaacatcgccgccaacctgcctgaatctcaaaagctgtccgatgaggaggtactcgctcagatcagtaacctgttatttgctggatatgaaacttcttcgacagtcttgacatggatgtttcaccgactctcagaagacaaagccgttcaggataaacttcgagaagaaatttgtcagatcgacacggatatgcctacgctagacgaacttaatgcgttgccttatctcgaagcgtttgttaaggagtctcttcgtctagaccctcctagtccgtatgctaaccgtgaatgcttaaaggatgaagacttcatcccacttgccgagcctgtcattggtcgagatgggtcggtcatcaacgaggtccggatcacgaaaggaacgatggtcatgcttccgttgttcaacatcaatcgttcaaagttcatttatggagaagatgcagaagaattcagaccggagaggtggcttgaggacgtaacagactcgctcaacagtattgaagcaccctatggacaccaggcgagctttatctctggacccagagcttgctttggttggcgatttgctgtcgccgagatgaaggccttcttgtttgtcactctccgtcgggtccagttcgagcccatcatctctcatccagagtacgagcacatcaccttgatcatttcccgtcctcgaatcgttggtagagagaaggaggggtaccagatgcgtttgcaggtcaagccggtcgaatga

SEQ ID NO: 128 - protein, 557 aa, unidentified
  source 1..557: note = Phaffia rhodozyma
MFILVLLTGALGLAAFSWASIAFFSLYLAPRRSSLYNLQGPNHTNYFTGNFLDILSARTGEEHAKYREKYGSTLRFAGIAGAPVLNSTDPKVFNHVMKEAYDYPKPGMAARVLRIATGDGVVTAEGEAHKRHRRIMIPSLSAQAVKSMVPIFLEKGMELVDKMMEDAAEKDMAVGESAGEKKATRLETEGVDVKDWVGRATLDVMALAGFDYKSDSLQNKTNELYVAFVGLTDGFAPTLDSFKAIMWDFVPYFRTMKRRHEIPLTQGLAVSRRVGIELMEQKKQAVLGSASDQAVDKKDVQGRDILSLLVRANIAANLPESQKLSDEEVLAQISNLLFAGYETSSTVLTWMFHRLSEDKAVQDKLREEICQIDTDMPTLDELNALPYLEAFVKESLRLDPPSPYANRECLKDEDFIPLAEPVIGRDGSVINEVRITKGTMVMLPLFNINRSKFIYGEDAEEFRPERWLEDVTDSLNSIEAPYGHQASFISGPRACFGWRFAVAEMKAFLFVTLRRVQFEPIISHPEYEHITLIISRPRIVGREKEGYQMRLQVKPVE

SEQ ID NO: 129 - DNA, 1000 bp, Synthetic construct
  source 1..1000: mol_type = other DNA
tgttgttgttagtgttgtccctgttcccggtcgtagatacgccgctagagagcatgcatatacagatgtgtttgagatgaacaaaggtgagagttggtccgggaggaacaatggggtcacacgttcaagataagcttggatagataaccgtaactcacgcagggcaagaattaaacataatatttttggactgtaaattcatcattctggaaatcgaacagagctgaagacggggaaggtgatgttgaggaaggagggaaggctaaagagcaaagctgaagaaggtatgcttggaggcttggtattgaacgttgtggttagttgatagacggtatccaagggggaacgagtgggcgaactggagtgttggccgagtatcagaatggcacatgtaacaggcgtagagagtataacgaacgtaacaggagggataccgagccaacgttcgacggggtgatcacacgtggggtgcgtcgggttaccatcgggggtacattgggggtgaacgggggtttgcatggggctcgcgcattcggacgcggtaacatgcaacagttgtaacatctggtgtacctgaggcggatgtttacattacgtaagtgcgttgagccagctgaatttcaggttgttctcgattttgaaagaattttgaaattcatcctttccttcaaattcttgacctcccatgacctttgtccccggaatctgacctttctagaaagcccaggcaaatcgatcagcttcccaggtgcagacacttgtactccccaagcaagttcctcagcaaacaagtgagccctttgatctgctatgttgcaagtctcgagagaaaacaagatgctatatgttatacaggcaacataagaaaccttaggaaaatcgatgctgacagagcggatcgttttctgcacttcaactagcttcttaggtcgacccgagtccgagacctaatctttccgccttcaccttaaaccctttttaaacactccctttattcaaa

SEQ ID NO: 130 - DNA, 413 bp, Synthetic construct
  source 1..413: mol_type = other DNA
gtgtgtatgtgtgtagtgtagttgggttgtcccatgtgcttcttcttatcatcatcatttctttaaaatctctacattgaatgtttaccggaacgggctttgatgatactacggaccacgttgtgtaaccagttcgattgagattacgattagatagccgatccgtcgatcagatctcgatctagagcgacatctggctcgatcggtccttgccgaaaatcagggcaccgatcagggcagaggaacgccgaggccgaacgagacagacacaccatcatcatcagccatgtcttttttgtgatcgtttttacatactacccgtcgattctaaccttctttcttcttctcttgccatctttgcattctctatctcgtgtaacatcgatccgattcttgccacctactttctccat

SEQ ID NO: 131 - DNA, 499 bp, Synthetic construct
  source 1..499: mol_type = other DNA
gcaagcgatgtgattttcaatacaatcccggactacaagctctctaatagagctctataatagaaggacaaaagtcgtcccactcctatctcccgcgcgttttaatagagccgattgtttttttccctaatgttttattttctttccccgatcggctcatttttcttctctccgcgtattcttcacacaacgctccctccatcttttttcttcttgttcctgttcctcttcgtctccttccattgtcttctttccttccttccttccttcttgcctctagccagcttcaacagcgacgtctctctctctctgtgtggtgatctccgactgtagtgtctctctcggtcactttcacgaatcaacttcgtttcttttctgatcgatcggtcgtctttccctcaatccgtgcatacactcacacttacactcacacccacacactcaaacacgctaaataatcagatccgtctccccttcttgatctccttcggcttaggca

SEQ ID NO: 132 - DNA, 1108 bp, Synthetic construct
  source 1..1108: mol_type = other DNA
cttgacttattagcatgctctgacgctggaagagcgtatatgtaccgtcatcccgccggtcaaacacggatatgcaaatgttaaggctatacgaggagacgttcctatgacagaagatggtccaccgatagtggtttgaaaggagatgggtggaatagagacaaatggggaattccagttttgcctttgacgagaaaggacactgggttggaaagagaagatggtacgttcttctccaccttgaatgtgttgcttactagacatgtttgacacgctaatgcatttctttccactttgacttttgaactatggtggttgggcgatccccaaaatcattagcttctacttcagctcattacctcgatctcatcttactaccaggtgttgcattctcacctacggcctcttctttgttctctcgactgggccatggaaaaggatattacgataaatacatcactcagtatcggtcgatctgtgcaggcaagaatcgacccgtccgaagctgagtacgcgtcttctcttttctcgatacccaacggacgctattttgtgacagaaggatgagactatccaacagctcaaacaaactaacgctcttgattaatcacccgctcaacttattgctcaactcagttggactggcgctgaaagaacagttcttagacaaaaacatggtccctataggagaatgggatgcgaatctggatgaagtgttggttggagatcacgtgaggacattatccgaggacaattaactacttaagatatatacatgatttatgtcgatcggcatccagccggggattgatcggctgatggccggaaatgtgatgatggtcgaaactcgatctctctttttttgttcatcttctcatccctcttctctctttctactgacatccatctccaactgtctagatcagttcggaaacaagaagtggacacagagagatctttgctgaagagttgtattccagaaagggaaaacaaaggaaagaagcgccgaagcacatcaccaacttcagcaagccggtccagcccgatctcggatagacatcatcttacccaactcgtatcatccccaacagatagagtttttgtcgca

SEQ ID NO: 133 - protein, 42 aa, Synthetic construct
MAVELWETLKDSITAYTGLAPTTFFTLVALALAFYYVVSELF

SEQ ID NO: 134 - protein, 42 aa, Synthetic construct
MALQQWDAWKESIIESTGLSPATIFTIVALAVAFYYAISGFS

SEQ ID NO: 135 - protein, 26 aa, Synthetic construct
MAPLQLWETFKEAITAYTGLSPATFF

SEQ ID NO: 136 - protein, 43 aa, Synthetic construct
MALQLWETLKEAIEAYTGLSPNTFFTLLALILALYYVVSGLFW

SEQ ID NO: 137 - protein, 42 aa, Synthetic construct
MALQLWETLKEAIVAYTGLSPPTFFTLLALILALYYVVSGLF

SEQ ID NO: 138 - protein, 42 aa, Synthetic construct
MAVQLWETLKEAIVAYTGFSPSTFFTVLALLFAVYYVVTGLF

SEQ ID NO: 139 - protein, 42 aa, Synthetic construct
MAVQLWETLKEAIVAYTGFSPSTFFTVLALLFAAYYVVTGLF

SEQ ID NO: 140 - protein, 42 aa, Synthetic construct
MALELWQTLKEAIHAYTGLSPVVFFTALALAFAIYQVISGWF

SEQ ID NO: 141 - protein, 42 aa, Synthetic construct
MVQQIWETLKETITAYTGLSPAAFFTVLALAFAVYQVVSGFF

SEQ ID NO: 142 - protein, 43 aa, Synthetic construct
VSGGMISKKVAELWAAARSASPVAVIAAVAGAAVVYKVGSSLL

SEQ ID NO: 143 - protein, 43 aa, Synthetic construct
MAVAELWETLKQSIVAYTGLSPAAFFTAVAVAAALYHVVSGLF

SEQ ID NO: 144 - protein, 43 aa, Synthetic construct
MAVVELWETLKQAIVAYTGLSPAAFFTAVAVAAALYHVVSGLF

SEQ ID NO: 145 - protein, 41 aa, Synthetic construct
MVAAAEWWEATIAAYTGLSPAAFFTAVAVAAALYVAVSGLL

SEQ ID NO: 146 - protein, 40 aa, Synthetic construct
MAFVNATLALIPQYTGLSAGTFFTIIGLVVGIYYLILGLF

SEQ ID NO: 147 - protein, 47 aa, Synthetic construct
MAGLDFVYGYWNATLSSIPQYTGLSASTFFTVVALVFGIYYLISVLF

SEQ ID NO: 148 - protein, 44 aa, Synthetic construct
MEFLSAFLNAISFYIPQYTGLSPITFFTIAALVCGIYFLISGLF

SEQ ID NO: 149 - protein, 36 aa, Synthetic construct
MEIAEALQAYTGLTPAAAATILALMVATYLLVSSLF

SEQ ID NO: 150 - protein, 39 aa, Synthetic construct
MAAQGIAEAVQAYTGLSPAAAVTILALMLATYLLVSSLF

SEQ ID NO: 151 - protein, 40 aa, Synthetic construct
MAFYSVFLEAIPAYTGLSPVAFFTTLALMVGVYHLVCALF

SEQ ID NO: 152 - protein, 41 aa, Synthetic construct
MVAFFAAVMDTVTTYTGLSPAAFFTILALMCVVYKTVCSMF

SEQ ID NO: 153 - protein, 40 aa, Synthetic construct
MGLYATAMETMEAYTGLSPAAFFTIAAVMVVVYKIVCSMF

SEQ ID NO: 154 - protein, 40 aa, Synthetic construct
MGLYTSVMEEISFYTGLSPAAFFTILAMMVVVYRTVSGMF

SEQ ID NO: 155 - protein, 41 aa, Synthetic construct
MATVWATTTEIIFEYTGLSATAFFTILAMMLVTYKVVCSMF

SEQ ID NO: 156 - protein, 40 aa, Synthetic construct
MELYWKAMEQIAWYTGLSPTAFFTILASMIFVFQMVSSMF

SEQ ID NO: 157 - skipped

SEQ ID NO: 158 - protein, 500 aa, Mirabilis jalapa
MTAIKMNTNGEGETQHILMIPFMAQGHLRPFLELAMFLYKRSHVIITLLTTPLNAGFLRHLLHHHSYSSSGIRIVELPFNSTNHGLPPGIENTDKLTLPLVVSLFHSTISLDPHLRDYISRHFSPARPPLCVIHDVFLGWVDQVAKDVGSTGVVFTTGGAYGTSAYVSIWNDLPHQNYSDDQEFPLPGFPENHKFRRSQLHRFLRYADGSDDWSKYFQPQLRQSMKSFGWLCNSVEEIETLGFSILRNYTKLPIWGIGPLIASPVQHSSSDNNSTGAEFVQWLSLKEPDSVLYISFGSQNTISPTQMMELAAGLESSEKPFLWVIRAPFGFDINEEMRPEWLPEGFEERMKVKKQGKLVYKLGPQLEILNHESIGGFLTHCGWNSILESLREGVPMLGWPLAAEQAYNLKYLEDEMGVAVELARGLEGEISKEKVKRIVEMILERNEGSKGWEMKNRAVEMGKKLKDAVNEEKELKGSSVKAIDDFLDAVMQAKLEPSLQ

SEQ ID NO: 159 - protein, 497 aa, Beta vulgaris
MDHATLAMILAIWFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTFEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 160 - protein, 489 aa, unidentified
  source 1..489: note = Cleretum bellidiforme
MGTHSTAPDLHVVFFPFLAHGHMIPSLDIAKLFAARGVKTTIITTPLNASMFTKAIEKTRKNTETQMEIEVFSFPSEEAGLPLGCENLEQAMAIGANNEFFNAANLLKEQLENFLVKTRPNCLVADMFFTWAADSTAKFNIPTLVFHGFSFFAQCAKEVMWRYKPYKAVSSDTEVFSLPFLPHEVKMTRLQVPESMRKGEETHFTKRTERIRELERKSYGVIVNSFYELEPDYADFLRKELGRRAWHIGPVSLCNRSIEDKAQRGRQTSIDEDECLKWLNSKKPDSVIYICFGSTGHLIAPQLHEIATALEASGQDFIWAVRGDHGQGNSEEWLPPGYEHRLQGKGLIIRGWAPQVLILEHEATGGFLTHCGWNSALEGISAGVPMVTWPTFAEQFHNEQLLTQILKVGVAVGSKKWTLKPSIEDVIKAEDIEKAVREVMVGEEGEERRRRAKKLKEMAWRAIEEGGSSYSDLSALIEELKGYHTSEKE

SEQ ID NO: 161 - protein, 280 aa, unidentified; identical to SEQ ID NO: 2

SEQ ID NO: 162 - protein, 45 aa, synthetic construct
MAAAVAELWETLKQAIVAYTGLSPAAFFTAVAAAAALYHVVSGIF

SEQ ID NO: 163 - protein, 45 aa, synthetic construct
MAMAMAEWWAAATEAVAVYTGLTPTAFFTAVAVAAALYVAVSGLF

SEQ ID NO: 164-246 - skipped (83 entries)

SEQ ID NO: 247 - protein, 759 aa, unidentified
  source 1..759: note = Siraitia grosvenorii
MWRLKVGAESVGENDEKWLKSISNHLGRQVWEFCPDAGTQQQLLQVHKARKAFHDDRFHRKQSSDLFITIQYGKEVENGGKTAGVKLKEGEEVRKEAVESSLERALSFYSSIQTSDGNWASDLGGPMFLLPGLVIALYVTGVLNSVLSKHHRQEMCRYVYNHQNEDGGWGLHIEGPSTMFGSALNYVALRLLGEDANAGAMPKARAWILDHGGATGITSWGKLWLSVLGVYEWSGNNPLPPEFWLFPYFLPFHPGRMWCHCRMVYLPMSYLYGKRFVGPITPIVLSLRKELYAVPYHEIDWNKSRNTCAKEDLYYPHPKMQDILWGSLHHVYEPLFTRWPAKRLREKALQTAMQHIHYEDENTRYICLGPVNKVLNLLCCWVEDPYSDAFKLHLQRVHDYLWVAEDGMKMQGYNGSQLWDTAFSIQAIVSTKLVDNYGPTLRKAHDFVKSSQIQQDCPGDPNVWYRHIHKGAWPFSTRDHGWLISDCTAEGLKAALMLSKLPSETVGESLERNRLCDAVNVLLSLQNDNGGFASYELTRSYPWLELINPAETFGDIVIDYPYVECTSATMEALTLFKKLHPGHRTKEIDTAIVRAANFLENMQRTDGSWYGCWGVCFTYAGWFGIKGLVAAGRTYNNCLAIRKACDFLLSKELPGGGWGESYLSCQNKVYTNLEGNRPHLVNTAWVLMALIEAGQAERDPTPLHRAARLLINSQLENGDFPQQEIMGVFNKNCMITYAAYRNIFPIWALGEYCHRVLTE

SEQ ID NO: 248-263 - skipped (16 entries)

SEQ ID NO: 264 - DNA, 1563 bp, Synthetic construct
  source 1..1563: mol_type = other DNA
atggaaatgtcctcaagtgtcgcagccacaatcagtatctggatggtcgtcgtatgtatcgtaggtgtaggttggagagtcgtaaattgggtttggttgctgccaaagaaattggaaaagagattgagagaacaaggtttggccggtaattcttacagattgttgttcggtgacttgaaggaaagagctgcaatggaagaacaagcaaattcaaagcctataaacttctcccatgacatcggtccaagagttttcccttcaatgtacaagaccatccaaaactacggtaaaaactcctacatgtggttaggtccataccctagagtccacatcatggatccacaacaattgaagaccgtttttactttggtctacgacattcaaaagccaaatttgaaccctttgattaaattcttgttagatggtatcgttacacatgaaggtgaaaagtgggctaagcacagaaagattattaacccagcattccatttggaaaagttgaaggatatgatacctgctttctttcactcatgtaatgaaatcgtcaacgaatgggaaagattgatttcaaaagaaggttcctgcgaattggatgtaatgccttatttgcaaaatttggccgctgacgccatttcaagaaccgcttttggttcttcatacgaagaaggtaaaatgatcttccaattgttgaaggaattgactgatttggttgtcaaggtagcttttggtgtttatattccaggttggagattcttgcctacaaagagtaacaacaaaatgaaggaaattaatagaaaaatcaagtctttgttgttgggtatcattaacaagagacaaaaggcaatggaagaaggtgaagccggtcaatctgatttgttgggtatattaatggaaagtaattctaacgaaatccaaggtgaaggtaataacaaggaagatggcatgtctattgaagacgtcatcgaagagtgtaaggtattttatataggtggtcaagaaactacagcaagattattgatctggactatgatattgttgtccagtcatacagaatggcaagaaagagccagaaccgaagtcttgaaggtatttggtaataagaaaccagatttcgacggtttgtcaagattgaaggtagttactatgatcttgaacgaagttttaagattgtacccacctgcttccatgttgacaagaatcatccaaaaggaaacaagagttggtaaattaaccttgccagcaggtgttatcttgataatgcctatcatcttgatacatagagatcacgacttgtggggtgaagatgctaacgagtttaaaccagaaagattcagtaaaggtgtttctaaggcagccaaagtccaaccagcctttttcccttttggttggggtcctagaatttgcatgggtcaaaacttcgctatgatcgaagctaagatggcattgagtttgatcttgcaaagattttctttcgaattgtcttcatcctacgttcatgcaccaactgtcgtcttcactacacaaccacaacacggtgcccacatcgttttgagaaagttataa

SEQ ID NO: 265 - DNA, 1422 bp, Synthetic construct
  source 1..1422: mol_type = other DNA
atgtggactgtcgtgctcggtttggcgacgctgtttgtcgcctactacatccattggattaacaaatggagagattccaagttcaacggagttctgccgccgggcaccatgggtttgccgctcatcggagagacgattcaactgagtcgacccagtgactccctcgacgttcaccctttcatccagaaaaaagttgaaagatacgggccgatcttcaaaacatgtctggccggaaggccggtggtggtgtcggcggacgcagagttcaacaactacataatgctgcaggaaggaagagcagtggaaatgtggtatttggatacgctctccaaatttttcggcctcgacaccgagtggctcaaagctctgggcctcatccacaagtacatcagaagcattactctcaatcacttcggcgccgaggccctgcgggagagatttcttccttttattgaagcatcctccatggaagcccttcactcctggtctactcaacctagcgtcgaagtcaaaaatgcctccgctctcatggtttttaggacctcggtgaataagatgttcggtgaggatgcgaagaagctatcgggaaatatccctgggaagttcacgaagcttctaggaggatttctcagtttaccactgaattttcccggcaccacctaccacaaatgcttgaaggatatgaaggaaatccagaagaagctaagagaggttgtagacgatagattggctaatgtgggccctgatgtggaagatttcttggggcaagcccttaaagataaggaatcagagaagttcatttcagaggagttcatcatccaactgttgttttctatcagttttgctagctttgagtccatctccaccactcttactttgattctcaagctccttgatgaacacccagaagtagtgaaagagttggaagctgaacacgaggcgattcgaaaagctagagcagatccagatggaccaattacttgggaagaatacaaatccatgacttttacattacaagtcatcaatgaaaccctaaggttggggagtgtcacacctgccttgttgaggaaaacagttaaagatcttcaagtaaaaggatacataatcccggaaggatggacaataatgcttgtcaccgcttcacgtcacagagacccaaaagtctataaggaccctcatatcttcaatccatggcgttggaaggacttggactcaattaccatccaaaagaacttcatgccttttgggggaggcttaaggcattgtgctggtgctgagtactctaaagtctacttgtgcaccttcttgcacatcctctgtaccaaataccgatggaccaaacttgggggaggaaggattgcaagagctcatatattgagttttgaagatgggttacatgtgaagttcacacccaaggaatga

SEQ ID NO: 266 - DNA, 2106 bp, Synthetic construct
  source 1..2106: mol_type = other DNA
atgaaggtgtcgccttttgagttcatgagtgccataattaaaggcagaatggacccgagcaattcatcttttgaatccacaggagaagtagcatctgtcatctttgagaaccgagaattggttgcgattttaaccacgtcaatagctgttatgattgggtgtttcgtggttcttatgtggcgtagggctggttccagaaaagtcaagaatgtcgaactacccaaaccactgatcgttcacgaaccagagcctgaagtagaagatggtaagaaaaaggtaagcatcttctttggtactcagacaggaactgccgagggcttcgcaaaagctttggcagatgaagctaaggcgcgctatgaaaaagccacttttagagttgtcgacctcgatgattacgcggctgacgatgaccaatatgaagagaaattaaaaaacgagtctttcgcagtatttctattggcaacatacggtgatggtgaaccaaccgataatgctgcaaggttctataagtggtttgccgaagggaaagagcgtggcgaatggttacaaaacttacattacgctgtttttggattgggtaatcggcagtatgaacattttaataaaattgcaaaggtggccgatgagcttttagaagctcaaggtgggaacagactcgtaaaagttggcctgggggatgacgatcaatgcatagaagacgatttctcagcttggagagaaagtttgtggcccgaacttgacatgctcttaagagatgaagatgatgctacaactgttacgaccccttacactgctgccgtcttggagtatcgagtggtgttccacgatagcgcagatgtcgctgcggaggacaagtcttggattaatgccaacggtcatgctgttcacgacgctcaacatcctttccgctctaatgtagttgtgagaaaagaactgcatacatccgcctctgatagaagttgttcgcatttagaatttaacatatcaggctctgcactaaattatgagactggagatcatgtaggtgtttactgcgaaaaccttacagaaaccgtagatgaagcattgaatttgctaggtctttccccggaaacctatttttcaatctatactgataacgaggacggaacaccactaggaggttcctcactaccccctccatttcccagctgtacgctaaggacggcattaacaaggtacgcggacttgttgaatagtccaaagaagagtgcacttctggctttagccgctcacgcatccaatccagttgaagcggatcgtttaagatacctcgcttccccagcagggaaagatgaatatgcccaatctgtgattggtagccagaaaagtttactcgaggtcatggcagagtttccctcagcgaagcctcctttgggcgttttctttgctgcggttgcacctcgactccaaccgcgtttctacagtatttcctctagtccacggatggcaccatctcgtatccatgttacttgtgcgttggtgtacgataagatgccgactggccgcatacacaaaggtgtctgctcaacttggatgaagaatagtgtaccaatggaaaaatctcatgagtgttcgtgggcaccaatattcgttaggcaatcaaattttaagcttccagctgaatccaaagtcccaattatcatggttggccctgggaccggtctagcaccttttagaggatttctacaagaaagactagccttaaaggaatctggtgtggaattgggcccgtctattcttttcttcggatgcagaaacagaagaatggattatatttatgaagatgagttaaacaattttgttgaaacaggggctctttcagaattagtaattgccttttcaagggaaggtcctaccaaagaatacgttcagcataaaatggccgagaaagcttcagacatctggaatttgatcagtgaaggtgcttatttgtacgtatgtggcgatgctaaaggtatggctaaggatgtgcataggacgcttcacactataatgcaagaacagggtagtttagacagttcgaaggccgaatctatggtgaagaacttgcaaatgaatggtagatatttgcgtgatgtgtggtaa

SEQ ID NO: 267 - DNA, 2079 bp, Synthetic construct
  source 1..2079: mol_type = other DNA
atgacctctgcattgtatgcttctgatcttttcaaacaattaaagagtataatgggtactgactccttgtctgatgatgtagttttggtaatagctactacatctttagctctagtcgctggttttgtggtcttgctttggaagaagactactgctgatagatccggtgaattgaaacctctgatgattcctaaatctttgatggctaaagatgaagacgatgatttggacctgggctctggtaagacaagagtttctattttttttggtactcaaactggtactgccgagggttttgctaaggctttgtctgaggaaattaaagctagatatgaaaaggccgcagttaaggtgattgatttggatgactatgctgctgatgatgatcaatacgaagaaaaattgaagaaagaaactttggcattcttctgtgtggctacttacggtgatggtgaaccaactgacaacgctgctagattttataaatggttcactgaggaaaatgaaagagatattaagttacaacaattggcttatggtgtttttgccctaggaaatagacaatacgagcattttaataaaatcggtatcgttttggacgaggaactatgtaagaaaggtgctaaaagattaattgaagttggtttaggtgatgacgatcaatcaattgaagatgactttaacgcttggaaagagtctttgtggtctgagttggataaactgttgaaggatgaagatgacaaatcagtagcaacaccatatactgctgttataccagaatatagagttgttactcatgatccaagatttactactcagaaatctatggagtctaatgttgctaacggtaatactactattgatattcatcatccatgcagagtagacgttgccgttcaaaaagaattgcatacccatgagtccgatagatcttgtatccatcttgagttcgatatttctaggactggtattacctatgaaactggtgaccatgtgggtgtttatgcagaaaatcatgtcgaaatagttgaagaagctggtaagttgttgggtcattccttggacctggtcttttctatccatgctgacaaagaagatggaagtccattggaatctgctgtccctccaccatttccaggaccatgcactttgggtactggtttggctagatatgcagacttgttgaatccaccaagaaaatcagcattggttgctctggctgcttatgcaaccgagcctagtgaagctgaaaaattgaagcatcttacttctccagatggtaaagatgaatactctcaatggattgttgcttctcaaagaagtttattagaagtaatggcagcatttccatctgctaaaccaccattaggtgttttctttgctgctatcgctccaagattacagccaagatattattcaatatcttcttcaccaagattggctccatctagggttcatgtcacttctgctctagtttacggtccaacaccaactggtagaatccataagggtgtttgttccacttggatgaagaatgctgttccagcagagaaatctcatgaatgctctggtgctccaatttttataagagcttctaactttaaattaccatctaatccatctactccaatagtaatggtaggaccaggtactggtttagcaccatttagaggtttccttcaagaaagaatggctttaaaagaggacggtgaagaactgggttcttctctgttattctttggttgtagaaatagacaaatggactttatttacgaagatgaattgaataattttgttgatcagggagtaatttccgagttgataatggctttttctagggaaggtgctcaaaaagagtacgtccaacataaaatgatggaaaaagctgctcaggtatgggatttaattaaagaagaaggttatttgtatgtatgcggtgacgctaaaggtatggcaagagatgtacatagaactttgcatactatagtgcaagaacaagaaggtgtgtcttctagtgaagctgaggccattgttaagaaactacaaactgaaggtagatatctgagagatgtatggtaa

SEQ ID NO: 268-279 - skipped (12 entries)

SEQ ID NO: 280 - protein, 520 aa, unidentified
  source 1..520: note = Siraitia grosvenorii
MEMSSSVAATISIWMVVVCIVGVGWRVVNWVWLRPKKLEKRLREQGLAGNSYRLLFGDLKERAAMEEQANSKPINFSHDIGPRVFPSMYKTIQNYGKNSYMWLGPYPRVHIMDPQQLKTVFTLVYDIQKPNLNPLIKFLLDGIVTHEGEKWAKHRKIINPAFHLEKLKDMIPAFFHSCNEIVNEWERLISKEGSCELDVMPYLQNLAADAISRTAFGSSYEEGKMIFQLLKELTDLVVKVAFGVYIPGWRFLPTKSNNKMKEINRKIKSLLLGIINKRQKAMEEGEAGQSDLLGILMESNSNEIQGEGNNKEDGMSIEDVIEECKVFYIGGQETTARLLIWTMILLSSHTEWQERARTEVLKVFGNKKPDFDGLSRLKVVTMILNEVLRLYPPASMLTRIIQKETRVGKLTLPAGVILIMPIILIHRDHDLWGEDANEFKPERFSKGVSKAAKVQPAFFPFGWGPRICMGQNFAMIEAKMALSLILQRFSFELSSSYVHAPTVVFTTQPQHGAHIVLRKL

SEQ ID NO: 281 - protein, 473 aa, unidentified
  source 1..473: note = Siraitia grosvenorii
MWTVVLGLATLFVAYYIHWINKWRDSKFNGVLPPGTMGLPLIGETIQLSRPSDSLDVHPFIQKKVERYGPIFKTCLAGRPVVVSADAEFNNYIMLQEGRAVEMWYLDTLSKFFGLDTEWLKALGLIHKYIRSITLNHFGAEALRERFLPFIEASSMEALHSWSTQPSVEVKNASALMVFRTSVNKMFGEDAKKLSGNIPGKFTKLLGGFLSLPLNFPGTTYHKCLKDMKEIQKKLREVVDDRLANVGPDVEDFLGQALKDKESEKFISEEFIIQLLFSISFASFESISTTLTLILKLLDEHPEVVKELEAEHEAIRKARADPDGPITWEEYKSMTFTLQVINETLRLGSVTPALLRKTVKDLQVKGYIIPEGWTIMLVTASRHRDPKVYKDPHIFNPWRWKDLDSITIQKNFMPFGGGLRHCAGAEYSKVYLCTFLHILCTKYRWTKLGGGRIARAHILSFEDGLHVKFTPKE

SEQ ID NO: 282 - protein, 701 aa, unidentified
  source 1..701: note = Siraitia grosvenorii
MKVSPFEFMSAIIKGRMDPSNSSFESTGEVASVIFENRELVAILTTSIAVMIGCFVVLMWRRAGSRKVKNVELPKPLIVHEPEPEVEDGKKKVSIFFGTQTGTAEGFAKALADEAKARYEKATFRVVDLDDYAADDDQYEEKLKNESFAVFLLATYGDGEPTDNAARFYKWFAEGKERGEWLQNLHYAVFGLGNRQYEHFNKIAKVADELLEAQGGNRLVKVGLGDDDQCIEDDFSAWRESLWPELDMLLRDEDDATTVTTPYTAAVLEYRVVFHDSADVAAEDKSWINANGHAVHDAQHPFRSNVVVRKELHTSASDRSCSHLEFNISGSALNYETGDHVGVYCENLTETVDEALNLLGLSPETYFSIYTDNEDGTPLGGSSLPPPFPSCTLRTALTRYADLLNSPKKSALLALAAHASNPVEADRLRYLASPAGKDEYAQSVIGSQKSLLEVMAEFPSAKPPLGVFFAAVAPRLQPRFYSISSSPRMAPSRIHVTCALVYDKMPTGRIHKGVCSTWMKNSVPMEKSHECSWAPIFVRQSNFKLPAESKVPIIMVGPGTGLAPFRGFLQERLALKESGVELGPSILFFGCRNRRMDYIYEDELNNFVETGALSELVIAFSREGPTKEYVQHKMAEKASDIWNLISEGAYLYVCGDAKGMAKDVHRTLHTIMQEQGSLDSSKAESMVKNLQMNGRYLRDVW

SEQ ID NO: 283 - protein, 692 aa, Arabidopsis thaliana [sequence omitted]

SEQ ID NO: 284-285 - skipped (2 entries)

SEQ ID NO: 286 - protein, 316 aa, unidentified
  source 1..316: note = Siraitia grosvenorii
MDQIEHITINTNGIKMHIASVGTGPVVLLLHGFPELWYSWRHQLLYLSSVGYRAIAPDLRGYGDTDSPASPTSYTALHIVGDLVGALDELGIEKVFLVGHDWGAIIAWYFCLFRPDRIKALVNLSVQFIPRNPAIPFIEGFRTAFGDDFYMCRFQVPGEAEEDFASIDTAQLFKTSLCNRSSAPPCLPKEIGFRAIPPPENLPSWLTEEDINYYAAKFKQTGFTGALNYYRAFDLTWELTAPWTGAQIQVPVKFIVGDSDLTYHFPGAKEYIHNGGFKKDVPLLEEVVVVKDACHFINQERPQEINAHIHDFINKF

SEQ ID NO: 287-295 - skipped (9 entries)

SEQ ID NO: 296 - DNA, 1563 bp, Synthetic construct
  source 1..1563: mol_type = other DNA
atggaaatgtcctcaagtgtcgcagccacaatcagtatctggatggtcgtcgtatgtatcgtaggtgtaggttggagagtcgtaaattgggtttggttgagaccaaagaaattggaaaagagattgagagaacaaggtttggccggtaattcttacagattgttgttcggtgacttgaaggaaagagctgcaatggaagaacaagcaaattcaaagcctataaacttctcccatgacatcggtccaagagttttcccttcaatgtacaagaccatccaaaactacggtaaaaactcctacatgtggttaggtccataccctagagtccacatcatggatccacaacaattgaagaccgtttttactttggtctacgacattcaaaagccaaatttgaaccctttgattaaattcttgttagatggtatcgttacacatgaaggtgaaaagtgggctaagcacagaaagattattaacccagcattccatttggaaaagttgaaggatatgatacctgctttctttcactcatgtaatgaaatcgtcaacgaatgggaaagattgatttcaaaagaaggttcctgcgaattggatgtaatgccttatttgcaaaatttggccgctgacgccatttcaagaaccgcttttggttcttcatacgaagaaggtaaaatgatcttccaattgttgaaggaattgactgatttggttgtcaaggtagcttttggtgtttatattccaggttggagattcttgcctacaaagagtaacaacaaaatgaaggaaattaatagaaaaatcaagtctttgttgttgggtatcattaacaagagacaaaaggcaatggaagaaggtgaagccggtcaatctgatttgttgggtatattaatggaaagtaattctaacgaaatccaaggtgaaggtaataacaaggaagatggcatgtctattgaagacgtcatcgaagagtgtaaggtattttatataggtggtcaagaaactacagcaagattattgatctggactatgatattgttgtccagtcatacagaatggcaagaaagagccagaaccgaagtcttgaaggtatttggtaataagaaaccagatttcgacggtttgtcaagattgaaggtagttactatgatcttgaacgaagttttaagattgtacccacctgcttccatgttgacaagaatcatccaaaaggaaacaagagttggtaaattaaccttgccagcaggtgttatcttgataatgcctatcatcttgatacatagagatcacgacttgtggggtgaagatgctaacgagtttaaaccagaaagattcagtaaaggtgtttctaaggcagccaaagtccaaccagcctttttcccttttggttggggtcctagaatttgcatgggtcaaaacttcgctatgatcgaagctaagatggcattgagtttgatcttgcaaagattttctttcgaattgtcttcatcctacgttcatgcaccaactgtcgtcttcactacacaaccacaacacggtgcccacatcgttttgagaaagttatga

SEQ ID NO: 297 - DNA, 2079 bp, Synthetic construct
  source 1..2079: mol_type = other DNA
atgacctctgcattgtatgcttctgatcttttcaaacaattaaagagtataatgggtactgactccttgtctgatgatgtagttttggtaatagctactacatctttagctctagtcgctggttttgtggtcttgctttggaagaagactactgctgatagatccggtgaattgaaacctctgatgattcctaaatctttgatggctaaagatgaagacgatgatttggacctgggctctggtaagacaagagtttctattttttttggtactcaaactggtactgccgagggttttgctaaggctttgtctgaggaaattaaagctagatatgaaaaggccgcagttaaggtgattgatttggatgactatgctgctgatgatgatcaatacgaagaaaaattgaagaaagaaactttggcattcttctgtgtggctacttacggtgatggtgaaccaactgacaacgctgctagattttataaatggttcactgaggaaaatgaaagagatattaagttacaacaattggcttatggtgtttttgccctaggaaatagacaatacgagcattttaataaaatcggtatcgttttggacgaggaactatgtaagaaaggtgctaaaagattaattgaagttggtttaggtgatgacgatcaatcaattgaagatgactttaacgcttggaaagagtctttgtggtctgagttggataaactgttgaaggatgaagatgacaaatcagtagcaacaccatatactgctgttataccagaatatagagttgttactcatgatccaagatttactactcagaaatctatggagtctaatgttgctaacggtaatactactattgatattcatcatccatgcagagtagacgttgccgttcaaaaagaattgcatacccatgagtccgatagatcttgtatccatcttgagttcgatatttctaggactggtattacctatgaaactggtgaccatgtgggtgtttatgcagaaaatcatgtcgaaatagttgaagaagctggtaagttgttgggtcattccttggacctggtcttttctatccatgctgacaaagaagatggaagtccattggaatctgctgtccctccaccatttccaggaccatgcactttgggtactggtttggctagatatgcagacttgttgaatccaccaagaaaatcagcattggttgctctggctgcttatgcaaccgagcctagtgaagctgaaaaattgaagcatcttacttctccagatggtaaagatgaatactctcaatggattgttgcttctcaaagaagtttattagaagtaatggcagcatttccatctgctaaaccaccattaggtgttttctttgctgctatcgctccaagattacagccaagatattattcaatatcttcttcaccaagattggctccatctagggttcatgtcacttctgctctagtttacggtccaacaccaactggtagaatccataagggtgtttgttccacttggatgaagaatgctgttccagcagagaaatctcatgaatgctctggtgctccaatttttataagagcttctaactttaaattaccatctaatccatctactccaatagtaatggtaggaccaggtactggtttagcaccatttagaggtttccttcaagaaagaatggctttaaaagaggacggtgaagaactgggttcttctctgttattctttggttgtagaaatagacaaatggactttatttacgaagatgaattgaataattttgttgatcagggagtaatttccgagttgataatggctttttctagggaaggtgctcaaaaagagtacgtccaacataaaatgatggaaaaagctgctcaggtatgggatttaattaaagaagaaggttatttgtatgtatgcggtgacgctaaaggtatggcaagagacgtacatagaactttgcatactatagtgcaagaacaagaaggtgtgtcttctagtgaagctgaggccattgttaagaaactacaaactgaaggtagatatctgagagacgtatggtaa

SEQ ID NO: 298 - DNA, 2106 bp, Synthetic construct; identical to SEQ ID NO: 266
  source 1..2106: mol_type = other DNA

SEQ ID NO: 299 - DNA, 2280 bp, Synthetic construct
  source 1..2280: mol_type = other DNA
atgtggaggcttaaagtaggcgcggaatcagtcggggagaatgacgaaaagtggctgaaaagcattagtaaccacttgggtcgccaggtgtgggaattttgcccagatgcaggtacgcaacaacaactattacaggttcataaggctagaaaagccttccatgatgaccgttttcacagaaagcaatcgtccgatttgttcatcaccatacaatatggaaaagaggttgaaaatggaggtaagacagctggcgtaaaactcaaggagggtgaagaagttagaaaggaggcagtcgaatcttctttggaacgggctttatcgttttactcttcaattcaaacttccgatggtaactgggccagtgacctgggtggtccgatgtttttactacccggcttggttatcgccctttatgtaaccggcgtcctgaatagcgtgctatccaaacatcataggcaggaaatgtgtcgatacgtttataaccaccaaaatgaggatgggggttggggattacatattgaaggaccttcaacaatgttcgggtctgcacttaattatgtcgctttgagactcttaggcgaagacgcaaacgctggtgccatgccaaaagcaagagcgtggatattggatcacggaggtgcgactggtatcacgtcatggggcaaactttggctctcggttttaggagtgtacgagtggagtggcaataatcctctaccacctgaattttggctgtttccttatttcttgccgttccatcccgggcgaatgtggtgtcattgcagaatggtatatttaccaatgagttacctctatggtaagcgttttgtaggtccaattactcctatagttttgtctttacgcaaagaactatacgctgtcccataccatgagatcgattggaacaagtcgcgtaatacatgtgctaaagaagatctttattacccccacccgaaaatgcaggacattctgtgggggtcattgcatcacgtttatgagcccttatttaccagatggccggcgaaaagactaagggaaaaggctttgcaaactgccatgcaacatatacactacgaagatgaaaacacaagatacatttgtttagggcctgtaaataaagtgttgaatttactttgctgttgggtagaagacccatatagcgatgcatttaagctccatttgcagagggttcacgactatttatgggtcgctgaagatggaatgaaaatgcaaggttataatggatctcaattgtgggacacagcatttagcattcaggccattgtctctactaaactcgttgataactacggcccaacgctacggaaggcccatgatttcgtgaaaagcagtcagatacaacaagattgccctggcgatccaaacgtgtggtataggcatatacacaagggtgcttggccattctcaacaagagatcatggttggctgatctccgattgtacagctgagggattaaaggcggcattaatgttgtctaaattaccgagtgaaaccgttggtgagagcttggaaagaaatagactgtgtgatgccgtgaatgtgttgctttccctgcaaaatgacaatggaggtttcgcgtcgtatgaactaactcgatcttatccttggttagaacttatcaaccccgcagaaacctttggggatattgttatcgattatccatacgtggagtgtacttcagcaaccatggaggctttgacactgtttaaaaaacttcatcctggacatcgtactaaggaaattgatacggccatagtacgcgcagctaactttctagaaaatatgcaaagaactgatggtagttggtacggttgctggggcgtttgtttcacttacgctggctggtttggaattaaaggtctagtagctgccggacgtacatataacaattgcttggccattagaaaggcttgtgactttctcttatcaaaagaactacctggtggtgggtggggagagtcttatcttagctgccagaacaaagtatatactaatttggaaggcaacaggccacaccttgttaacacagcatgggtattgatggctctgattgaagctggtcaagctgaacgtgacccaacgcccttacacagagcagcaagactgctgatcaattcacagttagaaaatggtgatttccctcagcaagagatcatgggcgtttttaacaagaattgtatgattacttacgccgcatatcgtaacatatttccgatttgggcattaggggaatactgccatagggtgcttactgaataa

SEQ ID NO: 300 - DNA, 951 bp, Synthetic construct
  source 1..951: mol_type = other DNA
atggatcagatcgagcacattactataaataccaacggcattaaaatgcatatagcgtcagtagggacaggaccagttgtcctgcttctacatggtttccccgaattgtggtactcttggcgtcaccaattgttatatttatcgagtgtgggttatagggcaatcgcccctgacctgagaggttacggcgatacggattccccggcatctccaactagctatacagctctccatattgttggagacttggtgggtgctttagatgaactaggaattgaaaaggtttttctagtcggtcatgactggggcgccataatcgcttggtatttttgccttttccgccctgatcgaattaaagctttggtaaatttatcagttcaatttataccgagaaacccagcaattcctttcatcgaaggttttcgtaccgcgtttggcgacgatttttacatgtgtagattccaggtcccaggagaggcagaggaagattttgcctctatagacactgctcaacttttcaagaccagtctatgtaatagatcctcagcgcccccttgcctcccaaaagaaattgggttcagggccattcccccacctgaaaatttgcctagctggctgacggaggaagatatcaactattacgctgcaaagtttaaacaaacaggtttcactggtgccctcaattactatagagcatttgatcttacttgggaattaacagctccatggactggtgctcaaatacaggttccagtaaaatttatcgtaggagattccgatttgacgtatcattttcccggagcaaaggagtacattcacaacgggggcttcaaaaaagacgtgcctttattggaagaggttgtggtagttaaggatgcatgtcacttcatcaaccaagaaagacctcaagaaattaatgctcatattcatgactttataaataagttctaa

SEQ ID NO: 301 - DNA, 951 bp, Synthetic construct
  source 1..951: mol_type = other DNA
atggagaagattgaacacacaacgatctcgaccaacggtataaatatgcatgtggcatcaattggctctggacccgccgtacttttcttgcatggttttccagaactatggtacagctggaggcaccagttactgtttttaagttccatggggtatagagcgatagctccggacttgcgcggattcggtgatactgatgctccaccttcgcctagttcatataccgcacatcatattgtcggggacttggttggcctattagatcaactcggtatcgatcaagtttttttggtcggacacgactggggtgccatgatggcgtggtacttttgtctgttccgtccagatcgagtgaaagctcttgttaatttatctgttcatttcctaagaagacacccatccattaaatttgtagatgggtttagggcacttttaggagatgacttctatttttgccaatttcaggaacctggcgtggcagaggctgattttggttccgtagacgttgccacaatgttaaagaaattcttgactatgcgggatccaagacccccaatgataccgaaggaaaaaggttttagagctttggaaacacctgatccgctccctgcgtggttaactgaagaagacatcgattacttcgccggtaaatttcgtaagacgggtttcactggaggctttaactattatcgcgctttcaatctgacatgggagctaacagcaccctggtcgggttctgaaataaaggtggcagctaaatttattgtcggagaccttgacttagtttaccattttcctggtgcgaaggagtatattcatggagggggcttcaaaaaagatgtacccttgctagaagaggtcgttgtcgtggatggcgctgcccattttataaatcaagaaagaccagcagagattagctctctaatctacgactttatcaaaaagttctaa

SEQ ID NO: 302 - DNA, 1335 bp, Synthetic construct
  source 1..1335: mol_type = other DNA
atgggaaagctattacaattggcattgcatccggtcgagatgaaggcagctttgaagctgaagttttgcagaacaccgctattctccatctatgatcagtccacgtctccatatctcttgcactgtttcgaactgttgaacttgacctccagatcgtttgctgctgtgatcagagagctgcatccagaattgagaaactgtgttactctcttttatttgattttaagggctttggataccatcgaagacgatatgtccatcgaacacgatttgaaaattgacttgttgcgtcacttccacgagaaattgttgttaactaaatggagtttcgacggaaatgcccccgatgtgaaggacagagccgttttgacagatttcgaatcgattcttattgaattccacaaattgaaaccagaatatcaagaagtcatcaaggagatcaccgagaaaatgggtaatggtatggccgactacatcttggatgaaaattacaacttgaatgggttgcaaaccgtccacgactacgacgtgtactgtcactacgtagctggtttggtcggtgatggtttgacccgtttgattgtcattgccaagtttgccaacgaatctttgtattctaatgagcaattgtatgaaagcatgggtcttttcctacaaaaaaccaacatcatcagagactacaatgaagatttggtcgatggtagatccttctggcccaaggaaatctggtcacaatacgctcctcagttgaaggacttcatgaaacctgaaaacgaacaactggggttggactgtataaaccacctcgtcttaaacgcattgagtcatgttatcgatgtgttgacttatttggccagtatccacgagcaatccactttccaattttgtgccattccccaagttatggccattgcaaccttggctttggtattcaacaaccgtgaagtgctacatggcaatgtaaagattcgtaagggtactacctgctatttaattttgaaatcaaggactttgcgtggctgtgtcgagatttttgactattacttacgtgatatcaaatctaaattggctgtgcaagatccaaatttcttaaaattgaacattcaaatctccaagatcgaacaattcatggaagaaatgtaccaggataaattacctcctaacgtgaagccaaatgaaactccaattttcttgaaagttaaagaaagatccagatacgatgatgaattggtcccaacccaacaagaagaagagtacaagttcaatatggttttatctatcatcttgtccgttcttcttgggttttattatatatacactttacacagagcgtga

SEQ ID NO: 303 - DNA, 1491 bp, Synthetic construct
  source 1..1491: mol_type = other DNA
atgtctgctgttaacgttgcacctgaattgattaatgccgacaacacaattacctacgatgcgattgtcatcggtgctggtgttatcggtccatgtgttgctactggtctagcaagaaagggtaagaaagttcttatcgtagaacgtgactgggctatgcctgatagaattgttggtgaattgatgcaaccaggtggtgttagagcattgagaagtctgggtatgattcaatctatcaacaacatcgaagcatatcctgttaccggttataccgtctttttcaacggcgaacaagttgatattccatacccttacaaggccgatatccctaaagttgaaaaattgaaggacttggtcaaagatggtaatgacaaggtcttggaagacagcactattcacatcaaggattacgaagatgatgaaagagaaaggggtgttgcttttgttcatggtagattcttgaacaacttgagaaacattactgctcaagagccaaatgttactagagtgcaaggtaactgtattgagatattgaaggatgaaaagaatgaggttgttggtgccaaggttgacattgatggccgtggcaaggtggaattcaaagcccacttgacatttatctgtgacggtatcttttcacgtttcagaaaggaattgcacccagaccatgttccaactgtcggttcttcgtttgtcggtatgtctttgttcaatgctaagaatcctgctcctatgcacggtcacgttattcttggtagtgatcatatgccaatcttggtttaccaaatcagtccagaagaaacaagaatcctttgtgcttacaactctccaaaggtcccagctgatatcaagagttggatgattaaggatgtccaacctttcattccaaagagtctacgtccttcatttgatgaagccgtcagccaaggtaaatttagagctatgccaaactcctacttgccagctagacaaaacgacgtcactggtatgtgtgttatcggtgacgctctaaatatgagacatccattgactggtggtggtatgactgtcggtttgcatgatgttgtcttgttgattaagaaaataggtgacctagacttcagcgaccgtgaaaaggttttggatgaattactagactaccatttcgaaagaaagagttacgattccgttattaacgttttgtcagtggctttgtattctttgttcgctgctgacagcgataacttgaaggcattacaaaaaggttgtttcaaatatttccaaagaggtggcgattgtgtcaacaaacccgttgaatttctgtctggtgtcttgccaaagcctttgcaattgaccagggttttcttcgctgtcgctttttacaccatttacttgaacatggaagaacgtggtttcttgggattaccaatggctttattggaaggtattatgattttgatcacagctattagagtattcaccccatttttgtttggtgagttgattggttaa

SEQ ID NO: 304 - DNA, 2196 bp, Synthetic construct
  source 1..2196: mol_type = other DNA
atgacagaattttattctgacacaatcggtctaccaaagacagatccacgtctttggagactgagaactgatgagctaggccgagaaagctgggaatatttaacccctcagcaagccgcaaacgacccaccatctacctttacacaatggctactgcaagatcccaaatttcctcaacctcatccagaaagaaataagcattcaccagatttttcagccttcgatgcgtgtcataatggtgcatcttttttcaaactgcttcaagagcctgactcaggtatttttccgtgtcaatataaaggacccatgttcatgacaatcggttacgtagccgtaaactatatcgccggtattgaaattcctgagcatgagagaatagaattaattagatacatcgtcaatactgctcaccctgtcgacggaggttggggtctacattctgttgacaaatccaccgtgtttggtacagtattgaactatgtaatcttacgtttattgggtctacccaaggaccacccggtttgcgccaaggcaagaagcacattgttaaggttaggcggtgctattggatcccctcactggggaaaaatttggctaagtgcactaaacttgtataaatgggaaggtgtgaaccctgcccctcctgaaacttggttacttccatattcactgcccatgcatccggggagatggtgggttcatactagaggtgtttacattccggtcagttacctgtcattggtcaaattttcttgcccaatgactcctcttcttgaagaactgaggaatgaaatttacactaaaccgtttgacaagattaacttctccaagaacaggaataccgtatgtggagtagacctatattacccccattctactactttgaatattgcgaacagccttgtagtattttacgaaaaatacctaagaaaccggttcatttactctctatccaagaagaaggtttatgatctaatcaaaacggagttacagaatactgattccttgtgtatagcacctgttaaccaggcgttttgcgcacttgtcactcttattgaagaaggggtagactcggaagcgttccagcgtctccaatataggttcaaggatgcattgttccatggtccacagggtatgaccattatgggaacaaatggtgtgcaaacctgggattgtgcgtttgccattcaatactttttcgtcgcaggcctcgcagaaagacctgaattctataacacaattgtctctgcctataaattcttgtgtcatgctcaatttgacaccgagtgcgttccaggtagttatagggataagagaaagggggcttggggcttctcaacaaaaacacagggctatacagtggcagattgcactgcagaagcaattaaagccatcatcatggtgaaaaactctcccgtctttagtgaagtacaccatatgattagcagtgaacgtttatttgaaggcattgatgtgttattgaacctacaaaacatcggatcttttgaatatggttcctttgcaacctatgaaaaaatcaaggccccactagcaatggaaaccttgaatcctgctgaagtttttggtaacataatggtagaatacccatacgtggaatgtactgattcatccgttctggggttgacatattttcacaagtacttcgactataggaaagaggaaatacgtacacgcatcagaatcgccatcgaattcataaaaaaatctcaattaccagatggaagttggtatggaagctggggtatttgttttacatatgccggtatgtttgcattggaggcattacacaccgtgggggagacctatgagaattcctcaacggtaagaaaaggttgcgacttcttggtcagtaaacagatgaaggatggcggttggggggaatcaatgaagtccagtgaattacatagttatgtggatagtgaaaaatcgctagtcgttcaaaccgcatgggcgctaattgcacttcttttcgctgaatatcctaataaagaagtcatcgaccgcggtattgaccttttaaaaaatagacaagaagaatccggggaatggaaatttgaaagtgtagaaggtgttttcaaccactcttgtgcaattgaatacccaagttatcgattcttattccctattaaggcattaggtatgtacagcagggcatatgaaacacatacgctttaa

SEQ ID NO: 305-309 - skipped (5 entries)

SEQ ID NO: 310 - protein, 316 aa, unidentified
  source 1..316: note = Siraitia grosvenorii
MEKIEHTTISTNGINMHVASIGSGPAVLFLHGFPELWYSWRHQLLFLSSMGYRAIAPDLRGFGDTDAPPSPSSYTAHHIVGDLVGLLDQLGIDQVFLVGHDWGAMMAWYFCLFRPDRVKALVNLSVHFLRRHPSIKFVDGFRALLGDDFYFCQFQEPGVAEADFGSVDVATMLKKFLTMRDPRPPMIPKEKGFRALETPDPLPAWLTEEDIDYFAGKFRKTGFTGGFNYYRAFNLTWELTAPWSGSEIKVAAKFIVGDLDLVYHFPGAKEYIHGGGFKKDVPLLEEVVVVDGAAHFINQERPAEISSLIYDFIKKF

SEQ ID NO: 311 - protein, 444 aa, Saccharomyces cerevisiae
MGKLLQLALHPVEMKAALKLKFCRTPLFSIYDQSTSPYLLHCFELLNLTSRSFAAVIRELHPELRNCVTLFYLILRALDTIEDDMSIEHDLKIDLLRHFHEKLLLTKWSFDGNAPDVKDRAVLTDFESILIEFHKLKPEYQEVIKEITEKMGNGMADYILDENYNLNGLQTVHDYDVYCHYVAGLVGDGLTRLIVIAKFANESLYSNEQLYESMGLFLQKTNIIRDYNEDLVDGRSFWPKEIWSQYAPQLKDFMKPENEQLGLDCINHLVLNALSHVIDVLTYLASIHEQSTFQFCAIPQVMAIATLALVFNNREVLHGNVKIRKGTTCYLILKSRTLRGCVEIFDYYLRDIKSKLAVQDPNFLKLNIQISKIEQFMEEMYQDKLPPNVKPNETPIFLKVKERSRYDDELVPTQQEEEYKFNMVLSIILSVLLGFYYIYTLHRA

SEQ ID NO: 312 - protein, 496 aa, Saccharomyces cerevisiae
MSAVNVAPELINADNTITYDAIVIGAGVIGPCVATGLARKGKKVLIVERDWAMPDRIVGELMQPGGVRALRSLGMIQSINNIEAYPVTGYTVFFNGEQVDIPYPYKADIPKVEKLKDLVKDGNDKVLEDSTIHIKDYEDDERERGVAFVHGRFLNNLRNITAQEPNVTRVQGNCIEILKDEKNEVVGAKVDIDGRGKVEFKAHLTFICDGIFSRFRKELHPDHVPTVGSSFVGMSLFNAKNPAPMHGHVILGSDHMPILVYQISPEETRILCAYNSPKVPADIKSWMIKDVQPFIPKSLRPSFDEAVSQGKFRAMPNSYLPARQNDVTGMCVIGDALNMRHPLTGGGMTVGLHDVVLLIKKIGDLDFSDREKVLDELLDYHFERKSYDSVINVLSVALYSLFAADSDNLKALQKGCFKYFQRGGDCVNKPVEFLSGVLPKPLQLTRVFFAVAFYTIYLNMEERGFLGLPMALLEGIMILITAIRVFTPFLFGELIG

SEQ ID NO: 313 - protein, 731 aa, Saccharomyces cerevisiae
MTEFYSDTIGLPKTDPRLWRLRTDELGRESWEYLTPQQAANDPPSTFTQWLLQDPKFPQPHPERNKHSPDFSAFDACHNGASFFKLLQEPDSGIFPCQYKGPMFMTIGYVAVNYIAGIEIPEHERIELIRYIVNTAHPVDGGWGLHSVDKSTVFGTVLNYVILRLLGLPKDHPVCAKARSTLLRLGGAIGSPHWGKIWLSALNLYKWEGVNPAPPETWLLPYSLPMHPGRWWVHTRGVYIPVSYLSLVKFSCPMTPLLEELRNEIYTKPFDKINFSKNRNTVCGVDLYYPHSTTLNIANSLVVFYEKYLRNRFIYSLSKKKVYDLIKTELQNTDSLCIAPVNQAFCALVTLIEEGVDSEAFQRLQYRFKDALFHGPQGMTIMGTNGVQTWDCAFAIQYFFVAGLAERPEFYNTIVSAYKFLCHAQFDTECVPGSYRDKRKGAWGFSTKTQGYTVADCTAEAIKAIIMVKNSPVFSEVHHMISSERLFEGIDVLLNLQNIGSFEYGSFATYEKIKAPLAMETLNPAEVFGNIMVEYPYVECTDSSVLGLTYFHKYFDYRKEEIRTRIRIAIEFIKKSQLPDGSWYGSWGICFTYAGMFALEALHTVGETYENSSTVRKGCDFLVSKQMKDGGWGESMKSSELHSYVDSEKSLVVQTAWALIALLFAEYPNKEVIDRGIDLLKNRQEESGEWKFESVEGVFNHSCAIEYPSYRFLFPIKALGMYSRAYETHTL

SEQ ID NO: 314 - DNA, 1422 bp, Synthetic construct
  source 1..1422: mol_type = other DNA
atgtggactgtcgtgctcggtttggcgacgctgtttgtcgcctactacatccattggattaacaaatggagagattccaagttcaacggagttctgccgccgggcaccatgggtttgccgctcatcggagaaacgattcaactgagtcgacccagtgactccctcgacgttcaccctttcatccagaaaaaagttgaaagatacgggccgatcttcaaaacatgtctggccggaaggccggtggtggtgtcggcggacgcagagttcaacaactacataatgctgcaggaaggaagagcagtggaaatgtggtatttggatacgctctccaaatttttcggcctcgacaccgagtggctcaaagctctgggcctcatccacaagtacatcagaagcattactctcaatcacttcggcgccgaggccctgcgggagagatttcttccttttattgaagcatcctccatggaagcccttcactcctggtctactcaacctagcgtcgaagtcaaaaatgcctccgctctcatggtttttaggacctcggtgaataagatgttcggtgaggatgcgaagaagctatcgggaaatatccctgggaagttcacgaagcttctaggaggatttctcagtttaccactgaattttcccggcaccacctaccacaaatgcttgaaggatatgaaggaaatccagaagaagctaagagaggttgtagacgatagattggctaatgtgggccctgatgtggaagatttcttggggcaagcccttaaagataaggaatcagagaagttcatttcagaggagttcatcatccaactgttgttttctatcagttttgctagctttgagtccatctccaccactcttactttgattctcaagctccttgatgaacacccagaagtagtgaaagagttggaagctgaacacgaggcgattcgaaaagctagagcagatccagatggaccaattacttgggaagaatacaaatccatgacttttacattacaagtcatcaatgaaaccctaaggttggggagtgtcacacctgccttgttgaggaaaacagttaaagatcttcaagtaaaaggatacataatcccggaaggatggacaataatgcttgtcaccgcttcacgtcacagagatccaaaagtctataaggaccctcatatcttcaatccatggcgttggaaggacttggactcaattaccatccaaaagaacttcatgccttttgggggaggcttaaggcattgtgctggtgctgagtactctaaagtctacttgtgcaccttcttgcacatcctctgtaccaaataccgatggaccaaacttgggggaggaaggattgcaagagctcatatattgagttttgaagatgggttacatgtgaagttcacgccaaaagaataa

SEQ ID NO: 315 - skipped

SEQ ID NO: 316 - DNA, 537 bp, Synthetic construct
  source 1..537: mol_type = other DNA
atggctctgcaagtctgggagactctcaaggaggccattgtcgcttacaccggtctgtctcccgctaccttcttcactgtgctcgctcttggtttggccgtctactacgtcatttcgggcttcttcggcacctcggactacggctctcactctcgagatttcggtgagcagatgcagcccctgcctcctcccgtccaggttggtgagatctctgaggaggagctcaagcagtacgacggcagtgactccaagaagcctcttctcatggccatcaagggccagatttacgatgtcagccagagccgaatgttctacggacccggtggcccttacgctctgtttgccggcaaggatgcttctcgagctctggccaaaatgtcatttgaagagaaggacctgactggtgacatttccggactcggcccattcgagcttgaggccctccaggattgggaatacaagttcatgtccaagtacgttaaggtcggcaccgttgagaagcccgtcagcgagacagtcgctgccaaggttgagtag

SEQ ID NO: 317 - DNA, 642 bp, Synthetic construct
  source 1..642: mol_type = other DNA
atggctctgcaagtctgggagactctcaaggaggccattgtcgcttacaccggtctgtctcccgctaccttcttcactgtgctcgctcttggtttggccgtctactacgtcatttcgggcttcttcggcacctcggactacggctctcactctcgagatttcggtgagcagatgcagcccctgcctcctcccgtccaggttggtgagatctctgaggaggagctcaagcagtacgacggcagtgactccaagaagcctcttctcatggccatcaagggccagatttacgatgtcagccagagccgaatgttctacggacccggtggcccttacgctctgtttgccggcaaggatgcttctcgagctctggccaaaatgtcatttgaagagaaggacctgactggtgacatttccggactcggcccattcgagcttgaggccctccaggattgggaatacaagttcatgtccaagtacgttaaggtcggcaccgttgagaagcccgtcagcgagacagacgctgctcccgccggtgagtctgctgagtccaccaacgccgaggtgtccaagcccgctgaggacggtccttccgagtcccaggccgacaaggctgaggagacagtcgctgccaaggttgagtag

SEQ ID NO: 318 - protein, 178 aa, Synthetic construct
MALQVWETLKEAIVAYTGLSPATFFTVLALGLAVYYVISGFFGTSDYGSHSRDFGEQMQPLPPPVQVGEISEEELKQYDGSDSKKPLLMAIKGQIYDVSQSRMFYGPGGPYALFAGKDASRALAKMSFEEKDLTGDISGLGPFELEALQDWEYKFMSKYVKVGTVEKPVSETVAAKVE

SEQ ID NO: 319 - DNA, 2280 bp, Synthetic construct
  source 1..2280: mol_type = other DNA
atgtggcgactgaaggttggtgctgagtccgtaggcgaaaatgacgagaagtggctcaaatctatcagtaaccatcttggaagacaagtgtgggagttttgccctgatgccgggactcagcagcagctgttgcaggtccacaaggcccgtaaggcattccacgacgaccgattccaccgaaagcagtcgtctgaccttttcattaccatccagtatggtaaggaggttgagaacggtggcaagaccgctggtgtcaagctgaaggagggtgaggaggtccgcaaggaggccgttgagtcctctctcgaacgagcgctctccttttactcctctattcagacctccgatggcaactgggccagcgatctgggtggtcccatgttcttactgcccggattggtcatcgccctctacgttacgggtgtgctaaactctgttctgtccaagcaccatcgacaggagatgtgtcggtacgtctacaaccaccaaaacgaggacggtggctggggtcttcacattgagggaccatctaccatgttcggttcagctctaaactacgtcgccctccgactgcttggggaggacgctaacgccggtgcaatgcccaaggctcgagcctggatcctcgaccacggcggtgctactggtatcacctcctggggtaagctctggctgagtgtgcttggcgtctacgagtggtccggcaacaaccccctccctcccgagttctggctgtttccctacttcctgcctttccatcccggaaggatgtggtgtcactgccgaatggtctacttgcccatgtcttatctctacggtaagcgattcgttggtcccatcacccctatcgtcctgtcccttcgaaaggagctttacgccgtcccgtaccacgagattgattggaacaagtcccgaaacacttgtgccaaggaggacctctactaccctcaccccaagatgcaggacattctgtggggctcccttcatcacgtgtacgagcccctgttcacccgatggcccgctaagcgacttcgagagaaggccttgcagacagccatgcagcacatccactacgaagacgaaaatacccgatacatctgcctgggtcccgtcaacaaggttctgaacctcttgtgttgttgggtcgaggatccctactctgatgctttcaaactccacctccagcgagttcacgactacctgtgggttgccgaggacggaatgaagatgcagggatacaacggttctcagctctgggatactgcattttcgattcaggccatcgtcagcaccaagctggtagacaactacggaccgacactccgaaaggctcacgactttgttaagtcttcccagatccaacaggactgccccggtgatcccaacgtctggtacagacacattcataaaggtgcctggcctttctccacccgtgaccacggctggctcatttctgattgtaccgctgagggccttaaggccgccctgatgctgtccaagctcccctctgagactgtgggtgagtcgctcgagcgaaaccgactttgcgacgccgtgaacgttctccttagtctccagaacgacaacggtggtttcgcttcctatgagctgacccgttcctacccctggcttgaactgattaaccctgcggagacattcggtgatatcgtcatcgactacccctacgttgagtgtacgtctgccaccatggaggctcttaccctgtttaagaagctccatcctggtcaccgaaccaaggagattgacaccgccatcgtccgagccgctaatttcctggagaacatgcagcgaaccgacgggtcatggtacggttgctggggagtctgttttacctacgccggatggttcggtattaagggtcttgtcgccgctggccgaacttacaacaactgtttggccatcagaaaggcctgcgacttcctcctgtctaaggagctgcccggaggtggctggggcgaatcctatctctcctgtcagaataaggtttacaccaacttagagggtaacaggccccacttggtgaatactgcttgggttcttatggcgctgatcgaggccggtcaggcggagcgagatcccacccccctacaccgagctgcccgactgcttatcaactcccagctcgagaacggtgacttccctcaacaggagattatgggtgtttttaacaagaactgcatgatcacgtacgccgcctaccgaaacatcttccctatctgggctcttggtgaatactgtcaccgagtcctgaccgagtag

SEQ ID NO: 320 - DNA, 1563 bp, Synthetic construct
  source 1..1563: mol_type = other DNA
atggagatgtcctcttcagtggctgccacgatttctatctggatggttgtcgtctgtatcgttggagtcggttggcgagtggtcaactgggtttggctgcggcccaaaaagcttgaaaagcgactccgtgagcagggtctggccggcaattcctaccgactcttatttggggacctaaaggagcgagcagctatggaggagcaagccaactcgaagccaattaacttctctcatgatatcggtccccgagtattcccttccatgtataagaccatccagaactacggtaagaactcctacatgtggctcggtccctacccccgagtccacattatggaccctcagcagcttaagacagttttcactctggtctacgacatccagaagccgaacctgaacccccttatcaagtttctgctggatggcatcgtgacccacgagggggagaagtgggcgaagcaccgaaagattatcaaccccgccttccacctcgaaaagctcaaggacatgatccctgctttcttccactcttgcaacgagattgtcaatgagtgggagcgactgatctccaaggagggttcgtgtgagctcgatgttatgccctacctgcaaaacttagccgctgacgccatttcccgaaccgcctttggtagttcttacgaggagggtaagatgattttccagctactgaaggaactcaccgaccttgtcgtcaaggtagctttcggcgtgtacattcccggatggaggttcctgcccaccaagtccaacaacaaaatgaaggagatcaaccgaaagattaagtccctccttcttggtatcatcaataagcgacagaaggccatggaggagggcgaggctggtcagtctgatctgcttggaattttgatggagtccaactccaacgagatccagggtgagggtaacaacaaggaggacggcatgtcaattgaggacgttatcgaagagtgcaaggtcttttacatcggtggacaggagactaccgccagactcttgatctggaccatgatcctgctctctagccacaccgagtggcaggaacgcgctcgaactgaggttttgaaggtctttggtaacaagaagcccgacttcgatggcctctctcggttgaaggttgtcaccatgatcctgaacgaggtcctgaggctctaccctcccgcgtccatgctgacccgaatcattcagaaggagactcgagtcggaaagcttaccctgcccgccggcgttatcttaatcatgcccattattctcatccaccgagatcatgacctctggggtgaggacgcgaacgagttcaagcctgagcgtttctccaagggcgtcagcaaggccgctaaggtgcagcccgccttctttccgttcggttggggtccccgaatctgtatgggtcaaaatttcgccatgattgaggccaagatggctctgtctttaatcctccagcgattttcgttcgagttgtcttcttcctacgttcatgccccaaccgttgtctttaccactcagccccagcacggtgcacacattgtgcttcggaaactgtag

SEQ ID NO: 321 - DNA, 1422 bp, Synthetic construct
  source 1..1422: mol_type = other DNA
atgtggactgtcgtgctgggtttagccaccctctttgttgcttactacattcattggatcaacaaatggcgggattctaagttcaatggcgtccttccccctggaacgatgggtctacccctcatcggtgaaaccatccagctgtccaggccgagtgactctctggacgttcaccccttcattcaaaagaaggtcgagcgatatggtcctatcttcaagacctgtcttgccgggcgacccgttgtcgtatccgcagatgctgagtttaacaactacattatgctccaggagggccgagccgtggagatgtggtacctggacaccctctccaagttcttcggtttggacactgagtggcttaaggccctgggactgatccacaagtacatccgatcaattaccctcaaccactttggtgccgaggctttaagagagcgattcctgcccttcatcgaagcgtcttccatggaggctcttcactcctggtctactcagccctccgtcgaggttaagaacgcctcggcccttatggttttccgtacctctgtcaacaagatgtttggcgaggacgcaaagaagctctccggtaacatccccggcaagttcaccaaactcctgggtggtttcctgtcgctccctctaaactttcccggtaccacttaccataagtgcctgaaggatatgaaggagatccagaagaagctccgagaggtcgttgacgaccgactggccaacgttggtccagatgtcgaggacttcctgggacaggcgcttaaggacaaggagtctgagaagttcattagcgaggagtttattatccagttgcttttctccatttccttcgccagtttcgaatctatttctaccactcttaccctcatcctcaagctgttggacgagcaccccgaggtggtcaaggagctcgaggccgaacacgaggctatccggaaggcccgagctgaccccgacggccccattacctgggaggagtacaagtccatgaccttcaccctgcaagtgatcaatgagactctgcgactaggctctgtaatgcctgctctcttacggaagacggtcaaggatctgcaagtcaaaggttacattatccccgagggctggaccattatgttagttaccgcctcccgacaccgagatcctaaggtctacaaggatccccacatcttcaacccatggcgatggaaggacctggactcaatcaccatccagaagaactttatgcccttcgggggtggcctccgacattgtgccggtgctgaatactccaaggtttatctttgcacattcctgcacatcctctgtacaaagtaccgatggactaaactgggaggtggtcgaatcgcccgagctcacattctgtcttttgaggacggtcttcatgtcaagttcacccctaaggagtag

SEQ ID NO: 322 - DNA, 951 bp, Synthetic construct
  source 1..951: mol_type = other DNA
atggaccaaattgagcacatcacgatcaatacaaacggtattaaaatgcatatcgcatccgtgggaaccggcccggttgtcttgcttctgcacggcttcccagaactgtggtattcgtggcgacatcagctcctctacctgagctctgtgggataccgtgccattgctcccgatctcagaggttacggagacaccgattcccccgcgtcgcctacttcttacactgccttgcacattgtcggcgaccttgttggagctctggacgagctgggtatcgagaaggtgtttctggtcggccatgattggggagccatcattgcttggtatttctgtctgtttcggcctgaccgaattaaggcgctcgttaacctttctgtgcagttcatcccccgaaaccccgccattccttttatcgagggtttccggaccgcctttggagatgacttctacatgtgccgattccaggttccgggcgaagctgaagaggacttcgcatccatcgacaccgctcagttgtttaagacgagcctctgtaacagatcgtctgccccaccttgcctgcccaaagagattggcttccgagccatcccacctccggaaaatctcccctcctggcttacagaggaggatattaactactacgctgccaagttcaagcaaactggctttaccggagcgttgaactattacagagcttttgatctgacgtgggagctcacagcaccctggactggagcccagattcaggtcccggtcaagttcattgtgggtgactctgaccttacgtatcacttccctggagcaaaggagtacatccacaacggtggtttcaagaaagacgtgcctctcctggaagaggtcgtcgtggtgaaggatgcctgccatttcatcaaccaagaacgaccgcaggagatcaatgcacacattcacgattttatcaacaaattctag

SEQ ID NO: 323 - DNA, 2079 bp, Synthetic construct
  source 1..2079: mol_type = other DNA
atgacatccgcgttgtatgcatcggacctgttcaagcagcttaaatctatcatgggtactgattccctcagcgacgatgttgtcctggtgattgctacgacctctctggctctcgtggccggctttgtcgttcttttgtggaagaagaccactgctgacagatcgggagaactcaaaccgcttatgatccctaagagcctgatggccaaggacgaggacgatgatttggacctcggctccggaaagacccgggtgtctattttctttggtactcaaaccggaacggcagagggcttcgctaaggccctgtccgaggaaatcaaggctcgatacgagaaagccgctgttaaggtcattgacctcgatgactacgcagccgacgacgatcagtacgaagagaagctcaagaaggagacattggcgtttttctgtgtcgcgacgtacggtgatggcgagcccaccgacaacgccgcccgattttataaatggttcactgaggaaaatgagcgtgacattaagctgcaacagctggcttacggagtgttcgctcttggcaaccgacagtacgagcattttaacaaaatcggcatcgtgctggacgaggagctgtgcaagaagggagccaagcgtcttattgaggttggtctgggcgatgatgatcagtcgattgaagacgactttaacgcgtggaaagaatctctgtggtctgagctcgataagctcctgaaggacgaggacgacaaatcggtcgcaaccccatacacggctgttatccccgaatacagagtggtgactcacgatcctcgattcacaacacagaagtccatggagagcaatgtcgccaacggaaacacaactatcgatatccaccatccgtgtcgggtggacgtcgccgttcaaaaggagctgcacacccatgagtctgatcgatcctgcattcacttggaatttgacattagcagaaccggtatcacctatgagacaggtgaccacgttggcgtgtacgctgaaaaccatgtggaaattgtcgaggaggcgggaaagctgctcggtcattctttggaccttgtcttctccatccacgcggacaaggaggatggtagccccctggagagcgccgttcctcccccgttccccggaccatgcacgctgggcaccggacttgcacgatacgccgacctcctgaacccccctagaaagtctgctctcgtcgccctcgctgcttacgccactgaaccttccgaggctgagaagctgaaacacctcacctcgcccgatggaaaagatgagtattcgcagtggattgttgcttcccagcgatccctgcttgaggtcatggcagccttcccctccgccaagcctccgttgggagtcttctttgctgcaatcgccccccgactgcaaccccgttactattctatttcgtcttctccacggctcgcccctagccgagttcatgtgactagcgcccttgtgtacggcccaactcctacaggcagaatccacaagggtgtgtgttcgacctggatgaagaatgcggtgccggcggagaaatcgcacgagtgttccggcgcacccatcttcattcgggcttcgaacttcaagctgccttctaacccctctaccccgattgtgatggttggccccggaacgggtctggcgccatttcggggatttctgcaagagcgaatggccctcaaggaggacggcgaagaactcggatcctcgctgctctttttcggttgccgtaaccgacagatggacttcatttacgaggacgaactgaataactttgtggaccagggagttatctctgaactgattatggccttttcccgagagggtgcccagaaggagtacgtgcagcataaaatgatggaaaaggctgcccaggtgtgggaccttatcaaggaggaaggttatctgtacgtctgcggtgacgccaagggcatggcgcgtgatgtccaccgaacgctccataccatcgtccaggagcaggagggagtgtcttccagcgaggcagaggctattgtgaagaagctgcaaactgagggacgatatcttagagatgtctggtag

SEQ ID NO: 324 - protein, 473 aa, Synthetic construct
MWTVVLGLATLFVAYYIHWINKWRDSKFNGVLPPGTMGLPLIGETIQLSRPSDSLDVHPFIQKKVERYGPIFKTCLAGRPVVVSADAEFNNYIMLQEGRAVEMWYLDTLSKFFGLDTEWLKALGLIHKYIRSITLNHFGAEALRERFLPFIEASSMEALHSWSTQPSVEVKNASALMVFRTSVNKMFGEDAKKLSGNIPGKFTKLLGGFLSLPLNFPGTTYHKCLKDMKEIQKKLREVVDDRLANVGPDVEDFLGQALKDKESEKFISEEFIIQLLFSISFASFESISTTLTLILKLLDEHPEVVKELEAEHEAIRKARADPDGPITWEEYKSMTFTLQVINETLRLGSVMPALLRKTVKDLQVKGYIIPEGWTIMLVTASRHRDPKVYKDPHIFNPWRWKDLDSITIQKNFMPFGGGLRHCAGAEYSKVYLCTFLHILCTKYRWTKLGGGRIARAHILSFEDGLHVKFTPKE

SEQ ID NO: 325 - skipped

SEQ ID NO: 326 - DNA, 1470 bp, Synthetic construct
  source 1..1470: mol_type = other DNA
atggttacgcaacagtccgccgctgagacttctgccacccagaccaacgaatatgacgtggtcatcgttggtgctggaattgcaggtcccgccctcgccgtcgctcttggcaatcagggtcggaaggtcctggttgtcgagcgagatctgtcggagcctgaccgtatcgttggggagttgctccagcccggtggtgtggccgcccttaagaccctgggcctcggctcttgcatcgaggatattgacgccattccatgtcagggttacaacgtcatctactccggagaggagtgtgtactgaaataccccaaggttcctcgagacatccagcaggactacaacgaattataccgatccggtaagtctgcggatatctccaacgaggctccccgaggtgtctcctttcaccatggccgcttcgtcatgaacctccgacgagcagctcgagacacccccaacgtgactctgctggaggccaccgttaccgaggtcgtaaagaacccctacactggtcacatcattggagtcaagaccttctctaagaccggtggtgccaagatttacaagcacttctttgctccgctcactgtggtctgcgatggcaccttctctaagttccgaaaggacttctccaccaacaagacctcggttcgaagtcactttgccggtcttatcttgaaggacgccgttctcccctccccccagcacggtcatgtcatcctgtctcccaactcatgtcctgttcttgtctaccaggtcggtgctcgagagactagaattttatgcgatatccaagggcccgtaccttccaacgccaccggtgctctaaaggagcacatggagaaaaatgtgatgccccacctaccaaagtctatccagccctcctttcaggccgcactcaaggaacagaccattcgagtcatgcctaactctttcctttccgcctcgaagaacgaccatcacggcctgatcctgctcggtgacgctctcaacatgcgacaccccctgactggtggaggaatgaccgttgcgcttaacgacgcccttctcctctcccgactgctgacaggtgtcaaccttgaggacacctacgctgtgtcttcagttatgagctcgcagttccactggcagcgaaagcatctggactccattgtcaacatcctctctatggccctgtactccctcttcgccgctgattccgactacctgcgaatccttcagctcggctgtttcaattatttcaagctgggcggtatttgcgtcgaccaccccgttatgctcctcgctggcgtgcttccccgaccgatgtacctctttacccacttcttcgtggtggctatctacggtggaatttgtaatatgcaggccaacggtatcgccaagctgcccgcctctctgctccaattcgtcgcctctcttgtcactgcttgtatcgttatttttccttacatctggtctgagctgacgtag

SEQ ID NO: 327 - DNA, 2229 bp, Synthetic construct
  source 1..2229: mol_type = other DNA
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccacacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctcagacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatcagaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggattcattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcacggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtag

SEQ ID NO: 328 - protein, 489 aa, unidentified
  source 1..489: note = Yarrowia lipolytica
MVTQQSAAETSATQTNEYDVVIVGAGIAGPALAVALGNQGRKVLVVERDLSEPDRIVGELLQPGGVAALKTLGLGSCIEDIDAIPCQGYNVIYSGEECVLKYPKVPRDIQQDYNELYRSGKSADISNEAPRGVSFHHGRFVMNLRRAARDTPNVTLLEATVTEVVKNPYTGHIIGVKTFSKTGGAKIYKHFFAPLTVVCDGTFSKFRKDFSTNKTSVRSHFAGLILKDAVLPSPQHGHVILSPNSCPVLVYQVGARETRILCDIQGPVPSNATGALKEHMEKNVMPHLPKSIQPSFQAALKEQTIRVMPNSFLSASKNDHHGLILLGDALNMRHPLTGGGMTVALNDALLLSRLLTGVNLEDTYAVSSVMSSQFHWQRKHLDSIVNILSMALYSLFAADSDYLRILQLGCFNYFKLGGICVDHPVMLLAGVLPRPMYLFTHFFVVAIYGGICNMQANGIAKLPASLLQFVASLVTACIVIFPYIWSELT

SEQ ID NO: 329 - protein, 742 aa, Synthetic construct
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDQKKQVSDKRKHTAIDLLLGMQNVGSFEPGSFASYEPIRASSMLEKINPAEVFGNIMVEYPYVECTDSVVLGLSYFRKYHDYRNEDVDRAISAAIGFIIREQQPDGGFFGSWGVCYCYAHMFAMEALETQNLNYNNCSTVQKACDFLAGYQEADGGWAEDFKSCETQMYVHGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 330 - DNA, 630 bp, Synthetic construct
  source 1..630: mol_type = other DNA
atggagctgtactggaaggccatggagcagatcgcctggtacactggtctgtcccccaccgccttcttcaccattctggcctccatgatctttgtgttccagatggtcagctccatgttcgtttctcccgaggagttcaacaagcctcccaccgtccccgtcagctcttccaaccccgccaactccaacctcttcgtcaacgactctgtggctgatgcttcccaggccgtccagattggtcagctcaccgagcagcagctccgtgcttatgacggcagtgaccccaacaagcctcttctcatggccatcaagggccagatctacgacgtttcttctggacgaatgttctacggaccgggctctccttacgctatgtttgccggcaaggatgcttctcgagctctggctctgctgtccttcaagcccgaggacatcaccggtaacatcgagggtttgtccgaggaggagctcgttatccttcaggactgggagtacaagttcatggagaagtacgtcaaggttggtgagcttgtccttgagggaggaggagaagggatgaacgagcactccgagaacggtcaccgaaactttgagattgaccaggaggaggagcgaatcgaggctcgatag

SEQ ID NO: 331 - DNA, 843 bp, Synthetic construct
  source 1..843: mol_type = other DNA
atggctctgcaagtctgggagactctcaaggaggccattgtcgcttacaccggtctgtctcccgctaccttcttcactgtgctcgctcttggtttggccgtctactacgtcatttcgggcttcttcggcacctcggactacggctctcactctcgagatttcggtgagcagatgcagcccctgcctcctcccgtccaggttggtgagatctctgaggaggagctcaagcagtacgacggcagtgactccaagaagcctcttctcatggccatcaagggccagatttacgatgtcagccagagccgaatgttctacggacccggtggcccttacgctctgtttgccggcaaggatgcttctcgagctctggccaaaatgtcatttgaagagaaggacctgactggtgacatttccggactcggcccattcgagcttgaggccctccaggattgggaatacaagttcatgtccaagtacgttaaggtcggcaccgttgagaagcccgtcagcgagacagacgctgctcccgccggtgagtctgctgagtccaccaacgccgaggtgtccaagcccgctgaggacggtccttccgagtcccaggccgacaaggctgaggagacagtcgctgccaagcagaagaaggagaagaagaagggccgacgacgagtcgagggtgttctcatggagatccgaggcgaggccgttggtggccgaaagggcgagggcatgtcgagggagattggccgaggtggtggtggtgccgacgacggcaacctcaagggtggtggtatcgaccacggtgagcgagaacgagagcgagaacgagagtccggcatgtcttag

SEQ ID NO: 332 - DNA, 2229 bp, Synthetic construct
  source 1..2229: mol_type = other DNA
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtgggagtatgacgataccacaccgctggaagagctggaaaagagggctaccgactacgtcaaatactcgctggagctgccgggatacgcgcccgtgactctggactccaagcccgtgaaaaatgcctacgaagcggctatcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggtcgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgtcctccattgtcatgtatctccatgaggggcccgatccggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgatggaggaaaattcctatcgagacgaccgcgtcggaggatggccctttagtaccaagacccaggggtatccagtctccgactgtactgccgaggctctcaaggccatcatcatggtccagaatacacctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggacccagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtag

SEQ ID NO: 333 - DNA, 2106 bp, Synthetic construct
  source 1..2106: mol_type = other DNA
atgaaggtttcccctttcgagtttatgtctgctattatcaaaggacggatggatccctcaaattccagcttcgaatccactggtgaggtggcctctgtcatcttcgagaacagggagctggtcgccattcttaccaccagtatcgcagtaatgattggttgttttgttgtcttgatgtggcgacgagctggctctcgaaaggttaagaacgtcgagctgcccaagccactcatcgtccacgagcccgagcctgaagtggaggacggtaagaagaaggtcagcatcttcttcggcacccagacgggtaccgccgaggggttcgccaaggctttagccgacgaggccaaggcgcgatacgagaaggctacttttcgagttgtggacctggatgactatgccgctgacgatgaccaatacgaggagaagctcaagaacgaatccttcgcagtttttctccttgcgacctacggtgacggagagcccacagacaacgctgcccgattctacaagtggttcgctgagggtaaagagcgaggtgagtggctgcaaaacctccattacgccgtctttggccttggtaaccgacagtacgagcacttcaacaagatcgccaaggttgccgatgagctgcttgaggcccagggcggtaatcgactggttaaggttggcctcggagatgacgaccagtgcattgaggacgacttctcggcttggcgcgagtccctgtggcccgagttggatatgctacttagagatgaagacgacgccaccaccgtcactaccccctacacagctgccgtcctggagtaccgtgtcgtgttccacgactctgccgatgttgctgccgaggataagtcctggatcaacgccaacggtcacgctgtccatgacgctcagcacccgttccgatctaacgtagtcgttcgaaaggagctacacacctccgcctctgaccgatcctgttctcacctggagtttaacatttccggttccgccctcaactacgagactggtgaccatgtcggcgtctactgcgaaaacctcaccgagactgtcgacgaggccctcaatttgctgggtctgtcccctgagacatatttctccatctacaccgacaacgaggacggtaccccccttggaggctcttctctcccccctccctttcccagctgtaccctgcgaaccgcgctgacccgatacgccgacttgctcaactcgcccaaaaagtctgctcttctcgctctggccgcacacgcgtccaaccccgtcgaggccgaccgactccgttacctggccagtccggctggaaaggatgaatacgcccagtccgttattggttcgcagaagtctctcctagaggtgatggctgagttcccctccgccaagccccctcttggtgtattctttgctgccgtcgcccctagactccagccacgattctactctatcagcagttccccccgaatggctccctctcggattcacgttacctgcgccctcgtgtacgacaagatgcccaccggtcgaatccacaagggagtgtgttccacttggatgaagaactctgtccccatggagaagtcccatgagtgctcctgggctcctatcttcgtccgacagtcgaacttcaagcttcccgcggagtctaaggttcccatcattatggtgggtcctggcaccggtctggctcccttccgtggatttctgcaagagcgactcgcccttaaggaatctggcgttgagctgggtccttctattctgtttttcggttgtcgaaaccgacgaatggattatatctacgaggacgagttgaacaacttcgtcgagactggagccctttccgagcttgtcattgccttcagccgagagggtcccaccaaagagtacgtgcagcacaagatggccgagaaggcatccgatatctggaatctgatctctgaaggtgcttacctctacgtttgtggggacgccaagggtatggccaaggacgtccaccgaaccttgcacaccatcatgcaggagcagggctccttagattccagtaaggctgagtctatggttaagaacctccagatgaacggtcgctacctgcgagatgtgtggtag

SEQ ID NO: 334 - DNA, 1563 bp, Synthetic construct
  source 1..1563: mol_type = other DNA
atggagatgtcgagctccgtggccgcaacgatttccatctggatggtcgttgtgtgtatcgtcggcgttggttggagagtggtcaattgggtctggctgcggccaaaaaagttggaaaagcgtcttcgagagcagggactcgcgggaaactcttaccgactgctcttcggcgacctgaaggagcgagccgctatggaagagcaagcaaactctaaaccgattaacttttcgcacgatatcggtccccgtgttttcccttctatgtataagacaattcagaactacggaaagaactcctacatgtggcttggcccttacccccgagtgcatatcatggacccccagcagctcaagactgtgtttaccctggtctatgatattcaaaagcccaatttgaacccgctgattaagttcctcctggacggaatcgtgacccacgaaggcgagaaatgggccaagcatagaaaaattatcaatcctgctttccacctggagaagctgaaggacatgattccagcttttttccacagctgcaacgagatcgttaacgagtgggagcggttgatctctaaggaaggttcgtgcgagttggatgtcatgccctaccttcagaacctcgccgccgacgctatttcccgaaccgcctttggatcttcgtacgaggaaggcaagatgatctttcagctccttaaagagctgacagacctggttgtgaaggttgcattcggtgtgtacattcctggctggcgatttttgccgactaagagcaacaacaagatgaaggagatcaatcgaaagatcaaatccctgctccttggaatcattaataagagacaaaaagcgatggaagagggtgaagctggccagtcggatctgcttggtatcctgatggagtctaacagcaacgaaattcagggtgagggaaacaacaaggaggacggcatgtccatcgaagatgtgattgaggaatgtaaggtcttctacattggtggacaggagactaccgccagactcctgatttggacaatgatccttctcagctctcataccgagtggcaggaacgggctcgaaccgaggtcttgaaagttttcggcaataagaagcctgactttgacggcttgtcgcgtcttaaggtggtgacgatgatcctcaacgaggtcctccgactgtatccccccgcgtccatgctgactagaattatccagaaggaaacccgtgtcggtaagctcaccctcccagcgggagttattttgatcatgcccatcatcctgattcatcgggatcacgacctgtggggagaggacgccaacgagttcaagcctgagcgattctccaagggcgtttctaaagctgcgaaggtccaaccggccttctttcccttcggctggggacctcgaatttgtatgggccagaatttcgcaatgatcgaggccaaaatggctttgtcgctcatcctgcaacggttttcctttgaactgtcctcttcttatgtgcacgcccctacggtggtctttactacacagccgcaacacggcgcacacatcgtcttgcgaaaactgtag

SEQ ID NO: 335 - DNA, 1563 bp, Synthetic construct
  source 1..1563: mol_type = other DNA
atggaaatgtcttcgtccgtcgcggccacgattagcatctggatggttgtggtgtgtatcgtcggcgtgggttggagagttgtgaactgggtctggcttcgacctaaaaagctggagaagcgtttgcgagagcagggactcgctggcaattcttaccggctgctgtttggagacttgaaggagcgagcagccatggaggaacaagctaactccaagcccattaacttctcgcatgatatcggtccgagagttttcccaagcatgtataagaccattcagaattacggaaagaactcgtacatgtggcttggcccctatcctcgagtccacattatggacccgcagcagctcaaaactgtgtttacactggtctacgatatccagaagcccaaccttaaccccctcatcaagttcctgctggacggcatcgtgacccacgagggtgaaaaatgggccaagcatcggaagattattaatcctgcttttcacctcgagaagctgaaggacatgatcccagcattctttcactcttgcaacgaaatcgttaacgagtgggagcgactcatttccaaagagggttcctgcgagctggacgtcatgccctacctgcaaaacctcgccgctgatgcgatctctcggactgccttcggatcttcctacgaagagggaaagatgatcttccagctcttgaaggagcttaccgatttggttgtgaaggtcgcttttggtgtgtacattcctggctggagattcctgccaacaaaatcgaataacaagatgaaggagattaaccgaaagattaaatcgttgctcctgggtatcattaataaacgtcagaaggccatggaagagggcgaggccggccagtccgacctgctcggaatccttatggaatccaactctaacgaaattcaaggagagggaaacaacaaggaggacggcatgagcattgaagatgtgatcgaggaatgtaaggtcttctacatcggaggccaagagactacggcgcgactcctgatttggaccatgatcctgttgtcttctcataccgagtggcaggagcgagctcgtacagaagtgttgaaggtgttcggcaacaaaaaacccgactttgacggtctgtcgcgactgaaggttgtgactatgatcctcaatgaggttctcagactgtaccctcccgcctctatgcttacccgaattattcagaaggagacacgtgtgggtaagcttacccttcccgcaggcgtcatcctcatcatgcctattatcctgatccaccgggaccacgatctctggggcgaggatgccaacgagtttaagcccgagagattttctaagggagtttctaaggctgccaaagtccagcccgcattcttccccttcggttggggacctcgaatctgtatgggacagaactttgcgatgattgaagcaaagatggctctcagcttgattcttcagagatttagctttgagctgtcctcgtcttatgtccatgcaccgactgttgtgttcaccacccaaccacagcatggcgcccacattgttctgcggaaattgtag

SEQ ID NO: 336 - protein, 742 aa, Synthetic construct
MGIHESVSKQFAKNGHSKYRSDRYGLPKTDLRRWTFHASDLGAQWWEYDDTTPLEELEKRATDYVKYSLELPGYAPVTLDSKPVKNAYEAAIKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMSSIVMYLHEGPDPANYKKIAARWPEFLSLNPSGMFMNGTNGLQVWDTAFAVQYACVCGFAELPQYQKTIRAAFDFLDRSQINEPMEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDPAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 337 - protein, 742 aa, unidentified
  source 1..742: note = Yarrowia lipolytica
MGIHESVSKQFAKNGHSKYRSDRYGLPKTDLRRWTFHASDLGAQWWKYDDTTPLEELEKRATDYVKYSLELPGYAPVTLDSKPVKNAYEAALKNWHLFASLQDPDSGAWQSEYDGPQFMSIGYVTACYFGGNEIPTPVKTEMIRYIVNTAHPVDGGWGLHKEDKSTCFGTSINYVVLRLLGLSRDHPVCVKARKTLLTKFGGAINNPHWGKTWLSILNLYKWEGVNPAPGELWLLPYFVPVHPGRWWVHTRWIYLAMGYLEAAEAQCELTPLLEELRDEIYKKPYSEIDFSKHCNSISGVDLYYPHTGLLKFGNALLRRYRKFRPQWIKEKVKEEIYNLCLREVSNTRHLCLAPVNNAMTSIVMYLHEGPDSANYKKIAARWPEFLSLNPSGMFMNGTNGLQVWDTAFAVQYACVCGFAELPQYQKTIRAAFDFLDRSQINEPTEENSYRDDRVGGWPFSTKTQGYPVSDCTAEALKAIIMVQNTPGYEDLKKQVSDKRKHTAIDLLLGMQNVGSFEPGSFASYEPIRASSMLEKINPAEVFGNIMVEYPYVECTDSVVLGLSYFRKYHDYRNEDVDRAISAAIGYIIREQQPDGGFFGSWGVCYCYAHMFAMEALETQNLNYNNCSTVQKACDFLAGYQEADGGWAEDFKSCETQMYVRGPHSLVVPTAMALLSLMSGRYPQEDKIHAAARFLMSKQMSNGEWLKEEMEGVFNHTCAIEYPNYRFYFVMKALGLYFKGYCQ

SEQ ID NO: 338 - DNA, 2229 bp, Synthetic construct
  source 1..2229: mol_type = other DNA
atgggaatccacgaaagtgtgtcgaaacagtttgcgaaaaacggacattccaagtaccgcagcgaccgatacggcttacctaagacggatctgcgacgatggacgttccacgcgtccgatctgggggcgcaatggtggaagtatgacgataccacaccgctggaagagctggaaaagagggctaccgactacgtcaaatactcgctggagctgccgggatacgcgcccgtgactctggactccaagcccgtgaaaaatgcctacgaagcggctctcaaaaactggcatctgtttgcgtcgctgcaagaccccgactccggcgcatggcagtcggaatacgacggaccgcagttcatgtcgatcggttatgtgacggcgtgctactttggcggcaacgagatccccacgccggtcaaaaccgaaatgatcagatacattgtcaacacagcccacccagttgacggaggctggggccttcacaaagaagacaagagcacctgtttcggtaccagcatcaactacgtggtcctgcgactactgggcctgtcacgggatcatccggtctgcgtcaaggcgcgcaaaacgctgctcaccaagtttggcggcgccatcaacaacccccattggggcaagacctggctgtcgattctcaatctctacaaatgggagggtgtgaatccggcccctggcgagctctggctgttgccctactttgttcctgttcatccgggccgatggtgggtccatacccggtggatctaccttgccatgggctatctggaggctgcggaggcccaatgcgaactcactccgttgctggaggagctccgagacgaaatctacaaaaagccctactcggagattgatttctccaaacattgcaactccatctccggagtcgacctctactatccccacaccggccttttgaagtttggcaacgcgcttctccgacgataccgcaagttcagaccgcagtggatcaaagaaaaggtcaaggaggaaatttacaacttgtgccttcgagaggtttccaacacacgacacttgtgtctcgctcccgtcaacaatgccatgacctccattgtcatgtatctccatgaggggcccgattcggcgaattacaaaaagattgcggcccgatggcccgaatttctgtctctgaatccgtcgggaatgtttatgaacggcaccaacggtctgcaggtctgggatactgcgtttgccgtgcaatacgcgtgtgtttgtggctttgccgaacttccccagtaccagaagacgatccgagcggcgtttgattttctcgatcggtcccagatcaacgagccgacggaggaaaattcctatcgagacgaccgcgtcggaggatggccctttagtaccaagacccaggggtatccagtctccgactgtactgccgaggctctcaaggccatcatcatggtccagaatacgcctggatacgaggatctgaagaaacaagtgtctgacaagcggaaacacactgccatcgatctacttttgggaatgcagaacgtgggctcgtttgaaccgggctctttcgcctcctatgagcctatccgggcgtcgtccatgctggagaagatcaatccggccgaggtgtttggaaacatcatggtggagtatccgtacgtggaatgcactgattctgttgttctgggtctgtcctactttcgaaagtaccacgattaccgcaacgaagacgtggaccgagccatctctgctgccattggatacattattcgagagcagcagcctgacggcggcttctttggctcctggggcgtgtgctactgctacgctcacatgtttgccatggaggctctggagacgcagaatctcaactataacaactgttccacggttcaaaaggcgtgcgactttctggcgggctaccaggaagcagatggaggctgggccgaggactttaagtcgtgcgagactcagatgtacgtgcgcggaccccattcgctggtcgtgcctactgccatggccctgttgagtttgatgagtggtcggtatccccaggaggacaagattcatgctgcggcccggtttctcatgagcaagcagatgagcaacggtgagtggctcaaggaggagatggagggggtgtttaaccatacttgtgccattgagtatcccaactaccggttttattttgtcatgaaggctttggggttgtatttcaagggatattgccagtga

(19) United States
(12) Patent Application Publication
(10) Pub. No.: US 2025/0075243 A1
(43) Pub. Date: Mar. 6, 2025

(54) BIOSYNTHESIS OF OXYGENATED HYDROCARBONS

(71) Applicant: Ginkgo Bioworks, Inc., Boston, MA (US)

(72) Inventors: Elena Brevnova, Winchester, MA (US); Bernardo Moura Torres da Costa, Medford, MA (US); Krupanandan Haranahalli, Cambridge, MA (US); Gabriel Rodriguez, Central, SC (US); Michael Torrens-Spence, Cambridge, MA (US); Justin Vento, Boston, MA (US)

(73) Assignee: Ginkgo Bioworks, Inc., Boston, MA (US)

(21) Appl. No.: 18/830,141

(22) Filed: Sep. 10, 2024

Related U.S. Application Data
(63) Continuation of application No. PCT/US2023/073892, filed on Sep. 11, 2023.
Application No. PCT/US2023/073892 is a continuation-in-part of application No. PCT/US2023/064106, filed on Mar. 10, 2023.
(60) Provisional application No. 63/422,105, filed on Nov. 3, 2022.
(60) Provisional application No. 63/406,614, filed on Sep. 14, 2022.
(60) Provisional application No. 63/319,213, filed on Mar. 11, 2022.

(51) Int. Cl. C12P 33/12; C07K 14/80; C12N 1/18; C12N 1/20; C12N 9/02; C12N 9/04; C12N 9/14; C12P 7/26; C12P 17/12; C12P 17/18; C12P 33/20; C12R 1/865

(52) U.S. Cl. CPC C12P 33/12; C07K 14/80; C12N 1/18; C12N 1/20; C12N 9/0006; C12N 9/001; C12N 9/0053; C12N 9/0079; C12N 9/14; C12P 7/26; C12P 17/12; C12P 17/182; C12P 33/20; C12R 2001/865; C12Y 101/01288; C12Y 103/03006; C12Y 109/03001; C12Y 114/14001; C12Y 114/15004; C12Y 303/0201

(57) ABSTRACT

Described herein are CB5 polypeptides for the biosynthesis of oxygenated hydrocarbons in modified host cells.

### CROSS-REFERENCE TO RELATED APPLICATIONS

This application claims the benefit under 35 U.S.C. § 119(e) of U.S. Provisional Application No. 63/406,614, filed Sep. 14, 2022, entitled “BIOSYNTHESIS OF OXYGENATED HYDROCARBONS,” and U.S. Provisional Application No. 63/422,105, filed Nov. 3, 2022, entitled “BIOSYNTHESIS OF OXYGENATED HYDROCARBONS.” the entire disclosure of each which is hereby incorporated by reference in its entirety.

### REFERENCE TO AN ELECTRONIC SEQUENCE LISTING

The contents of the electronic sequence listing (G091970104WO00-SEQ-FL.xml; Size: 382.723 bytes; and Date of Creation: Sep. 7, 2023) is herein incorporated by reference in its entirety.

### FIELD OF THE INVENTION

The present disclosure relates to the production of oxygenated hydrocarbons in recombinant cells.

### BACKGROUND

Many natural products that have useful properties are oxygenated hydrocarbons produced by plants in biosynthetic pathways that include one or more cytochrome P450 enzymes (CYP). These natural products include isoprenoids and derivatives of aromatic amino acids, which can be useful as flavoring agents, fragrance compounds, antioxidants, food dyes, dietary supplements, and medicinal compounds. The structural complexity of these oxygenated hydrocarbons often limits their availability as de novo chemical synthesis can be challenging and extraction from natural sources can be costly or labor intensive.

### SUMMARY

The present disclosure provides, in some embodiments, for the production of natural products which are oxygenated hydrocarbons using recombinant cells, thereby, providing, in some embodiments, an alternative source of such natural products.

Aspects of the present disclosure provide host cells and methods useful for the production of oxygenated hydrocarbons. In some embodiments, the CB5 comprises a sequence that is at least 70%, 75%, 80%, 85%, 90%, or 95% identical to any one of SEQ ID NOs: 1-6, 10, 30, 33-34, and 318.

In some embodiments, the CB5 comprises a sequence that is at least 95% identical to any one of SEQ ID NOs: 1-6, 10, 30, 33-34, and 318.

In some embodiments, CB5 comprises the sequence of any one of SEQ ID NOs: 1-6, 10, 30, 33-34, and 318.

In some embodiments, the heterologous polynucleotide comprises a sequence that is at least 90% identical to any one of SEQ ID NOs: 11-24, 36-40, 95-99, 100-106, 316-317, and 330-331.

In some embodiments, the heterologous polynucleotide comprises the sequence of any one of SEQ ID NOs: 11-24, 36-40, 95-99, 100-106, 316-317, and 330-331.

In some embodiments, the heterologous polynucleotide comprises a sequence that is at least 90% identical to any one of SEQ ID NOs: 11, 12, 13, 14, 15, 22, 23, 36, 37, 96, 97, 99, 100, 101, 102, 103, 104, 105, 106, 316, 317, and 331.

In some embodiments, the heterologous polynucleotide comprises a sequence selected from SEQ ID NOs: 11, 12, 13, 14, 15, 22, 23, 36, 37, 96, 97, 99, 100, 101, 102, 103, 104, 105, 106, 316, 317, and 331.

Further aspects of the present disclosure provide host cells that comprise: (a) a heterologous polynucleotide encoding a cytochrome b5 (CB5), wherein the CB5 comprises an amino acid sequence that is at least 70%, 75%, 80%, 85%, 90%, or 95% identical to any one of SEQ ID NOs: 1-6, 10, 30, 33-34, and 318; and (b) a heterologous polynucleotide encoding a cytochrome P450 (CYP), wherein the host cell is capable of producing more of an oxygenated hydrocarbon than a control host cell that does not comprise the heterologous polynucleotide encoding the CB5.

In some embodiments, the CYP is a C11 hydroxylase, ABA1, ABA2, T16H2, T3O, PPDS, AD1, AD5, AD6, C28 oxidase, C16 oxidase, C23 oxidase, and/or CrtS.

In some embodiments, the CYP comprises an amino acid sequence that is at least 90% identical to a sequence selected from SEQ ID NOs: 43, 89, 90, 117, 120, 128, 280, 281, and 324.

Further aspects of the present disclosure provide host cells that comprise a heterologous polynucleotide encoding a cytochrome b5 (CB5), wherein the host cell is capable of producing more of an oxygenated hydrocarbon than a control host cell that does not comprise the heterologous polynucleotide, and wherein the CB5 comprises: the amino acid sequence YTGLSP (SEQ ID NO: 47); the amino acid sequence KPLLMAIKGQIYDVS (SEQ ID NO: 48); the amino acid sequence LQDWEYKFM (SEQ ID NO: 49); and/or the amino acid sequence X₁X₂X₃EX₄GX₅X₆X₇X₈X₉X₁₀D (SEQ ID NO: 53), wherein: X₁ is the amino acid K or E; X₂ is the amino acid P or H; X₃ is the amino acid A or S; X₄ is the amino acid D or N; X₅ is the amino acid P or H; X₆ is the amino acid S or R; X₇ is the amino acid E or N; X₈ is the amino acid S or F; X₉ is the amino acid Q or E; and/or X₁₀ is the amino acid A or I; and/or the amino acid sequence DATX₁X₂FX₃X₄X₅VGHS (SEQ ID NO: 31), wherein: X₁ is the amino acid E, D, or N; X₂ is the amino acid A or D; X₃ is the amino acid E or D; X₄ is the amino acid D or N; and/or X₅ is the amino acid V or A.

In some embodiments, the CB5 comprises: the amino acid sequence X₁X₂X₃X₄X₅X₆X₇EX₈X₉X₁₀YTGLSPX₁₁X₁₂FFTX₁₃LAX₁₄X₁₅X₁₆X₁₇VX₁₈X₁₉X₂₀X₂₁SX₂₂X₂₃FX₂₄X₂₅X₂₆X₂₇X₂₈X₂₉X₃₀X₃₁ (SEQ ID NO: 50), wherein: X₁ is the amino acid E or Q; X₂ is the amino acid L or V; X₃ is the amino acid Y or W; X₄ is the amino acid W or E; X₅ is the amino acid K or T; X₆ is the amino acid A or L; X₇ is the amino acid M or K; X₈ is the amino acid Q or A; X₉ is the amino acid A or V; X₁₀ is the amino acid W or A; X₁₁ is the amino acid T or A; X₁₂ is the amino acid A or T; X₁₃ is the amino acid I or V; X₁₄ is the amino acid S or L; X₁₅ is the amino acid M or G; X₁₆ is the amino acid I or L; X₁₇ is the amino acid F or A; X₁₈ is the amino acid F or Y; X₁₉ is the amino acid Q or Y; X₂₀ is the amino acid M or V; X₂₁ is the amino acid V or I; X₂₂ is the amino acid S or G; X₂₃ is the amino acid M or F; X₂₄ is the amino acid V or G; X₂₅ is the amino acid S or T; X₂₆ is the amino acid P or S; X₂₇ is the amino acid E or D; X₂₈ is the amino acid E or Y; X₂₉ is the amino acid F or G; X₃₀ is the amino acid N or S; and/or X₃₁ is the amino acid K or H; the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51), wherein: X₁ is the amino acid P or A; X₂ is the amino acid V or I; X₃ is the amino acid E or Q; X₄ is the amino acid I or L; X₅ is the amino acid S or T; X₆ is the amino acid E or Q; X₇ is the amino acid E or Q; X₈ is the amino acid K or R; X₉ is the amino acid Q or A; X₁₀ is the amino acid S or P; X₁₁ is the amino acid K or N; X₁₂ is the amino acid Q or S; and/or X₁₃ is the amino acid S or G; the amino acid sequence LAX₁X₂SFX₃X₄X₅DX₆TGX₇IX₈GLX₉X₁₀X₁₁ELX₁₂X₁₃ LQDWEYKFMX₁₄KYVKVGX₁₅X₁₆ (SEQ ID NO: 52), wherein: X₁ is the amino acid K or L; X₂ is the amino acid M or L; X₃ is the amino acid E or K; X₄ is the amino acid E or P; X₅ is the amino acid K or E; X₆ is the amino acid L or I; X₇ is the amino acid D or N; X₈ is the amino acid S or E; X₉ is the amino acid G or S; X₁₀ is the amino acid P or E; X₁₁ is the amino acid F or E; X₁₂ is the amino acid E or V; X₁₃ is the amino acid A or I; X₁₄ is the amino acid S or E; X₁₅ is the amino acid T or E; and/or X₁₆ is the amino acid V or L; the amino acid sequence X₁X₂X₃EX₄GX₅X₆X₇X₈X₉X₁₀D (SEQ ID NO: 53), wherein: X₁ is the amino acid K or E; X₂ is the amino acid P or H; X₃ is the amino acid A or S; X₄ is the amino acid D or N; X₅ is the amino acid P or H; X₆ is the amino acid S or R; X₇ is the amino acid E or N; X₈ is the amino acid S or F; X₉ is the amino acid Q or E; and/or X₁₀ is the amino acid A or I; and/or the amino acid sequence DATX₁X₂FX₃X₄X₅VGHS (SEQ ID NO: 31), wherein: X₁ is the amino acid E, D, or N; X₂ is the amino acid A or D; X₃ is the amino acid E or D; X₄ is the amino acid D or N; and/or X₅ is the amino acid V or A.

In some embodiments, the CB5 comprises: the amino acid sequence X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃FX₂₄X₂₅X₂₆X₂₇X₂₈X₂₉X₃₀X₃₁ (SEQ ID NO: 50), wherein: X₁ is the amino acid E or Q; X₂ is the amino acid L or V; X₃ is the amino acid Y or W; X₄ is the amino acid W or E; X₅ is the amino acid K or T; X₆ is the amino acid A or L; X₇ is the amino acid M or K; X₈ is the amino acid Q or A; X₉ is the amino acid A or V; X₁₀ is the amino acid W or A; X₁₁ is the amino acid T or A; X₁₂ is the amino acid A or T; X₁₃ is the amino acid I or V; X₁₄ is the amino acid S or L; X₁₅ is the amino acid M or G; X₁₆ is the amino acid I or L; X₁₇ is the amino acid F or A; X₁₈ is the amino acid F or Y; X₁₉ is the amino acid Q or Y; X₂₀ is the amino acid M or V; X₂₁ is the amino acid V or I; X₂₂ is the amino acid S or G; X₂₃ is the amino acid M or F; X₂₄ is the amino acid V or G; X₂₅ is the amino acid S or T; X₂₆ is the amino acid P or S; X₂₇ is the amino acid E or D; X₂₈ is the amino acid E or Y; X₂₉ is the amino acid F or G; X₃₀ is the amino acid N or S; and/or X₃₁ is the amino acid K or H; the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51), wherein: X₁ is the amino acid P or A; X₂ is the amino acid V or I; X₃ is the amino acid E or Q; X₄ is the amino acid I or L; X₅ is the amino acid S or T; X₆ is the amino acid E or Q; X₇ is the amino acid E or Q; X₈ is the amino acid K or R; X₉ is the amino acid Q or A; X₁₀ is the amino acid S or P; X₁₁ is the amino acid K or N; X₁₂ is the amino acid Q or S; and/or X₁₃ is the amino acid S or G; and/or the amino acid sequence LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52), wherein: X₁ is the amino acid K or L; X₂ is the amino acid M or L; X₃ is the amino acid E or K; X₄ is the amino acid E or P; X₅ is the amino acid K or E; X₆ is the amino acid L or I; X₇ is the amino acid D or N; X₈ is the amino acid S or E; X₉ is the amino acid G or S; X₁₀ is the amino acid P or E; X₁₁ is the amino acid F or E; X₁₂ is the amino acid E or V; X₁₃ is the amino acid A or I; X₁₄ is the amino acid S or E; X₁₅ is the amino acid T or E; and/or X₁₆ is the amino acid V or L.

In some embodiments, CB5 comprises one or more of the following amino acid sequences; QVWETLKEAIVAYTGLSPATFFTVLALGLAVYYVISGFFGTSDYGSH (SEQ ID NO: 58) or ELYWKAMEQIAWYTGLSPTAFFTILASMIFVFQMVSSMFVSPEEFNK (SEQ ID NO: 59); PVQVGEISEEELKQYDGSDSKKPLLMAIKGQIYDVSQSRMF (SEQ ID NO: 60) or AVQIGQLTEQQLRAYDGSDPNKPLLMAIKGQIYDVSSGRMF (SEQ ID NO: 61); LAKMSFEEKDLTGDISGLGPFELEALQDWEYKFMSKYVKVGTV (SEQ ID NO: 62) or LALLSFKPEDITGNIEGLSEEELVILQDWEYKFMEKYVKVGEL (SEQ ID NO: 63); KPAEDGPSESQAD (SEQ ID NO: 64) or EHSENGHRNFEID (SEQ ID NO: 65); and DATEAFEDVGHS (SEQ ID NO: 108), DATDDFENVGHS (SEQ ID NO: 109), DATDDFEDVGHS (SEQ ID NO: 110), DATDDFEDAGHS (SEQ ID NO: 111), or DATNDFDDVGHS (SEQ ID NO: 112).

In some embodiments, the CB5 comprises: the amino acid sequence YTGLSP (SEQ ID NO: 47) at residues corresponding to positions 16-21 in SEQ ID NO: 1; the amino acid sequence KPLLMAIKGQIYDVS (SEQ ID NO: 48) at residues corresponding to positions 85-99 in SEQ ID NO: 1; and/or the amino acid sequence LQDWEYKFM (SEQ ID NO: 49) at residues corresponding to positions 148-156 in SEQ ID NO: 1.

In some embodiments, the CB5 comprises the amino acid sequence X₁X₂X₃EX₄GX₅X₆X₇X₈X₉X₁₀D (SEQ ID NO: 53) at residues corresponding to positions 190-202 of SEQ ID NO: 1.

In some embodiments, the CB5 comprises: the amino acid sequence X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃FX₂₄X₂₅X₂₆X₂₇X₂₈X₂₉X₃₀X₃₁ (SEQ ID NO: 50) at residues corresponding to positions 4-50 of SEQ ID NO: 1: the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51) at residues corresponding to positions 64-104 of SEQ ID NO: 1; and/or the amino acid sequence LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52) at residues corresponding to positions 123-165 of SEQ ID NO: 1.

In some embodiments, the CB5 comprises the amino acid sequence DATX₁X₂FEX₃VGHS (SEQ ID NO: 31) at residues corresponding to positions 53-64 in SEQ ID NO: 30.

In some embodiments, the CB5 comprises at most one histidine in one or more of the following regions: a region corresponding to positions 64-104 of SEQ ID NO: 1; a region corresponding to positions 105-122 of SEQ ID NO: 1; and/or a region corresponding to positions 123-165 of SEQ ID NO: 1.

In some embodiments, the CB5 comprises no histidine residues in: a region corresponding to positions 64-104 of SEQ ID NO: 1; a region corresponding to positions 105-122 of SEQ ID NO: 1; and/or a region corresponding to positions 123-165 of SEQ ID NO: 1.

In some embodiments, the CB5 comprises a sequence that is at least 90% identical to any one of SEQ ID NOs: 1-6, 10, 30 and 318.

In some embodiments, the CB5 comprises the sequence of any one of SEQ ID NOs: 1-6, 10, 30 and 318.

In some embodiments, the heterologous polynucleotide comprises a sequence that is at least 90% identical to any one of SEQ ID NOs: 11-17, 21-24, 36-37, 95-96, 99, 100, 102-103, 105-106, 316-317, and 330-331.

In some embodiments, the heterologous polynucleotide comprises the sequence of any one of SEQ ID NOs: 11-17, 21-24, 36-37, 95-96, 99, 100, 102-103, 105-106, 316-317, and 330-331.

Further aspects of the present disclosure provide host cells that comprise a heterologous polynucleotide encoding a cytochrome b5 (CB5), wherein the CB5 comprises a sequence that is at least 90% identical to any one of SEQ ID NOs: 1-10, 29, 30, 32, 33, 34, 77, 78, 79, 80, 81, 82, 118, and 318 and wherein the host cell is capable of producing an oxygenated hydrocarbon.

In some embodiments, the CB5 comprises the sequence of any one of SEQ ID NOs: 1-10, 29, 30, 32, 33, 34, 77, 78, 79, 80, 81, 82, 118, and 318.

Further aspects of the present disclosure provide host cells that comprise a heterologous polynucleotide encoding a cytochrome b5 (CB5), wherein the CB5 comprises a sequence that is at least 90% identical to any one of SEQ ID NOs: 1-6, 10, 30, 33-34, and 318 and wherein the host cell is capable of producing more of an oxygenated hydrocarbon than a control host cell that does not comprise the heterologous polynucleotide.

Further aspects of the present disclosure provide host cells that comprise a heterologous polynucleotide encoding a cytochrome b5 (CB5), wherein the heterologous polynucleotide comprises a sequence that is at least 90% identical to any one of SEQ ID NOs: 11-24, 35-40, 83-88, 95-107, 316-317, and 330-331, and wherein the host cell is capable of producing an oxygenated hydrocarbon.

In some embodiments, the heterologous polynucleotide comprises the sequence of any one of SEQ ID NOs: 11-24, 35-40, 83-88, 95-107, 316-317, and 330-331.

Further aspects of the present disclosure provide host cells that comprise a heterologous polynucleotide encoding a cytochrome b5 (CB5), wherein the CB5 comprises: the amino acid sequence ILRVSFRKYRKAIEQ (SEQ ID NO: 54); the amino acid sequence RAFRPSIRFKKSHSTVPT (SEQ ID NO: 55); the amino acid sequence KNTLYVGG (SEQ ID NO: 56); and/or the amino acid sequence DQATQKHRSFGFVTFLEKED (SEQ ID NO: 57) and wherein the host cell is capable of producing more of an oxygenated hydrocarbon than a control host cell that does not comprise the heterologous polynucleotide.

In some embodiments, the CB5 comprises: the amino acid sequence ILRVSFRKYRKAIEQ (SEQ ID NO: 54) at residues corresponding to positions 23-37 of SEQ ID NO: 4; the amino acid sequence RAFRPSIRFKKSHSTVPT (SEQ ID NO: 55) at residues corresponding to positions 53-70 of SEQ ID NO: 4; the amino acid sequence KNTLYVGG (SEQ ID NO: 56) at residues corresponding to positions 168-175 of SEQ ID NO: 4; and/or the amino acid sequence DQATQKHRSFGFVTFLEKED (SEQ ID NO: 57) at residues corresponding to positions 203-222 of SEQ ID NO: 4.

In some embodiments, the CB5 comprises a sequence that is at least 90% identical to SEQ ID NO: 4.

In some embodiments, the CB5 comprises SEQ ID NO: 4.

In some embodiments, the heterologous polynucleotide comprises a sequence that is at least 90% identical to SEQ ID NO: 15 or 96.

In some embodiments, the heterologous polynucleotide comprises SEQ ID NO: 15 or 96.

In some embodiments, the host cell further comprises a heterologous polynucleotide encoding a cytochrome P450 (CYP).

In some embodiments, the CYP is a C11 hydroxylase, ABA1, ABA2, T16H2, T3O, PPDS, AD1, AD5, AD6, C28 oxidase, C16 oxidase, C23 oxidase, and/or CrtS.

In some embodiments, the oxygenated hydrocarbon is mogrol, a ginsenoside, abscisic acid, a vinca alkaloid, a betaxanthin, or quillaic acid.

In some embodiments, the oxygenated hydrocarbon is an oxygenated isoterpenoid.

In some embodiments, the oxygenated hydrocarbon is a terpenoid glycoside, a terpenoid indole alkaloid, or a sesquiterpene.

In some embodiments, the terpenoid glycoside is a mogroside or a ginsenoside.

In some embodiments, the terpenoid indole alkaloid is a vinca alkaloid.

In some embodiments, the vinca alkaloid is vinblastine, vincristine, or vinorelbine.

In some embodiments, the sesquiterpene is abscisic acid.

In some embodiments, the oxygenated hydrocarbon is a betalain.

In some embodiments, the betalain is a betacyanin or a betaxanthin.

In some embodiments, the oxygenated hydrocarbon is astaxanthin.

In some embodiments, the host cell is capable of producing more than 13.5 mg/L mogrol.

In some embodiments, the host cell is capable of producing more than 5,000 μg/L abscisic acid.

In some embodiments, the host cell is capable of producing more than 1 nM of a vinca alkaloid or a precursor thereof.

In some embodiments, the vinca alkaloid precursor is vindoline.

In some embodiments, the host cell is capable of producing more than 70 mg/L of a ginsenoside.

In some embodiments, the ginsenoside is ginsenoside Rh2.

In some embodiments, the host cell is capable of producing more than 180 mg/L of a betalain, optionally more than 1,950 mg/L of a betalain.

In some embodiments, the host cell is capable of producing more than 180 mg/L of a betacyanin, optionally more than 200 mg/L of a betacyanin.

In some embodiments, the betalain is a betaxanthin.

In some embodiments, the host cell is capable of producing more than 0.3 mg/L astaxanthin.

In some embodiments, the host cell further comprises one or more heterologous polynucleotides encoding one or more mogrol and/or mogroside synthesis enzymes.

In some embodiments, the host cell further comprises one or more heterologous polynucleotides encoding one or more ABA synthesis enzymes.

In some embodiments, the host cell further comprises one or more heterologous polynucleotides encoding one or more vinca alkaloid synthesis enzymes.

In some embodiments, the host cell further comprises one or more heterologous polynucleotides encoding one or more ginsenoside synthesis enzymes.

In some embodiments, the host cell further comprises one or more heterologous polynucleotides encoding one or more betalain synthesis enzymes.

In some embodiments, the host cell further comprises one or more heterologous polynucleotides encoding one or more astaxanthin synthesis enzymes.

In some embodiments, the heterologous polynucleotide encoding the CB5 comprises a sequence that is at least 90% identical to any one of SEQ ID NOs: 129-132.

In some embodiments, the CYP comprises an amino acid sequence that is at least 90% identical to any one of SEQ ID NOs: 41, 43, 89, 90, 117, 120, 128, 280, 281, and 324.

In some embodiments, the host cell is a yeast cell, a plant cell, or a bacterial cell.

In some embodiments, the host cell is a yeast cell. In some embodiments, the yeast cell is a Saccharomyces cerevisiae cell. In some embodiments, the yeast cell is a Xanthophyllomyces dendrorhous cell. In some embodiments, the yeast cell is a Yarrowia lipolytica cell.

In some embodiments, the host cell is a bacterial cell. In some embodiments, the bacterial cell is an E. coli cell.

Further aspects of the present disclosure provide methods of producing an oxygenated hydrocarbon comprising culturing any of the host cells of the disclosure.

Further aspects of the present disclosure provide methods of producing an oxygenated hydrocarbon comprising culturing any of the host cells of the disclosure.

In some embodiments, oxygenated hydrocarbon is selected from the group consisting of: mogrol, a mogroside, ABA or an ABA precursor, a vinca alkaloid or a vinca alkaloid precursor, a ginsenoside or a ginsenoside precursor, a betalain or a betalain precursor, quillaic acid or a quillaic acid precursor, or astaxanthin or an astaxanthin precursor.

Further aspects of the present disclosure provide bioreactors for producing an oxygenated hydrocarbon, wherein the bioreactor comprises any of the host cells of the disclosure.

Further aspects of the present disclosure provide non-naturally occurring polynucleotides comprising a sequence that is at least 90% identical to any one of SEQ ID NOs: 11-17, 21-24, 36-37, 39-40, 95-97, 99-103, 105-106, 316-317, and 330-331.

In some embodiments, the polynucleotide encodes a cytochrome b5 (CB5) comprising a sequence that is at least 90% identical to any one of SEQ ID NOs: 1-10, 29, 30, 32, 33, 34, 77, 78, 79, 80, 81, 82, 118, and 318.

Further aspects of the present disclosure provide expression vectors comprising any of the non-naturally occurring polynucleotides of the disclosure.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIGS. 1A-1D include schematic overviews of putative mogrol biosynthesis pathways. SQS indicates squalene synthase, EPD indicates epoxidase, P450 indicates C11 hydroxylase, EPH indicates epoxide hydrolase, and CDS indicates cucurbitadienol synthase. FIG. 1A and FIG. 1B show putative mogrol biosynthesis pathways. FIG. 1C shows non-limiting examples of primary UGT activity. FIG. 1D shows non-limiting examples of secondary UGT activity.

FIG. 2 shows an abscisic acid (ABA) biosynthesis pathway. FIG. 2 is based on FIG. 1 of Otto et al., Microb Cell Fact. 2019; 18: 205.

FIG. 3 shows a biosynthetic pathway from dehydrosecodine to vinblastine. As shown in this application, in some embodiments, the addition of CB5 enzymes accelerated tabersonine 16-hydroxylase 2 (T16H2) and tabersonine 3-oxygenase (T3O)/tabersonine 3-reductase (T3R) product substrate depletion and enhanced the accumulation of terminal metabolite, vindoline.

FIG. 4 shows a biosynthetic pathway from 2,3-oxidosqualene to ginsenoside Rg3. As shown in this application, in some embodiments, the addition of CB5 enzyme accelerated 20(S)-protopanaxadiol (PPD) product formation and enhanced the accumulation of ginsenoside Rh2.

FIG. 5 shows a biosynthetic pathway from tyrosine to betaxanthin and betanin.

FIG. 6 is a schematic showing the astaxanthin pathway in Xanthophyllomyces dendrorhous (Phafia rhodozyma). CrtS is the final enzyme responsible for astaxanthin formation and is a P450. Without being bound by a particular theory, CrtS may benefit from CYB5 overexpression.

FIGS. 7A-7B include graphs depicting mogrol production by strains comprising candidate proteins involved in mogroside biosynthesis that were included in a library that was screened as described in Example 1. Parent strain 669889 is the control base strain without a candidate protein. FIG. 7A is a graph showing results for all strains in the screen. FIG. 7B is a graph showing mogrol production by strains comprising a cytochrome b5 (CB5).

FIGS. 8A-8B include graphs depicting mogrol production by Y. lipolytica strains. FIG. 8A is a graph depicting mogrol production by Y. lipolytica strains expressing a CB5 protein with a sequence corresponding to SEQ ID NO: 1 (strain 994375) or a truncated form of the same CB5 protein with a sequence corresponding to SEQ ID NO: 318 (strain 934903). Strain 974137 lacks any S. grosvenorii cytochrome b5 protein and was used as a negative control. FIG. 8B includes a graph depicting mogrol production by Y. lipolytica strains expressing a CB5 proteins with a sequence corresponding to SEQ ID NO: 1 (strain 1338488), SEQ ID NO: 3 (strain 1338489), or SEQ ID NO: 2 (strain 1338490). Strain 1419596 lacks any S. grosvenorii cytochrome b5 protein and was used as a negative control.

FIG. 9 is a graph showing ABA-diol production using a Y. lipolytica parent strain (parent strain 1) that expresses the following three ABA synthesis enzymes: aba1, aba2, and aba3. The parent strain was transformed with one of 6 fungal CB5 candidate genes (mined from Myrothecium indicum) or one of 3 S. grosvenorii CB5 genes. ABA-diol production by each strain was measured by LC-MS. The parent strain produces ABA-diol.

FIG. 10 is a graph showing abscisic acid (ABA) production using a Y. lipolytica parent strain (parent strain 1) that expresses the following three ABA synthesis enzymes: aba1, aba2, and aba3. The parent strain was transformed with one of 6 fungal CB5 candidate genes (mined from Myrothecium indicum) or one of 3 S. grosvenorii CB5 genes. Production of the stereoisomer S-ABA by each strain was measured by LC-MS. FIG. 10 depicts data from the same samples analyzed in FIG. 9.

FIG. 11 is a graph showing ABA production using a Y. lipolytica parent strain (parent strain 2) that expresses the following four ABA synthesis enzymes: aba1, aba2, aba3, and aba4. The parent strain was transformed with either of two S. grosvenorii CB5 genes (SEQ ID NO: 1, which is referred to in this application as “alpha” or “CB5 alpha” and SEQ ID NO: 2, which is referred to in this application as “gamma” or “CB5 gamma”). SEQ ID NO: 3 is referred to in this application as “beta” or “CB5 beta.” Production of the stereoisomer S-ABA by each strain was measured by LC-MS. The parent strain produces ABA.

FIG. 12 is a graph showing ABA production using a Y. lipolytica parent strain (parent strain 3) that expresses the following ABA synthesis enzymes: aba1, aba2, aba3, and aba4 and also a Cytochrome P450 reductase (Myrothecium indicum CPR). The parent strain was transformed with either of two S. grosvenorii CB5 genes (SEQ ID NO: 1 or 2). Production of the stereoisomer S-ABA by each strain was measured by LC-MS. The parent strain produces ABA.

FIG. 13 is a graph showing relative tabersonine substrate remaining between strains after five days. Strain WT is wild-type yeast and 1485239 contains the core pathway from tabersonine to vindoline. The other strains contain the core vindoline pathway in addition to a CB5. Each of the other strains was engineered to express one of the following CB5s: SEQ ID NO: 1 (strain 1522722); SEQ ID NO: 2 (strain 1522728); and SEQ ID NO: 3 (strain 1522736). Individual data points for the three technical replicates are shown as circles, squares and plus shapes, respectively. The average is shown as a horizontal bar.

FIG. 14 is a graph showing relative intermediate 16-MOH-tabersonine substrate remaining between strains after five days. Strain WT is wild-type yeast and 1485239 contains the core pathway from tabersonine to vindoline. The other strains contain the core vindoline pathway in addition to a CB5. Each of the other strains was engineered to express one of the following CB5s: SEQ ID NO: 1 (strain 1522722); SEQ ID NO: 2 (strain 1522728); and SEQ ID NO: 3 (strain 1522736). Individual data points for the three technical replicates are shown as circles, squares and plus shapes, respectively. The average is shown as a horizontal bar.

FIG. 15 is a graph showing relative product vindoline accumulation between strains after five days. Strain WT is wild-type yeast and 1485239 contains the core pathway from tabersonine to vindoline. The other strains contain the core vindoline pathway in addition to a CB5. Each of the other strains was engineered to express one of the following CB5 sequences: SEQ ID NO: 1 (strain 1522722); SEQ ID NO: 2 (strain 1522728); and SEQ ID NO: 3 (strain 1522736). Individual data points for the three technical replicates are shown as circles, squares and plus shapes, respectively. The average is shown as a horizontal bar.

FIG. 16 is a graph showing relative ginsenoside Rh2 (Rh2) product accumulation between strains. Strain 1469661 includes the core Rh2 pathway while the other strains include the core Rh2 pathway in addition to a CB5. The strains were engineered to express one of the following CB5 sequences: SEQ ID NO: 2 (strain 1478749); SEQ ID NO: 3 (strain 1478753); SEQ ID NO: 1 (strain 1478750); SEQ ID NO: 3 (strain 1478751); SEQ ID NO: 30 (strain 1478762); SEQ ID NO: 30 (strain 1478761); SEQ ID NO: 32 (strain 1478760); SEQ ID NO: 33 (strain 1478758); SEQ ID NO: 6 (strain 1478754); SEQ ID NO: 4 (strain 1478755); SEQ ID NO: 8 (strain 1478759); SEQ ID NO: 34 (strain 1478748); and SEQ ID NO: 5 (strain 1478752). Error bars represent the standard error of the mean amongst three technical replicates.

FIG. 17 includes graphs showing reactive product profiles between strains where strain 1469661 lacks a CB5 while 1478749 includes an accelerating CB5 (SEQ ID NO: 2).

FIG. 18 is a graph showing relative dammarenediol-II (DII), 20(S)-protopanaxadiol (PPD), and Rh2 product accumulation between strains. Strain 1469661 contains the core Rh2 pathway while the other strains contain the core Rh2 pathway in addition to a CB5. The strains were engineered to express one of the following CB5 sequences: SEQ ID NO: 2 (strain 1478749); SEQ ID NO: 3 (strain 1478753); SEQ ID NO: 1 (strain 1478750); SEQ ID NO: 3 (strain 1478751): SEQ ID NO: 30 (strain 1478762); SEQ ID NO: 30 (strain 1478761); SEQ ID NO: 32 (strain 1478760); SEQ ID NO: 33 (strain 1478758); SEQ ID NO: 6 (strain 1478754); SEQ ID NO: 4 (strain 1478755); SEQ ID NO: 8 (strain 1478759); SEQ ID NO: 34 (strain 1478748); and SEQ ID NO: 5 (strain 1478752). Individual data points for the three technical replicates are shown as circles, squares and plus shapes. The average is shown as a horizontal bar.

FIG. 19 is a graph showing the impact of expressing CB5s on betaxanthin production. The strains were engineered to express one of the following CB5 sequences: SEQ ID NO: 118 (strain 1586358); SEQ ID NO: 3 (molecule ID m4037591, strain 1586404); SEQ ID NO: 3 (molecule ID m4037591, strain 1586407); SEQ ID NO: 4 (molecule ID m4037592, strain 1586409): SEQ ID NO: 4 (molecule ID m4037592, strain 1586411); SEQ ID NO: 8 (molecule ID m4037594, strain 1586416); SEQ ID NO: 8 (molecule ID m4037594, strain 1586417); SEQ ID NO: 8 (molecule ID m4037594, strain 1586419); SEQ ID NO: 34 (molecule ID m4037593, strain 1586429); SEQ ID NO: 6 (molecule ID m4037595, strain 1586432): SEQ ID NO: 6 (molecule ID m4037595, strain 1586434); SEQ ID NO: 6 (molecule ID m4037595, strain 1586437); SEQ ID NO: 33 (molecule ID m4037598, strain 1586438); SEQ ID NO: 10 (molecule ID m4037600, strain 1586447); SEQ ID NO: 5 (molecule ID m4037599, strain 1586452); SEQ ID NO: 3 (molecule ID m4037603, strain 1586464); SEQ ID NO: 32 (molecule ID m4037840, strain 1586471); and SEQ ID NO: 32 (molecule ID m4037840, strain 1586472).

FIG. 20 is a sequence alignment of the N-terminal portion of CB5s including TU.tig00153756.33 (SEQ ID NO: 2, CB5 gamma), TU.tig00005590.1 (SEQ ID NO: 1, CB5 alpha), and TU.tig00153145.177 (SEQ ID NO: 3, CB5 beta). From top to bottom, SEQ ID NOs: 113-115, 115, 133-142, 162, 143, 144,163, 145-156, and 156 are illustrated.

FIG. 21 is a graph showing the astaxanthin titers of various Phaffia rhodozyma transformants of SgCYB5 (SEQ ID NO: 1). Recode 1 is recoded for S. cerevisiae codon usage. Recode 2 is recoded for Y. lipolytica codon usage. Total carotenoids measured by absorbance at 492 nm (top). LC-MS measurement of Astaxanthin (bottom).

FIG. 22 is a graph showing the confirmation of biological and technical replicates of select isolates of Phafia rhodozyma transformants of SgCYB5 (CB5 alpha, SEQ ID NO: 1). Recode 1 is recoded for S. cerevisiae codon usage. Recode 2 is recoded for Y. lipolytica codon usage. Wildtype and CEN.PK S. cerevisiae strains were used as negative controls. Total carotenoids measured by absorbance at 492 nm (top). LC-MS measurement of Astaxanthin (bottom).

FIG. 23 is a graph showing the impact of expressing CB5s on betaxanthin production. The strains were engineered to express one of the following CB5 sequences: SEQ ID NO: 118 (strain 1630095); SEQ ID NO: 3 (molecule ID m4037591, strain 1630106): SEQ ID NO: 3 (molecule ID m4037591, strain 1630109); SEQ ID NO: 4 (molecule ID m4037592, strain 1630116); SEQ ID NO: 34 (molecule ID m4037593, strain 1630120); SEQ ID NO: 34 (molecule ID m4037593, strain 1630122); SEQ ID NO: 8 (molecule ID m4037594, strain 1630125); SEQ ID NO: 6 (molecule ID m4037595, strain 1630130); SEQ ID NO: 6 (molecule ID m4037595, strain 1630132); SEQ ID NO: 6 (molecule ID m4037595, strain 1630133): SEQ ID NO: 6 (molecule ID m4037595, strain 1630134); SEQ ID NO: 33 (molecule ID m4037598, strain 1630136); SEQ ID NO: 5 (molecule ID m4037599, strain 1630143); SEQ ID NO: 5 (molecule ID m4037599, strain 1630146); SEQ ID NO: 10 (molecule ID m4037600, strain 1630147); SEQ ID NO: 10 (molecule ID m4037600, strain 1630150); SEQ ID NO: 2 (molecule ID m4037604, strain 1630159); SEQ ID NO: 2 (molecule ID m4037604, strain 1630160); SEQ ID NO: 2 (molecule ID m4037604, strain 1630163): SEQ ID NO: 2 (molecule ID m4037604, strain 1630164); SEQ ID NO: 32 (molecule ID m4037840, strain 1630165); SEQ ID NO: 32 (molecule ID m4037840, strain 1630168); and SEQ ID NO: 32 (molecule ID m4037840, strain 1630169).

### DETAILED DESCRIPTION

Many natural products are oxygenated hydrocarbons useful as flavoring agents, dyes, dietary supplements, or fragrance compounds or useful in the production of pharmaceutical drugs. However, availability of natural products that are oxygenated hydrocarbons can be limited as purification of oxygenated hydrocarbons from natural sources and de novo chemical synthesis often have high production costs and low yield.

This disclosure is premised, in part, on the unexpected finding that several cytochrome B5 (CB5) enzymes can be used to increase production of different types of oxygenated hydrocarbons by recombinant cells.

### Oxygenated Hydrocarbons

The term “oxygenated hydrocarbon” refers to a small molecule organic compound (i.e., containing carbon) that comprises at least one oxygen atom. The oxygen atom may be present as any functional group containing an oxygen atom, including but not limited to, hydroxyls, carbonyls, carboxyls, ketones, aldehydes, ethers, amides, esters, and the like. The oxygenated hydrocarbon may contain one or more oxygen atoms present as one or more different functional groups (e.g., an oxygenated hydrocarbon containing a hydroxyl group and another functional group containing an oxygen). The oxygenated hydrocarbon may be branched or linear, cyclic or acyclic, saturated or unsaturated and may also contain multiple carbon-carbon bonds, stereocenters, and other non-oxygen containing functional groups (e.g., alkynes, alkenes, amines, sulfides, guanidines, and heterocyclic rings, etc.). Non-limiting examples of oxygenated hydrocarbons are provided herein.

The term “small molecule” refers to molecules, whether naturally-occurring or artificially created (e.g., via chemical synthesis) that have a relatively low molecular weight. In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible.

In some embodiments, a cytochrome B5 enzyme increases production of an oxygenated hydrocarbon in a cytochrome P450-catalyzed oxygenation reaction.

Thus, in some embodiments, oxygenated hydrocarbons are produced by cytochrome P450 (CYP) of the enzyme clade CYP76.

In some embodiments, use of one or more CB5s disclosed herein increases production of one or more oxygenated hydrocarbons. Without being bound by a particular theory, a CB5 disclosed herein may be used to increase production of oxygenated hydrocarbons in biosynthetic pathways that comprise a cytochrome P450. In some embodiments, use of one or more CB5s disclosed herein increases the amount of an oxygenated hydrocarbon produced by a cytochrome P450.

In some embodiments, an oxygenated hydrocarbon is a product produced by a cytochrome P450. In some embodiments, the cytochrome P450 is a C11 hydroxylase, ABAL, ABA2, T16H2, T3O, PPDS, AD1, AD5, AD6, CrtS, C28 oxidase, C16 oxidase, or C23 oxidase. Non-limiting examples of oxygenated hydrocarbons include 11-hydroxycucurbitadienol, 11-hydroxy-24,25-epoxycucurbitadienol, α-ionylideneacetic acid (α-IAA), 1′,4′-trans-dihydroxy-α-ionylideneacetic acid (DH-α-IAA or ABA-diol), 16-OH-tabersonine, 3-OH-16-MOH-2,3-2H-tabersonine, protopanaxadiol, L-DOPA, cyclo-DOPA, and derivatives of 11-hydroxycucurbitadienol, 11-hydroxy-24,25-epoxycucurbitadienol, α-ionylideneacetic acid (α-IAA), 1′,4′-trans-dihydroxy-α-ionylideneacetic acid (DH-α-IAA or ABA-diol), 16-OH-tabersonine, 3-OH-16-MOH-2,3-2H-tabersonine, protopanaxadiol, L-DOPA, and cyclo-DOPA. In some embodiments, a derivative of 11-hydroxycucurbitadienol and 11-hydroxy-24,25-epoxycucurbitadienol is mogrol. In some embodiments, a derivative of 11-hydroxycucurbitadienol and 11-hydroxy-24,25-epoxycucurbitadienol is a mogroside. In some embodiments, a derivative of α-ionylideneacetic acid (α-IAA), 1′,4′-trans-dihydroxy-α-ionylideneacetic acid (DH-α-IAA or ABA-diol) is abscisic acid. In some embodiments, a derivative of 16-OH-tabersonine and 3-OH-16-MOH-2,3-2H-tabersonine is vindoline or vinblastine. In some embodiments, a derivative of protopanaxadiol is a ginsenoside. In some embodiments, a derivative of L-DOPA and cyclo-DOPA is a betalain.

In some embodiments, an oxygenated hydrocarbon is an oxygenated isoprenoid or an oxygenated isoprenoid precursor. In some embodiments, an oxygenated hydrocarbon is a betalain or a betalain precursor.

In some embodiments, an oxygenated hydrocarbon is beta-amyrin, erythrodiol, oleanolic acid, hederagenin, gypsogenin, or quillaic acid. In some embodiments, an oxygenated isoprenoid is beta-amyrin, erythrodiol, oleanolic acid, hederagenin, gypsogenin, or quillaic acid.

In some embodiments, a host cell comprising one or more proteins described herein (e.g., a cytochrome b5 (CB5) and/or any protein associated with the disclosure) is capable of producing at least 1%, at least 2%, at least 3%, at least 4%, at least 5%, at least 6%, at least 7%, at least 8%, at least 9%, at least 10%, at least I1%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 75%, at least 80%, at least 85%, at least 90%, at least 95%, at least 100%, at least 125%, at least 150%, at least 175%, at least 200%, at least 225%, at least 250%, at least 275%, at least 300%, at least 325%, at least 350%, at least 375%, at least 400%, at least 425%, at least 450%, at least 475%, at least 500%, at least 1,000%, at least 2,000%, at least 3,000%, at least 4,000%, at least 5,000%, at least 6,000%, at least 7,000%, at least 8,000%, at least 9,000%, or at least 10,000% more of an oxygenated hydrocarbon or a precursor thereof as compared to a control host cell not comprising the cytochrome B5 and/or the protein associated with the disclosure. In some embodiments, the control host cell is a host cell that is the same species as the host cell comprising the cytochrome B5 and/or protein associated with the disclosure.

Any methods known in the art, including mass spectrometry (e.g., gas chromatography-mass spectrometry), may be used to identify an oxygenated hydrocarbon of interest.

### Isoprenoid and Isoprenoid Precursors

The term “isoprenoid” refers to any organic compound comprising isoprene (i.e., C₅H₈) units and derivatives thereof. In particular, isoprenoid compounds include, without limitation, monoterpenes, diterpenes, triterpenes, sesquiterpenes, terpenoid indole alkaloids, terpenoid glycosides, and sterols.

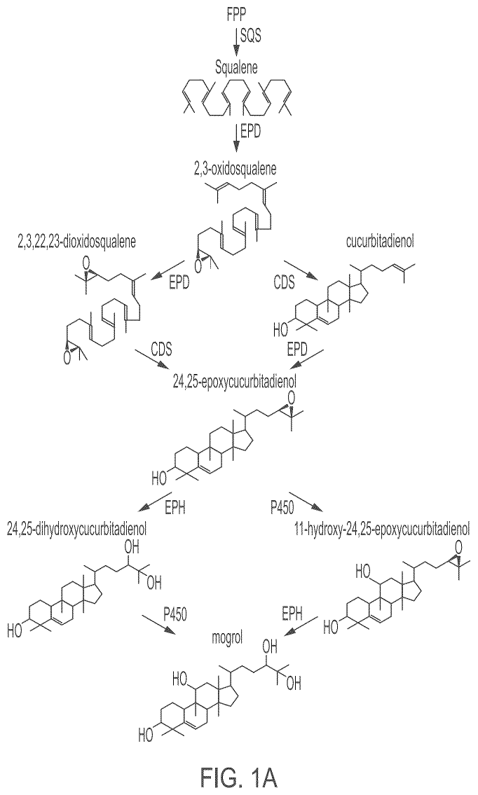

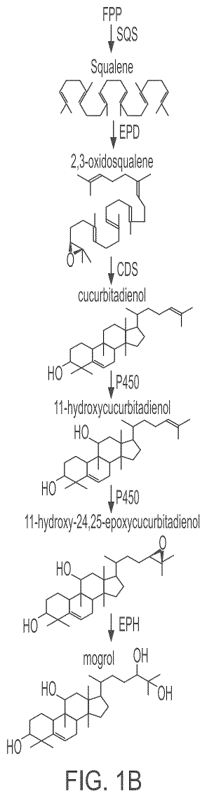

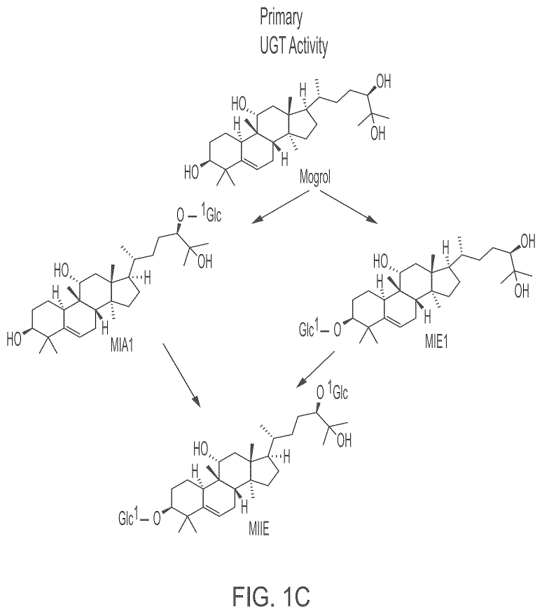

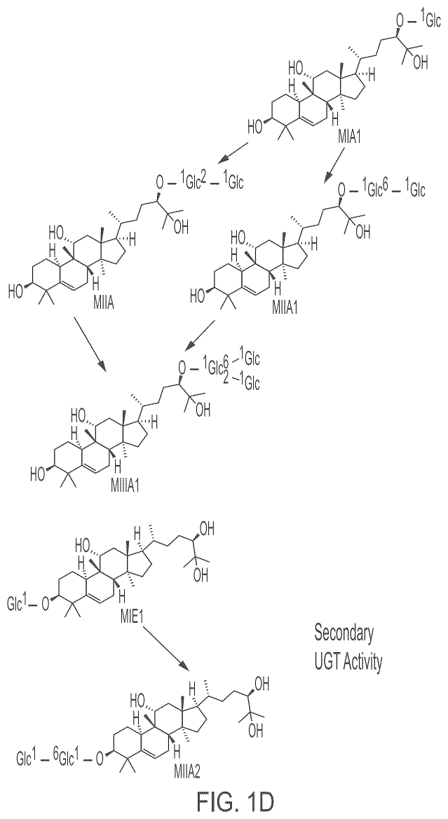

Isoprenoid precursors include but are not limited to acetyl-CoA, acetoacetyl-CoA, HMG-CoA, mevalonate, mevalonate-5-phosphate, mevalonate pyrophosphate, isopentenyl pyrophosphate (IPP), dimethylallyl pyrophosphate (DMAPP), geranyl pyrophosphate (GPP), farnesyl diphosphate (FPP), squalene, and 2-3-oxidosqualene. In some embodiments, an isoprenoid precursor is a compound shown in FIGS. 1A-4.

The terms “isoprenoid,” “terpene,” and “terpenoid” are used interchangeably in this application. For example, isoprenoids include pure hydrocarbons with the molecular formula (C₅H₈)ₙ, in which n represents the number of isoprene subunits. An isoprenoid may include carbon atoms in multiples of five. As a non-limiting example, an isoprenoid may comprise 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1,000, or more than 1,000 carbons. In some embodiments, an isoprenoid is an irregular isoprenoid. In some embodiments, an isoprenoid is an oxygenated isoprenoid that comprises at least one oxygen atom. Isoprenoids are structurally diverse compounds and, for example, may be cyclic (e.g., monocyclic, multi-cyclic, homocyclic and heterocyclic compounds) or acyclic (e.g., linear and branched compounds). In some embodiments, an isoprenoid may have a flavor and/or odor. As used herein, an aroma compound refers to a compound that has a scent.

Non-limiting examples of isoprenoids include monoterpenes, sesquiterpenes, diterpenes, sesterterpenes, triterpenes, and tetraterpenes. Monoterpenes comprise ten carbons. Non-limiting examples of monoterpenes include, but are not limited to, myrcene, methanol, carvone, hinokitiol, linalool, limonene, sabinene, thujene, carene, borneol, eucalyptol and camphene. Sesquiterpenes comprise 15 carbons. As used herein, sesquiterpenes include sesquiterpene hydrocarbons and sesquiterpene alcohols (sesquiterpenols). Non-limiting examples of sesquiterpenes include but are not limited to, abscisic acid, cedrene, bulnesene, guaiene, vatirenene, seychellane, cubebene, sesquiphellandrene, germacrene, himachalane, caryophyllene, delta-cadinene, epi-cubenol, tau-cadinol, alpha-cadinol, gamma-selinene, 10-epi-gamma-eudesmol, gamma-eudesmol, alpha/beta-eudesmol, juniper camphor, 7-epi-alpha-eudesmol, cryptomeridiol isomer 1, cryptomeridiol isomer 2, cryptomeridiol isomer 3, humulene, alpha-guaiene, delta-guaiene, zingiberene, beta-bisabolene, beta-farnesene, beta-sesquiphellandrene, cubenol, alpha-bisabolol, alpha-curcumene, trans-nerolidol, gamma, bisabolene, beta-caryophyllene, trans-Sesquisabinene hydrate, delta-elemene, cis-eudesm-6-en-11-ol, daucene, isodaucene, trans-bergamotene, alpha-zingiberene, sesquisabinene hydrate, and 8-Isopropenyl-1,5-dimethyl-1,5-cyclodecadiene. Diterpenes comprise 20 carbons. Non-limiting examples of diterpenes include, but are not limited to, cembrene and sclareol. Sesterterpenes comprise 25 carbons. A non-limiting example of a sesterterpene is geranylfarnesol. Triterpenes comprise 30 carbons. Non-limiting examples of triterpenes include quillaic acid, squalene, polypodatetraene, malabaricane, lanostane, cucurbitacin, hopane, oleanane, and ursolic acid. Tetraterpenes comprise 40 carbons. Non-limiting examples of tetraterpenes include carotenoids. Examples of carotenoids include but are not limited to astaxanthin, β-carotene, zeaxanthin, xanthophylls, carotenes, canthaxanthin, β-cryptoxanthin, lycopene, lutein, and apo-carotenoids (e.g., retinal, beta-ionone, and bixin). See also, e.g., WO 2019/161141. In some embodiments, an isoprenoid is a cannabinoid. See, e.g., WO 2020/176547.

In some embodiments, an isoprenoid is a mogrol, a mogroside, abscisic acid, a vinca alkaloid, quillaic acid, or a ginsenoside.

In some embodiments, an isoprenoid is a terpenoid glycoside, terpenoid indole alkaloid, or sesquiterpene. As used herein, the term “glycoside” refers to a molecule in which at least one sugar is bonded to a non-sugar compound via a glycosidic bond. The term “terpenoid glycoside” refers to any compound having a terpenoid linked by a glycosidic bond to a sugar.

Non-limiting examples of terpenoid glycosides include triterpenoid glycosides (e.g., mogrol glycosides (e.g., mogroside (e.g., mogroside I-A1 (MIA1), mogroside I-E (MIE), mogroside II-A1 (MIIA1), mogroside II-A2 (MIIA2), mogroside III-A1 (MIIIA1), mogroside II-E (MIIE), mogroside III (MIII), siamenoside I, mogroside III-E (MIIIE), mogroside IV, mogroside IVa, isomogroside IV, mogroside V, or mogroside VI), ginsenosides (e.g., PPD (protopanaxadiol)-type ginsenosides, PPT (protopanaxatriol)-type ginsenosides, etc. (e.g., PPD, PPT, Ra3, Rb1, Rb2, Rb3, Rc, Rd, Re, Rg1, Rg2, Rg3, Rh1, Rh2, Rs1, C—O, C—Y, C-Mc1, C-Mc, F1, F2, compound K, gypenoside XVII, gypenoside LXXV, Rs2, PPD, Re, Rg1, Rf, F1, Rg2, PPT and Rh1))).

In some embodiments, a terpenoid indole alkaloid is a vinca alkaloid. In some embodiments, a vinca alkaloid is vinblastine, vincristine, or vinorelbine.

Any methods known in the art, including mass spectrometry (e.g., gas chromatography-mass spectrometry), may be used to identify an isoprenoid precursor or isoprenoid of interest.

### Synthesis of Mogrol and Mogrosides

FIGS. 1A-1B show putative mogrol synthesis pathways. An early step in the pathway involves conversion of squalene to 2,3-oxidoqualene. As shown in FIG. 1A, 2,3-oxidosqualene can be first cyclized to cucurbitadienol followed by epoxidation to form 24,25-epoxycucurbitadienol, or 2,3-oxidosqualene can be epoxidized to 2,3,22,23-dioxidosqualene and then cyclized to 24,25-epoxycucurbitadienol. Next, the 24,25-epoxycucurbitadienol can be converted to mogrol (an aglycone of mogrosides) following epoxide hydrolysis and then oxidation, or oxidation and then epoxide hydrolysis. As shown in FIG. 1B, 2,3-oxidosqualene can be first cyclized to cucurbitadienol, which is then converted to 11-hydroxycucurbitadienol by a cytochrome P450 C11 hydroxylase. Then, a cytochrome P450 C11 hydroxylase may convert 11-hydroxycucurbitadienol to 11-hydroxy-24,25-epoxycucurbitadienol. 11-hydroxy-24,25-epoxycucurbitadienol may be converted to mogrol by epoxide hydrolase. C11 hydroxylases act in conjunction with cytochrome P450 reductases (not shown in FIGS. 1A-1B).

Mogrol can be distinguished from other cucurbitane triterpenoids by oxygenations at C3, C11, C24, and C25. Glycosylation of mogrol, for example at C3 and/or C24, leads to the formation of mogrosides.

In some embodiments, a mogrol precursor is a substrate of a mogrol synthesis enzyme. Non-limiting examples of mogrol synthesis enzymes include cytochrome P450 (e.g., a C11 hydroxylase), a cytochrome P450 reductase, a cucurbitadienol synthase (CDS), an epoxide hydrolase (EPH), a squalene synthase (SQS), and an epoxidase (EPD). Mogrol precursors include but are not limited to squalene, 2-3-oxidosqualene, 2,3,22,23-dioxidosqualene, cucurbitadienol, 24, 25-expoxycucurbitadienol, 11-hydroxycucurbitadienol, 11-hydroxy-24,25-epoxycucurbitadienol, 11-hydroxy-cucurbitadienol, 11-oxo-cucurbitadienol, and 24,25-dihydroxycucurbitadienol. The term “dioxidosqualene” may be used to refer to 2,3,22,23-diepoxy squalene or 2,3,22,23-dioxido squalene. The term “2,3-epoxysqualene” may be used interchangeably with the term “2-3-oxidosqualene.”

FIGS. 1C-1D show putative mogroside synthesis pathways. As used in this disclosure, a “UGT” refers to an enzyme that is capable of catalyzing the addition of the glycosyl group from a UTP-sugar to a compound (e.g., mogroside or mogrol). A UGT may be a primary and/or a secondary UGT.

A “primary” UGT, or a UGT that has “primary glycosylation activity,” refers to a UGT that is capable of catalyzing the addition of a glycosyl group to a position on a compound that does not comprise a glycosyl group. For example, a primary UGT may be capable of adding a glycosyl group to the C3 and/or C24 position of an isoprenoid substrate (e.g., mogrol). See, e.g., FIG. 1C.

A “secondary” UGT, or a UGT that has “secondary glycosylation activity,” refers to a UGT that is capable of catalyzing the addition of a glycosyl group to a position on a compound that already comprises a glycosyl group. See, e.g., FIG. 1D. As a non-limiting example, a secondary UGT may add a glycosyl group to a mogroside I-A1 (MIA1), mogroside I-E (MIE), mogroside II-A1 (MIIA1), mogroside II-A2 (MITA2), mogroside III-A1 (MIIIA1), mogroside II-E (MITE), mogroside III (MIII), siamenoside I, mogroside III-E (MIIIE), mogroside IV, mogroside IVa, isomogroside IV, mogroside V. and/or mogroside VI.

In some embodiments, a mogroside precursor is a substrate of a mogroside synthesis enzyme. Non-limiting examples of mogroside synthesis enzymes include a UDP-glycosyltransferase (UGT), a cytochrome P450 reductase, a cytochrome P450 (e.g., a C11 hydroxylase), a cucurbitadienol synthase (CDS), an epoxide hydrolase (EPH), a squalene synthase (SQS), or an epoxidase (EPD). As used in this application, mogroside precursors include mogrol precursors, mogrol, and mogrosides.

Examples of mogrosides include, but are not limited to, mogroside I-A1 (MIA1), mogroside IE (MIE or MIE), mogroside II-A1 (MIIA1 or M2A1), mogroside II-A2 (MIIA2 or M2A2), mogroside III-A1 (MIIIA1 or M3A1), mogroside II-E (MIIE or M2E), mogroside III (MIII or M3), siamenoside I, mogroside IV (MIV or M4), mogroside IVa (MIVA or M4A), isomogroside IV, mogroside III-E (MIHE or M3E), mogroside V (MV or M5), and mogroside VI (MVI or M6). In some embodiments, the mogroside produced is siamenoside 1, which may be referred to as Siam. In some embodiments, the mogroside produced is MIIE. Unless otherwise noted, when used in the plural, the terms “M is”, “MIs”, “M2s”, “MIIs”, “M3s”, “MIIIs”, “M4s”, “MIVs”, “MVs”, “M5s”, “M6s”, and “MVIs” each refer to a class of mogrosides. As a non-limiting example, M2s or M1s may include MIIA1, MIIA, MIIA2, and/or MIIE.

In other embodiments, a mogroside is a compound of Formula 1:

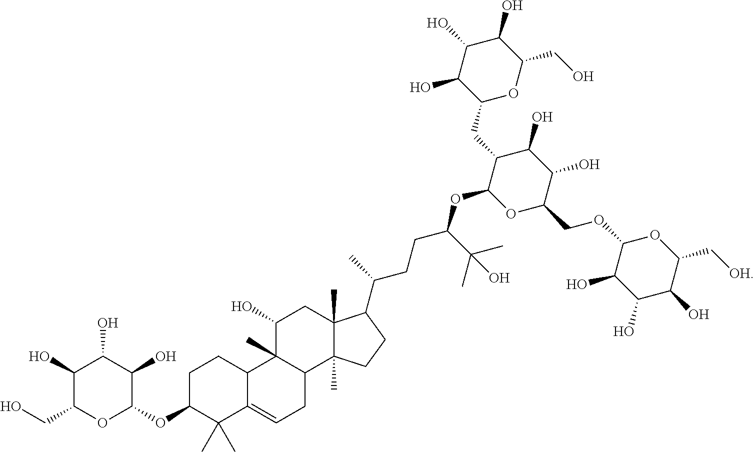 [SMILES: C[C@H](CC[C@@H](O[C@@H]1O[C@H](CO[C@@H]2O[C@H](CO)[C@@H](O)[C@H](O)[C@H]2O)[C@@H](O)[C@H](O)[C@H]1C[C@H]1O[C@@H](CO)[C@H](O)[C@@H](O)[C@@H]1O)C(C)(C)O)C1CC[C@@]2(C)C3CC=C4C(CC[C@H](O[C@@H]5O[C@H](CO)[C@@H](O)[C@H](O)[C@H]5O)C4(C)C)[C@]3(C)[C@H](O)C[C@]12C]

In some embodiments, the methods described in this application may be used to produce any of the compounds described in and incorporated by reference from US 2019/0071705 (which granted as U.S. Pat. No. 11,060,124), including compounds 1-20 as disclosed in US 2019/0071705. In some embodiments, the methods described in this application may be used to produce variants of any of the compounds described in and incorporated by reference from US 2019/0071705, including variants of compounds 1-20 as disclosed in US 2019/0071705. For example, a variant of a compound described in US 2019/0071705 can comprise a substitution of one or more alpha-glucosyl linkages in a compound described in US 2019/0071705 with one or more beta-glucosyl linkages. In some embodiments, a variant of a compound described in US 2019/0071705 comprises a substitution of one or more beta-glucosyl linkages in a compound described in US 2019/0071705 with one or more alpha-glucosyl linkages. In some embodiments, a variant of a compound described in US 2019/0071705 is a compound of Formula 1 shown above.

In some embodiments, a host cell comprising one or more proteins described herein (e.g., a cytochrome b5 (CB5), a UDP-glycosyltransferase (UGT) enzyme, a cucurbitadienol synthase (CDS) enzyme, a C11 hydroxylase enzyme, a cytochrome P450 reductase enzyme, an epoxide hydrolase enzyme (EPH), a squalene epoxidase enzyme (SQE) and/or any proteins associated with the disclosure) is capable of producing at least 0.005 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, at least 10,000 mg/L, at least 11,000 mg/L, at least 12,000 mg/L, at least 13,000 mg/L, at least 14,000 mg/L, at least 15,000 mg/L, at least 16,000 mg/L, at least 17,000 mg/L, at least 18,000 mg/L, at least 19,000 mg/L, at least 20,000 mg/L, at least 21,000 mg/L, at least 22,000 mg/L, at least 23,000 mg/L, at least 24,000 mg/L, at least 25,000 mg/L, at least 26,000 mg/L, at least 27,000 mg/L, at least 28,000 mg/L, at least 29,000 mg/L, at least 30,000 mg/L, at least 31,000 mg/L, at least 32,000 mg/L, at least 33,000 mg/L, at least 34,000 mg/L, at least 35,000 mg/L, at least 36,000 mg/L, at least 37,000 mg/L, at least 38,000 mg/L, at least 39,000 mg/L, at least 40,000 mg/L, at least 41,000 mg/L, at least 42,000 mg/L, at least 43,000 mg/L, at least 44,000 mg/L, at least 45,000 mg/L, at least 46,000 mg/L, at least 47,000 mg/L, at least 48,000 mg/L, at least 49,000 mg/L, at least 50,000 mg/L, at least 51,000 mg/L, at least 52,000 mg/L, at least 53,000 mg/L, at least 54,000 mg/L, at least 55,000 mg/L, at least 56,000 mg/L, at least 57,000 mg/L, at least 58,000 mg/L, at least 59,000 mg/L, at least 60,000 mg/L, at least 61,000 mg/L, at least 62,000 mg/L, at least 63,000 mg/L, at least 64,000 mg/L, at least 65,000 mg/L, at least 66,000 mg/L, at least 67,000 mg/L, at least 68,000 mg/L, at least 69,000 mg/L, at least 70,000 mg/L, at least 71,000 mg/L, at least 72,000 mg/L, at least 73,000 mg/L, at least 74,000 mg/L, at least 75,000 mg/L, at least 76,000 mg/L, at least 77,000 mg/L, at least 78,000 mg/L, at least 79,000 mg/L, at least 80,000 mg/L, at least 81,000 mg/L, at least 82,000 mg/L, at least 83,000 mg/L, at least 84,000 mg/L, at least 85,000 mg/L, at least 86,000 mg/L, at least 87,000 mg/L, at least 88,000 mg/L, at least 89,000 mg/L, at least 90,000 mg/L, at least 91.000 mg/L, at least 92,000 mg/L, at least 93,000 mg/L, at least 94,000 mg/L, at least 95,000 mg/L, at least 96,000 mg/L, at least 97,000 mg/L, at least 98,000 mg/L, at least 99,000 mg/L, or at least 100,000 mg/L of one or more mogrosides and/or mogroside precursors. In some embodiments, the mogroside is mogroside I-AI (MIA1), mogroside IE (MIE or MIE), mogroside II-A1 (MIIA1 or M2A1), mogroside II-A2 (MIIA2 or M2A2), mogroside III-A1 (MIIIA1 or M3A1), mogroside 11-E (MIIE or M2E), mogroside III (MIII or M3), siamenoside I, mogroside IV (MIV or M4), mogroside IVa (MIVA or M4A), isomogroside IV, mogroside III-E (MIIIE or M3E), mogroside V (MV or M5), or mogroside VI (MVI or M6).

### Synthesis of Abscisic Acid (ABA) and ABA Precursors

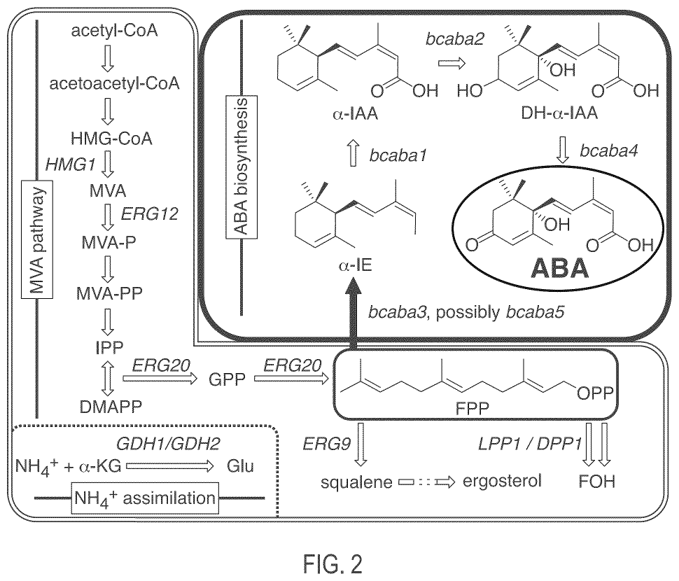

FIG. 2 shows a putative ABA biosynthesis pathway in a eukaryotic cell. An early step in the biosynthesis of ABA is the mevalonate pathway. As shown in FIG. 2, the mevalonate pathway converts acetyl-CoA to two five carbon building blocks called isopentenyl pyrophosphate (IPP) and dimethylallyl pyrophosphate (DMAPP). Two acetyl-CoA molecules are condensed by acetoacetyl-CoA thiolase to yield acetoacetyl-CoA. Acetoacetyl-CoA is further condensed by HMG-CoA synthase to yield 3-hydroxy-3-methyl-glutaryl-CoA (HMG-CoA). HMG-CoA is then reduced by HMG-CoA reductase to yield mevalonate. At this juncture, mevalonate is either converted to mevalonate-3-phosphate or mevalonate-5-phosphate. In ABA biosynthesis, mevalonate is phosphorylated twice at the 5-OH position to yield mevalonate-5-phosphate then mevalonate pyrophosphate (MVA-PP). MVA-PP is then decarboxylated by mevalonate pyrophosphate decarboxylase to yield IPP. IPP is isomerized to DMAPP via isopentenyl pyrophosphate isomerase, concluding the mevalonate pathway.

Following the mevalonate pathway, ABA biosynthesis continues when IPP and DMAPP are condensed by geranyl pyrophosphate synthase to yield geranyl pyrophosphate (GPP). GPP is then converted to farnesyl pyrophosphate (FPP), which can enter one of many downstream pathways, including the ABA biosynthesis pathway.

FPP enters the ABA biosynthesis pathway once it undergoes direct cyclization by α-ionylide neethane synthase 3 (aba3) to yield α-ionylideneethane (α-IE). ABA3 first converts FPP to β-farnesene and allofarnesene before cyclizing allofarnesene to yield α-IE. Following cyclization, α-IE interacts with the P450 monooxygenase ABA1 to undergo an oxidation reaction to yield α-ionylideneacetic acid (α-LAA), followed by further oxidation by the P450 monooxygenase aba2 to yield 1′,4′-trans-dihydroxy-α-ionylideneacetic acid (DH-α-IAA or ABA-diol). Finally, the P450 monooxygenase aba4 oxidizes DH-α-IAA to produce ABA. Overall, FPP undergoes direct cyclization to yield α-IE, followed by three oxidation steps to yield ABA.

In some embodiments, an ABA precursor is a substrate of an ABA synthesis enzyme. In some embodiments, an ABA synthesis enzyme is ABA1, ABA2, ABA3, or ABA4. In some embodiments, an ABA precursor is a substrate of a mevalonate pathway enzyme. In some embodiments, a MVA pathway enzyme is ERG1, ERG20, or HMG1. See, e.g., FIG. 2. ABA precursors include but are not limited to acetyl-CoA, acetoacetyl-CoA, HMG-CoA, mevalonate, mevalonate-5-phosphate, MVA-PP, IPP, DMAPP, GPP, FPP, α-IE, α-IAA, and DH-α-IAA.

In some embodiments, ABA is a compound of Formula 2:

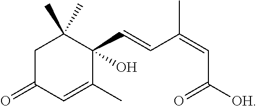

In some embodiments, ABA is S-ABA.

In some embodiments, a host cell comprising one or more proteins described herein (e.g., a cytochrome b5 (CB5), a ABA synthesis enzyme, and/or any proteins associated with the disclosure) is capable of producing at least 0.005 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, at least 10,000 mg/L, at least 11,000 mg/L, at least 12,000 mg/L, at least 13,000 mg/L, at least 14,000 mg/L, at least 15,000 mg/L, at least 16,000 mg/L, at least 17,000 mg/L, at least 18,000 mg/L, at least 19,000 mg/L, at least 20,000 mg/L, at least 21.000 mg/L, at least 22,000 mg/L, at least 23,000 mg/L, at least 24,000 mg/L, at least 25,000 mg/L, at least 26,000 mg/L, at least 27,000 mg/L, at least 28,000 mg/L, at least 29,000 mg/L, at least 30,000 mg/L, at least 31,000 mg/L, at least 32,000 mg/L, at least 33,000 mg/L, at least 34,000 mg/L, at least 35,000 mg/L, at least 36,000 mg/L, at least 37,000 mg/L, at least 38,000 mg/L, at least 39,000 mg/L, at least 40,000 mg/L, at least 41,000 mg/L, at least 42,000 mg/L, at least 43,000 mg/L, at least 44,000 mg/L, at least 45,000 mg/L, at least 46,000 mg/L, at least 47,000 mg/L, at least 48,000 mg/L, at least 49,000 mg/L, at least 50,000 mg/L, at least 51,000 mg/L, at least 52,000 mg/L, at least 53,000 mg/L, at least 54,000 mg/L, at least 55,000 mg/L, at least 56,000 mg/L, at least 57,000 mg/L, at least 58,000 mg/L, at least 59,000 mg/L, at least 60,000 mg/L, at least 61,000 mg/L, at least 62,000 mg/L, at least 63,000 mg/L, at least 64,000 mg/L, at least 65,000 mg/L, at least 66,000 mg/L, at least 67,000 mg/L, at least 68,000 mg/L, at least 69,000 mg/L, at least 70,000 mg/L, at least 71,000 mg/L, at least 72,000 mg/L, at least 73,000 mg/L, at least 74,000 mg/L, at least 75,000 mg/L, at least 76,000 mg/L, at least 77,000 mg/L, at least 78,000 mg/L, at least 79,000 mg/L, at least 80,000 mg/L, at least 81,000 mg/L, at least 82,000 mg/L, at least 83,000 mg/L, at least 84,000 mg/L, at least 85,000 mg/L, at least 86,000 mg/L, at least 87,000 mg/L, at least 88,000 mg/L, at least 89,000 mg/L, at least 90,000 mg/L, at least 91,000 mg/L, at least 92,000 mg/L, at least 93,000 mg/L, at least 94,000 mg/L, at least 95,000 mg/L, at least 96,000 mg/L, at least 97,000 mg/L, at least 98,000 mg/L, at least 99,000 mg/L, or at least 100,000 mg/L of ABA or a precursor thereof.

### Synthesis of Vinca Alkaloids

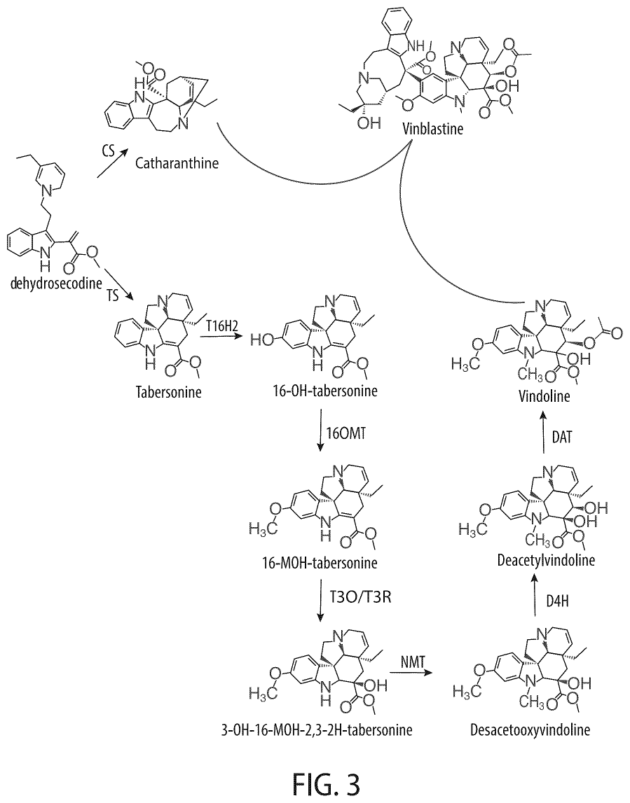

FIG. 3 shows a putative vinca alkaloid pathway from a plant cell. Vinca alkaloids (e.g., vinblastine, vincristine, and vinorelbine) serve as anti-cancer drugs and are listed in the World Health Organization Model List of Essential Medicines. Additional non-limiting examples of vinca alkaloids include vindesine, vincaminol, vineridine, vincamajine, and vinburnine. Structurally, vinca alkaloids comprise an indole nucleus (catharanthine) or a dihydroindole nucleus (vindoline). For example, as shown in FIG. 3, these monoterpene indole alkaloids (MIAs) are made from upstream MIA precursors catharanthine and vindoline. Both precursors are made from stemmadenine and go through an unstable intermediate dehydrosecodine, which is hydrolyzed by either catharanthine synthase (CS) to make catharanthine or tabersonine synthase (TS) to make tabersonine. The tabersonine to vindoline pathway includes seven enzymatic steps to make vindoline. First, tabersonine undergoes hydroxylation at the C-16 position by a cytochrome P450 (CYP) enzyme tabersonine 16-hydroxylase 2 (T16H2) followed by O-methylation by 16-hydroxytabersonine O-methyltransferase (16OMT). Then, cytochrome p450 (CYP) enzyme tabersonine 3-oxygenase (T30) together with tabersonine 3-reductase (T3R) catalyzes hydroxylation of intermediate 16-methoxytabersonine to make 3-OH-16-MOH-2,3-2H-tabersonine which is then methylated by 3-hydroxy-16-methoxy-2,3-dihydrotabersonine-N-methyltransferase (NMT), hydroxylated by desacetoxyvindolie-4-hydroxylase (D4H), and finally acetylated by deacetyvindoline-4-O-acetyltransferase (DAT) to make vindoline.

T16H2 and T3O, like other CYPs, utilize a cytochrome P450 reductase (CPR) to facilitate the two electron transport from NADPH to the heme iron center of P450 to activate molecular oxygen in the reaction cycle.

Without being bound by a particular theory, in some embodiments, in addition to CPRs, cytochrome b5 (Cb5s) may be used to facilitate the electron transport required for activity. As shown in Example 4, several CB5 enzymes were identified that accelerated T16H2 and T3O/T3R activity and enhanced accumulation of vindoline utilizing Saccharomyces cerevisiae host strains harboring the tabersonine to vindoline pathway.

In some embodiments, a vinca alkaloid precursor is a substrate of a vinca alkaloid synthesis enzyme. In some embodiments, a vinca alkaloid synthesis enzyme is a CS, TS, T16H2, 16OMT, T3O, T3R, NMT, D4H, or DAT enzyme. See, e.g., FIG. 3. Non-limiting examples of vinca alkaloid precursors include catharanthine, vindoline, stemmadenine, dehydrosecodine, tabersonine, 16-OH-tabersonine, 16-MOH-tabersonine, 3-OH-16-MOH-2,3-2H-tabersonine, desacetooxyvindoline, and deacetylvindoline.

In some embodiments, a host cell comprising one or more proteins described herein (e.g., a cytochrome b5 (CB5), a vinca alkaloid synthesis enzyme, and/or any proteins associated with the disclosure) is capable of producing at least 0.0001 nM, at least 0.005 nM, at least 0.01 nM, at least 0.02 nM, at least 0.03 nM, at least 0.04 nM, at least 0.05 nM, at least 0.06 nM, at least 0.07 nM, at least 0.08 nM, at least 0.09 nM, at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at least 11 nM, at least 12 nM, at least 13 nM, at least 14 nM, at least 15 nM, at least 16 nM, at least 17 nM, at least 18 nM, at least 19 nM, at least 20 nM, at least 21 nM, at least 22 nM, at least 23 nM, at least 24 nM, at least 25 nM, at least 26 nM, at least 27 nM, at least 28 nM, at least 29 nM, at least 30 nM, at least 31 nM, at least 32 nM, at least 33 nM, at least 34 nM, at least 35 nM, at least 36 nM, at least 37 nM, at least 38 nM, at least 39 nM, at least 40 nM, at least 41 nM, at least 42 nM, at least 43 nM, at least 44 nM, at least 45 nM, at least 46 nM, at least 47 nM, at least 48 nM, at least 49 nM, at least 50 nM, at least 51 nM, at least 52 nM, at least 53 nM, at least 54 nM, at least 55 nM, at least 56 nM, at least 57 nM, at least 58 nM, at least 59 nM, at least 60 nM, at least 61 nM, at least 62 nM, at least 63 nM, at least 64 nM, at least 65 nM, at least 66 nM, at least 67 nM, at least 68 nM, at least 69 nM, at least 70 nM, at least 75 nM, at least 80 nM, at least 85 nM, at least 90 nM, at least 95 nM, at least 100 nM, at least 125 nM, at least 150 nM, at least 175 nM, at least 200 nM, at least 225 nM, at least 250 nM, at least 275 nM, at least 300 nM, at least 325 nM, at least 350 nM, at least 375 nM, at least 400 nM, at least 425 nM, at least 450 nM, at least 475 nM, at least 500 nM, at least 1,000 nM, at least 2,000 nM, at least 3,000 nM, at least 4,000 nM, at least 5,000 nM, at least 6,000 nM, at least 7,000 nM, at least 8,000 nM, at least 9,000 nM, or at least 10,000 nM of a vinca alkaloid or a precursor thereof.

In some embodiments, a host cell comprising one or more proteins described herein (e.g., a cytochrome b5 (CB5), a vinca alkaloid synthesis enzyme, and/or any proteins associated with the disclosure) is capable of producing at least 0.005 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, at least 10,000 mg/L, at least 11,000 mg/L, at least 12,000 mg/L, at least 13,000 mg/L, at least 14,000 mg/L, at least 15,000 mg/L, at least 16,000 mg/L, at least 17,000 mg/L, at least 18,000 mg/L, at least 19,000 mg/L, at least 20,000 mg/L, at least 21,000 mg/L, at least 22,000 mg/L, at least 23,000 mg/L, at least 24,000 mg/L, at least 25,000 mg/L, at least 26,000 mg/L, at least 27.000 mg/L, at least 28,000 mg/L, at least 29,000 mg/L, at least 30,000 mg/L, at least 31,000 mg/L, at least 32,000 mg/L, at least 33,000 mg/L, at least 34,000 mg/L, at least 35,000 mg/L, at least 36,000 mg/L, at least 37,000 mg/L, at least 38,000 mg/L, at least 39,000 mg/L, at least 40,000 mg/L, at least 41,000 mg/L, at least 42,000 mg/L, at least 43,000 mg/L, at least 44,000 mg/L, at least 45,000 mg/L, at least 46,000 mg/L, at least 47,000 mg/L, at least 48,000 mg/L, at least 49,000 mg/L, at least 50,000 mg/L, at least 51,000 mg/L, at least 52,000 mg/L, at least 53,000 mg/L, at least 54,000 mg/L, at least 55,000 mg/L, at least 56,000 mg/L, at least 57,000 mg/L, at least 58,000 mg/L, at least 59,000 mg/L, at least 60,000 mg/L, at least 61,000 mg/L, at least 62,000 mg/L, at least 63,000 mg/L, at least 64,000 mg/L, at least 65,000 mg/L, at least 66,000 mg/L, at least 67,000 mg/L, at least 68,000 mg/L, at least 69,000 mg/L, at least 70,000 mg/L, at least 71,000 mg/L, at least 72,000 mg/L, at least 73,000 mg/L, at least 74,000 mg/L, at least 75,000 mg/L, at least 76,000 mg/L, at least 77,000 mg/L, at least 78,000 mg/L, at least 79,000 mg/L, at least 80,000 mg/L, at least 81,000 mg/L, at least 82,000 mg/L, at least 83,000 mg/L, at least 84,000 mg/L, at least 85,000 mg/L, at least 86,000 mg/L, at least 87,000 mg/L, at least 88,000 mg/L, at least 89,000 mg/L, at least 90,000 mg/L, at least 91,000 mg/L, at least 92,000 mg/L, at least 93,000 mg/L, at least 94,000 mg/L, at least 95,000 mg/L, at least 96,000 mg/L, at least 97,000 mg/L, at least 98,000 mg/L, at least 99,000 mg/L, or at least 100,000 mg/L of a vinca alkaloid or a precursor thereof.

### Synthesis of Ginsenosides

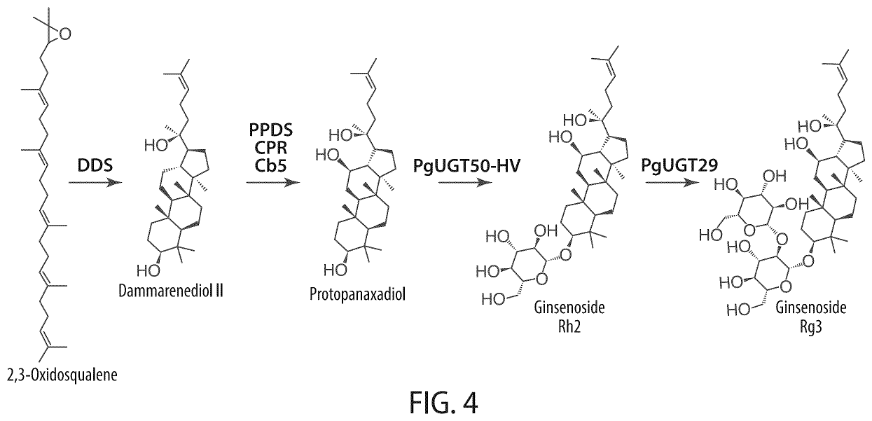

FIG. 4 shows a putative ginsenoside biosynthesis pathway. Ginsenosides are bioactive compounds found in Panax ginseng and other closely related plants with uses as traditional medicines and nutraceuticals. Non-limiting examples of ginsenosides include members of the dammarane family. Dammaranes are tetracyclic triterpenoids and include protopanaxadiols and protopanaxatriols. In some embodiments, a ginsenoside is a dammarane-type saponin derived from ginseng, or a derivative thereof. In some embodiments, a ginsenoside is ginsenoside Rg2 (Rg2) or ginsenoside Rg3 (Rg3). Rg3 is a bioactive ginseng ginsenoside with possible roles as an apoptosis inducer, an antineoplastic agent, and an angiogenesis modulating agent. As shown in FIG. 4, the Rg3 pathway proceeds from the ergosterol pathway intermediate 2,3 oxidosqualene via dammarenediol-II synthase (DDS), which converts 2,3 oxidosqualene to dammarenediol-II (DII). The DII triterpenoid product is subsequently hydroxylated to form the ginsenoside aglycone 20(S)-Protopanaxadiol (PPD) by a cytochrome P450 (CYP), i.e. PPD synthase (PPDS). PPD is next glycosylated at the C3-OH position by a UDP-glycosyltransferase (UGT), i.e. PgUGT50-HV, to form the ginsenoside Rh2. Finally, a second UGT, i.e., PgUGT29, transfers a glucose moiety to the C3 glucose of Rh2 via a 1-2-glycosidic bond.

PPDS, like other CYPs, utilizes a cytochrome P450 reductase (CPR) to facilitate the two-electron transport from NADPH to the heme iron center of the CYP to activate molecular oxygen in the reaction cycle. Without being bound by a particular theory, in addition to CPRs, cytochrome b5 (Cb5s) may be used to facilitate the PPDS reaction. As shown in Example 5, several CB5 enzymes were identified that accelerated PPDS activity in the enhanced accumulation of Rh2.

In some embodiments, a ginsenoside precursor is a substrate of a ginsenoside synthesis enzyme. In some embodiments, a ginsenoside synthesis enzyme is a dammarenediol-II synthase (DDS), a CYP, cytochrome P450 reductase or a UGT. See, e.g., FIG. 4. In some embodiments, a CYP for ginsenoside synthesis is PPDS. In some embodiments, a UGT for ginsenoside synthesis is PgUGT50-HV and/or PgUGT29.

In some embodiments, a host cell comprising one or more proteins described herein (e.g., a cytochrome b5 (CB5), a ginsenoside synthesis enzyme, and/or any proteins associated with the disclosure) is capable of producing at least 0.005 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, at least 10,000 mg/L, at least 11,000 mg/L, at least 12,000 mg/L, at least 13,000 mg/L, at least 14,000 mg/L, at least 15,000 mg/L, at least 16,000 mg/L, at least 17,000 mg/L, at least 18,000 mg/L, at least 19,000 mg/L, at least 20,000 mg/L, at least 21,000 mg/L, at least 22,000 mg/L, at least 23,000 mg/L, at least 24,000 mg/L, at least 25,000 mg/L, at least 26,000 mg/L, at least 27,000 mg/L, at least 28,000 mg/L, at least 29,000 mg/L, at least 30,000 mg/L, at least 31.000 mg/L, at least 32,000 mg/L, at least 33,000 mg/L, at least 34,000 mg/L, at least 35,000 mg/L, at least 36,000 mg/L, at least 37,000 mg/L, at least 38,000 mg/L, at least 39,000 mg/L, at least 40,000 mg/L, at least 41.000 mg/L, at least 42,000 mg/L, at least 43,000 mg/L, at least 44,000 mg/L, at least 45,000 mg/L, at least 46,000 mg/L, at least 47,000 mg/L, at least 48,000 mg/L, at least 49,000 mg/L, at least 50,000 mg/L, at least 51,000 mg/L, at least 52,000 mg/L, at least 53,000 mg/L, at least 54,000 mg/L, at least 55,000 mg/L, at least 56,000 mg/L, at least 57,000 mg/L, at least 58,000 mg/L, at least 59,000 mg/L, at least 60,000 mg/L, at least 61,000 mg/L, at least 62,000 mg/L, at least 63,000 mg/L, at least 64,000 mg/L, at least 65,000 mg/L, at least 66,000 mg/L, at least 67,000 mg/L, at least 68,000 mg/L, at least 69,000 mg/L, at least 70,000 mg/L, at least 71,000 mg/L, at least 72,000 mg/L, at least 73,000 mg/L, at least 74,000 mg/L, at least 75,000 mg/L, at least 76,000 mg/L, at least 77,000 mg/L, at least 78,000 mg/L, at least 79,000 mg/L, at least 80,000 mg/L, at least 81,000 mg/L, at least 82,000 mg/L, at least 83,000 mg/L, at least 84,000 mg/L, at least 85,000 mg/L, at least 86,000 mg/L, at least 87,000 mg/L, at least 88,000 mg/L, at least 89,000 mg/L, at least 90,000 mg/L, at least 91,000 mg/L, at least 92,000 mg/L, at least 93,000 mg/L, at least 94,000 mg/L, at least 95,000 mg/L, at least 96,000 mg/L, at least 97,000 mg/L, at least 98,000 mg/L, at least 99,000 mg/L, or at least 100,000 mg/L of a ginsenoside or a precursor thereof.

### Synthesis of Betalains

Betalains are pigments that are derivatives of betalamic acid and may be classified into two groups: the purple-red betacyanins and the yellow betaxanthins. In some embodiments, betalains are water-soluble pigments present in vacuoles of plants of the order Caryophyllales and in mushrooms of the genera Amanita, Hygrocybe and Hygrophorus. A betacyanin is a derivative of betalamic acid that has a conjugated substituted aromatic nucleus to the 1,7-diazaheptamethinium chromophore. See, e.g., Goldman et al. J. Amer. Soc. Hort. Sci. 121(1):23-26, 1996. In some embodiments, betacyanins are derivatives of betanidin, which is an iminium adduct of cyclodioxyphenylalnine (cyclo-DOPA). In some embodiments, a betacyanin is a compound of Formula 3:

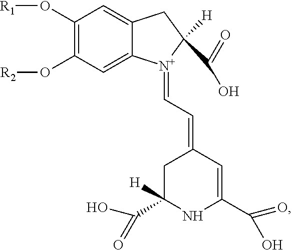

in which R1 and R2 are hydrogen or sugar moieties. Non-limiting examples of betacyanins include betanin, isobetanin, probetanin, and neobetanin. In some embodiments, a betacyanin is amaranthine, iso-amarathine, bougainvillein-r-I, betanin, iso-bougainvillein-r-I, iso-betanin, 2-apiosyl-betanin, betanidin, 2-apiosyl-isoisobetanin, phyllocactin, 4-malonyl-betanin, neobetanin, isophyllocactin, 4-malonyl-iso-betanin, 2-apiosyl-phyllocactin, 2-apiosyl-isophyllocactin, sinapoyl-apiosyl-betanin, sinapoyl-apiosyl-betanin-isomer, glycosyl-glycosyl-(caffeoyl-glycosyl)-betanidin, Glycosyl-glycosyl-(caffeoyl-glycosyl)-betanidin-isomer, (caffeoyl-glucosyl)-betanidin, feruloyl-glycosyl-betanin, glycosyl-(Caffeoyl-glycosyl)-betanidin, caffeoyl-glycosyl-(coumaroyl-glycosyl)-betanin-type, caffeoyl-glycosyl-(coumaroyl-glycosyl)-betanin-type, glycosyl-(caffeoyl-glycosyl)-betanidin-isomer, (caffeoyl-glucosyl)-betanidin-isomer, glycosyl-(coumaroyl-glycosyl)-betanidin, caffeoyl-glycosyl-(coumaroyl-glycosyl)-betanin-type, glycosyl-(coumaroyl-glycosyl)-betanidin-isomer, caffeoyl-glycosyl-(coumaroyl-glycosyl)-betanin-type, betanidin-6-O-(6′-O-trans-4-coumaroyl-glycosyl)-b-sophoroside, lampranthin II, betanidin-6-O-(6′-O-trans-4-coumaroyl-glycosyl)-b-sophoroside-isomer, or isolampranthin II. A betaxanthin is a derivative of betalamic acid that does not have a conjugated substituted aromatic nucleus to the 1,7-diazaheptamethinium chromophore. In some embodiments, betaxanthins are condensation products of betalamic acid with alpha-amino acids or amines. Non-limiting examples of betaxanthins include indicaxanthin, portulacaxanthin 11, and phenylalanine-betaxanthin. See also, e.g., U.S. Pat. No. 6,353,156. In some embodiments, a betaxanthin is a compound of Formula 4:

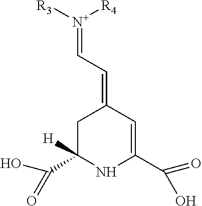

in which R3 is an amine or amino acid group and R4 is usually hydrogen. In some embodiments, a betaxanthin is glutamine-betaxanthin, glutamic acid-betaxanthin, proline-betaxanthin, dopa-betaxanthin I, dopa-betaxanthin II, tyrosine-betaxanthin, dopamine-betaxanthin, valine-betaxanthin, tyramine-betaxanthin, 3-methoxytyramine-betaxanthin, iso-leucine-betaxanthin, leucine-betaxanthin, phenylalanine-betaxanthin, or tryptophan-betaxanthin. Pigments from the betalain family may be found in plants from the order Caryophyllales, including beetroot (Beta vulgaris) and cactus fruit (Opuntia ficus-indica).

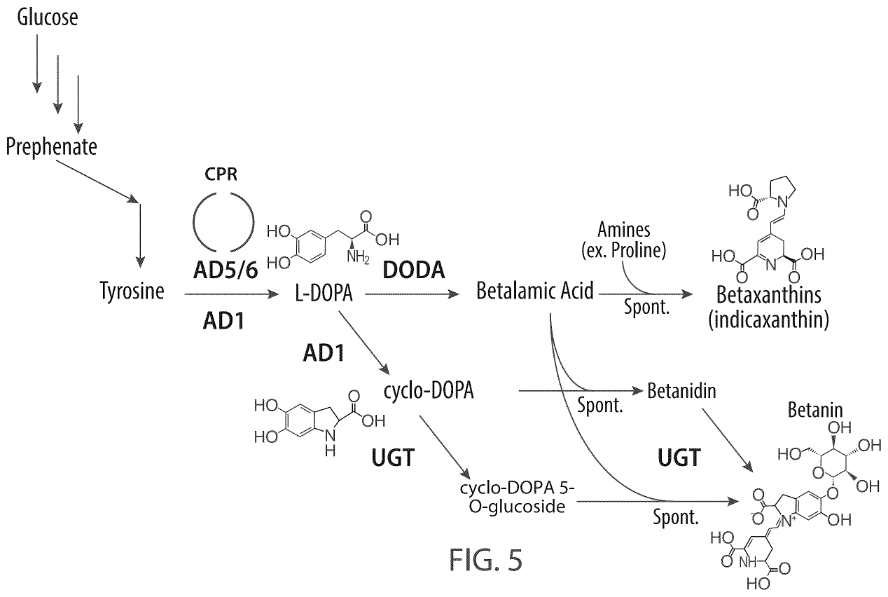

FIG. 5 shows a putative betalain biosynthesis pathway. As shown in FIG. 5, the betalains pathway is downstream of the tyrosine production pathway. CYP76AD I is also referred to as AD1. CYP76AD5 is also referred to as AD5. CYP76AD6 is also referred to as AD6. Production of betaxanthin involves three enzymes downstream of tyrosine. AD5 or AD6 (AD5/6) converts tyrosine to L-3,4-dihydroxyphenylalanine (L-DOPA) and uses a cytochrome P450 reductase (CPR) to recharge the AD5/6 P450 enzyme. Then L-DOPA is converted to betalamic acid by a DOPA 4,5-dioxygenase (DODA). The resulting betalamic acid spontaneously reacts with amines, such as amino acids, to form betaxanthins. The betanin pathway involves four enzymes downstream of tyrosine. ADI has dual function activity converting tyrosine to L-DOPA and then L-DOPA to cyclo-DOPA. CPR recharges AD1. DODA converts L-DOPA to betalamic acid. Then, via various possible routes, betanin is produced via a UDP-glycosyltransferases (UGT) reaction and spontaneous condensation with betalamic acid. As shown in Example 6, several CB5s were identified that increased betaxanthin production. Without being bound by a particular theory, the CB5s may act as p450 reductase partners to the AD5/6 P450 enzyme.

In some embodiments, a betalain precursor is a betaxanthin and/or betanin precursor. In some embodiments, a betalain precursor is a substrate for a betalain synthesis enzyme. In some embodiments, a betalain synthesis enzyme is a AD5, AD6, a DODA, a cytochrome P450 reductase, UGT, or AD1. Non-limiting examples of betalain precursors include glucose, an amine, prephenate, tyrosine, L-DOPA, betalamic acid, cyclo-DOPA, betanidin, and cyclo-DOPA 5-O-glucoside.

In some embodiments, a host cell comprising one or more proteins described herein (e.g., a cytochrome b5 (CB5), a betalain synthesis enzyme, and/or any proteins associated with the disclosure) is capable of producing at least 0.005 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 180 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 1,950 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, at least 10,000 mg/L, at least 11,000 mg/L, at least 12,000 mg/L, at least 13,000 mg/L, at least 14,000 mg/L, at least 15,000 mg/L, at least 16,000 mg/L, at least 17,000 mg/L, at least 18,000 mg/L, at least 19,000 mg/L, at least 20,000 mg/L, at least 21,000 mg/L, at least 22,000 mg/L, at least 23,000 mg/L, at least 24,000 mg/L, at least 25,000 mg/L, at least 26,000 mg/L, at least 27,000 mg/L, at least 28,000 mg/L, at least 29,000 mg/L, at least 30,000 mg/L, at least 31,000 mg/L, at least 32,000 mg/L, at least 33,000 mg/L, at least 34,000 mg/L, at least 35,000 mg/L, at least 36,000 mg/L, at least 37,000 mg/L, at least 38,000 mg/L, at least 39,000 mg/L, at least 40,000 mg/L, at least 41.000 mg/L, at least 42,000 mg/L, at least 43,000 mg/L, at least 44,000 mg/L, at least 45,000 mg/L, at least 46,000 mg/L, at least 47,000 mg/L, at least 48,000 mg/L, at least 49,000 mg/L, at least 50,000 mg/L, at least 51,000 mg/L, at least 52,000 mg/L, at least 53,000 mg/L, at least 54,000 mg/L, at least 55,000 mg/L, at least 56,000 mg/L, at least 57,000 mg/L, at least 58,000 mg/L, at least 59,000 mg/L, at least 60,000 mg/L, at least 61,000 mg/L, at least 62,000 mg/L, at least 63,000 mg/L, at least 64,000 mg/L, at least 65,000 mg/L, at least 66,000 mg/L, at least 67,000 mg/L, at least 68,000 mg/L, at least 69,000 mg/L, at least 70,000 mg/L, at least 71,000 mg/L, at least 72,000 mg/L, at least 73,000 mg/L, at least 74,000 mg/L, at least 75,000 mg/L, at least 76,000 mg/L, at least 77,000 mg/L, at least 78,000 mg/L, at least 79,000 mg/L, at least 80,000 mg/L, at least 81,000 mg/L, at least 82,000 mg/L, at least 83,000 mg/L, at least 84,000 mg/L, at least 85,000 mg/L, at least 86,000 mg/L, at least 87,000 mg/L, at least 88,000 mg/L, at least 89,000 mg/L, at least 90,000 mg/L, at least 91,000 mg/L, at least 92,000 mg/L, at least 93,000 mg/L, at least 94,000 mg/L, at least 95,000 mg/L, at least 96,000 mg/L, at least 97,000 mg/L, at least 98,000 mg/L, at least 99,000 mg/L, or at least 100,000 mg/L of a betalain or a precursor thereof.

### Synthesis of Astaxanthin

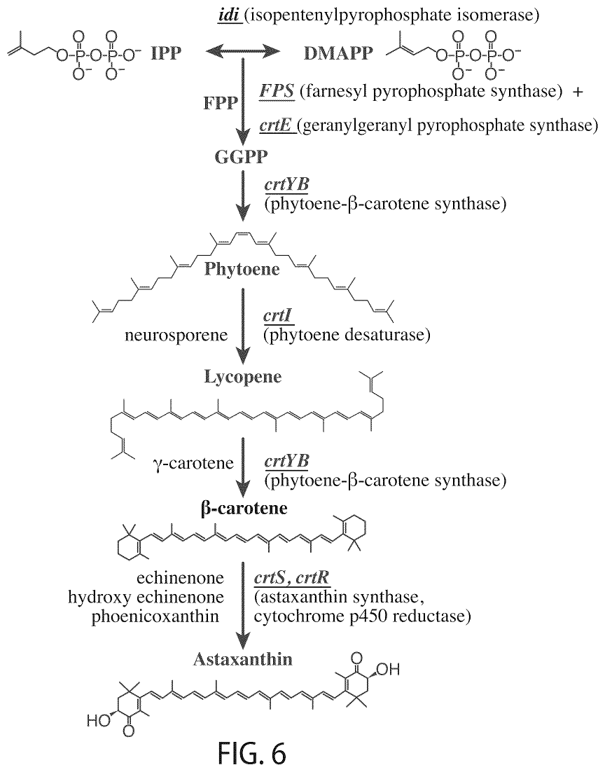

Astaxanthin is a carotenoid that has numerous uses including use as a feed supplement and as an antioxidant. This optically active compound is the main carotenoid produced by Xanthophyllomyces dendrorhous (Phaffia rhodozyma). Astaxanthin synthesis in X. dendrorhous begins with the mevalonate pathway, which produces isopentenylpyrophosphate (IPP). As shown in FIG. 6, eight molecules of IPP are used to form phytoene through the use of a farnesyl pyrophosphate synthase, a geranylgeranyl pyrophosphate synthase, and a phytoene-β-carotene synthase. Then, phytoene is converted into β-carotene through four dehydrogenation and two cyclization reactions. Phytoene desaturase converts phytoene into lycopene. Then lycopene is converted into β-carotene by a phytoene-β-carotene synthase. Finally, β-carotene is oxidized to form astaxanthin by a cytochrome P450 and cytochrome P450 reductase. In X. dendrorhous, the cytochrome P450 is astaxanthin synthase. A non-limiting example of an astaxanthin synthase from X. dendrorhous is provided as SEQ ID NO: 128. As shown in Example 7, CB5 alpha (SEQ ID NO: 1) was found to increase astaxanthin production. Without being bound by a particular theory, CB5 may increase astaxanthin production by promoting activity of the astaxanthin synthase, crtS.

In some embodiments, an astaxanthin precursor is geranylgeranyl-pyrophosphate (GGPP), phytoene, lycopene, or β-carotene. In some embodiments, an astaxanthin precursor is a substrate for an astaxanthin synthesis enzyme. In some embodiments, an astaxanthin synthesis enzyme is an isopentenylpyrophosphate isomerase, farnesyl pyrophosphate synthase, geranylgeranyl pyrophosphate synthase, phytoene-beta-carotene synthase, phytoene desaturase, phytoene-beta-carotene synthase, an astaxanthin synthase, or a cytochrome p450 reductase. In some embodiments, an astaxanthin synthesis enzyme is crtE, crtYB, crtI, crtYB, crtS, or crtR.

In some embodiments, a host cell comprising one or more proteins described herein (e.g., a cytochrome b5 (CB5), an astaxanthin synthesis enzyme, and/or any proteins associated with the disclosure) is capable of producing at least 0.001 mg/L, at least 0.002 mg/L, at least 0.003 mg/L, at least 0.004 mg/L, at least 0.005 mg/L, at least 0.006 mg/L, at least 0.007 mg/L, at least 0.008 mg/L, at least 0.009 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, at least 10,000 mg/L, at least 11,000 mg/L, at least 12,000 mg/L, at least 13,000 mg/L, at least 14,000 mg/L, at least 15,000 mg/L, at least 16,000 mg/L, at least 17,000 mg/L, at least 18,000 mg/L, at least 19,000 mg/L, at least 20,000 mg/L, at least 21,000 mg/L, at least 22,000 mg/L, at least 23,000 mg/L, at least 24,000 mg/L, at least 25,000 mg/L, at least 26,000 mg/L, at least 27,000 mg/L, at least 28,000 mg/L, at least 29,000 mg/L, at least 30,000 mg/L, at least 31,000 mg/L, at least 32,000 mg/L, at least 33,000 mg/L, at least 34,000 mg/L, at least 35,000 mg/L, at least 36,000 mg/L, at least 37,000 mg/L, at least 38,000 mg/L, at least 39,000 mg/L, at least 40,000 mg/L, at least 41,000 mg/L, at least 42,000 mg/L, at least 43,000 mg/L, at least 44,000 mg/L, at least 45,000 mg/L, at least 46,000 mg/L, at least 47,000 mg/L, at least 48,000 mg/L, at least 49,000 mg/L, at least 50,000 mg/L, at least 51,000 mg/L, at least 52,000 mg/L, at least 53,000 mg/L, at least 54,000 mg/L, at least 55,000 mg/L, at least 56,000 mg/L, at least 57,000 mg/L, at least 58,000 mg/L, at least 59,000 mg/L, at least 60,000 mg/L, at least 61,000 mg/L, at least 62,000 mg/L, at least 63,000 mg/L, at least 64,000 mg/L, at least 65,000 mg/L, at least 66,000 mg/L, at least 67,000 mg/L, at least 68,000 mg/L, at least 69,000 mg/L, at least 70,000 mg/L, at least 71.000 mg/L, at least 72,000 mg/L, at least 73,000 mg/L, at least 74,000 mg/L, at least 75,000 mg/L, at least 76,000 mg/L, at least 77,000 mg/L, at least 78,000 mg/L, at least 79,000 mg/L, at least 80,000 mg/L, at least 81,000 mg/L, at least 82,000 mg/L, at least 83,000 mg/L, at least 84,000 mg/L, at least 85,000 mg/L, at least 86,000 mg/L, at least 87,000 mg/L, at least 88,000 mg/L, at least 89,000 mg/L, at least 90,000 mg/L, at least 91,000 mg/L, at least 92,000 mg/L, at least 93,000 mg/L, at least 94,000 mg/L, at least 95,000 mg/L, at least 96,000 mg/L, at least 97,000 mg/L, at least 98,000 mg/L, at least 99.000 mg/L, or at least 100,000 mg/L of astaxanthin or a precursor thereof.

Synthesis of Quillaic acid

Quillaic acid (QA) is derived from the triterpene β-amyrin. β-amyrin synthase (“BAS”) cyclizes 2,3-oxidosqualene (OS) to produce β-amyrin. A series of three cytochrome P450 oxidases oxidize β-amyrin with a carboxylic acid, alcohol and aldehyde at the C28, C16 and C23 positions, respectively, to produce quillaic acid. A C28 oxidase oxidizes β-amyrin to produce erythrodiol. A C28 oxidase oxidizes erythrodiol to produce oleanolic acid. A C23 oxidase oxidizes oleanolic acid to produce hederagenin. A C23 oxidase oxidizes hederagenin to produce gypsogenin. A C16 oxidase oxidizes gypsogenin to produce quillaic acid. Quillaic acid forms the trierpene core of QS-21. Without being bound by a particular theory, CB5 may increase quillaic acid production by promoting activity of the one or more cytochrome P450 oxidases that oxidize p-amyrin (e.g., one or more of the C28, C16, and C23 oxidases).

In some embodiments, a quillaic acid precursor is 2,3-oxidosqualene, β-amyrin, erythrodiol, oleanolic acid, hederagenin, or gypsogenin. In some embodiments, a quillaic acid precursor is a substrate for a quillaic acid synthesis enzyme. In some embodiments, a quillaic acid synthesis enzyme is a BAS. C28 oxidase, C16 oxidase, or C23 oxidase.

In some embodiments, a host cell comprising one or more proteins described herein (e.g., a cytochrome b5 (CB5), a quillaic synthesis enzyme, and/or any proteins associated with the disclosure) is capable of producing at least 0.001 mg/L, at least 0.002 mg/L, at least 0.003 mg/L, at least 0.004 mg/L, at least 0.005 mg/L, at least 0.006 mg/L, at least 0.007 mg/L, at least 0.008 mg/L, at least 0.009 mg/L, at least 0.01 mg/L, at least 0.02 mg/L, at least 0.03 mg/L, at least 0.04 mg/L, at least 0.05 mg/L, at least 0.06 mg/L, at least 0.07 mg/L, at least 0.08 mg/L, at least 0.09 mg/L, at least 0.1 mg/L, at least 0.2 mg/L, at least 0.3 mg/L, at least 0.4 mg/L, at least 0.5 mg/L, at least 0.6 mg/L, at least 0.7 mg/L, at least 0.8 mg/L, at least 0.9 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L, at least 16 mg/L, at least 17 mg/L, at least 18 mg/L, at least 19 mg/L, at least 20 mg/L, at least 21 mg/L, at least 22 mg/L, at least 23 mg/L, at least 24 mg/L, at least 25 mg/L, at least 26 mg/L, at least 27 mg/L, at least 28 mg/L, at least 29 mg/L, at least 30 mg/L, at least 31 mg/L, at least 32 mg/L, at least 33 mg/L, at least 34 mg/L, at least 35 mg/L, at least 36 mg/L, at least 37 mg/L, at least 38 mg/L, at least 39 mg/L, at least 40 mg/L, at least 41 mg/L, at least 42 mg/L, at least 43 mg/L, at least 44 mg/L, at least 45 mg/L, at least 46 mg/L, at least 47 mg/L, at least 48 mg/L, at least 49 mg/L, at least 50 mg/L, at least 51 mg/L, at least 52 mg/L, at least 53 mg/L, at least 54 mg/L, at least 55 mg/L, at least 56 mg/L, at least 57 mg/L, at least 58 mg/L, at least 59 mg/L, at least 60 mg/L, at least 61 mg/L, at least 62 mg/L, at least 63 mg/L, at least 64 mg/L, at least 65 mg/L, at least 66 mg/L, at least 67 mg/L, at least 68 mg/L, at least 69 mg/L, at least 70 mg/L, at least 75 mg/L, at least 80 mg/L, at least 85 mg/L, at least 90 mg/L, at least 95 mg/L, at least 100 mg/L, at least 125 mg/L, at least 150 mg/L, at least 175 mg/L, at least 200 mg/L, at least 225 mg/L, at least 250 mg/L, at least 275 mg/L, at least 300 mg/L, at least 325 mg/L, at least 350 mg/L, at least 375 mg/L, at least 400 mg/L, at least 425 mg/L, at least 450 mg/L, at least 475 mg/L, at least 500 mg/L, at least 1,000 mg/L, at least 2,000 mg/L, at least 3,000 mg/L, at least 4,000 mg/L, at least 5,000 mg/L, at least 6,000 mg/L, at least 7,000 mg/L, at least 8,000 mg/L, at least 9,000 mg/L, at least 10,000 mg/L, at least 11,000 mg/L, at least 12,000 mg/L, at least 13,000 mg/L, at least 14,000 mg/L, at least 15,000 mg/L, at least 16,000 mg/L, at least 17,000 mg/L, at least 18,000 mg/L, at least 19,000 mg/L, at least 20,000 mg/L, at least 21,000 mg/L, at least 22,000 mg/L, at least 23,000 mg/L, at least 24,000 mg/L, at least 25,000 mg/L, at least 26,000 mg/L, at least 27,000 mg/L, at least 28.000 mg/L, at least 29,000 mg/L, at least 30,000 mg/L, at least 31,000 mg/L, at least 32,000 mg/L, at least 33,000 mg/L, at least 34,000 mg/L, at least 35,000 mg/L, at least 36,000 mg/L, at least 37,000 mg/L, at least 38,000 mg/L, at least 39,000 mg/L, at least 40,000 mg/L, at least 41,000 mg/L, at least 42,000 mg/L, at least 43,000 mg/L, at least 44,000 mg/L, at least 45,000 mg/L, at least 46,000 mg/L, at least 47,000 mg/L, at least 48,000 mg/L, at least 49,000 mg/L, at least 50,000 mg/L, at least 51,000 mg/L, at least 52,000 mg/L, at least 53,000 mg/L, at least 54,000 mg/L, at least 55,000 mg/L, at least 56,000 mg/L, at least 57,000 mg/L, at least 58,000 mg/L, at least 59,000 mg/L, at least 60,000 mg/L, at least 61,000 mg/L, at least 62,000 mg/L, at least 63,000 mg/L, at least 64,000 mg/L, at least 65,000 mg/L, at least 66,000 mg/L, at least 67,000 mg/L, at least 68,000 mg/L, at least 69,000 mg/L, at least 70,000 mg/L, at least 71,000 mg/L, at least 72,000 mg/L, at least 73,000 mg/L, at least 74,000 mg/L, at least 75,000 mg/L, at least 76,000 mg/L, at least 77,000 mg/L, at least 78,000 mg/L, at least 79,000 mg/L, at least 80,000 mg/L, at least 81,000 mg/L, at least 82,000 mg/L, at least 83,000 mg/L, at least 84.000 mg/L, at least 85,000 mg/L, at least 86,000 mg/L, at least 87,000 mg/L, at least 88,000 mg/L, at least 89,000 mg/L, at least 90,000 mg/L, at least 91,000 mg/L, at least 92,000 mg/L, at least 93,000 mg/L, at least 94,000 mg/L, at least 95,000 mg/L, at least 96,000 mg/L, at least 97,000 mg/L, at least 98,000 mg/L, at least 99,000 mg/L, or at least 100,000 mg/L of quillaic acid or a precursor thereof.

### Cytochrome BSs (CB5s)

Aspects of the present disclosure provide cytochrome b5 (CB5) proteins, which may be useful in promoting production of oxygenated hydrocarbons. As used herein, a “cytochrome b5” or “CB5” refers to a protein that comprises a lipid binding domain or cytochrome b5-like heme binding domain. In some embodiments, a lipid binding domain is a steroid binding domain.

CB5 proteins are heme- or lipid-binding proteins. For example, a CB5 may be a steroid binding protein. Some have been implicated in electron transport and enzymatic redox reactions. CB5 proteins generally harbor a conserved CB5 domain (e.g., a cytochrome b5-like heme or steroid binding domain). The tertiary structure of the CB5 domain is highly conserved and the domain folds around two hydrophobic residue cores on each side of a beta sheet. Without wishing to be bound by any theory, one hydrophobic core may include the heme or lipid binding domain, while the other hydrophobic core may promote formation of the proper conformation. In some embodiments, a lipid binding domain is a steroid binding domain.

Without being bound by a particular theory, two histidine residues may be required for a CB5 to interact with the iron in heme and CB5s that do not comprise these conserved histidine residues may comprise a lipid binding domain (e.g., a steroid binding domain) instead of a heme-binding domain. In some embodiments, a CB5 that is capable of increasing production of an oxygenated hydrocarbon does not comprise two histidine residues in a region corresponding to positions 64-104 of SEQ ID NO: 1, in a region corresponding to positions 105-122 of SEQ ID NO: 1, and/or in a region corresponding to positions 123-165 of SEQ ID NO: 1. In some embodiments, a CB5 that is capable of increasing production of an oxygenated hydrocarbon comprises at most one histidine in a region corresponding to positions 64-104 of SEQ ID NO: 1, in a region corresponding to positions 105-122 of SEQ ID NO: 1, and/or in a region corresponding to positions 123-165 of SEQ ID NO: 1. In some embodiments, a CB5 that is capable of increasing production of an oxygenated hydrocarbon comprises no histidine residues in a region corresponding to positions 64-104 of SEQ ID NO: 1, in a region corresponding to positions 105-122 of SEQ ID NO: 1, and/or in a region corresponding to positions 123-165 of SEQ ID NO: 1.

A non-limiting example of a CB5 domain is provided under Pfam Accession No. PF00173. The CB5 domain may form a majority of the protein's structure. See e.g., SEQ ID NOs: 1-3 or 318. In some embodiments, additional domains such as a fatty acid desaturase and/or a FMN-dependent dehydrogenase are also present.

CB5 proteins may serve as an electron transfer component of a redox reaction. For example, a CB5 may function as an obligate electron donor in an oxidative reaction. In some embodiments, a CB5 serves as an electron-delivery partner for a cytochrome P450 (e.g., a C11 hydroxylase). In some embodiments, a CB5 catalyzes or promotes electron transfer from NADPH to a cytochrome P450 enzyme (e.g., a C11 hydroxylase).

In some embodiments, a CB5 plays an allosteric role to promote production of an oxygenated hydrocarbon. As a non-limiting example, a CB5 may be involved in binding and positioning of cucurbitadienol or cucurbitadienol-like molecules to support P450 enzyme activity. In some embodiments, a CB5 sterically interacts with the P450 enzyme to support an enzyme conformation that promotes higher activity, without a direct enzymatic role of the CB5 itself.

The rate of an enzymatic redox reaction may be assessed by any suitable method, including determination of the change in product concentration over a period of time. Any suitable method including mass spectrometry may be used to measure the presence of a substrate or product. See also, e.g., Schenkman et al., Pharmacology & Therapeutics 97 (2003) 139-152; Gou et al., Plant Cell. 2019 June; 31(6):1344-1366; Interpro Accession No. IPR001199; Interpro Accession No. IPR018506; Lederer Biochimie. 1994; 76(7):674-92; GenBank Accession No. AF332415; UniProt Accession No. P40312.

In some embodiments, a CB5 is 200-300 amino acids in length (e.g., 210-290 amino acids in length, 205-215 amino acids in length, or 275-295 amino acids in length).

In some embodiments, a CB5 of the present disclosure comprises a sequence (e.g., nucleic acid or amino acid sequence) that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs: 1-24, 29-30, 32-40, 77-88, 95-107, 118, 316-318, 330-331, or a CB5 sequence in Table 14, or any CB5 sequence disclosed in this application or known in the art. In some embodiments, a CB5 of the present disclosure comprises a sequence that is a conservatively substituted version of any one of SEQ ID NOs: 1-10, 29-30, 32-34, 77-82, 118, and 318.

In some embodiments, a CB5 comprises one or more motifs. As a non-limiting example, a motif may distinguish a CB5 that is capable of increasing production of an oxygenated hydrocarbon from a CB5 that does not increase production of an oxygenated hydrocarbon relative to a control.

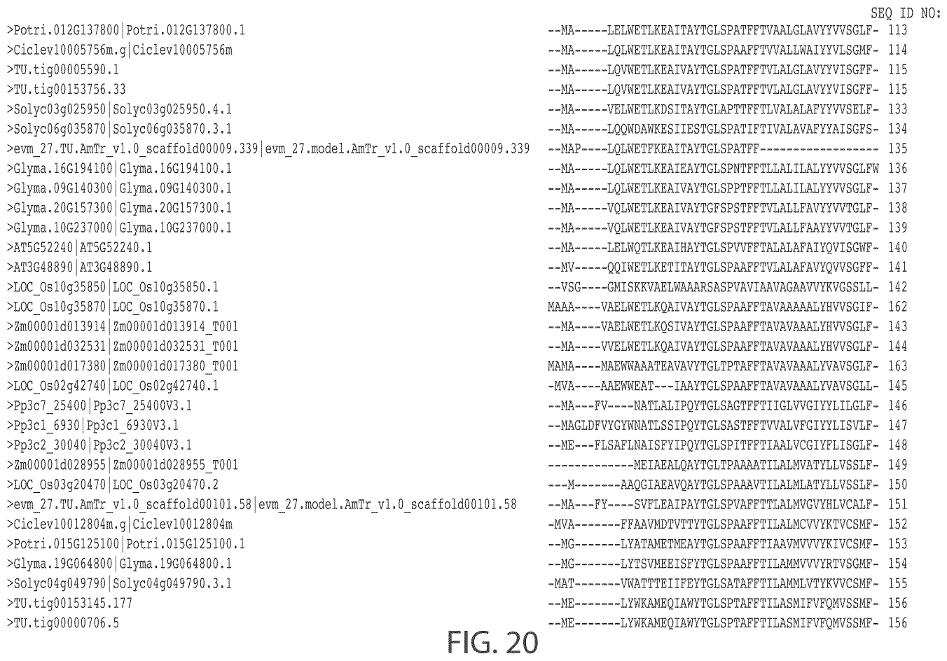

Without wishing to be bound by a particular theory, a motif shown in FIG. 20 may be present in a CB5 that promotes production of an oxygenated hydrocarbon. In some embodiments, a CB5 comprises a motif that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to a motif shown in FIG. 20. In some embodiments, a CB5 comprises all or a portion of a motif shown in FIG. 20.

In some embodiments, a CB5 comprises the amino acid sequence YTGLSP (SEQ ID NO: 47); the amino acid sequence KPLLMAIKGQIYDVS (SEQ ID NO: 48); and/or the amino acid sequence LQDWEYKFM (SEQ ID NO: 49). In some embodiments, the CB5 comprises the amino acid sequence YTGLSP (SEQ ID NO: 47) at residues corresponding to positions 16-21 in SEQ ID NO: 1; the amino acid sequence KPLLMAIKGQIYDVS (SEQ ID NO: 48) at residues corresponding to positions 85-99 in SEQ ID NO: 1; and/or the amino acid sequence LQDWEYKFM (SEQ ID NO: 49) at residues corresponding to positions 148-156 in SEQ ID NO: 1. In some embodiments, a CB5 comprises the amino acid sequence YTGLSP (SEQ ID NO: 47); the amino acid sequence KPLLMAIKGQIYDVS (SEQ ID NO: 48); and/or the amino acid sequence LQDWEYKFM (SEQ ID NO: 49). In some embodiments, the CB5 comprises the amino acid sequence YTGLSP (SEQ ID NO: 47) at residues corresponding to positions 16-21 in SEQ ID NO: 1; the amino acid sequence KPLLMAIKGQIYDVS (SEQ ID NO: 48) at residues corresponding to positions 85-99 in SEQ ID NO: 1; and the amino acid sequence LQDWEYKFM (SEQ ID NO: 49) at residues corresponding to positions 148-156 in SEQ ID NO: 1.

In some embodiments, a CB5 comprises the amino acid sequence X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃FX₂₄X₂₅X₂₆X₂₇X₂₈X₂₉X₃₀X₃₁ (SEQ ID NO: 50), in which X₁ is the amino acid E or Q; X₂ is the amino acid L or V; X₃ is the amino acid Y or W; X₄ is the amino acid W or E; X₅ is the amino acid K or T; X₆ is the amino acid A or L; X₇ is the amino acid M or K; X₈ is the amino acid Q or A; X₉ is the amino acid A or V; X₁₀ is the amino acid W or A; X₁₁ is the amino acid T or A; X₁₂ is the amino acid A or T; X₁₃ is the amino acid I or V; X₁₄ is the amino acid S or L; X₁₅ is the amino acid M or G; X₁₆ is the amino acid I or L; X₁₇ is the amino acid F or A; X₁₈ is the amino acid F or Y; X₁₉ is the amino acid Q or Y; X₂₀ is the amino acid M or V; X₂₁ is the amino acid V or I; X₂₂ is the amino acid S or G; X₂₃ is the amino acid M or F; X₂₄ is the amino acid V or G; X₂₅ is the amino acid S or T; X₂₆ is the amino acid P or S; X₂₇ is the amino acid E or D; X₂₈ is the amino acid E or Y; X₂₉ is the amino acid F or G; X₃₀ is the amino acid N or S; and/or X₃₁ is the amino acid K or H. As a non-limiting example, a CB5 comprising SEQ ID NO: 50 may comprise the amino acid sequence QVWETLKEAIVAYTGLSPATFFFVLALGLAVYYVISGFFGTSDYGSH (SEQ ID NO: 58) or the amino acid sequence ELYWKAMEQIAWYTGLSPTAFFTILASMIFVFQMVSSMFVSPEEFNK (SEQ ID NO: 59). In some embodiments, a CB5 may comprise SEQ ID NO: 50 at residues corresponding to positions 4-50 of SEQ ID NO: 1.

In some embodiments, a CB5 comprises the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51), wherein: X₁ is the amino acid P or A; X₂ is the amino acid V or I; X₃ is the amino acid E or Q; X₄ is the amino acid I or L; X₅ is the amino acid S or T; X₆ is the amino acid E or Q; X₇ is the amino acid E or Q; X₈ is the amino acid K or R; X₉ is the amino acid Q or A; X₁₀ is the amino acid S or P; X₁₁ is the amino acid K or N; X₁₂ is the amino acid Q or S; and/or X₁₃ is the amino acid S or G. As a non-limiting example, a CB5 comprising SEQ ID NO: 51 may comprise the amino acid sequence PVQVGEISEEELKQYDGSDSKKPLLMAIKGQIYDVSQSRMF (SEQ ID NO: 60) or AVQIGQLTEQQLRAYDGSDPNKPLLMAIKGQIYDVSSGRMF (SEQ ID NO: 61). In some embodiments, a CB5 may comprise SEQ ID NO: 51 at residues corresponding to positions 64-104 of SEQ ID NO: 1.

In some embodiments, a CB5 comprises the amino acid sequence LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52), in which: X₁ is the amino acid K or L; X₂ is the amino acid M or L; X₃ is the amino acid E or K; X₄ is the amino acid E or P; X₅ is the amino acid K or E; X₆ is the amino acid L or I; X₇ is the amino acid D or N; X₈ is the amino acid S or E; X₉ is the amino acid G or S; X₁₀ is the amino acid P or E; X₁₁ is the amino acid F or E; X₁₂ is the amino acid E or V; X₁₃ is the amino acid A or I; X₁₄ is the amino acid S or E; X₁₅ is the amino acid T or E; and/or X₁₆ is the amino acid V or L. In some embodiments, a CB5 comprising SEQ ID NO: 52 may comprise LAKMSFEEKDLTGDISGLGPFELEALQDWEYKFMSKYVKVGTV (SEQ ID NO: 62) or LALLSFKPEDITGNIEGLSEEELVILQDWEYKFMEKYVKVGEL (SEQ ID NO: 63). In some embodiments, a CB5 comprises SEQ ID NO: 52 at residues corresponding to positions 123-165 of SEQ ID NO: 1.

In some embodiments, a CB5 comprises the amino acid sequence X₁X₂X₃EX₄GX₅X₆X₇X₈X₉X₁₀D (SEQ ID NO: 53), in which: X₁ is the amino acid K or E; X₂ is the amino acid P or H; X₃ is the amino acid A or S; X₄ is the amino acid D or N; X₅ is the amino acid P or H; X₆ is the amino acid S or R; X₇ is the amino acid E or N; X₈ is the amino acid S or F; X₉ is the amino acid Q or E; and/or X₁₀ is the amino acid A or 1. In some embodiments, a CB5 comprising SEQ ID NO: 53 comprises KPAEDGPSESQAD (SEQ ID NO: 64) or EHSENGHRNFEID (SEQ ID NO: 65). In some embodiments, a CB5 comprises SEQ ID NO: 53 at residues corresponding to positions 190-202 of SEQ ID NO: 1.

In some embodiments, a CB5 comprises the amino acid sequence the amino acid sequence DATX₁X₂FX₃X₄X₅VGHS (SEQ ID NO: 31), wherein: X₁ is the amino acid E, D, or N; X₂ is the amino acid A or D; X₃ is the amino acid E or D; X₄ is the amino acid D or N; and/or X₈ is the amino acid V or A. In some embodiments, the CB5 comprises the amino acid sequence DATX₁X₂FEX₃VGHS (SEQ ID NO: 31) at residues corresponding to positions 53-64 in SEQ ID NO: 30. In some embodiments, a CB5 comprising SEQ ID NO: 31 comprises DATEAFEDVGHS (SEQ ID NO: 108), DATDDFENVGHS (SEQ ID NO: 109), DATDDFEDVGHS (SEQ ID NO: 110), DATDDFEDAGHS (SEQ ID NO: 111), or DATNDFDDVGHS (SEQ ID NO: 112).

In some embodiments, a CB5 comprises the amino acid sequence X₁X₂X₃X₄X₅X₆X₇EXSIX₉X₁₀YTGLSPX₁₁X₁₂FFTX₁₃LAX₁₄X₁₅X₁₆X₁₇VX₁₈X₁₉X₂₀X₂₁SX₂₂X₂₃FX₂₄X₂₅X₂₆X₂₇X₂₈X₂₉X₃₀X₃₁ (SEQ ID NO: 50). in which X₁ is the amino acid E or Q; X₂ is the amino acid L or V; X₃ is the amino acid Y or W; X₄ is the amino acid W or E; X₅ is the amino acid K or T; X₆ is the amino acid A or L; X₇ is the amino acid M or K; X₅ is the amino acid Q or A; X₉ is the amino acid A or V; X₁₀ is the amino acid W or A; X₁₁ is the amino acid T or A; X₁₂ is the amino acid A or T; X₁₃ is the amino acid I or V; X₁₄ is the amino acid S or L; X₁₅ is the amino acid M or G; X₁₆ is the amino acid I or L; X₁₇ is the amino acid F or A; X₁₈ is the amino acid F or Y; X₁₉ is the amino acid Q or Y; X₂₀ is the amino acid M or V; X₂₁ is the amino acid V or I; X₂₂ is the amino acid S or G; X₂₃ is the amino acid M or F; X₂₄ is the amino acid V or G; X₂₅ is the amino acid S or T; X₂₆ is the amino acid P or S; X₂₇ is the amino acid E or D; X₂₈ is the amino acid E or Y; X₂₉ is the amino acid F or G; X₃₀ is the amino acid N or S; and/or X₃₁ is the amino acid K or H; the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51), wherein: X₁ is the amino acid P or A; X₂ is the amino acid V or I; X₃ is the amino acid E or Q; X₄ is the amino acid I or L; X₅ is the amino acid S or T; X₆ is the amino acid E or Q; X₇ is the amino acid E or Q; X₈ is the amino acid K or R; X₉ is the amino acid Q or A; X₁₀ is the amino acid S or P; X₁₁ is the amino acid K or N; X₁₂ is the amino acid Q or S; and/or X₁₃ is the amino acid S or G; and the amino acid sequence LAX₁X₂SFX₃X₄X₅DX₆TGX₇IX₈GLX₉X₁₀X₁₁ELX₁₂X₁₃LQDWEYKFMX₁₄KYVKVGX₁₅X₁₆ (SEQ ID NO: 52), in which: X₁ is the amino acid K or L; X₂ is the amino acid M or L; X₃ is the amino acid E or K; X₄ is the amino acid E or P; X₅ is the amino acid K or E; X₆ is the amino acid L or I; X₇ is the amino acid D or N; X₈ is the amino acid S or E; X₉ is the amino acid G or S; X₁₀ is the amino acid P or E; X₁₁ is the amino acid F or E; X₁₂ is the amino acid E or V; X₁₃ is the amino acid A or 1; X₁₄ is the amino acid S or E; X₁₉ is the amino acid T or E; and/or X₁₆ is the amino acid V or L. In some embodiments, the CB5 further comprises the amino acid sequence X₁X₂X₃EX₄GXSX₆X₇X₈X₉X₁₀D (SEQ ID NO: 53), in which: X₁ is the amino acid K or E; X₂ is the amino acid P or H; X₃ is the amino acid A or S; X₄ is the amino acid D or N; X₅ is the amino acid P or H; X₆ is the amino acid S or R; X₇ is the amino acid E or N; X₈ is the amino acid S or F; X₉ is the amino acid Q or E; and/or X₁₀ is the amino acid A or I.

In some embodiments, a CB5 comprises the amino acid sequence QVWETLKEAIVAYTGLSPATFFFVLALGLAVYYVISGFFGTSDYGSH (SEQ ID NO: 58) or the amino acid sequence ELYWKAMEQIAWYTGLSPTAFFTILASMIFVFQMVSSMFVSPEEFNK (SEQ ID NO: 59); the amino acid sequence PVQVGEISEEELKQYDGSDSKKPLLMAIKGQIYDVSQSRMF (SEQ ID NO: 60) or AVQIGQLTEQQLRAYDGSDPNKPLLMAIKGQIYDVSSGRMF (SEQ ID NO: 61); and the amino acid sequence LAKMSFEEKDLTGDISGLGPFELEALQDWEYKFMSKYVKVGTV (SEQ ID NO: 62) or LALLSFKPEDITGNIEGLSEEELVILQDWEYKFMEKYVKVGEL (SEQ ID NO: 63). In some embodiments, the CB5 further comprises KPAEDGPSESQAD (SEQ ID NO: 64) or EHSENGHRNFEID (SEQ ID NO: 65).

In some embodiments, a CB5 comprises the amino acid sequence ILRVSFRKYRKAIEQ (SEQ ID NO: 54); the amino acid sequence RAFRPSIRFKKSHSTVPT (SEQ ID NO: 55); the amino acid sequence KNTLYVGG (SEQ ID NO: 56); and/or the amino acid sequence DQATQKHRSFGFVTFLEKED (SEQ ID NO: 57). In some embodiments, a CB5 comprises the amino acid sequence ILRVSFRKYRKAIEQ (SEQ ID NO: 54) at residues corresponding to positions 23-37 of SEQ ID NO: 4; the amino acid sequence RAFRPSIRFKKSHSTVPT (SEQ ID NO: 55) at residues corresponding to positions 53-70 of SEQ ID NO: 4; the amino acid sequence KNTLYVGG (SEQ ID NO: 56) at residues corresponding to positions 168-175 of SEQ ID NO: 4; and/or the amino acid sequence DQATQKHRSFGFVTFLEKED (SEQ ID NO: 57) at residues corresponding to positions 203-222 of SEQ ID NO: 4. In some embodiments, a CB5 comprises the amino acid sequence ILRVSFRKYRKAIEQ (SEQ ID NO: 54); the amino acid sequence RAFRPSIRFKKSHSTVPT (SEQ ID NO: 55); the amino acid sequence KNTLYVGG (SEQ ID NO: 56); and the amino acid sequence DQATQKHRSFGFVTFLEKED (SEQ ID NO: 57).

In some embodiments, a CB5 is capable of increasing production of an oxygenated hydrocarbon by a host cell by at least 0.01%, at least 0.05%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%, including all values in between relative to production of the oxygenated hydrocarbon by a host cell that does not comprise the CB5. In some embodiments, a CB5 is capable of increasing production of an oxygenated hydrocarbon by a host cell at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95%, at most 100%, at most 150%, at most 200%, at most 250%, at most 300%, at most 350%, at most 400%, at most 450%, at most 500%, at most 550%, at most 600%, at most 650%, at most 700%, at most 750%, at most 800%, at most 850%, at most 900%, at most 950%, or at most 1000%, including all values in between relative to production of the oxygenated hydrocarbon by a host cell that does not comprise the CB5. In some embodiments, a CB5 is capable of increasing production of an oxygenated hydrocarbon by a host cell between 0.01% and I %, between 1% and 10%, between 10% and 20%, between 10% and 50%, between 50% and 100%, between 100% and 200%, between 200% and 300%, between 300% and 400%, between 400% and 500%, between 500% and 600%, between 600% and 700%, between 700% and 800%, between 800% and 900%, between 900% and 1000%, between 1% and 50%, between 1% and 100%, between 1% and 500%, or between 1% and 1,000%, including all values in between relative to production of the oxygenated hydrocarbon by a host cell that does not comprise the CB5.

In some embodiments, a host cell comprising a CB5 is capable of producing at least 0.01 mg/L, at least 0.05 mg/L, at least 1 mg/L, at least 5 mg/L, at least 10 mg/L, at least 15 mg/L, at least 20 mg/L, at least 25 mg/L, at least 30 mg/L, at least 35 mg/L, at least 40 mg/L, at least 45 mg/L, at least 50 mg/L, at least 55 mg/L, at least 60 mg/L, at least 65 mg/L, at least 70 mg/L, at least 75 mg/L, at least 80 mg/L, at least 85 mg/L, at least 90 mg/L, at least 95 mg/L, at least 100 mg/L, at least 150 mg/L, at least 200 mg/L, at least 250 mg/L, at least 300 mg/L, at least 350 mg/L, at least 400 mg/L, at least 450 mg/L, at least 500 mg/L, at least 550 mg/L, at least 600 mg/L, at least 650 mg/L, at least 700 mg/L, at least 750 mg/L, at least 800 mg/L, at least 850 mg/L, at least 900 mg/L, at least 950 mg/L, or at least 1000 mg/L, including all values of an oxygenated hydrocarbon. In some embodiments, a host cell comprising a CB5 is capable of producing at most 5 mg/L, at most 10 mg/L, at most 15 mg/L, at most 20 mg/L, at most 25 mg/L, at most 30 mg/L, at most 35 mg/L, at most 40 mg/L, at most 45 mg/L, at most 50 mg/L, at most 55 mg/L, at most 60 mg/L, at most 65 mg/L, at most 70 mg/L, at most 75 mg/L, at most 80 mg/L, at most 85 mg/L, at most 90 mg/L, at most 95 mg/L, at most 100 mg/L, at most 150 mg/L, at most 200 mg/L, at most 250 mg/L, at most 300 mg/L, at most 350 mg/L, at most 400 mg/L, at most 450 mg/L, at most 500 mg/L, at most 550 mg/L, at most 600 mg/L, at most 650 mg/L, at most 700 mg/L, at most 750 mg/L, at most 800 mg/L, at most 850 mg/L, at most 900 mg/L, at most 950 mg/L, or at most 1000 mg/L of an oxygenated hydrocarbon. In some embodiments, a host cell comprising a CB5 is capable of producing between 0.01 mg/L and 1 mg/L, between 1 mg/L and 10 mg/L, between 10 mg/L and 20 mg/L, between 10 mg/L and 50 mg/L, between 50 mg/L and 100 mg/L, between 100 mg/L and 200 mg/L, between 200 mg/L and 300 mg/L, between 300 mg/L and 400 mg/L, between 400 mg/L and 500 mg/L, between 500 mg/L and 600 mg/L, between 600 mg/L and 700 mg/L, between 700 mg/L and 800 mg/L, between 800 mg/L and 900 mg/L, between 900 mg/L and 1000 mg/L, between 1 mg/L and 50 mg/L, between 1 mg/L and 100 mg/L, between 1 mg/L and 500 mg/L, or between 1 mg/L and 1,000 mg/L, including all values in between of an oxygenated hydrocarbon. As a non-limiting example, a CB5 may be capable of increasing production of an oxygenated hydrocarbon by a host cell that comprises one or more squalene synthases, epoxidases, cytochrome P450 reductases, C11 hydroxylases, epoxide hydrolases, and/or cucurbitadienol synthases. In some instances, a CB5 is capable of increasing production of an oxygenated hydrocarbon by a host cell that comprises one or more squalene synthases, epoxidases, cytochrome P450 reductases, C11 hydroxylases, epoxide hydrolases, cucurbitadienol synthases, and/or UDP-glycosyltransferases. In some embodiments, a host cell further comprises a CB5 reductase. In some embodiments, a host cell further comprises a glucanase.

### Cytochrome P450 Enzymes (CYP)

Aspects of the present disclosure provide cytochrome P450 enzymes (CYPs), which may be useful, for example, in the production of oxygenated hydrocarbons. Cytochrome P450s catalyze oxidation reactions. In some embodiments, cytochrome P450s catalyze oxygenation reactions, i.e., adding one or more oxygens to produce an oxygenated hydrocarbon. In some embodiments, a CYP functions as a monooxygenases. Structurally, CYPs includes an active site with a heme-iron center. Iron is bound to CYPs through a cysteine thiolate ligand. The cysteine thiolate ligand and flanking residues are often highly conserved in CYPs. The active site may comprise the motif [FW]-[SGNH]-x-[GD]-{F}-[RKHPT]-{P}-C-[LIVMFAP]-[GAD], in which acceptable amino acids for a given position are indicated between square parentheses “[ ]” and amino acids that are not accepted at a given position are indicated between curly brackets “{ }”. See, e.g., Prosite Accession No. PS00086. Non-limiting examples of CYPs include C11 hydroxylases, ABAI, ABA2, T16H2, T3O, PPDS, AD1, AD5, AD6, C28 oxidase, C16 oxidase, C23 oxidase, and CrtS.

A CYP of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a CYP sequence (e.g., nucleic acid or amino acid sequence), with a sequence set forth as SEQ ID NO: 25, 41, 43, 69, 71, 89, 90, 94, 117, 120, 124, 127, 128, 264, 265, 280, 281, 296, 314, 320, 321, 324, 334, or 335 or a CYP disclosure in Table 14 or to any CYP sequence disclosed in this application or known in the art. In some embodiments, a CYP comprises a sequence that is a conservatively substituted version of any one of SEQ ID NOs: 41, 43, 89, 90, 117, 120, 128, 280, 281, and 324.

In some embodiments, a CYP is a C11 hydroxylase. In some embodiments, a C11 hydroxylase of the present disclosure is capable of oxidizing mogrol precursors (e.g., cucurbitadienol, 11-hydroxycucurbitadienol, 24,25-dihydroxy-cucurbitadienol, and/or 24,25-epoxy-cucurbitadienol). In some embodiments, a C11 hydroxylase of the present disclosure catalyzes the formation of mogrol.

In some embodiments, a CYP of the present disclosure is capable of oxidizing α-ionylideneethane (α-IE) and/or α-ionylideneacetic acid (α-IAA). In some embodiments, a CYP of the present disclosure catalyzes the formation of α-IAA and/or DH-α-IAA. In some embodiments, a CYP of the present disclosure is aba1 or aba2.

In some embodiments, a CYP of the present disclosure is capable of catalyzing hydroxylation of tabersonine and/or 16-methoxytabersonine. In some embodiments, a CYP of the present disclosure catalyzes the formation of 16-OH-tabersonine and/or 3-OH-16-MOH-2,3-2H-tabersonine. In some embodiments, a CYP of the present disclosure is T16H2 or T3O.

In some embodiments, a CYP of the present disclosure is capable of catalyzing hydroxylation of dammarenediol-II (DII). In some embodiments, a CYP of the present disclosure catalyzes the formation of 20(S)-Protopanaxadiol (PPD). In some embodiments, a CYP of the present disclosure is PPDS.

In some embodiments, a CYP of the present disclosure is capable of converting tyrosine to L-3,4-dihydroxyphenylalanine (L-DOPA). In some embodiments, a CYP of the present disclosure is AD5. AD6, or AD1.

It should be appreciated that activity, such as specific activity, of a CYP can be determined by any means known to one of ordinary skill in the art. In some embodiments, activity (e.g., specific activity) of a CYP may be measured as the concentration of an oxygenated hydrocarbon produced per unit of enzyme per unit time. In some embodiments, a CYP of the present disclosure has an activity (e.g., specific activity) of at least 0.0001-0.001 μmol/min/mg, at least 0.001-0.01 μmol/min/mg, at least 0.01-0.1 μmol/min/mg, or at least 0.1-1 μmol/min/mg, including all values in between.

In some embodiments, the activity, such as specific activity, of a CYP is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control CYP.

### Cytochrome P450 Reductase Enzymes

Aspects of the present disclosure provide cytochrome P450 reductase enzymes, which may be useful, for example, in the production of an oxygenated hydrocarbon. Cytochrome P450 reductase is also referred to as NADPH:ferrihemoprotein oxidoreductase, NADPH:hemoprotein oxidoreductase, NADPH:P450 oxidoreductase, P450 reductase, POR, CPR, and CYPOR. These reductases can promote cytochrome P450 (CYP) activity by catalyzing electron transfer from NADPH to a C11 hydroxylase.

Cytochrome P450 reductases of the present disclosure may comprise a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, with a cytochrome P450 reductase sequence (e.g., nucleic acid or amino acid sequence) in Table 14, with a sequence set forth as SEQ ID NO: 28, 44, 68, 72, 76, 93, 121, 125, 282, 283, 297, 298, 323, or 333 or to any cytochrome p450 reductase disclosed in this application or known in the art. In some embodiments, a cytochrome P450 reductase comprises a sequence that is a conservatively substituted version of any one of SEQ ID NOs: 44, 68, 93, 121, 282, and 283.

In some embodiments, a cytochrome P450 reductase of the present disclosure is capable of promoting oxidation of an oxygenated hydrocarbon. In some embodiments, a P450 reductase of the present disclosure catalyzes the formation of an oxygenated hydrocarbon.

It should be appreciated that activity (e.g., specific activity) of a cytochrome P450 reductase can be measured by any means known to one of ordinary skill in the art. In some embodiments, activity (e.g., specific activity) of a recombinant cytochrome P450 reductase may be measured as the concentration of an oxygenated hydrocarbon produced per unit enzyme per unit time in the presence of a CYP. In some embodiments, a cytochrome P450 reductase of the present disclosure has an activity (e.g., specific activity) of at least 0.0001-0.001 μmol/min/mg, at least 0.001-0.01 μmol/min/mg, at least 0.01-0.1 μmol/min/mg, or at least 0.1-1 μmol/min/mg, including all values in between.

In some embodiments, the activity (e.g., specific activity) of a cytochrome P450 reductase is at least 1.1 fold (e.g., at least 1.3 fold, at least 1.5 fold, at least 1.7 fold, at least 1.9 fold, at least 2 fold, at least 2.5 fold, at least 3 fold, at least 4 fold, at least 5 fold, at least 10 fold, at least 20 fold, at least 30 fold, at least 40 fold, at least 50 fold, at least 100 fold, at least 1000 fold or at least 10000 fold, including all values in between) greater than that of a control cytochrome P450 reductase.

### Variants

Aspects of the disclosure relate to polynucleotides encoding any of the polypeptides described, including CB5s, cytochrome P450s, cytochrome P450 reductases, mogrol synthesis enzymes, mogroside synthesis enzymes, ABA synthesis enzymes, vinca alkaloid synthesis enzymes, ginsenoside synthesis enzymes, betalain synthesis enzymes, and astaxanthin synthesis enzymes, and any other polypeptides associated with the disclosure. Variants of polynucleotide or polypeptide sequences described in this application are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity” refers to the relatedness of the sequences of two polypeptides or polynucleotides when the sequences are aligned, and the term “percent identity” refers to the percentage of residues (amino acids or nucleotides) that are identical when two or more polypeptide or polynucleotide sequences are aligned. In some embodiments, sequence identity and/or percent identity is determined across the entire length of a sequence, while in other embodiments, sequence identity and/or percent identity is determined over a region of a sequence.

Percent identity of related polypeptide or polynucleotide sequences can be calculated by any of the methods known to one of ordinary skill in the art. For example, percent identity can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3. Where gaps exist between two sequences, Gapped BLAST® can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

A second example of a local alignment technique is based on the Smith-Waterman algorithm (Smith, T. F. & Waterman, M. S. (1981) J. Mol. Biol. 147:195-197). An example of a global alignment technique is the Needleman-Wunsch algorithm (Needleman. S. B. & Wunsch, C. D. (1970) J. Mol. Biol. 48:443-453), which is based on dynamic programming. A further example of a global alignment technique is the Fast Optimal Global Sequence Alignment Algorithm (FOGSAA).

In some embodiments, the identity of two polypeptide sequences is determined by aligning the two amino acid sequences of the polypeptides, calculating the number of identical amino acids, and dividing by the length of one of the polypeptide sequences. In some embodiments, the identity of two polynucleotide sequences is determined by aligning the two nucleotide sequences of the polynucleotides, calculating the number of identical nucleotide and dividing by the length of one of the polynucleotide sequences.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST® programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443-453).

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA).

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539).

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using sequence alignment tools known in the art.

Variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between) and include but are not limited to paralogous sequences, orthologous sequences, or sequences arising from convergent evolution. Paralogous sequences arise from duplication of a gene within a genome of a species, while orthologous sequences diverge after a speciation event. Two different species may have evolved independently but may each comprise a sequence that shares a certain percent identity with a sequence from the other species as a result of convergent evolution.

In some embodiments, a polypeptide variant (e.g., CB5, cytochrome P450, cytochrome P450 reductase, mogrol synthesis enzyme, mogroside synthesis enzyme, ABA synthesis enzyme, vinca alkaloid synthesis enzyme, ginsenoside synthesis enzyme, betalain synthesis enzyme, and astaxanthin synthesis enzyme variant or variant of any other polypeptide associated with the disclosure) comprises a domain that shares a secondary structure (e.g., alpha helix, beta sheet) with a reference polypeptide (e.g., a reference CB5, cytochrome P450, cytochrome P450 reductase, mogrol synthesis enzyme, mogroside synthesis enzyme, ABA synthesis enzyme, vinca alkaloid synthesis enzyme, ginsenoside synthesis enzyme, betalain synthesis enzyme, and astaxanthin synthesis enzyme, or any enzyme or other polypeptide associated with the disclosure). In some embodiments, a polypeptide variant (e.g., CB5, cytochrome P450, cytochrome P450 reductase, mogrol synthesis enzyme, mogroside synthesis enzyme, ABA synthesis enzyme, vinca alkaloid synthesis enzyme, ginsenoside synthesis enzyme, betalain synthesis enzyme, and astaxanthin synthesis enzyme variant or variant of any other polypeptide associated with the disclosure) shares a tertiary structure with a reference polypeptide (e.g., a reference CB5, cytochrome P450, cytochrome P450 reductase, mogrol synthesis enzyme, mogroside synthesis enzyme, ABA synthesis enzyme, vinca alkaloid synthesis enzyme, ginsenoside synthesis enzyme, betalain synthesis enzyme, and astaxanthin synthesis enzyme, or any other polypeptide associated with the disclosure). As a non-limiting example, a variant polypeptide may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets, or have the same tertiary structure as a reference polypeptide. For example, a loop may be located between a beta sheet and an alpha helix, between two alpha helices, or between two beta sheets. Homology modeling may be used to compare two or more tertiary structures.

One or more mutations can be made in a nucleotide sequence using any method known to a person of ordinary skill in the art. For example, one or more mutations can be made by gene editing tools, PCR, site-directed mutagenesis (e.g., according to Kunkel, Proc. Nat. Acad. Sci. U.S.A. 82: 488-492, 1985), chemical synthesis of a gene or polypeptide, or by insertions, such as insertion of a tag (e.g., a HIS tag or a GFP tag). Mutations can include, for example, substitutions, deletions, additions, insertions, fusions, and translocations, generated by any method known in the art.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) compared to the linear sequence of the polypeptide before it was circularized and severed as determined by linear sequence alignment methods (e.g., Clustal Omega or BLAST). Topological analysis of the two polypeptides, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce a polypeptide with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a polypeptide that has undergone circular permutation, the linear amino acid sequence of the polypeptide would differ from a reference polypeptide that has not undergone circular permutation. However, one of ordinary skill in the art would be able to determine which residues in the polypeptide that has undergone circular permutation correspond to residues in the reference polypeptide that has not undergone circular permutation by, for example, aligning the sequences and detecting conserved motifs, and/or by comparing the structures or predicted structures of the polypeptides, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example. RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

Functional variants of the CB5s, cytochrome P450s, cytochrome P450 reductases, mogrol synthesis enzymes, mogroside synthesis enzymes, ABA synthesis enzymes, vinca alkaloid synthesis enzymes, ginsenoside synthesis enzymes, betalain synthesis enzymes, and astaxanthin synthesis enzymes, and any other polypeptides disclosed in this application are also encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins.

Putative functional variants may also be identified by searching for polypeptides with functionally annotated domains. Databases including Pfam (Sonnhammer et al., Proteins. 1997 July; 28(3):405-20) may be used to identify polypeptides with a particular domain. This leucine residue has been implicated in determining the product specificity of the CDS enzyme; mutation of this residue can, for instance, result in cycloartenol or parkeol as a product (Takase et al., Org Biomol Chem. 2015 Jul. 13(26):7331-6).

Additional UGT enzymes may be identified, for example, by searching for polypeptides with a UDPGT domain (PROSITE accession number PS00375).

Homology modeling may also be used to identify amino acid residues that are amenable to mutation without affecting function. A non-limiting example of such a method may include use of position-specific scoring matrix (PSSM) and an energy minimization protocol. See, e.g.,Stormo et al., Nucleic Acids Res. 1982 May 11:10(9):2997-3011.

PSSM may be paired with calculation of a Rosetta energy function, which determines the difference between the wild-type and a mutant, such as a point mutant. Without being bound by a particular theory, potentially stabilizing mutations can be desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21:63(2):337-346. doi: 10.1016/j.molcel.2016.06.012.

In some embodiments, a polynucleotide sequence encoding a CB5, cytochrome P450, cytochrome P450 reductase, mogrol synthesis enzyme, mogroside synthesis enzyme, ABA synthesis enzyme, vinca alkaloid synthesis enzyme, ginsenoside synthesis enzyme, betalain synthesis enzyme, and astaxanthin synthesis enzyme coding sequence or a polynucleotide sequence encoding any other polypeptide associated with the disclosure comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 nucleotide positions corresponding to a reference sequence. In some embodiments, the polynucleotide sequence encoding a CB5, cytochrome P450, cytochrome P450 reductase, mogrol synthesis enzyme, mogroside synthesis enzyme, ABA synthesis enzyme, vinca alkaloid synthesis enzyme, ginsenoside synthesis enzyme, betalain synthesis enzyme, and astaxanthin synthesis enzyme coding sequence or the polynucleotide sequence encoding any other polypeptide associated with the disclosure comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of a coding sequence relative to a reference coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence relative to the amino acid sequence of a reference polypeptide.

In some embodiments, the one or more mutations in a polynucleotide sequence encoding a CB5, cytochrome P450, cytochrome P450 reductase, mogrol synthesis enzyme, mogroside synthesis enzyme, ABA synthesis enzyme, vinca alkaloid synthesis enzyme, ginsenoside synthesis enzyme, betalain synthesis enzyme, and astaxanthin synthesis enzyme sequence or the polynucleotide sequence encoding any other polypeptide associated with the disclosure alter the amino acid sequence of the polypeptide relative to the amino acid sequence of a reference polypeptide. In some embodiments, the one or more mutations alter the amino acid sequence of the polypeptide relative to the amino acid sequence of a reference polypeptide and alter (enhance or reduce) an activity of the polypeptide relative to the reference polypeptide.

The activity, including specific activity, of any of the polypeptides described in this application may be measured using methods known in the art. As a non-limiting example, a polypeptide's activity may be determined by measuring its substrate specificity, product(s) produced, the concentration of product(s) produced, or any combination thereof. As used in this application, “specific activity” of a polypeptide refers to the amount (e.g., concentration) of a particular product produced for a given amount (e.g., concentration) of the polypeptide per unit time.

One or more mutations in a polypeptide coding sequence may result in one or more conservative amino acid substitutions. As used in this application, a “conservative amino acid substitution” or “conservatively substituted amino acid” refers to an amino acid substitution that does not alter the relative charge or size characteristics or functional activity of the protein in which the amino acid substitution is made.

In some instances, an amino acid is characterized by its R group (see, e.g., Table 1). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

A functionally equivalent variant of a polypeptide may include one or more conservative amino acid substitutions. Non-limiting examples of a conservative substitution of an amino acid include the substitution of one amino acid for another amino acid within each of the following groups: (a) M, I, L, and V; (b) F, Y, and W; (c) K, R, and H; (d) A and G; (e) S and T; (f) Q and N; and (g) E and D. Thus, a conservative substitution can be: substitution of a M residue with an I, L, or V residue, substitution of an I residue with a M, L, or V residue, substitution of a L residue with a M, I, or V residue, substitution of a V residue with a M, I, or L residue; substitution of a F residue with a Y or W residue, substitution of a Y residue with a F or W residue, substitution of a W residue with a F or Y residue; substitution of a K residue with a R or H residue, substitution of a R residue with a K or H residue, substitution of a H residue with a K or R residue; substitution of an A residue with a G residue or substitution of a G residue with an A residue; substitution of a S residue with a T residue or substitution of a T residue with a S residue; substitution of a Q residue with a N residue or substitution of an N residue with a Q residue; and/or substitution of an E residue with a D residue or substitution of a D residue with an E residue. Additional non-limiting examples of conservative amino acid substitutions are provided in Table 1.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions.

TABLE 1
Non-limiting examples of conservative amino acid substitutions

| Original | | Conservative Amino |
|---|---|---|
| Residue | R Group Type | Acid Substitutions |
| Ala (A) | nonpolar aliphatic R group | Cys, Gly, Ser |
| Arg (R) | positively charged R group | His, Lys |
| Asn (N) | polar uncharged R group | Asp, Gln, Glu |
| Asp (D) | negatively charged R group | Asn, Gln, Glu |
| Cys (C) | polar uncharged R group | Ala, Ser |
| Gln (Q) | polar uncharged R group | Asn, Asp, Glu |
| Glu (E) | negatively charged R group | Asn, Asp, Gln |
| Gly (G) | nonpolar aliphatic R group | Ala, Ser |
| His (H) | positively charged R group | Arg, Tyr, Trp |
| Ile (I) | nonpolar aliphatic R group | Leu, Met, Val |
| Leu (L) | nonpolar aliphatic R group | Ile, Met, Val |
| Lys (K) | positively charged R group | Arg, His |
| Met (M) | nonpolar aliphatic R group | Ile, Leu, Phe, Val |
| Pro (P) | polar uncharged R group | |
| Phe (F) | nonpolar aromatic R group | Met, Trp, Tyr |
| Ser (S) | polar uncharged R group | Ala, Gly, Thr |
| Thr (T) | polar uncharged R group | Ala, Asn, Ser |
| Trp (W) | nonpolar aromatic R group | His, Phe, Tyr, Met |
| Tyr (Y) | nonpolar aromatic R group | His, Phe, Trp |
| Val (V) | nonpolar aliphatic R group | Ile, Leu, Met, Thr |

One or more amino acid substitutions in the amino acid sequence of a polypeptide to produce a polypeptide variant having a desired property and/or activity can be made by alteration of the coding sequence of the polypeptide. Similarly, one or more conservative amino acid substitutions in the amino acid sequence of a polypeptide that produce a functionally equivalent variant of the polypeptide typically can be made by altering the coding sequence of the polypeptide (e.g., CB5, cytochrome P450, cytochrome P450 reductase, mogrol synthesis enzyme, mogroside synthesis enzyme, ABA synthesis enzyme, vinca alkaloid synthesis enzyme, ginsenoside synthesis enzyme, betalain synthesis enzyme, and astaxanthin synthesis enzyme, or any other polypeptide associated with the disclosure).

### Expression of Polynucleotides in Host Cells

Aspects of the present disclosure relate to the recombinant expression of polynucleotides encoding polypeptides, functional modifications and variants thereof, as well as uses relating thereto. For example, the methods described in this application may be used to produce oxygenated hydrocarbons.

The term “heterologous” with respect to a polynucleotide, such as a polynucleotide comprising a gene, is used interchangeably with the term “exogenous” or “recombinant” and refers to: a polynucleotide that has been artificially supplied to a biological system such as a cell; a polynucleotide that has been modified within a biological system; or a polynucleotide whose expression or regulation has been manipulated within a biological system. A heterologous polynucleotide that is introduced into or expressed in a host cell may be a synthetic polynucleotide, a polynucleotide that comes from a different organism or species from the host cell, or a polynucleotide that results from modification or selective editing within the host cell of a polynucleotide that is endogenous to the host cell. A polynucleotide that is endogenous to a host cell also may be considered heterologous when it is, for example: situated non-naturally in the host cell; expressed recombinantly in the host cell, either stably or transiently; present in a copy number that differs from the naturally occurring copy number within the host cell; or expressed in a non-natural way or at a non-natural level within the host cell, such as through manipulation of regulatory regions that control expression of the polynucleotide. In some embodiments, a heterologous polynucleotide is a polynucleotide that is endogenous to a host cell but whose expression is driven by a promoter that does not naturally regulate expression of the polynucleotide. In other embodiments, a heterologous polynucleotide is a polynucleotide that is endogenous to the host cell and whose expression is driven by a promoter that does naturally regulate expression of the polynucleotide, but the promoter driving its expression or another regulatory region regulating its expression has been modified. In some embodiments, the promoter is recombinantly activated or repressed. For example, gene-editing techniques may be used to regulate expression of a polynucleotide, including an endogenous polynucleotide, from a promoter, including an endogenous promoter. See, e.g., Chavez ei al., Nat Methods. 2016 July; 13(7): 563-567. A heterologous polynucleotide may comprise a wild-type sequence or a mutant sequence as compared with a reference polynucleotide sequence.

A polynucleotide encoding any of the polypeptides, such as CB5s, cytochrome P450s, cytochrome P450 reductases, mogrol synthesis enzymes, mogroside synthesis enzymes. ABA synthesis enzymes, vinca alkaloid synthesis enzymes, ginsenoside synthesis enzymes, betalain synthesis enzymes, and astaxanthin synthesis enzymes, or any other polypeptides associated with the disclosure may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

The vector may be a cloning vector, such as a plasmid, fosmid, phagemid, virus genome or artificial chromosome.

As used in this application, the terms “expression vector” or “expression construct” refer to a nucleic acid construct, generated recombinantly or synthetically, with a series of specified nucleic acid elements that permit transcription of a particular polynucleotide in a host cell, such as a yeast cell. In some embodiments, a polynucleotide associated with the disclosure is inserted into an expression vector or expression construct such that it is operably joined to regulatory sequences and, in some embodiments, expressed as an RNA transcript. In some embodiments, the expression vector or expression construct contains one or more markers, such as a selectable marker, to identify cells transformed or transfected with the expression vector or expression construct. A polynucleotide encoding a polypeptide associated with the disclosure is “operably joined” or “operably linked” to a regulatory sequence when the polynucleotide and the regulatory sequence are covalently linked and the expression or transcription of the polynucleotide is under the influence or control of the regulatory sequence.

In some embodiments, a polynucleotide encoding any of the polypeptides described in this application is under the control of regulatory sequences (e.g., enhancer sequences). In some embodiments, a polynucleotide (e.g., a polynucleotide comprising a gene) is expressed under the control of a promoter. In some embodiments, the promoter is a native promoter, corresponding to the promoter of the gene in its endogenous context. In other embodiments, the promoter is not the native promoter of the gene, e.g., the promoter is different from the promoter of the gene in its endogenous context.

In some embodiments, the promoter is a eukaryotic promoter. Non-limiting examples of eukaryotic promoters include P_40s, CtrYB, P_CrtS, and P_HMG1, TDH3, PGK1, PKC1, PDC1, TEF1, TEF2, RPL18B, SSA1, TDH2, PYKI, TPII GAL1, GAL10, GAL7, GAL3, GAL2, MET3, MET25, HXT3, HXT7, ACT1, ADH1, ADH2, CUP1-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls Icon, T3, T7, SP6, and PL. Non-limiting examples of bacterial promoters include Pbad, PmgrB, Ptrc2, Plac/ara, Ptac, and Pm.

In some embodiments, a promoter comprises a sequence that is at least that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identical, including all values in between, to any one of SEQ ID NOs: 129-132.

In some embodiments, the promoter is an inducible promoter. As used in this application, an “inducible promoter” is a promoter controlled by the presence or absence of a molecule. Non-limiting examples of inducible promoters include chemically-regulated promoters and physically-regulated promoters. For chemically-regulated promoters, the transcriptional activity can be regulated by one or more compounds, such as alcohol, an antibiotic such as tetracycline, a carbon source such as galactose, a steroid, a metal, or other compounds. For physically-regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination thereof.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKCI, PDC1, TEFI, TEF2, RPL18B, SSA1. TDH2, PYK1, TPI1, HXT3, HXT7, ACT1, ADH I, ADH2, ENO2, and SOD.

Other inducible promoters or constitutive promoters known to one of ordinary skill in the art are also contemplated.

In some embodiments, introduction of a polynucleotide, such as a polynucleotide encoding a polypeptide associated with the disclosure, into a host cell results in genomic integration of the polynucleotide. In some embodiments, a host cell comprises at least 1 copy, at least 2 copies, at least 3 copies, at least 4 copies, at least 5 copies, at least 6 copies, at least 7 copies, at least 8 copies, at least 9 copies, at least 10 copies, at least 11 copies, at least 12 copies, at least 13 copies, at least 14 copies, at least 15 copies, at least 16 copies, at least 17 copies, at least 18 copies, at least 19 copies, at least 20 copies, at least 21 copies, at least 22 copies, at least 23 copies, at least 24 copies, at least 25 copies, at least 26 copies, at least 27 copies, at least 28 copies, at least 29 copies, at least 30 copies, at least 31 copies, at least 32 copies, at least 33 copies, at least 34 copies, at least 35 copies, at least 36 copies, at least 37 copies, at least 38 copies, at least 39 copies, at least 40 copies, at least 41 copies, at least 42 copies, at least 43 copies, at least 44 copies, at least 45 copies, at least 46 copies, at least 47 copies, at least 48 copies, at least 49 copies, at least 50 copies, at least 60 copies, at least 70 copies, at least 80 copies, at least 90 copies, at least 100 copies, or more, including any values in between, of a polynucleotide sequence, such as a polynucleotide sequence encoding any of the polypeptides described in this application, in its genome.

In some embodiments, the sequence of a polynucleotide (e.g., a polynucleotide comprising a gene) is codon-optimized. Codon optimization may increase expression of a gene by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not codon-optimized.

### Host Cells

Any of the polynucleotides or polypeptides of the disclosure may be expressed in a host cell. As used in this application, the term “host cell” refers to a cell that can be used to express a polynucleotide, such as a polynucleotide that encodes a polypeptide used in production of oxygenated hydrocarbons.

Any suitable host cell may be used to produce any of the recombinant polypeptides, including CB5s, cytochrome P450s, cytochrome P450 reductases, mogrol synthesis enzymes, mogroside synthesis enzymes, ABA synthesis enzymes, vinca alkaloid synthesis enzymes, ginsenoside synthesis enzymes, betalain synthesis enzymes, and astaxanthin synthesis enzymes, and any other polypeptides disclosed in this application, including eukaryotic cells or prokaryotic cells. Suitable host cells include, but are not limited to, fungal cells (e.g., yeast cells), bacterial cells (e.g., E. coli cells), algal cells, plant cells, insect cells, and animal cells, including mammalian cells.

Suitable yeast host cells include, but are not limited to, Candida, Escherichia, Hansenula, Saccharomyces (e.g., S. cerevisiae), Xanthophyllomyces, Schizosaccharomyces, Pichia, Kluyveromyces (e.g., K. lactis), and Yarrowia (e.g., Y. lipolytica). In some embodiments, the yeast cell is Hansenula polymorpha, Saccharomyces cerevisiae, Xanthophyllomyces dendrorhous. Saccharomyces carlsbergensis, Saccharomyces diastaticus, Saccharomyces norbensis, Saccharomyces kluyveri, SchizosaccharomYces pombe, Pichia finlandica, Pichia trehalophila, Pichia kodamae, Pichia membranaefaciens, Pichia opuntiae, Pichia thermotolerans, Pichia salictaria. Pichia quercuum, Pichia pijperi, Pichia stipitis, Pichia methanolica, Pichia angusta, Komagataella phaffii, Komagataella pastoris. Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus. Arthrobacter, Azobacter, Bacillus, Bifidobacterium, Brevibacterium, Butyrivibrio, Buchnera, Campestris, Campylobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus. Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Saccharopolyspora, Staphylococcus, Serratia, Salmonella, Shigella, Thermoanaerobacterium, Tropheryma, Tularensis, Temecula, Thermosynechococcus. Thermococcus, Ureaplasma, Xanthomonas, Xylella, Yersinia, and Zymomonas.

In some embodiments, the bacterial host cell is of the Agrobacterium species (e.g., A. radiobacter, A. rhizogenes, A. rubi), the Arthrobacter species (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), or the Bacillus species (e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii. B. stearothermophilus, B. halodurans, B. amyloliquefaciens. In particular embodiments, the host cell is an industrial Bacillus strain including but not limited to B. subtilis, B. pumilus, B. licheniformis, B. megaterium, B. clausii, B. stearothermophilus and B. amyloliquefaciens. In some embodiments, the host cell is an industrial Clostridium species (e.g., C. acetobutylicum, C. tetani E88, C. lituseburense, C saccharobutylicum, C. perfringens, C. beijerinckii). In some embodiments, the host cell is an industrial Corynebacterium species (e.g., C. glutamicum, C. acetoacidophilum). In some embodiments, the host cell is an industrial Escherichia species (e.g., E. coli). In some embodiments, the host cell is an industrial Erwinia species (e.g., E. uredovora, E. caromovora, E. ananas, E. herbicola, E. punctata, E. terreus). In some embodiments, the host cell is an industrial Pantoea species (e.g., P. citrea, P. agglomerans). In some embodiments, the host cell is an industrial Pseudomonas species, (e.g., P. putida, P. aeruginosa, P. mevalonii). In some embodiments, the host cell is an industrial Streptococcus species (e.g., S. equisimiles, S. pyogenes, S. uberis). In some embodiments, the host cell is an industrial Streptomyces species (e.g., S. ambofaciens. S. achromogenes. S. avermitilis. S. coelicolor, S. aureofaciens, S. aureus, S. fungicidicus, S. griseus, S. lividans). In some embodiments, the host cell is an industrial Zymomonas species (e.g., Z. mobilis, Z. lipolytica).

The present disclosure is also suitable for use with a variety of animal cell types, including mammalian cells, for example, human (including 293, HeLa. W138, PER.C6 and Bowes melanoma cells), mouse (including 3T3, NS0, NS1, Sp2/0), hamster (CHO, BHK), monkey (COS, FRhL, Vero), and hybridoma cell lines.

The present disclosure is also suitable for use with a variety of plant cell types.

The term “cell,” as used in this application, may refer to a single cell or a population of cells, such as a population of cells belonging to the same cell line or strain. Use of the singular term “cell” should not be construed to refer explicitly to a single cell rather than a population of cells.

The host cell may comprise genetic modifications relative to a wild-type counterpart. As a non-limiting example, a host cell (e.g., S. cerevisiae or Y. lipolytica) may be modified to reduce or inactivate one or more of the following genes: hydroxymethylglutaryl-CoA (HMG-CoA) reductase (HMG1), acetyl-CoA C-acetyltransferase (acetoacetyl-CoA thiolase) (ERG10), 3-hydroxy-3-methylglutaryl-CoA (HMG-CoA) synthase (ERG13), farnesyl-diphosphate farnesyl transferase (squalene synthase) (ERG9), may be modified to overexpress squalene epoxidase (ERGi), or may be modified to downregulate lanosterol synthase (ERG7). In some embodiments, a host cell is modified to reduce or eliminate expression of one or more transporter genes, such as PDR I or PDR3, and/or the glucanase gene EXG1.

In some embodiments, a host cell is modified to reduce or inactivate at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 genes.

In some embodiments, a host cell is modified to reduce or inactivate 1, 2, 3, 4, 5, 6, 7, 8, 9, 10, 11, 12, 13, 14, 15, 16, 17, 18, 19, or 20 genes. A vector or polynucleotide encoding any of the polypeptides described in this application may be introduced into a suitable host cell using any method known in the art. Non-limiting examples of yeast transformation protocols are described in Gietz et al., Yeast transformation can be conducted by the LiAc/SS Carrier DNA/PEG method. Methods Mol Biol. 2006; 313:107-20, which is incorporated by reference in its entirety. Host cells may be cultured under any suitable conditions as would be understood by one of ordinary skill in the art. For example, any media, temperature, and incubation conditions known in the art may be used. For host cells carrying an inducible vector, cells may be cultured with an appropriate inducible agent to promote expression.

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cells. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place, involving a living organism, part of a living organism, or purified proteins. Any type of bioreactor or fermenter known in the art may be compatible with aspects of the disclosure.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., oxygenated hydrocarbon) may display some differences from the substrate in terms of solubility, toxicity, cellular accumulation and secretion and in some embodiments can have different fermentation kinetics.

The methods described in this application encompass production of the oxygenated hydrocarbons using a host cell, cell lysate or isolated recombinant polypeptides.

Oxygenated hydrocarbons produced by any of the host cells disclosed in this application may be identified and extracted using any method known in the art. Mass spectrometry (e.g., LC-MS, GC-MS) is a non-limiting example of a method for identification and may be used to help identify a compound of interest.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1. Identification and Functional Characterization of CB5 Proteins that Increase Mogrol Production

This Example describes the screening of S. grosvenorii proteins in S. cerevisiae to identify proteins that promote mogrol production. The library included approximately 333 S. grosvenorii proteins whose expression correlated with expression and/or matched enzyme class of proteins involved in mogroside biosynthesis. Transcriptomic data from Xia et al. Gigascience. 2018 Jun. 1:7(6):giy067 was used for the analysis. The entire library was screened for mogrol production to determine whether proteins whose expression correlated with expression and/or matched enzyme class of one or more proteins involved in mogroside biosynthesis could be used to increase mogrol production.

S. cerevisiae host cells were used for the screens. The host cell base strain was engineered to express one or more copies of CYPI798, CYP5491, AtCPR, CPR4497, SgCDS, EPH3, and AtEPH2, as well as to upregulate expression of ERG9 and ERG1 and downregulate expression of ERG7. The base strain also had several copies of pPGKL_X_tSSA1 integrated into the genome. “X” corresponds to the F-Cphl recognition site, which is 24 bp and has the sequence GATGCACGAGCGCAACGCTCACAA (SEQ ID NO: 46).

To test the protein library for enhanced mogrol production, an in vivo plate assay was combined with LC-MS analysis. Plasmids carrying individual genes were transformed and integrated into the chromosome of a S. cerevisiae chassis strain that produces mogrol. A strain lacking any additional plant protein was used as a negative control.

Single colonies resulting from transformation were grown as pre-cultures containing culturing media in a shaking incubator at 26° C. for 96 hours at 1000 rpm. After 48 hours, pre-cultures were transferred into production media and grown in a shaking incubator at 26° C. for 96 hours at 1000 rpm. After 96 hours, cultures were extracted with an organic solvent and product formation was tested by LC-MS analysis to evaluate mogrol and mogroside production. A Thermo Scientific Q Exactive Focus MS with a LX2 multiplexed columns setup was used. Thermo Scientific Accucore PFP columns (2.6 μm, 2.1 mm×100 mm) with 12.5 mM ammonium acetate pH 8.0 in water running buffer and acetonitrile ramp were used for separation in negative mode using full scan.

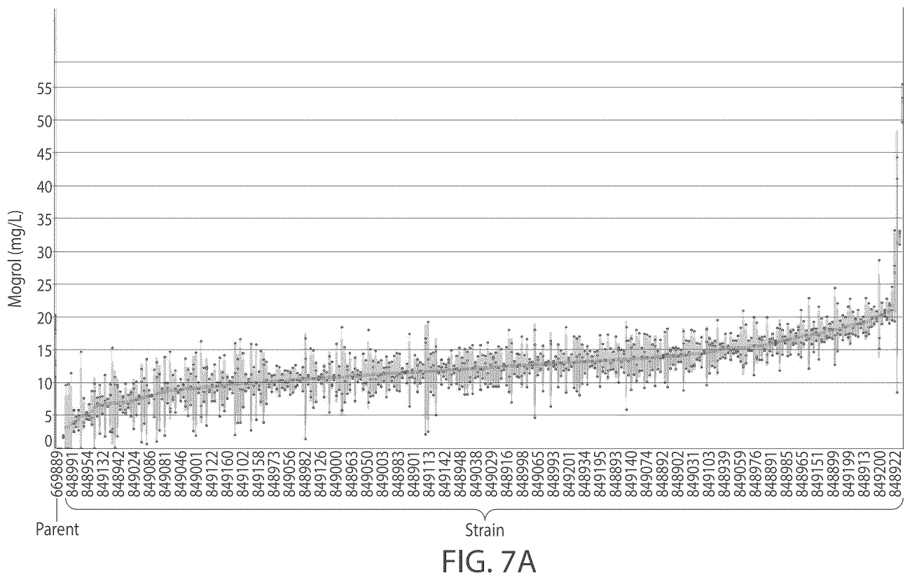

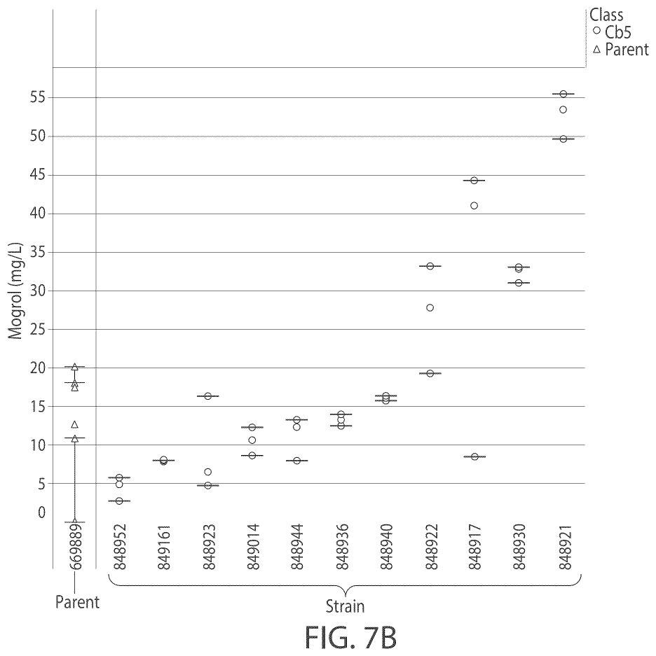

Initially, a short analytical run was performed to identify product species based on mass. Based on this screen, several proteins were identified that increased mogrol production of the parental strain (Table 2 and FIGS. 7A-7B). In particular, eleven cytochrome b5 (CB5) proteins were included in the screen (FIG. 7B). Several of these CB5 proteins were found to increase mogrol production of the parental strains, including the CB5 proteins expressed by strains 848921, 848930, 848917, 848922, and 848940.

Analysis of CB5 proteins in the screen using a motif identification software identified multiple sequence motifs that were enriched in CB5 proteins that increased mogrol production as compared to CB5 proteins that did not increase mogrol production.

The following motifs, corresponding to SEQ ID NOs: 47-49, are present in the CB5 sequences expressed in strains 848917, 848921, 848922, and 848930:
a) the amino acid sequence YTGLSP (SEQ ID NO: 47);
b) the amino acid sequence KPLLMAIKGQIYDVS (SEQ ID NO: 48); and
c) the amino acid sequence LQDWEYKFM (SEQ ID NO: 49).

The following motifs, corresponding to SEQ ID NOs: 50-53, are also present in the CB5 sequences expressed in strains 848917, 848921, 848922, and 848930:
a) the amino acid sequence X₁X₂X₃X₄X₅X₆X₇EX₈IX₉X₁₀YTGLSPX₁₁X₁₂FFfX₁₃LAX₁₄X₁₅X₁₆X₁₇VX₁₈X₁₉X₂₀X₂₁SX₂₂X₂₃FX₂₄X₂₅X₂₆X₂₇X₂₈X₂₉X₃₀X₃₁ (SEQ ID NO: 50), in which:
   (i) X₁ is the amino acid E or Q;
   (ii) X₂ is the amino acid L or V;
   (iii) X₃ is the amino acid Y or W;
   (iv) X₄ is the amino acid W or E;
   (v) X₈ is the amino acid K or T;
   (vi) X₆ is the amino acid A or L;
   (vii) X₇ is the amino acid M or K;
   (viii) X₈ is the amino acid Q or A;
   (ix) X₉ is the amino acid A or V;
   (x) X₁₀ is the amino acid W or A;
   (xi) X₁₁ is the amino acid T or A;
   (xii) X₁₂ is the amino acid A or T;
   (xiii) X₁₃ is the amino acid I or V;
   (xiv) X₁₄ is the amino acid S or L;
   (xv) X₁₅ is the amino acid M or G;
   (xvi) X₁₆ is the amino acid 1 or L;
   (xvii) X₁₇ is the amino acid F or A;
   (xviii) X₁₈ is the amino acid F or Y;
   (xix) X₁₉ is the amino acid Q or Y;
   (xx) X₂₀ is the amino acid M or V;
   (xxi) X₂₁ is the amino acid V or I;
   (xxii) X₂₂ is the amino acid S or G;
   (xxiii) X₂₃ is the amino acid M or F;
   (xxiv) X₂₄ is the amino acid V or G;
   (xxv) X₂₅ is the amino acid S or T;
   (xxvi) X₂₆ is the amino acid P or S;
   (xxvii) X₂₇ is the amino acid E or D;
   (xxviii) X₂₈ is the amino acid E or Y;
   (xxix) X₂₉ is the amino acid F or G;
   (xxx) X₃₀ is the amino acid N or S; and/or
   (xxxi) X₃₁ is the amino acid K or H;
b) the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51), in which:
   (i) X₁ is the amino acid P or A;
   (ii) X₂ is the amino acid V or I;
   (iii) X₃ is the amino acid E or Q;
   (iv) X₄ is the amino acid I or L;
   (v) X₅ is the amino acid S or T;
   (vi) X₆ is the amino acid E or Q;
   (vii) X₇ is the amino acid E or Q;
   (viii) X₈ is the amino acid K or R;
   (ix) X₉ is the amino acid Q or A;
   (x) X₁₀ is the amino acid S or P;
   (xi) X₁₁ is the amino acid K or N;
   (xii) X₁₂ is the amino acid Q or S; and/or
   (xiii) X₁₃ is the amino acid S or G;
c) the amino acid sequence LAX₁X₂SFX₃X₄X₅DX₆TGX₇IX₈GLX₉X₁₀X₁₁ELX₁₂X₁₃LQDWEYKFMX₁₄KYVKV GX₁₅X₁₆ (SEQ ID NO: 52), in which:
   (i) X₁ is the amino acid K or L;
   (ii) X₂ is the amino acid M or L;
   (iii) X₃ is the amino acid E or K;
   (iv) X₄ is the amino acid E or P;
   (v) X₅ is the amino acid K or E;
   (vi) X₆ is the amino acid L or I;
   (vii) X₇ is the amino acid D or N;
   (viii) X₈ is the amino acid S or E;
   (ix) X₉ is the amino acid G or S;
   (x) X₁₀ is the amino acid P or E;
   (xi) X₁₁ is the amino acid F or E;
   (xii) X₁₂ is the amino acid E or V;
   (xiii) X₁₃ is the amino acid A or I;
   (xiv) X₁₄ is the amino acid S or E;
   (xv) X₁₅ is the amino acid T or E; and/or
   (xvi) X₁₆ is the amino acid V or L; and
d) the amino acid sequence X₁X₂X₃EX₄GXSX₆X₇X₈X₉X₁₀D (SEQ ID NO: 53), in which:
   (i) X₁ is the amino acid K or E;
   (ii) X₂ is the amino acid P or H;
   (iii) X₃ is the amino acid A or S;
   (iv) X₄ is the amino acid D or N;
   (v) X₅ is the amino acid P or H;
   (vi) X₆ is the amino acid S or R;
   (vii) X₇ is the amino acid E or N;
   (viii) X₈ is the amino acid S or F;
   (ix) X₉ is the amino acid Q or E; and/or
   (x) X₁₀ is the amino acid A or I.

The following motifs, corresponding to SEQ ID NOs: 58, 60, 62, and 64, are present in the CB5 sequences expressed in strains 848917 and 848921:
a) QVWETLKEAIVAYTGLSPATFFTVLALGLAVYYVISGFFGTSDYGSH (SEQ ID NO: 58);
b) PVQVGEISEEELKQYDGSDSKKPLLMAIKGQIYDVSQSRMF (SEQ ID NO: 60);
c) LAKMSFEEKDLTGDISGLGPFELEALQDWEYKFMSKYVKVGTV (SEQ ID NO: 62); and
d) KPAEDGPSESQAD (SEQ ID NO: 64).

The following motifs, corresponding to SEQ ID NOs: 59, 61, 63, and 65, are present in the CB5 sequences expressed in strains 848922 and 848930:
a) ELYWKAMEQIAWYTGLSPTAFFTILASMIFVFQMVSSMFVSPEEFNK (SEQ ID NO: 59);
b) AVQIGQLTEQQLRAYDGSDPNKPLLMAIKGQIYDVSSGRMF (SEQ ID NO: 61);
c) LALLSFKPEDITGNIEGLSEEELVILQDWEYKFMEKYVKVGEL (SEQ ID NO: 63); and
d) EHSENGHRNFEID (SEQ ID NO: 65).

The following motifs, corresponding to SEQ ID NOs: 54-57, are present in the CB5 sequence expressed in strain 848940:
a) the amino acid sequence ILRVSFRKYRKAIEQ (SEQ ID NO: 54);
b) the amino acid sequence RAFRPSIRFKKSHSTVPT (SEQ ID NO: 55);
c) the amino acid sequence KNTLYVGG (SEQ ID NO: 56); and/or
d) the amino acid sequence DQATQKHRSFGFVTFLEKED (SEQ ID NO: 57).

TABLE 2
Mogrol production by strains comprising CB5s

| | SEQ ID of | | |
|---|---|---|---|
| | CB5 expressed | | |
| Strain | in each strain | Mogrol (mg/L) | STDEV |
| Parent | N/A | 13.31 | 6.80 |
| 848921 | 1 | 52.76 | 2.91 |
| 848930 | 3 | 32.30 | 1.12 |
| 848917 | 2 | 31.29 | 19.73 |
| 848922 | 3 | 26.77 | 6.98 |
| 848940 | 4 | 16.16 | 0.29 |
| 848936 | 6 | 13.30 | 0.73 |
| 848944 | 7 | 11.28 | 2.79 |
| 849014 | 8 | 10.62 | 1.82 |
| 848923 | 5 | 9.27 | 6.22 |
| 849161 | 9 | 8.10 | 0.12 |
| 848952 | 10 | 4.58 | 1.55 |

As shown in Table 2 and in FIG. 7B, multiple CB5 proteins, which may interact with cytochrome P450 enzymes as well as cytochrome P450 reductase partners, were identified in this screen that resulted in enhanced mogrol production.

### Example 2. Increased Mogrol Production by CB5 Proteins in Y. lipolytica

This Example describes testing representative S. grosvenorii cytochrome b5 proteins identified in Example 1 in Y. lipolytica to confirm that the proteins enhance mogrol production in multiple cell types.

Three CB5 proteins identified in Example 1 (corresponding to SEQ ID NO: 1, SEQ ID NO: 2, and SEQ ID NO: 3), and a truncated version of SEQ ID NO: 1 and SEQ ID NO: 2 (corresponding to SEQ ID NO: 318), were expressed in Y. lipolytica host cells to determine whether the proteins enhanced mogrol or mogroside production in Y. lipolytica.

Two different host cell base strains were engineered to express one or more copies of CYP1798, CYP5491-T351M, AtCPR, SgCDS, and EPH3, as well as to upregulate expression of ERG1 and downregulate expression of ERG7.

To test the strains expressing the CB5 proteins for enhanced mogrol production, an in vivo plate assay was combined with LC-MS analysis. Plasmids carrying individual genes were transformed and integrated into the chromosome of Y. lipolytica parent strains that produce mogrol. Parent strains lacking any S. grosvenorii cytochrome b5 protein were used as negative controls, corresponding to strains 974137 and 1419596. Single colonies resulting from transformation were grown as pre-cultures containing culturing media in a shaking incubator at 30° C. for 96 hours at 1000 rpm. After 48 hours, pre-cultures were transferred into production media and grown in a shaking incubator at 30° C. for 96 hours at 1000 rpm. After 96 hours, cultures were extracted with an organic solvent and product formation was tested by LC-MS analysis to evaluate mogrol and mogroside production. A Thermo Scientific Q Exactive Focus MS with a LX2 multiplexed columns setup was used. Thermo Scientific Accucore PFP columns (2.6 μm, 2.1 mm×100 mm) with 12.5 mM ammonium acetate pH 8.0 in water running buffer and acetonitrile ramp were used for separation in negative mode using full scan. Initially, a short analytical run was performed to identify product species based on mass.

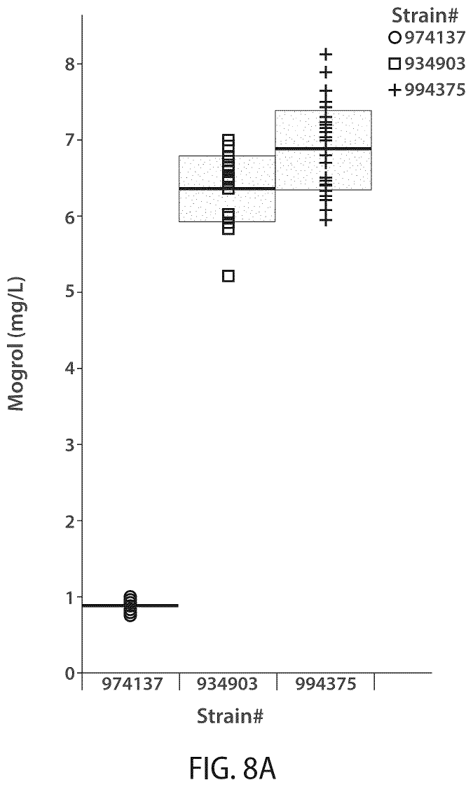

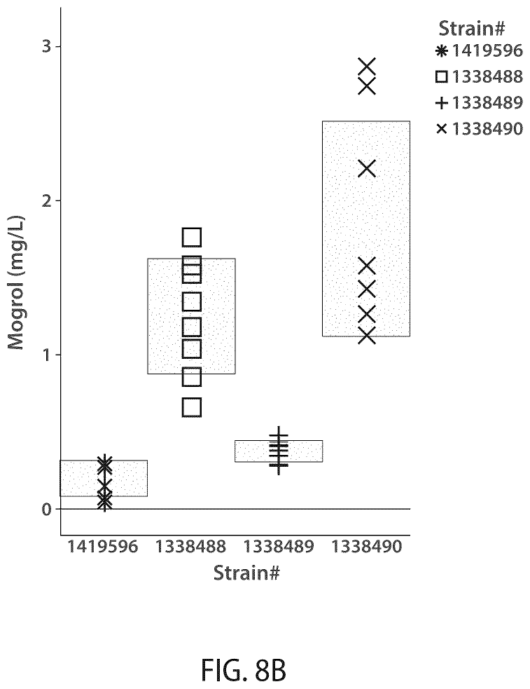

The CB5 protein with a sequence corresponding to SEQ ID NO: 1 as well as the truncated form, CB5-trunc, with a sequence corresponding to SEQ ID NO: 318, expressed in strains 994375 and 934903 respectively, were observed to increase mogrol production relative to the parental strain 974137 in a first strain background (Table 3 and FIG. 8A).

In a second strain background, the CB5 proteins with a sequence corresponding to SEQ ID NO: 1, SEQ ID NO: 2, or SEQ ID NO: 3 expressed in strains 1338488, 1338490, and 1338489, respectively, were observed to increase mogrol production relative to the parental strain 1419596 (Table 3 and FIG. 8B).

Motifs corresponding to SEQ ID NOs: 47-49, 50-52, 58, 60, and 62, discussed above, are present in the CB5 sequences expressed in strains 994375, 934903, and 1338490.

TABLE 3
Mogrol production by Y. lipolytica strains comprising CB5 proteins

| | | | |
|---|---|---|---|
| | SEQ ID of CB5 | | |
| | expressed in each | | |
| Strain | strain | Mogrol (mg/L) | STDEV |

Parent 1

| | | | |
|---|---|---|---|
| Parent 1 (974137) | N/A | 0.90 | 0.03 |
| 934903 | 318 | 6.38 | 0.47 |
| 994375 | 1 | 6.88 | 0.52 |

Parent 2

| | | | |
|---|---|---|---|
| Parent 2 (1419596) | N/A | 0.20 | 0.12 |
| 1338488 | 1 | 1.24 | 0.38 |
| 1338489 | 3 | 0.37 | 0.07 |
| 1338490 | 2 | 1.81 | 0.70 |

As shown in Table 3 and in FIGS. 8A-8B, this Example shows that representative CB5 proteins identified in Example 1, and a non-naturally occurring truncated form of a CB5 protein sharing similarity to CB5 proteins identified in Example 1, were able to enhance mogrol production in Y. lipolytica host cells, confirming that this effect is not limited to S. cerevisiae cells. These data indicate that the identified CB5 proteins are able to enhance mogrol production through interactions with the heterologous pathways expressed in the host cells and that this effect is not host-dependent.

### Example 3. Identification and Functional Characterization of CB5 Proteins that Increase Abscisic Acid (ABA) Production

This Example describes the screening of 3 Siraitia grosvenorii and 6 Myrothecium indicum proteins with homology to Cytochrome b5 (CB5) proteins to identify proteins that promote abscisic acid (ABA) production in host cells. Y. lipolytica host cell strains engineered to express at least three enzymes within an ABA biosynthetic pathway (e.g., abscisic acid biosynthetic genes aba1, aba2, aba3, aba4, and cytochrome P450 reductase (CPR, SEQ ID NO: 93) that includes two Cytochrome P450 (CYP) enzymes (genes aba1 and aba2)) were used to evaluate the effect of expression of CB5 proteins. Linear constructs carrying expression cassettes for CB5 genes were transformed and integrated into the genome of the Y. lipolytica hosts. The hosts (parental strains) lacking any additional CB5 protein were used as negative controls. Single colonies resulting from transformation were inoculated in culture media and grown in a shaking incubator at 30° C. for 48 hours at 1000 rpm. After 48 hours, culture supernatants were analyzed by LC-MS to evaluate sesquiterpene (abscisic acid or ABA-diol) production.

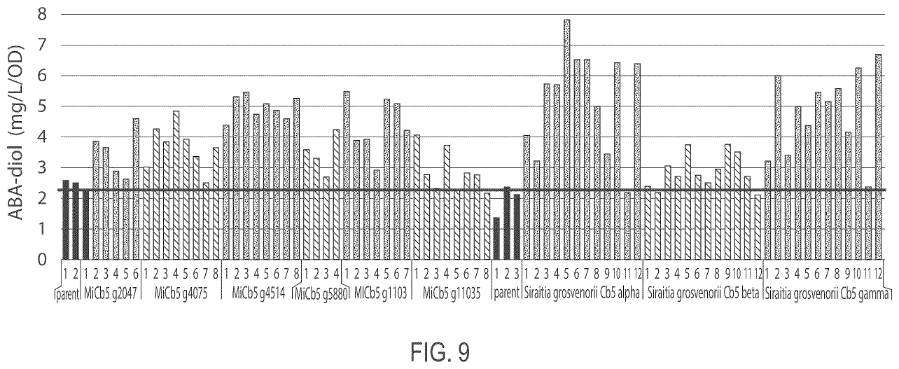

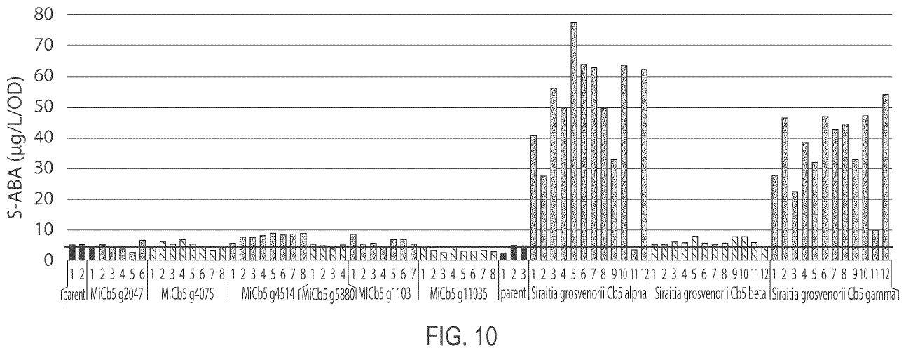

In FIGS. 9-10, a Y. lipolytica parent strain (parent strain 1) that expresses the following three ABA synthesis enzymes: aba1, aba2, and aba3 was used. As shown in FIG. 9, parent strain 1 produces the ABA precursor, ABA-diol. Parent strain 1 was transformed with one of six fungal CB5 candidate genes (mined from Myrothecium indicum) and three S. grosvenorii CB5 genes. ABA-diol production by each strain was measured by LC-MS. Expression of Siraitia grosvenorii CB5 alpha (SEQ ID NO: 1) and Siraitia grosvenorii CB5 gamma (SEQ ID NO: 2) increased ABA-diol production of parent strain 1 (FIG. 9 and Table 4). Notably, CB5s comprising SEQ ID NO: 1 or SEQ ID NO: 2 outperformed fungal CB5s mined from Myrothecium indicum and increased abscisic acid production by parent strain 1 (FIG. 10 and Table 5).

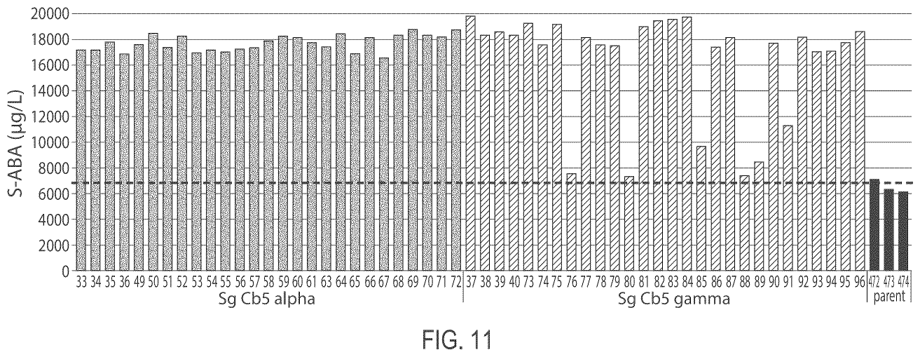

In FIG. 11, a Y. lipolytica parent strain (parent strain 2) that expresses all four of the following ABA synthesis enzymes: aba1, aba2, aba3, and aba4 was used. As shown in FIG. 11, parent strain 2 produces ABA. Parent strain 2 was transformed with either of two S. grosvenorii CB5 genes (alpha and gamma, SEQ ID NOs: 1 and 2, respectively). ABA production was measured by LC-MS. In particular, production of the stereoisomer S-ABA was measured. Expression of Siraitia grosvenorii CB5 alpha (SEQ ID NO: 1) and Siraitia grosvenorii CB5 gamma (SEQ ID NO: 2) increased ABA production of parent strain 2 (FIG. 11 and Table 6).

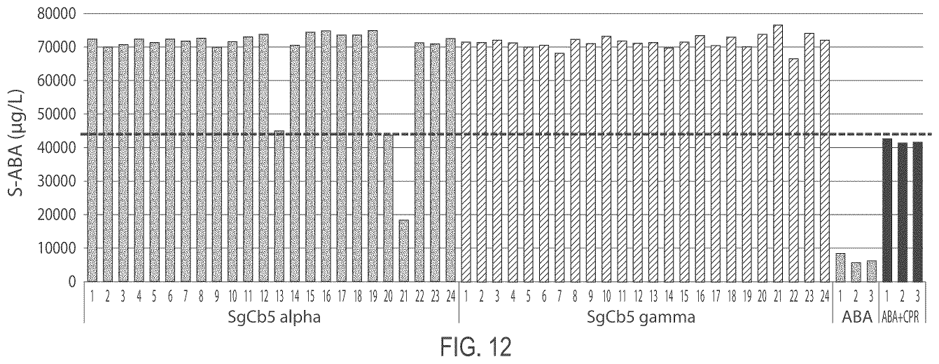

In FIG. 12, a Y. lipolytica parent strain (strain 3) that expresses all four of the following ABA synthesis enzymes: aba1, aba2, aba3, and aba4 and also an accessory Cytochrome P450 Reductase (CPR) from Myrotheciumn indicum was used. Parent strain 3 was transformed with either of two S. grosvenorii CB5 genes (alpha and gamma, SEQ ID NOs: 1 and 2, respectively). ABA production was measured by LC-MS. In particular, production of the stereoisomer S-ABA was measured. Expression of Siraitia grosvenorii CB5 alpha (SEQ ID NO: 1) and Sirairia grosvenorii CB5 gamma (SEQ ID NO: 2) increased ABA production of parent strain 3 (FIG. 12 and Table 7).

TABLE 4
ABA-diol production by parent strain 1 comprising CB5s

| | | ABA-diol in mg/L/OD |
|---|---|---|
| | | (Measured Conc. |
| CB5 protein expressed | | Normalized to OD |
| in strain | Isolate # | (mg/L/OD)) |
| N/A (parent strain) | 1 | 2.61 |
| | 2 | 2.53 |
| MiCB5 g2047 | 1 | 2.29 |
| | 2 | 3.90 |
| | 3 | 3.68 |
| | 4 | 2.91 |
| | 5 | 2.66 |
| | 6 | 4.61 |
| MiCB5 g4075 | 1 | 3.03 |
| | 2 | 4.28 |
| | 3 | 3.87 |
| | 4 | 4.87 |
| | 5 | 3.94 |
| | 6 | 3.38 |
| | 7 | 2.51 |
| | 8 | 3.67 |
| MiCB5 g4514 | 1 | 4.41 |
| | 2 | 5.33 |
| | 3 | 5.49 |
| | 4 | 4.78 |
| | 5 | 5.11 |
| | 6 | 4.90 |
| | 7 | 4.63 |
| | 8 | 5.29 |
| MiCB5 g5880 | 1 | 3.61 |
| | 2 | 3.32 |
| | 3 | 2.72 |
| | 4 | 4.26 |
| MICB5 g1103 | 1 | 5.51 |
| | 2 | 3.92 |
| | 3 | 3.95 |
| | 4 | 2.94 |
| | 5 | 5.26 |
| | 6 | 5.11 |
| | 7 | 4.24 |
| MiCB5 g11035 | 1 | 4.09 |
| | 2 | 2.79 |
| | 3 | 2.34 |
| | 4 | 3.74 |
| | 5 | 2.27 |
| | 6 | 2.85 |
| | 7 | 2.79 |
| | 8 | 2.18 |
| parent | 1 | 1.40 |
| | 2 | 2.39 |
| | 3 | 2.15 |
| Siraitia grosvenorii CB5 | 1 | 4.08 |
| alpha | 2 | 3.24 |
| | 3 | 5.76 |
| | 4 | 5.73 |
| | 5 | 7.84 |
| | 6 | 6.55 |
| | 7 | 6.55 |
| | 8 | 5.02 |
| | 9 | 3.48 |
| | 10 | 6.46 |
| | 11 | 2.21 |
| | 12 | 6.43 |
| Siraitia grosvenorii CB5 | 1 | 2.42 |
| beta | 2 | 2.20 |
| | 3 | 3.08 |
| | 4 | 2.74 |
| | 5 | 3.75 |
| | 6 | 2.77 |
| | 7 | 2.52 |
| | 8 | 2.97 |
| | 9 | 3.77 |
| | 10 | 3.54 |
| | 11 | 2.74 |
| | 12 | 2.14 |
| Siraitia grosvenorii CB5 | 1 | 3.30 |
| gamma | 2 | 6.01 |
| | 3 | 3.42 |
| | 4 | 5.01 |
| | 5 | 4.40 |
| | 6 | 5.46 |
| | 7 | 5.18 |
| | 8 | 5.59 |
| | 9 | 4.18 |
| | 10 | 6.27 |
| | 11 | 2.40 |
| | 12 | 6.72 |

TABLE 5
Abscisic acid production by parent strain 1 comprising CB5s

| | | S-ABA |
|---|---|---|
| | | (Measured Conc. |
| CB5 protein | | Normalized to OD |
| expressed in strain | Isolate # | (μg/L/OD)) |
| N/A (parent strain) | 1 | 5.46 |
| | 2 | 5.53 |
| MiCB5 g2047 | 1 | 4.19 |
| | 2 | 5.51 |
| | 3 | 5.06 |
| | 4 | 4.01 |
| | 5 | 2.98 |
| | 6 | 6.95 |
| MiCB5 g4075 | 1 | 4.89 |
| | 2 | 6.46 |
| | 3 | 5.63 |
| | 4 | 7.08 |
| | 5 | 5.69 |
| | 6 | 4.05 |
| | 7 | 3.67 |
| | 8 | 5.04 |
| MiCB5 g4514 | 1 | 6.11 |
| | 2 | 7.98 |
| | 3 | 8.02 |
| | 4 | 8.57 |
| | 5 | 9.43 |
| | 6 | 8.73 |
| | 7 | 9.06 |
| | 8 | 9.33 |
| MiCB5 g5880 | 1 | 5.67 |
| | 2 | 5.19 |
| | 3 | 4.01 |
| | 4 | 5.54 |
| MICB5 g1103 | 1 | 8.86 |
| | 2 | 5.88 |
| | 3 | 6.15 |
| | 4 | 4.55 |
| | 5 | 7.24 |
| | 6 | 7.25 |
| | 7 | 5.80 |
| MiCB5 g11035 | 1 | 5.06 |
| | 2 | 3.57 |
| | 3 | 3.04 |
| | 4 | 4.70 |
| | 5 | 3.46 |
| | 6 | 3.46 |
| | 7 | 3.65 |
| | 8 | 3.36 |
| N/A (parent strain) | 1 | 2.87 |
| | 2 | 5.44 |
| | 3 | 5.25 |
| Siraitia grosvenorii | 1 | 41.30 |
| CB5 alpha | 2 | 28.01 |
| | 3 | 56.77 |
| | 4 | 49.95 |
| | 5 | 76.96 |
| | 6 | 64.44 |
| | 7 | 63.37 |
| | 8 | 49.85 |
| | 9 | 33.36 |
| | 10 | 64.20 |
| | 11 | 3.84 |
| | 12 | 62.81 |
| Siraitia grosvenorii | 1 | 5.56 |
| CB5 beta | 2 | 5.47 |
| | 3 | 6.63 |
| | 4 | 6.30 |
| | 5 | 8.31 |
| | 6 | 6.09 |
| | 7 | 5.47 |
| | 8 | 6.14 |
| | 9 | 8.23 |
| | 10 | 8.21 |
| | 11 | 6.20 |
| | 12 | 4.46 |
| Siraitia grosvenorii | 1 | 28.13 |
| CB5 gamma | 2 | 47.00 |
| | 3 | 22.99 |
| | 4 | 39.18 |
| | 5 | 32.46 |
| | 6 | 47.63 |
| | 7 | 43.27 |
| | 8 | 45.10 |
| | 9 | 33.56 |
| | 10 | 47.75 |
| | 11 | 10.32 |
| | 12 | 54.71 |

TABLE 6
Abscisic acid production by parent strain 2 comprising CB5s

| CB5 | | |
|---|---|---|
| protein | | S-ABA |
| expressed | | (LCMS |
| in strain | Isolate # | titers (μg/L)) |
| Sg CB5 | 33 | 17160.1 |
| alpha | 34 | 17123.0 |
| | 35 | 17846.6 |
| | 36 | 16911.9 |
| | 49 | 17662.4 |
| | 50 | 18534.4 |
| | 51 | 17438.3 |
| | 52 | 18265.8 |
| | 53 | 17010.2 |
| | 54 | 17185.6 |
| | 55 | 17076.3 |
| | 56 | 17314.1 |
| | 57 | 17425.2 |
| | 58 | 17928.6 |
| | 59 | 18292.3 |
| | 60 | 18158.9 |
| | 61 | 17843.4 |
| | 63 | 17468.7 |
| | 64 | 18441.8 |
| | 65 | 16912.5 |
| | 66 | 18169.4 |
| | 67 | 16596.8 |
| | 68 | 18370.0 |
| | 69 | 18815.8 |
| | 70 | 18370.7 |
| | 71 | 18254.3 |
| | 72 | 18782.0 |
| Sg CB5 | 37 | 19814.5 |
| gamma | 38 | 18388.2 |
| | 39 | 18591.2 |
| | 40 | 18356.3 |
| | 73 | 19261.8 |
| | 74 | 17618.7 |
| | 75 | 19190.7 |
| | 76 | 7573.8 |
| | 77 | 18208.5 |
| | 78 | 17601.9 |
| | 79 | 17543.4 |
| | 80 | 7344.1 |
| | 81 | 19017.8 |
| | 82 | 19440.1 |
| | 83 | 19599.0 |
| | 84 | 19753.1 |
| | 85 | 9661.2 |
| | 86 | 17406.7 |
| | 87 | 18139.2 |
| | 88 | 7434.7 |
| | 89 | 8446.6 |
| | 90 | 17748.6 |
| | 91 | 11307.9 |
| | 92 | 18191.5 |
| | 93 | 17039.9 |
| | 94 | 17100.1 |
| | 95 | 17775.6 |
| | 96 | 18652.8 |
| N/A | 472 | 7153.1 |
| (parent | 473 | 6388.0 |
| strain) | 474 | 6161.6 |

TABLE 7
Abscisic acid production by parent strain 3 comprising CB5s

| CB5 protein | | S-ABA |
|---|---|---|
| expressed in strain | Isolate # | (Measured Conc. (ug/L))) |
| SgCB5 alpha | 1 | 72315.789 |
| | 2 | 70120.697 |
| | 3 | 70888.780 |
| | 4 | 72794.316 |
| | 5 | 71591.873 |
| | 6 | 72202.734 |
| | 7 | 71924.394 |
| | 8 | 72937.610 |
| | 9 | 70076.481 |
| | 10 | 71893.328 |
| | 11 | 73247.690 |
| | 12 | 73905.353 |
| | 13 | 45254.091 |
| | 14 | 70753.838 |
| | 15 | 74608.069 |
| | 16 | 74990.104 |
| | 17 | 73856.718 |
| | 18 | 73846.452 |
| | 19 | 75118.926 |
| | 20 | 43913.639 |
| | 21 | 18548.726 |
| | 22 | 71397.156 |
| | 23 | 71009.569 |
| | 24 | 72779.741 |
| SgCB5 gamma | 1 | 71571.670 |
| | 2 | 71437.245 |
| | 3 | 72226.700 |
| | 4 | 71307.903 |
| | 5 | 69842.500 |
| | 6 | 70517.397 |
| | 7 | 68206.488 |
| | 8 | 72372.218 |
| | 9 | 71071.767 |
| | 10 | 73277.428 |
| | 11 | 71927.239 |
| | 12 | 71123.307 |
| | 13 | 71485.895 |
| | 14 | 69800.094 |
| | 15 | 71450.385 |
| | 16 | 73599.584 |
| | 17 | 70397.054 |
| | 18 | 72998.693 |
| | 19 | 70146.253 |
| | 20 | 73781.083 |
| | 21 | 76616.986 |
| | 22 | 66516.512 |
| | 23 | 74153.345 |
| | 24 | 72135.717 |
| N/A (parent strain 2) | 1 | 8670.449 |
| | 2 | 5801.528 |
| | 3 | 6522.594 |
| N/A (parent strain 3) | 1 | 42783.086 |
| | 2 | 41517.895 |
| | 3 | 41819.178 |

### Example 4: Identification and Functional Characterization of CB5 Proteins that Increase Vindoline Production

This Example describes the testing of S. grosvenorii CB5 proteins in S. cerevisiae to probe whether these enzymes promote monoterpene indole alkaloid (MIA) vindoline product formation. The three S. grosvenorii CB5 proteins used in this Example are the same as the CB5 alpha, CB5 gamma, and CB5 beta proteins used in Examples 1 and 2. Strain 1522722 expresses CB5 alpha, strain 1522728 expresses CB5 gamma, and strain 1522736 expresses CB5 beta. Two of the three CB5's increased vindoline yields by about 10 times compared to a parent strain (1485239).

The CB5 enzymes were integrated into an existing vindoline producing S. cerevisiae host strain to determine whether these proteins could be used to increase vindoline production. The host cell base strain was engineered to express T16H2, 16OMT, T3O, T3R, NMT, D4H, and DAT for the bioconversion of tabersonine to vindoline. This base strain also had copies of cytochrome P450 helper enzymes AtCPR and CrCB5.

To test CB5 proteins for enhanced vindoline production, an in vivo plate assay was combined with LC-MS analysis. The CB5 genes were integrated into a vindoline-producing S. cerevisiae strain by amplifying the genes from a plasmid with primers containing homology arms for the integration loci, and transforming the linear PCR product alongside a Cas protein and a gRNA to promote homologous recombination. A strain lacking any additional plant protein (1485239) was used as a negative control along with the wild-type (WT) yeast strain.

For each sequence-confirmed integrant or control strain, three individual S. cerevisiae colonies were grown as pre-cultures containing Bird medium in a shaking incubator at 30° C. for 48 hours at 1000 rpm. These pre-cultures were then transferred into production media and grown in a shaking incubator at 30° C. for 24 hours at 1000 rpm, and then fed 50 mg/L tabersonine and grown for an additional 120 hours.

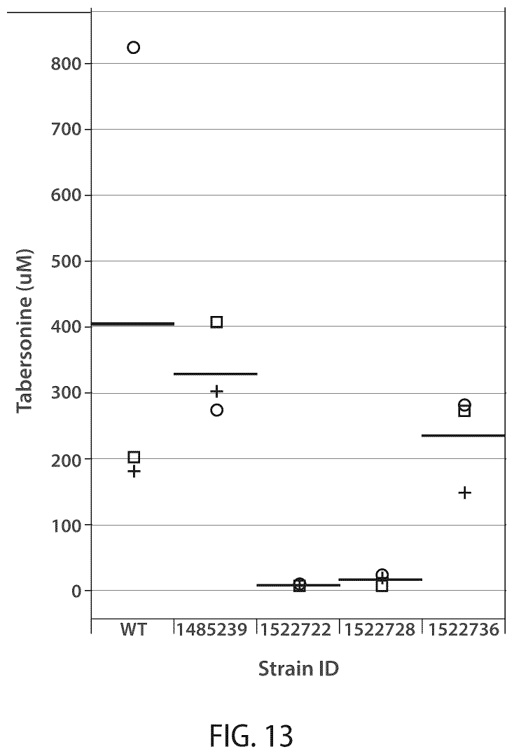

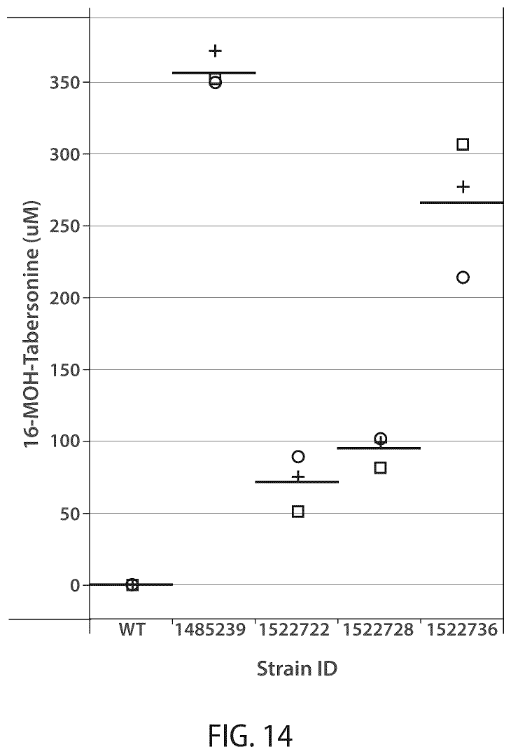

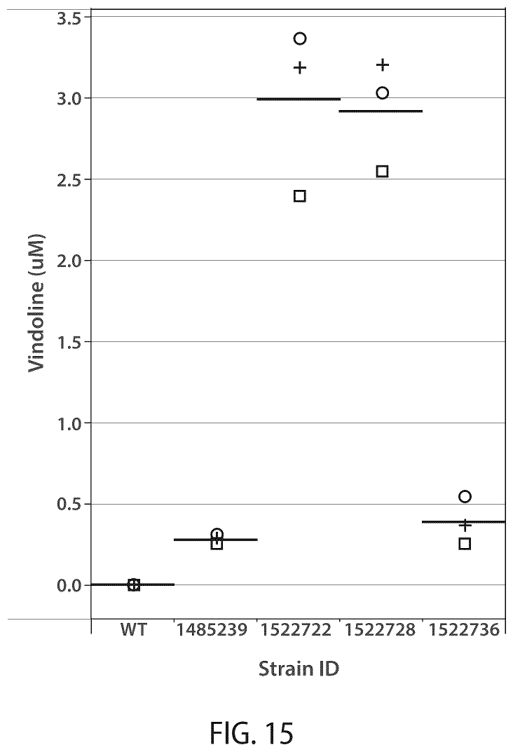

After 120 hours, cultures were diluted with 3 volumes of 66% methanol, frozen overnight at −20° C., and filtered before LC-MS analysis. Here, a Thermo Scientific Orbitrap Exploris 120 was paired with a C18 column (Thermo Scientific Accucore C18 2.6 μm, 2.1 mm×100 mm, 17126-102130) and water/acetonitrile (formic acid as modifier) as a running buffer gradient for separation. Data acquisition was performed in the positive mode using a product ion scan method. Based on this screen, CB5 alpha and CB5 gamma (Strains 1522722 and 1522728) showed enhanced utilization of tabersonine and 16-MOH-tabersonine substrates and improved vindoline production compared to the parental strain (FIGS. 13-15 and Table 8).

TABLE 8
Tabersonine, 16-MOH-tabersonine, and Vindoline Production

| | Tabersonine | | 16-MOH-tabersonine | | Vindoline | | |
|---|---|---|---|---|---|---|---|
| | (μM) | | (μM) | | (μM) | | |
| Strain | Average | STDEV | Average | STDEV | Average | STDEV | Cb5 |
| WT | 401.72 | 365.84 | 0.53 | 0.53 | 0.00 | 0.00 | N/A |
| 1485239 | 328.19 | 70.04 | 357.28 | 12.03 | 0.28 | 0.03 | N/A |
| 1522722 | 6.02 | 1.64 | 71.34 | 19.14 | 2.98 | 0.52 | CB5 |
| | | | | | | | alpha |
| 1522728 | 15.08 | 6.39 | 94.34 | 11.74 | 2.93 | 0.34 | CB5 |
| | | | | | | | gamma |
| 1522736 | 233.25 | 74.54 | 265.61 | 46.92 | 0.39 | 0.15 | CB5 |
| | | | | | | | beta |

### Example 5. Identification and Functional Characterization of CB5 Proteins that Increase Ginsenoside Production

This Example describes the evaluation of S. grosvenorii and F. fujikuroi CB5s in S. cerevisiae to identify enzymes that promote ginsenoside Rh2 product formation. The library of proteins evaluated included 11 S. grosvenorii proteins and a F. fujikuroi protein (SEQ ID NOs: 1-6, 8, 30, and 32-34). The amino acid sequences of these proteins and their nucleic acid sequences are provided in Table 14.

The proteins were evaluated in an existing Rh2 producing S. cerevisiae host strain to determine whether candidate proteins could be used to increase Rh2 production. The host cell base strain was engineered to express one or more copies of PqDDS, PgPPDS, PgCPR, and PgUGT50-HV for the bioconversion of 2,3 oxidosqualene to Rh2.

To test the protein library for enhanced Rh2 production, an in vivo plate assay was combined with LC-MS analysis. Plasmids carrying individual genes were transformed and integrated into the chromosome of a S. cerevisiae chassis strain that produces Rh2. A strain lacking any additional plant protein (t1469661) was used as a negative control.

For each sequence-confirmed library member or control, three individual S. cerevisiae colonies were grown as pre-cultures containing culturing media in a shaking incubator at 30° C. for 48 hours at 1000 rpm. After 72 hours, pre-cultures were transferred into production media and grown in a shaking incubator at 30° C. for 48 hours at 1000 rpm.

After 72 hours, cultures were diluted with 4 volumes of 80% methanol, mixed for I minute at room temperature, and filtered before LC-MS analysis.

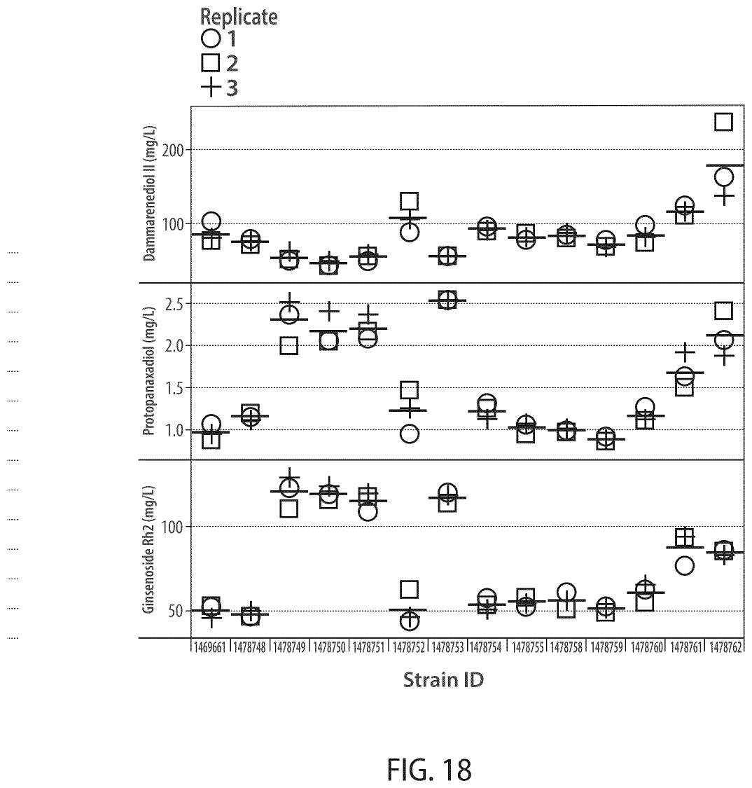

LC-MS analysis was performed using a Thermo Scientific Exploris 120 paired with a PFP column (Thermo Scientific Accucore PFP 2.6 μm, 2.1 mm×100 mm, 17426-102130) and water/acetonitrile (formic acid as modifier) was used as a running buffer gradient for separation. Data acquisition was performed in both positive and negative mode using full scan. Full results are shown in FIG. 18 and Table 9.

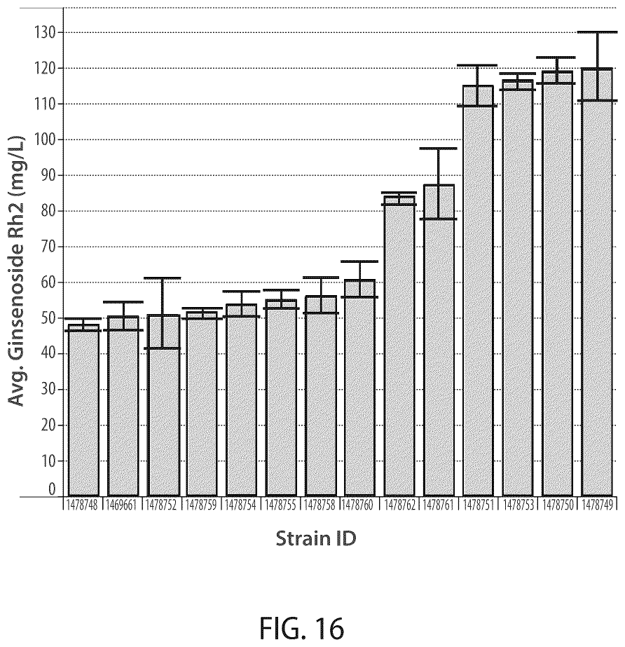

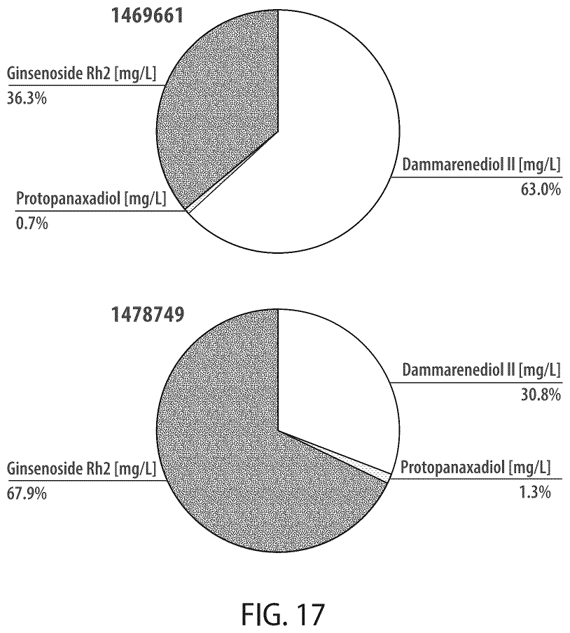

Based on this screen, several proteins were identified that increased Rh2 production of the parental strain (FIGS. 16-17). Several of these CB5 proteins were found to increase Rh2 production of the parental strains, including the CB5s expressed by strains 1478762, 1478761, 1478751, 1478753, 1478750, and 1478749. SEQ ID NO: 30, which is expressed by strain 1478762, comprises the amino acid sequence the amino acid sequence DATX₁X₂FX₃X₄X₅VGHS (SEQ ID NO: 31), wherein: X₁ is the amino acid E, D, or N; X₂ is the amino acid A or D; X₃ is the amino acid E or D; X₄ is the amino acid D or N; and/or X₅ is the amino acid V or A.

TABLE 9
Production of Dammarenediol II, Protopanaxadiol,
and Ginsenoside Rh2

| | Dammarenediol II | | Protopanaxadiol | | Ginsenoside Rh2 | |
|---|---|---|---|---|---|---|
| | titer [mg/L] | | titer [mg/L] | | [mg/L] | |
| Strain ID | Average | STDEV | Average | STDEV | Average | STDEV |
| 1469661 | 87.38 | 13.02 | 0.96 | 0.09 | 50.32 | 3.96 |
| 1478748 | 76.67 | 2.44 | 1.16 | 0.03 | 47.76 | 1.77 |
| 1478749 | 54.74 | 9.03 | 2.29 | 0.27 | 120.41 | 9.77 |
| 1478750 | 46.41 | 4.24 | 2.17 | 0.20 | 119.25 | 3.79 |
| 1478751 | 55.33 | 3.87 | 2.20 | 0.15 | 115.01 | 5.62 |
| 1478752 | 108.78 | 21.14 | 1.22 | 0.26 | 51.08 | 9.84 |
| 1478753 | 55.93 | 1.93 | 2.53 | 0.00 | 116.92 | 2.42 |
| 1478754 | 93.56 | 1.93 | 1.22 | 0.10 | 53.77 | 3.37 |
| 1478755 | 81.42 | 5.01 | 1.04 | 0.07 | 55.30 | 2.56 |
| 1478758 | 84.38 | 5.17 | 1.00 | 0.03 | 56.00 | 4.93 |
| 1478759 | 72.75 | 4.73 | 0.88 | 0.04 | 51.38 | 1.36 |
| 1478760 | 84.55 | 11.94 | 1.17 | 0.08 | 60.57 | 5.05 |
| 1478761 | 116.99 | 5.80 | 1.67 | 0.22 | 87.56 | 9.90 |
| 1478762 | 179.59 | 51.80 | 2.11 | 0.27 | 84.19 | 1.72 |

### Example 6: Identification and Functional Characterization of CB5 Proteins that Increase Betaxanthin Production

A Saccharomyces cerevisiae strain producing betaxanthin, expressing MjDODA and BvCYP76AD5, was used to evaluate 13 CB5s from Siraitia grosvenorii for increasing betaxanthin production. CB5s were expressed under the control of promoter pGAL4_USA from Saccharomyces paradoxus. In addition, a strain expressing a CYB5 gene codon-optimized for optimal expression in S. cerevisiae from pGAL1_S. paradoxus was used as a control. Constructed strains were sequence verified and screened for betaxanthin production in replicate plates.

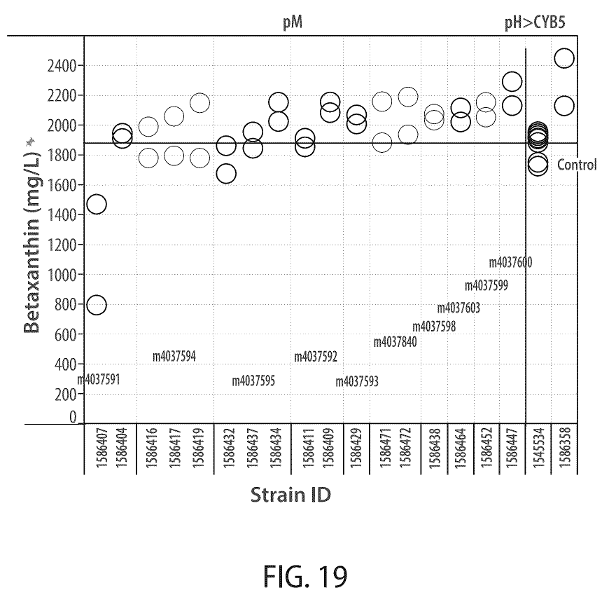

The results are shown in FIG. 19 and Table 10. The strain with expression of m4037600 (1586447) seems to have improved betaxanthin production, while strain expressing m4037599 (1586452) seems to have slight improvement on betaxanthin production. The strains with expression of m4037598 (1586438), m4037603 (1586464), m4037593 (1586429), m4037595 (1586434) and m4037592 (1586409) also seem to have a slight improvement on betaxanthin production—as replicates from at least one colony exhibited betaxanthin levels above that of the control. SEQ ID NOs: 4, 5, 6, and 10 comprise the amino acid sequence the amino acid sequence DATX₁X₂FX₃X₄X₅VGHS (SEQ ID NO: 31), wherein: X₁ is the amino acid E, D, or N; X₂ is the amino acid A or D; X₃ is the amino acid E or D; X₄ is the amino acid D or N; and/or X₅ is the amino acid V or A.

TABLE 10
Production of betaxanthin

| mID | Strain | Plate | Betaxanthin (mg/L) |
|---|---|---|---|
| Control | 1545534 | 1 | 1905 |
| Control | 1545534 | 2 | 1946 |
| Control | 1545534 | 2 | 1721 |
| Control | 1545534 | 1 | 1899 |
| Control | 1545534 | 2 | 1744 |
| Control | 1545534 | 1 | 1921 |
| Control | 1545534 | 1 | 1874 |
| Control | 1545534 | 2 | 1938 |
| Control | 1586358 | 1 | 2123 |
| CYB5 | | | |
| Control | 1586358 | 2 | 2439 |
| CYB5 | | | |
| m4037591 | 1586404 | 2 | 1904 |
| m4037591 | 1586404 | 1 | 1935 |
| m4037591 | 1586407 | 1 | 787 |
| m4037591 | 1586407 | 2 | 1467 |
| m4037592 | 1586409 | 1 | 2075 |
| m4037592 | 1586409 | 2 | 2146 |
| m4037592 | 1586411 | 2 | 1851 |
| m4037592 | 1586411 | 1 | 1908 |
| m4037594 | 1586416 | 1 | 1774 |
| m4037594 | 1586416 | 2 | 1984 |
| m4037594 | 1586417 | 1 | 1791 |
| m4037594 | 1586417 | 2 | 2052 |
| m4037594 | 1586419 | 1 | 1771 |
| m4037594 | 1586419 | 2 | 2138 |
| m4037593 | 1586429 | 2 | 2002 |
| m4037593 | 1586429 | 1 | 2057 |
| m4037595 | 1586432 | 2 | 1668 |
| m4037595 | 1586432 | 1 | 1855 |
| m4037595 | 1586434 | 1 | 2019 |
| m4037595 | 1586434 | 2 | 2144 |
| m4037595 | 1586437 | 1 | 1841 |
| m4037595 | 1586437 | 2 | 1947 |
| m4037598 | 1586438 | 2 | 2027 |
| m4037598 | 1586438 | 1 | 2065 |
| m4037600 | 1586447 | 1 | 2122 |
| m4037600 | 1586447 | 2 | 2285 |
| m4037599 | 1586452 | 1 | 2050 |
| m4037599 | 1586452 | 2 | 2145 |
| m4037603 | 1586464 | 2 | 2018 |
| m4037603 | 1586464 | 1 | 2110 |
| m4037840 | 1586471 | 1 | 1879 |
| m4037840 | 1586471 | 2 | 2153 |
| m4037840 | 1586472 | 1 | 1932 |
| m4037840 | 1586472 | 2 | 2185 |

### Example 7: Functional Characterization of CB5 Proteins that Increase Astaxanthin Production

This Example demonstrates that CB5 alpha (SEQ ID NO: 1) could increase astaxanthin production by Xanthophyllomyces dendrorhous cells.

Xanthophyllomyces dendrorhous (Phaffia rhodozyma) (Strain ATCC 96594) was grown in 25 mL YPD in a 125 mL baffled shake flask at 25° C., 250 RPM, overnight. The next day, the culture was diluted to OD 0.2-0.3 in a new 125 mL baffled flask containing 12 mL YPD and 12 mL I M sorbitol. The culture was grown to an OD of 0.6-0.8. Cells were pelleted in 50 mL falcon tubes at 4000 g for 5 min and resuspended in 3 mL of 50 mM potassium phosphate buffer (pH 7.0) containing 25 mM DTT. Cells were incubated in the same 50 mL falcon tube for 15 minutes at room temperature. Next, cells were pelleted and washed twice with 1 mL STM (270 mM sucrose, 10 mM Tris HCl pH 7.5, 1 mM MgCl₂; pH 7.0). Then cells were pelleted and re-suspended in STM (50 μL/transformation). Then 50 μL of cells were mixed with 5 μL of DNA in pre-chilled 1.5 mL eppendorf tubes. The entire mixture was transferred to pre-chilled 2 mm electroporation cuvettes and electroporated at 0.8 kV, 1000 ohms, 25 μF after which 1 mL of chilled YPD was added immediately to the cuvette and returned to ice. Transformations were recovered for 24 h at 23° C., then plated onto YPD agar plates containing 10 μg/mL cycloheximide and 100 μg/mL carbenicillin (to deter bacterial growth in case of contamination). Plates were incubated at 25° C. and colonies became visible after 4 days.

For the astaxanthin production assay, Phaffia colonies were inoculated into 350 μL YPD in 96-deep well plates and grown at 21° C., 1000 RPM, for 4 days. On day 2, glycerol stocks were made by adding 50 μL culture to 50 μL of 60% glycerol in matrix tubes for stocking into matrix tube racks. Then the remaining culture was used for total carotenoids measurement by UV-Vis and astaxanthin quantitation by LC-MS.

To determine total carotenoid titer, first, 13 mg butylated hydroxytoluene (BHT) was added to 130 mL DMSO (100 mg/L final concentration) to serve as an antioxidant to prevent or slow carotenoid degradation during extraction and quantification. The DMSO+BHT mixture was distributed into 24-well plates, 5 mL/well and pre-heated to 65° C. Then, 10 μL of culture was added to 150 μL water in a 96-flat bottom UV-Vis plate (Corning 3370) and cell density was measured at 600 nm wavelength using a BioTek 96-well UV-vis spectrophotometer. Next, 100 μL of culture was transferred into a new 96-deep well plate and covered with an aluminum foil seal. Cells were spun down for 2 mins at 4000 RPM. Supernatant was discarded and the remaining liquid was removed by tapping the plate upside down on paper towels. Then, 500 μL of hot DMSO+BHT was added and mixed thoroughly to resuspend the pellets and extract the carotenoids. The cell suspension was then spun down for 2 minutes at 4000 RPM, and 200 μL of supernatant was transferred to a new 96-well flat bottom plate. The absorbance at 492 nm was used for total carotenoids measurement using a BioTek 96-well UV-vis spectrophotometer. The remaining carotenoids extraction was used for LC-MS quantification.

For the LC-MS measurement of astaxanthin, an analytical method was developed on an Q Exactive orbitrap 120 mass spectrometer using a C30 Accucore (100×2 mm, 2.5 μm particle size) column. The method was set to a 100-1000 m/z scan range in the positive mode, with RF lens at 70%, 60 000 orbitrap resolution and 6 s of expected LC peak width. Ion transfer tube temperature was set to 350° C., vaporizer temperature 400° C., Positive Ion voltage at 4000 V, sheath gas 15 Arb, Auxiliary gas 15 Arb and Sweep gas at 2 Arb.

Separation was achieved with an isocratic method with 90% acetonitrile, 10% water with 0.1% (v/v) formic acid, total acquisition of 5 minutes and astaxanthin eluting at 2.91 minutes. Autosampler temperature was set to 4° C., injection volume to 10 μL and flow-rate at 0.4 mL/min. The parent mass of 597.3901 m/z was used for quantification. Astaxanthin analytical standard was dissolved in analytical grade DMSO and was made fresh prior to analysis. Calibration curve was run between 2.5-0.04 mg/L. All samples were diluted with a diluent consisting of 50% acetonitrile, 50% water+0.1% (v/v) formic acid and submitted extracted samples were diluted 50×.

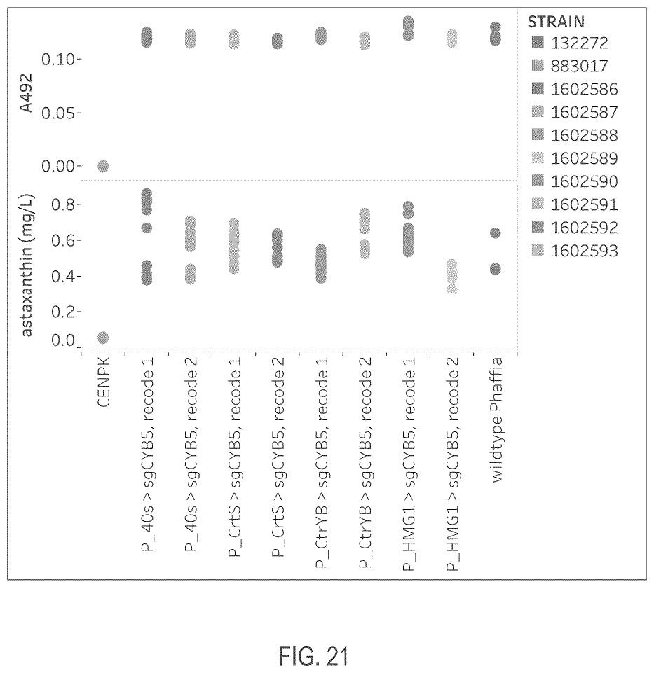

FIG. 21 and Table 11 shows the astaxanthin titers of various transformants of Phaffia rhodozyma along with the control wild type strain and negative control S. cerevisiae CEN.PK which is unable to produce astaxanthin. Several promoters were tested (P_40s, P_CrtS, P_CrtYB, P_HMG1) and two recodings of SgCYB5 were also tested (Yarrowia lipolylica and S. cerevisiae recodings). Some transformants produced 10-30% more astaxanthin than the wild type strain. Each transformant is expected to have varying copy numbers of the SgCYB5 due to the nature of the transformation at the rDNA sites. The presence of the CYB5 integration into the genome was confirmed by colony PCR and DNA sequencing of PCR products.

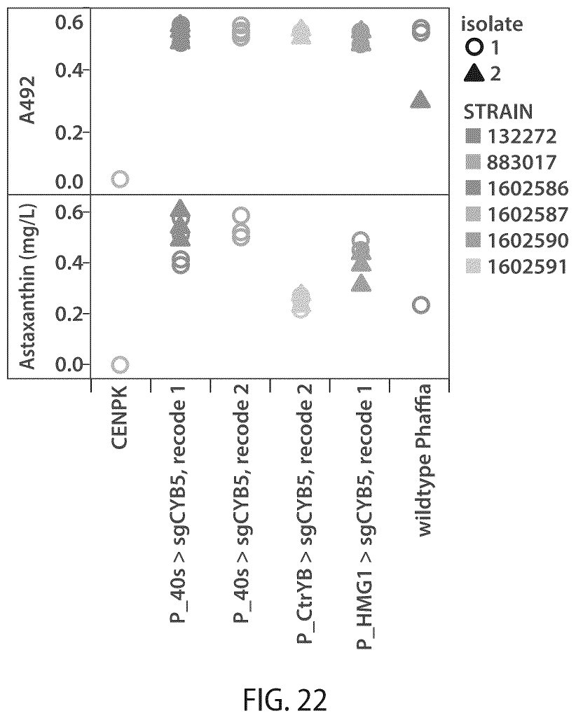

Transformants with seemingly increased astaxanthin titers were then retested with higher replication (n=4) to confirm their titer improvement (Table 12). The improved astaxanthin producing transformants demonstrated between 1.6-fold (t1602590-2) and 2.3-fold (t1602586-2) improvement in final astaxanthin titers as compared to the wild type strain (FIG. 22).

TABLE 11
Astaxanthin production

| | Astaxanthin Titer (mg/L) | | |
|---|---|---|---|
| Strain | Average (n = 11) | STDEV | CYB5 |
| S. cerevisiae WT | 0.05 | 0.00 | N/A |
| X. dendrorhous WT | 0.49 | 0.10 | N/A |
| 1602586 | 0.62 | 0.21 | 4037597 |
| 1602587 | 0.53 | 0.13 | 4705611 |
| 1602588 | 0.47 | 0.05 | 4037597 |
| 1602589 | 0.42 | 0.04 | 4705611 |
| 1602590 | 0.65 | 0.08 | 4037597 |
| 1602591 | 0.65 | 0.08 | 4705611 |
| 1602592 | 0.56 | 0.06 | 4037597 |
| 1602593 | 0.58 | 0.08 | 4705611 |

TABLE 12
Astaxanthin production

| | Astaxanthin Titer (mg/L) | | |
|---|---|---|---|
| Strain | Average (n = 4) | STDEV | CYB5 |
| S. cerevisiae WT | 0 | 0 | N/A |
| X. dendrorhous WT | 0.24 | 0.01 | N/A |
| 1602586, isolate 1 | 0.47 | 0.08 | 4037597 |
| 1602586, isolate 2 | 0.55 | 0.04 | 4037597 |
| 1602587 | 0.54 | 0.04 | 4705611 |
| 1602590, isolate 1 | 0.46 | 0.02 | 4037597 |
| 1602590, isolate 2 | 0.40 | 0.05 | 4037597 |
| 1602591, isolate 1 | 0.24 | 0.02 | 4705611 |
| 1602591, isolate 2 | 0.24 | 0.02 | 4705611 |

### Example 8: Identification and Functional Characterization of CB5 Proteins that Increase Betacyanin Production

A Saccharomyces cerevisiae strain producing betacyanin, expressing MjDODA, Db5GT, MjcDOPA5GT and BvCYP76AD1, was used to evaluate 10 CBSs from Siraitia grosvenorii as potential p450 reductase partners. CB5s were expressed under the control of promoter pGAL4_USA from Saccharomyces paradoxus. In addition, a strain expressing a CYB5 gene codon-optimized for optimal expression in S. cerevisiae from the pGAL1_S. paradoxus was used as a control. Constructed strains were sequence verified and screened for betacyanin production in replicate plates.

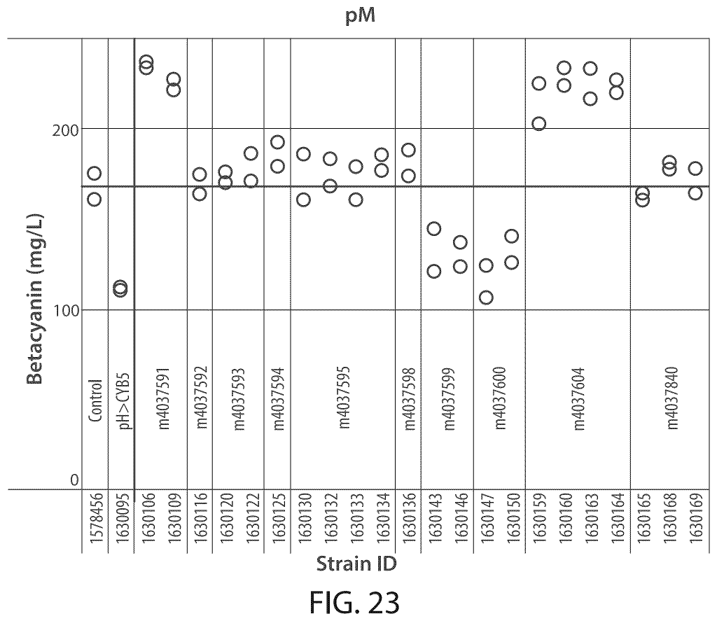

The results are shown in FIG. 23 and Table 13. The strains with expression of m4037591 (1630106, 1630109) or m4037604 (1630159, 1630160, 1630163, 1630164) or m4037594 (1630125) were observed to exhibit improved betacyanin production. The strains with expression of m4037593 (1630120, 1630122), m4037595 (1630130, 1630132, 1630133, 1630134), m4037598 (1630136) and m4037840 (1630168) also were observed to exhibit a slight improvement on betacyanin production—as replicates from at least one colony exhibited betacyanin levels above that of the control. SEQ ID NOs: 4, 5, 6, and 10 comprise the amino acid sequence DATX₁X₂FX₃X₄X₅VGHS (SEQ ID NO: 31), wherein: X₁ is the amino acid E, D, or N; X₂ is the amino acid A or D; X₃ is the amino acid E or D; X₄ is the amino acid D or N; and/or X₅ is the amino acid V or A.

TABLE 13
Production of betacyanins

| mID | Strain | Plate | Betacyanin(mg/L) |
|---|---|---|---|
| Control | 1578456 | 1 | 175.6 |
| Control | 1578456 | 2 | 160.0 |
| CYB5 | 1630095 | 2 | 112.0 |
| CYB5 | 1630095 | 1 | 109.6 |
| m4037591 | 1630106 | 2 | 236.3 |
| m4037591 | 1630106 | 1 | 233.0 |
| m4037591 | 1630109 | 2 | 226.2 |
| m4037591 | 1630109 | 1 | 221.2 |
| m4037592 | 1630116 | 1 | 174.8 |
| m4037592 | 1630116 | 2 | 163.7 |
| m4037593 | 1630120 | 2 | 175.9 |
| m4037593 | 1630120 | 1 | 169.2 |
| m4037593 | 1630122 | 1 | 185.6 |
| m4037593 | 1630122 | 2 | 170.6 |
| m4037594 | 1630125 | 1 | 191.8 |
| m4037594 | 1630125 | 2 | 179.4 |
| m4037595 | 1630132 | 1 | 183.3 |
| m4037595 | 1630132 | 2 | 168.5 |
| m4037595 | 1630134 | 1 | 185.3 |
| m4037595 | 1630134 | 2 | 176.1 |
| m4037595 | 1630130 | 1 | 185.8 |
| m4037595 | 1630130 | 2 | 160.4 |
| m4037595 | 1630133 | 1 | 179.0 |
| m4037595 | 1630133 | 2 | 160.0 |
| m4037598 | 1630136 | 2 | 188.1 |
| m4037598 | 1630136 | 1 | 173.9 |
| m4037599 | 1630143 | 1 | 144.2 |
| m4037599 | 1630143 | 2 | 119.9 |
| m4037599 | 1630146 | 1 | 136.8 |
| m4037599 | 1630146 | 2 | 123.3 |
| m4037600 | 1630150 | 1 | 139.8 |
| m4037600 | 1630150 | 2 | 126.0 |
| m4037600 | 1630147 | 1 | 124.2 |
| m4037600 | 1630147 | 2 | 105.5 |
| m4037604 | 1630159 | 1 | 224.4 |
| m4037604 | 1630159 | 2 | 202.1 |
| m4037604 | 1630164 | 2 | 226.2 |
| m4037604 | 1630164 | 1 | 219.6 |
| m4037604 | 1630160 | 1 | 233.4 |
| m4037604 | 1630160 | 2 | 223.5 |
| m4037604 | 1630163 | 2 | 232.8 |
| m4037604 | 1630163 | 1 | 216.2 |
| m4037840 | 1630169 | 1 | 178.0 |
| m4037840 | 1630169 | 2 | 164.6 |
| m4037840 | 1630165 | 1 | 163.4 |
| m4037840 | 1630165 | 2 | 159.9 |
| m4037840 | 1630168 | 2 | 181.4 |
| m4037840 | 1630168 | 1 | 177.3 |

TABLE 14
Non-limiting examples of CB5 sequences

| Strain | | |
|---|---|---|
| number(s) | Nucleotide | Protein |
| and/or | SEQ ID | SEQ |
| protein | NO | ID NO |
| 848921 | 11 | 1 |
| 848917 | 12 | 2 |
| 848922 | 13 | 3 |
| 848930 | 14 | 3 |
| 848940 | 15 | 4 |
| 848923 | 16 | 5 |
| 848936 | 17 | 6 |
| 848944 | 18 | 7 |
| 849014 | 19 | 8 |
| 849161 | 20 | 9 |
| 848952 | 21 | 10 |
| CB5 alpha | 22 | 1 |
| CB5 gamma | 23 | 2 |
| CB5 beta | 24 | 3 |
| 934903 | 316 | 318 |
| 994375 | 317 | 1 |
| 1338488 | 317 | 1 |
| 1338489 | 330 | 3 |
| 1338490 | 331 | 2 |
| CB5 g2047 | 83 | 77 |
| CB5 g4075 | 84 | 78 |
| CB5 g4514 | 85 | 79 |
| CB5 g5880 | 86 | 80 |
| CB5 g1103 | 87 | 81 |
| CB5 g11035 | 88 | 82 |
| CrCB5 | 35 | 29 |
| 1478749 | 12 | 2 |
| 1478753 | 14 | 3 |
| 1478750 | 11 | 1 |
| 1478751 | 13 | 3 |
| 1478762 | 36 | 30 |
| 1478761 | 37 | 30 |
| 1478760 | 38 | 32 |
| 1478758 | 39 | 33* |
| 1478754 | 17 | 6 |
| 1478755 | 15 | 4 |
| 1478759 | 19 | 8 |
| 1478748 | 40 | 34 |
| 1478752 | 16 | 5 |
| 1586407; | 95 | 3 |
| 1630109; | | |
| 1586404; | | |
| 1630106 | | |
| 1586411; | 96 | 4 |
| 1586409; | | |
| 1630116 | | |
| 1586429; | 97 | 34 |
| 1630120; | | |
| 1630122 | | |
| 1586416; | 98 | 8 |
| 1586417; | | |
| 1586419; | | |
| 1630125 | | |
| 1586432; | 99 | 6 |
| 1630130; | | |
| 1586434; | | |
| 1630132; | | |
| 1586437; | | |
| 1630133; | | |
| 1630134 | | |
| N/A | 100 | 1 |
| 1586438; | 101 | 33* |
| 1630136 | | |
| 1586452; | 102 | 5 |
| 1630143; | | |
| 1630146 | | |
| 1586447; | 103 | 10 |
| 1630147 | | |
| 1630150 | | |
| N/A | 104 | 7 |
| 1586464 | 105 | 3 |
| N/A | 106 | 2 |
| 1586471; | 107 | 32 |
| 1586472; | | |
| 1630165; | | |
| 1630168; | | |
| 1630169 | | |
| CYB5 (strain | | 118 |
| 1586358; | | |
| strain | | |
| 1630095) | | |
| 1522722 | 11 | 1 |
| 1522728 | 12 | 2 |
| 1522736 | 14 | 3 |
| 1602586; | 100 | 1 |
| 1602588; | | |
| 1602590; | | |
| 1602592 | | |
| 1602587; | 22 | 1 |
| 1602589; | | |
| 1602591; | | |
| 1602593 | | |
| 1630159; | 106 | 2 |
| 1630160; | | |
| 1630163; | | |
| 1630164 | | |

*In SEQ ID NO: 33, dashes (—) indicate a stop codon.

TABLE 15
Additional Sequences Associated with the Disclosure

| | Nucleic Acid | Protein |
|---|---|---|
| Enzyme | SEQ ID NO | SEQ ID NO |
| C11 hydroxylase | 264 | 280 |
| C11 hydroxylase (cucurbitadienol | 265 | 281 |
| oxidase) | | |
| Cytochrome P450 reductase | 266 | 282 |
| Cytochrome P450 reductase | 267 | 283 |
| CYP1798 | 296 | 280 |
| AtCPR1 | 297 | 283 |
| CPR4497 | 298 | 282 |
| sgCDS | 299 | 247 |
| EPH3 | 300 | 286 |
| atEPH2 | 301 | 310 |
| ERG9 | 302 | 311 |
| ERG1 | 303 | 312 |
| ERG7 | 304 | 313 |
| CYP5491 | 314 | 281 |
| SgCDS | 319 | 247 |
| CYP1798 | 320 | 280 |
| CYP5491-T351M | 321 | 324 |
| SgEPH3 | 322 | 286 |
| AtCPR | 323 | 283 |
| ERG1 | 326 | 328 |
| ERG7 (comprising L491Q, Y586F, and | 327 | 329 |
| R660H relative to SEQ ID NO: 337) | | |
| ERG7(comprising K47E, L92I, T360S, | 332 | 336 |
| S372P, T444M, and R578P relative to | | |
| SEQ ID NO: 337) | | |
| CPR4497 | 333 | 282 |
| CYP1798 | 334 | 280 |
| CYP1798 | 320 | 280 |
| CYP1798 | 335 | 280 |
| ERG7 | 338 | 337 |
| aba1 | 94 | 89 |
| aba2 | 25 | 90 |
| aba3 | 26 | 91 |
| aba4 | 27 | 92 |
| CPR | 28 | 93 |
| T16H2 | 69 | 41 |
| 16OMT | 70 | 42 |
| T3O | 71 | 43 |
| T3R | 72 | 44 |
| NMT | 73 | 45 |
| D4H | 74 | 66 |
| DAT | 75 | 67 |
| AtCPR | 76 | 68 |
| MjDODA | | 116 |
| BvCYP76AD5 | | 117 |
| PgDDS (a dammarenediol-II synthase | 123 | 119 |
| (DDS)) | | |
| PgPPDS (a PPD synthase (PPDS)) | 124 | 120 |
| CPR2 or PgCPR | 125 | 121 |
| UGTPn50-HV or PgUGT50-H | 126 | 122 |
| CrtS | 127 | 128 |
| P_40s | 129 | |
| P_CrtYB | 130 | |
| P_HMG1 | 131 | |
| P_CrtS | 132 | |
| MjcDOPA5GT | | 158 |
| BvCYP76AD1 | | 159 |
| Db5GT | | 160 |

### EQUIVALENTS

Those skilled in the art will recognize, or be able to ascertain using no more than routine experimentation, many equivalents to the specific embodiments of the invention described in this application. Such equivalents are intended to be encompassed by the following claims.

All references, including patent documents, disclosed in this application are incorporated by reference in their entirety, particularly for the disclosure referenced in this application.

1.-10. (canceled)

11. A host cell that comprises a heterologous polynucleotide encoding a cytochrome b5 (CB5), wherein the host cell is capable of producing more of an oxygenated hydrocarbon than a control host cell that does not comprise the heterologous polynucleotide, and wherein the CB5 comprises:
a) the amino acid sequence X₁X₂X₃X₄X₅X₆X₇EX₈IX₉X₁₀YTGLSPX₁₁X₁₂FFTX₁₃LAX₁₄X₁₅X₁₆X₁₇VX₁₈X₁₉X₂₀X₂₁SX₂₂X₂₃FX₂₄X₂₅X₂₆X₂₇X₂₈X₂₉X₃₀X₃₁ (SEQ ID NO: 50), wherein:
(i) X₁ is the amino acid E or Q;
(ii) X₂ is the amino acid L or V;
(iii) X₃ is the amino acid Y or W;
(iv) X₄ is the amino acid W or E;
(v) X₅ is the amino acid K or T;
(vi) X₆ is the amino acid A or L;
(vii) X₇ is the amino acid M or K;
(viii) X₈ is the amino acid Q or A;
(ix) X₉ is the amino acid A or V;
(x) X₁₀ is the amino acid W or A;
(xi) X₁₁ is the amino acid T or A;
(xii) X₁₂ is the amino acid A or T;
(xiii) X₁₃ is the amino acid I or V;
(xiv) X₁₄ is the amino acid S or L;
(xv) X₁₅ is the amino acid M or G;
(xvi) X₁₆ is the amino acid I or L;
(xvii) X₁₇ is the amino acid F or A;
(xviii) X₁₅ is the amino acid F or Y;
(xix) X₁₉ is the amino acid Q or Y;
(xx) X₂₀ is the amino acid M or V;
(xxi) X₂₁ is the amino acid V or I;
(xxii) X₂₂ is the amino acid S or G;
(xxiii) X₂₃ is the amino acid M or F;
(xxiv) X₂₄ is the amino acid V or G;
(xxv) X₂₅ is the amino acid S or T;
(xxvi) X₂₆ is the amino acid P or S;
(xxvii) X₂₇ is the amino acid E or D;
(xxviii) X₂₈ is the amino acid E or Y;
(xxix) X₂₉ is the amino acid F or G;
(xxx) X₃₀ is the amino acid N or S; and/or
(xxxi) X₃₁ is the amino acid K or H;
b) the amino acid sequence X₁VQX₂GX₃X₄X₅EX₆X₇LX₈X₉YDGSDX₁₀X₁₁KPLLMAIKGQIYDVSX₁₂X₁₃RMF (SEQ ID NO: 51), wherein:
(i) X₁ is the amino acid P or A;
(ii) X₂ is the amino acid V or I;
(iii) X₃ is the amino acid E or Q;
(iv) X₄ is the amino acid I or L;
(v) X₅ is the amino acid S or T;
(vi) X₆ is the amino acid E or Q;
(vii) X₇ is the amino acid E or Q;
(viii) X₈ is the amino acid K or R;
(ix) X₉ is the amino acid Q or A;
(x) X₁₀ is the amino acid S or P;
(xi) X₁₁ is the amino acid K or N;
(xii) X₁₂ is the amino acid Q or S; and/or
(xiii) X₁₃ is the amino acid S or G;
c) the amino acid sequence LAX₁X₂SFX₃X₄X₅DX₆TGX₇IX₈GLX₉X₁₀X₁₁ELX₁₂X₁₃LQDWEYKFMX₁₄KYVKVGX₁₅X_{1 6} (SEQ ID NO: 52), wherein:
(i) X₁ is the amino acid K or L;
(ii) X₂ is the amino acid M or L;
(iii) X₃ is the amino acid E or K;
(iv) X₄ is the amino acid E or P;
(v) X₅ is the amino acid K or E;
(vi) X₆ is the amino acid L or I;
(vii) X₇ is the amino acid D or N;
(viii) X₈ is the amino acid S or E;
(ix) X₉ is the amino acid G or S;
(x) X₁₀ is the amino acid P or E;
(xi) X₁₁ is the amino acid F or E;
(xii) X₁₂ is the amino acid E or V;
(xiii) X₁₃ is the amino acid A or I;
(xiv) X₁₄ is the amino acid S or E;
(xv) X₁₅ is the amino acid T or E; and/or
(xvi) X₁₆ is the amino acid V or L;
d) the amino acid sequence X₁X₂X₃EX₄GX₅X₆X₇X₈X₉X₁₀D (SEQ ID NO: 53), wherein:
(i) X₁ is the amino acid K or E;
(ii) X₂ is the amino acid P or H;
(iii) X₃ is the amino acid A or S;
(iv) X₄ is the amino acid D or N;
(v) X₅ is the amino acid P or H;
(vi) X₆ is the amino acid S or R;
(vii) X₇ is the amino acid E or N;
(viii) X₈ is the amino acid S or F;
(ix) X₉ is the amino acid Q or E; and/or
(x) X₁₀ is the amino acid A or I; and/or
e) the amino acid sequence DATX₁X₂FX₃X₄X₅VGHS (SEQ ID NO: 31), wherein:
(i) X₁ is the amino acid E, D, or N;
(ii) X₂ is the amino acid A or D;
(iii) X₃ is the amino acid E or D;
(iv) X₄ is the amino acid D or N; and/or
(v) X₅ is the amino acid V or A.

12. (canceled)

13. The host cell of claim 11, wherein the CB5 comprises one or more of the following amino acid sequences:
a) QVWETLKEAIVAYTGLSPATFFTVLALGLAVYYVISGFFGTSDYGSH (SEQ ID NO: 58) or ELYWKAMEQIAWYTGLSPTAFFTILASMIFVFQMVSSMFVSPEEFNK (SEQ ID NO: 59);
b) PVQVGEISEEELKQYDGSDSKKPLLMAIKGQIYDVSQSRMF (SEQ ID NO: 60) or AVQIGQLTEQQLRAYDGSDPNKPLLMAIKGQIYDVSSGRMF (SEQ ID NO: 61);
c) LAKMSFEEKDLTGDISGLGPFELEALQDWEYKFMSKYVKVGTV (SEQ ID NO: 62) or LALLSFKPEDITGNIEGLSEEELVILQDWEYKFMEKYVKVGEL (SEQ ID NO: 63);
d) KPAEDGPSESQAD (SEQ ID NO: 64) or EHSENGHRNFEID (SEQ ID NO: 65); and
e) DATEAFEDVGHS (SEQ ID NO: 108), DATDDFENVGHS (SEQ ID NO: 109), DATDDFEDVGHS (SEQ ID NO: 110), DATDDFEDAGHS (SEQ ID NO: 111), or DATNDFDDVGHS (SEQ ID NO: 112).

14.-17. (canceled)

18. The host cell of claim 11, wherein the CB5 comprises at most one histidine in one or more of the following regions:
a) a region corresponding to positions 64-104 of SEQ ID NO: 1;
b) a region corresponding to positions 105-122 of SEQ ID NO: 1; and/or
c) a region corresponding to positions 123-165 of SEQ ID NO: 1.

19.-23. (canceled)

24. A host cell that comprises a heterologous polynucleotide encoding a cytochrome b5 (CB5), wherein the CB5 comprises a sequence that is at least 90% identical to any one of SEQ ID NOs: 1-10, 29, 30, 32, 33, 34, 77, 78, 79, 80, 81, 82, 118, and 318 and wherein the host cell is capable of producing an oxygenated hydrocarbon.

25.-26. (canceled)

27. The host cell of claim 24, wherein the heterologous polynucleotide comprises a sequence that is at least 90% identical to any one of SEQ ID NOs: 11-24, 35-40, 83-88, 95-107, 316-317, and 330-331, and wherein the host cell is capable of producing an oxygenated hydrocarbon.

28. (canceled)

29. A host cell that comprises a heterologous polynucleotide encoding a cytochrome b5 (CB5), wherein the CB5 comprises:
a) the amino acid sequence ILRVSFRKYRKAIEQ (SEQ ID NO: 54);
b) the amino acid sequence RAFRPSIRFKKSHSTVPT (SEQ ID NO: 55);
c) the amino acid sequence KNTLYVGG (SEQ ID NO: 56); and/or
d) the amino acid sequence DQATQKHRSFGFVTFLEKED (SEQ ID NO: 57)
and wherein the host cell is capable of producing more of an oxygenated hydrocarbon than a control host cell that does not comprise the heterologous polynucleotide.

30.-34. (canceled)

35. The host cell of claim 24, wherein the host cell further comprises a heterologous polynucleotide encoding a cytochrome P450 (CYP).

36. The host cell of claim 35, wherein the CYP is a C11 hydroxylase, ABA1, ABA2, T16H2, T3O, PPDS, AD1, AD5, AD6, C28 oxidase, C16 oxidase, C23 oxidase, and/or CrtS.

37. The host cell of claim 24, wherein the oxygenated hydrocarbon is mogrol, a ginsenoside, abscisic acid, a vinca alkaloid, a betaxanthin, or quillaic acid.

38. The host cell of claim 24, wherein the oxygenated hydrocarbon is an oxygenated isoterpenoid.

39. The host cell of claim 24, wherein the oxygenated hydrocarbon is a terpenoid glycoside, a terpenoid indole alkaloid, or a sesquiterpene, optionally wherein the sesquiterpene is abscisic acid.

40. The host cell of claim 39, wherein the terpenoid glycoside is a mogroside or a ginsenoside and/or the terpenoid indole alkaloid is a vinca alkaloid, optionally wherein the vinca alkaloid is vinblastine, vincristine, or vinorelbine.

41.-43. (canceled)

44. The host cell of claim 24, wherein the oxygenated hydrocarbon is a betalain, optionally wherein the betalain is a betacyanin or a betaxanthin.

45. (canceled)

46. The host cell of claim 24, wherein the oxygenated hydrocarbon is astaxanthin.

47.-56. (canceled)

57. The host cell of claim 24, wherein the host cell further comprises one or more heterologous polynucleotides encoding:
(a) one or more mogrol and/or mogroside synthesis enzymes;
(b) one or more ABA synthesis enzymes;
(c) one or more vinca alkaloid synthesis enzymes;
(d) one or more ginsenoside synthesis enzymes;
(e) one or more betalain synthesis enzymes; and/or
(f) one or more astaxanthin synthesis enzymes.

58.-62. (canceled)

63. The host cell of claim 24, wherein the heterologous polynucleotide encoding the CB5 comprises a sequence that is at least 90% identical to any one of SEQ ID NOs: 129-132.

64. The host cell of claim 35, wherein the CYP comprises an amino acid sequence that is at least 90% identical to any one of SEQ ID NOs: 41, 43, 89, 90, 117, 120, 128, 280, 281, and 324.

65. The host cell of claim 24, wherein the host cell is a yeast cell, a plant cell, or a bacterial cell, optionally wherein the yeast cell is a Saccharomyces cerevisiae, Xanthophyllomyces dendrorhous, or Yarrowia lipolytica cell.

66.-71. (canceled)

72. A method of producing an oxygenated hydrocarbon comprising culturing the host cell of claim 24.

73.-74. (canceled)

75. A bioreactor for producing an oxygenated hydrocarbon, wherein the bioreactor comprises a host cell of claim 24.

76.-78. (canceled)
